(19) United States
(12) Patent Application Publication
(10) Pub. No.: US 2010/0199382 A1
(43) Pub. Date: Aug. 5, 2010

(54) Plants Having Increased Yield-Related Traits And A Method For Making The Same

(75) Inventors: Valerie Frankard, Waterloo (BE); Christophe Reuzeau, Tocan Saint Apre (FR)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/678,918

(22) PCT Filed: Sep. 19, 2008
(86) PCT No.: PCT/EP2008/062540

§ 371 Date: Mar. 18, 2010

Related U.S. Application Data
(60) Provisional application No. 60/975,882, filed on Sep. 28, 2007.

(30) Foreign Application Priority Data
Sep. 21, 2007 (EP) 07116988.2

(51) Int. Cl. A01H 1/00; A01H 5/00; C12N 15/74

(52) U.S. Cl. 800/278; 800/295; 435/320.1

(57) ABSTRACT

The present invention relates generally to the field of molecular biology and concerns a method for increasing various plant yield-related traits by increasing expression in a plant of: (i) a nucleic acid sequence encoding a Growth-RegulatingFactor (GRF) polypeptide; and of (ii) a nucleic acid sequence encoding a synovial sarcoma translocation (SYT) polypeptide, wherein said yield-related traits are increased relative to plants having increased expression of one of: (i) a nucleic acid sequence encoding a GRF polypeptide, or (ii) a nucleic acid sequence encoding a SYT polypeptide. The present invention also concerns plants having increased expression of (i) a nucleic acid sequence encoding a GRF polypeptide; and of (ii) a nucleic acid sequence encoding a SYT polypeptide, wherein said plants have increased yield-related traits relative to plants having increased expression of one of: (i) a nucleic acid sequence encoding a GRF polypeptide; or (ii) a nucleic acid sequence encoding a SYT polypeptide. The invention also provides constructs useful in the methods of the invention.

The present invention relates generally to the field of molecular biology and concerns a method for increasing various plant yield-related traits by increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, wherein said yield-related traits are increased relative to plants having increased expression of one of: (i) a nucleic acid sequence encoding a GRF polypeptide, or (ii) a nucleic acid sequence encoding a SYT polypeptide. The present invention also concerns plants having increased expression of (i) a nucleic acid sequence encoding a GRF polypeptide; and of (ii) a nucleic acid sequence encoding a SYT polypeptide, wherein said plants have increased yield-related traits relative to plants having increased expression of one of: (i) a nucleic acid sequence encoding a GRF polypeptide; or (ii) a nucleic acid sequence encoding a SYT polypeptide. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Plant biomass is yield for forage crops like alfalfa, silage corn and hay. Many proxies for yield have been used in grain crops. Chief amongst these are estimates of plant size. Plant size can be measured in many ways depending on species and developmental stage, but include total plant dry weight, above-ground dry weight, above-ground fresh weight, leaf area, stem volume, plant height, rosette diameter, leaf length, root length, root mass, tiller number and leaf number. Many species maintain a conservative ratio between the size of different parts of the plant at a given developmental stage. These allometric relationships are used to extrapolate from one of these measures of size to another (e.g. Tittonell et al 2005 Agric Ecosys & Environ 105: 213). Plant size at an early developmental stage will typically correlate with plant size later in development. A larger plant with a greater leaf area can typically absorb more light and carbon dioxide than a smaller plant and therefore will likely gain a greater weight during the same period (Fasoula & Tollenaar 2005 Maydica 50:39). This is in addition to the potential continuation of the micro-environmental or genetic advantage that the plant had to achieve the larger size initially. There is a strong genetic component to plant size and growth rate (e.g. ter Steege et al 2005 Plant Physiology 139:1078), and so for a range of diverse genotypes plant size under one environmental condition is likely to correlate with size under another (Hittalmani et al 2003 Theoretical Applied Genetics 107:679). In this way a standard environment is used as a proxy for the diverse and dynamic environments encountered at different locations and times by crops in the field.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

Harvest index, the ratio of seed yield to aboveground dry weight, is relatively stable under many environmental conditions and so a robust correlation between plant size and grain yield can often be obtained (e.g. Rebetzke et al 2002 Crop Science 42:739). These processes are intrinsically linked because the majority of grain biomass is dependent on current or stored photosynthetic productivity by the leaves and stem of the plant (Gardener et al 1985 Physiology of Crop Plants. Iowa State University Press, pp 68-73). Therefore, selecting for plant size, even at early stages of development, has been used as an indicator for future potential yield (e.g. Tittonell et al 2005 Agric Ecosys & Environ 105: 213). When testing for the impact of genetic differences on stress tolerance, the ability to standardize soil properties, temperature, water and nutrient availability and light intensity is an intrinsic advantage of greenhouse or plant growth chamber environments compared to the field. However, artificial limitations on yield due to poor pollination due to the absence of wind or insects, or insufficient space for mature root or canopy growth, can restrict the use of these controlled environments for testing yield differences. Therefore, measurements of plant size in early development, under standardized conditions in a growth chamber or greenhouse, are standard practices to provide indication of potential genetic yield advantages.

Another trait of importance is that of improved abiotic stress tolerance. Abiotic stress is a primary cause of crop loss worldwide, reducing average yields for most major crop plants by more than 50% (Wang et al. (2003) Planta 218: 1-14). Abiotic stresses may be caused by drought, salinity, extremes of temperature, chemical toxicity, excess or deficiency of nutrients (macroelements and/or microelements), radiation and oxidative stress. The ability to increase plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increase yield-related traits (seed yield and/or biomass) in plants may be through modification of the inherent growth mechanisms of a plant, such as the cell cycle or various signalling pathways involved in plant growth or in defense mechanisms.

It has now been found that various yield-related traits may be increased in plants by increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, wherein said yield-related traits are increased relative to plants having increased expression of one of: (i) a nucleic acid sequence encoding a GRF polypeptide; or (ii) a nucleic acid sequence encoding a SYT polypeptide. The increased yield-related traits comprise one or more of: increased early vigour, increased aboveground biomass, increased total seed yield per plant, increased seed filling rate, increased number of (filled) seeds, increased harvest index and increased thousand kernel weight (TKW).

### Background Relating to Growth-Regulating Factor (GRF) Polypeptides

DNA-binding proteins are proteins that comprise any of many DNA-binding domains and thus have a specific or general affinity to DNA. DNA-binding proteins include for example transcription factors that modulate the process of transcription, nucleases that cleave DNA molecules, and histones that are involved in DNA packaging in the cell nucleus.

Transcription factors are usually defined as proteins that show sequence-specific DNA binding affinity and that are capable of activating and/or repressing transcription. The Arabidopsis thaliana genome codes for at least 1533 transcriptional regulators, accounting for ˜5.9% of its estimated total number of genes (Riechmann et al. (2000) Science 290: 2105-2109). The Database of Rice Transcription Factors (DRTF) is a collection of known and predicted transcription factors of Oryza sativa L. ssp. indica and Oryza sativa L. ssp. japonica, and currently contains 2,025 putative transcription factors (TF) gene models in indica and 2,384 in japonica, distributed in 63 families (Gao et al. (2006) Bioinformatics 2006, 22(10):1286-7).

One of these families is the Growth-Regulating Factor (GRF) family of transcription factors, which is specific to plants. At least nine GRF polypeptides have been identified in Arabidopsis thaliana (Kim et al. (2003) Plant J 36: 94-104), and at least twelve in Oryza sativa (Choi et al. (2004) Plant Cell Physiol 45(7): 897-904). The GRF polypeptides are characterized by the presence in their N-terminal half of at least two highly conserved domains, named after the most conserved amino acids within each domain: (i) a QLQ domain (InterPro accession IPR014978, PFAM accession PF08880), where the most conserved amino acids of the domain are Gln-Leu-Gln; and (ii) a WRC domain (InterPro accession IPR014977, PFAM accession PF08879), where the most conserved amino acids of the domain are Trp-Arg-Cys. The WRC domain further contains two distinctive structural features, namely, the WRC domain is enriched in basic amino acids Lys and Arg, and further comprises three Cys and one His residues in a conserved spacing (CX₉CX₁₀CX₂H), designated as the Effector of Transcription (ET) domain (Ellerstrom et al. (2005) Plant Molec Biol 59: 663-681). The conserved spacing of cysteine and histidine residues in the ET domain is reminiscent of zinc finger (zinc-binding) proteins. In addition, a nuclear localisation signal (NLS) is usually comprised in the GRF polypeptide sequences.

Interaction of some GRF polypeptides with a small family of transcriptional coactivators, GRF-interacting factors (GIF1 to GIF3, also called synovial sarcoma translocation SYT1 to SYT3), has been demonstrated using a yeast two-hyrid interaction assay (Kim & Kende (2004) Proc Natl Acad Sci 101: 13374-13379).

The name GRF has also been given to another type of polypeptides, belonging to the 14-3-3 family of polypeptides (de Vetten & Ferl (1994) Plant Physiol 106: 1593-1604), that are totally unrelated the GRF polypeptides useful in performing the methods of the invention.

Transgenic Arabidopsis thaliana plants transformed with a rice GRF (OsGRF1) polypeptide under the control of a viral constitutive 35S CaMV promoter displayed curly leaves, severely reduced elongation of the primary inflorescence, and delayed bolting (van der Knapp et al. (2000) Plant Physiol 122: 695-704). Transgenic Arabidopsis thaliana plants transformed with either one of two Arabidopsis GRF polypeptides (AtGRF1 and AtGRF2) developed larger leaves and cotyledons, were delayed in bolting, and were partially sterile (due to lack of viable pollen), compared to wild type plants (Kim et al. (2003) Plant J 36: 94-104).

In US patent application US2006/0048240, an Arabidopsis thaliana GRF polypeptide is identified as SEQ ID NO: 33421. In US patent application US 2007/0022495, an Arabidopsis thaliana GRF polypeptide is identified as SEQ ID NO: 1803 (also therein referred to as G1438). Transgenic Arabidopsis plants overexpressing G1438 using the 35S CaMV promoter present dark green leaves.

Background Relating to synovial Sarcoma Translocation (SYT) polypeptides SYT is a transcriptional co-activator that, in plants, forms a functional complex with transcription activators of the GRF (growth-regulating factor) family of proteins (Kim H J, Kende H (2004) Proc Nat Acad Sc 101: 13374-9). SYT is called GIF for GRF-interacting factor in this paper, and AN3 for angustifolia3 in Horiguchi et al. (2005) Plant J 43: 68-78. The GRF transcription activators share structural domains (in the N-terminal region) with the SWI/SNF proteins of the chromatin-remodelling complexes in yeast (van der Knaap E et al., (2000) Plant Phys 122: 695-704). Transcriptional co-activators of these complexes are proposed to be involved in recruiting SWI/SNF complexes to enhancer and promoter regions to effect local chromatin remodelling (review Näär et al., (2001) Annu Rev Biochem 70: 475-501). The alteration in local chromatin structure modulates transcriptional activation. More precisely, SYT is proposed to interact with plant SWI/SNF complex to affect transcriptional activation of GRF target gene(s) (Kim H J, Kende H (2004) Proc Nat Acad Sc 101: 13374-9).

SYT belongs to a gene family of three members in Arabidopsis. The SYT polypeptide shares homology with the human SYT. The human SYT polypeptide was shown to be a transcriptional co-activator (Thaete et al. (1999) Hum Molec Genet 8: 585-591). Three domains characterize the mammalian SYT polypeptide:
(i) the N-terminal SNH (SYT N-terminal homology) domain, conserved in mammals, plants, nematodes and fish;
(ii) the C-terminal QPGY-rich domain, composed predominantly of glycine, proline, glutamine and tyrosine, occurring at variable intervals;
(iii) a methionine-rich (Met-rich) domain located between the two previous domains.

In plant SYT polypeptides, the SNH domain is well conserved. The C-terminal domain is rich in glycine and glutamine, but not in proline or tyrosine. It has therefore been named the QG-rich domain in contrast to the QPGY domain of mammals. As with mammalian SYT, a Met-rich domain may be identified N-terminally of the QG domain. The QG-rich domain may be taken to be substantially the C-terminal remainder of the polypeptide (minus the SHN domain); the Met-rich domain is typically comprised within the first half of the QG-rich (from the N-terminus to the C-terminus). A second Met-rich domain may precede the SNH domain in plant SYT polypeptides (see FIG. 1).

A SYT loss-of function mutant and transgenic plants with reduced expression of SYT was reported to develop small and narrow leaves and petals, which have fewer cells (Kim H J, Kende H (2004) Proc Nat Acad Sc 101: 13374-9).

Overexpression of AN3 in Arabidopsis thaliana resulted in plants with leaves that were 20-30% larger than those of the wild type (Horiguchi et al. (2005) Plant J 43: 68-78).

In Japanese patent application 2004-350553, a method for controlling the size of leaves in the horizontal direction is described, by controlling the expression of the AN3 gene.

Surprisingly, it has now been found that increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide gives plants having increased yield-related traits relative to plants having increased expression of one of: (i) a nucleic acid sequence encoding a GRF polypeptide; or (ii) a nucleic acid sequence encoding a SYT polypeptide. According to one embodiment, there is provided a method for increasing various plant yield-related traits by increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, wherein said yield-related traits are increased relative to plants having increased expression of one of: (i) a nucleic acid sequence encoding a GRF polypeptide, or (ii) a nucleic acid sequence encoding a SYT polypeptide. The increased yield-related traits comprise one or more of: increased early vigour, increased aboveground biomass, increased total seed yield per plant, increased seed filling rate, increased number of (filled) seeds, increased harvest index or increased thousand kernel weight (TKW).

### DEFINITIONS

### Polypeptide(s)/Protein(s)

The terms “polypeptide” and “protein” are used interchangeably herein and refer to amino acids in a polymeric form of any length, linked together by peptide bonds.

Polynucleotide(s)/Nucleic Acid(s)/Nucleic Acid Sequence (s)/Nucleotide Sequence(s)

The terms “polynucleotide(s)”, “nucleic acid sequence(s)”, “nucleotide sequence(s)”, “nucleic acid(s)” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. A “control plant” as used herein refers not only to whole plants, but also to plant parts, including seeds and seed parts.

### Homoloque(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W. H. Freeman and Company (Eds) and Table 1 below).

TABLE 1
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

### Ortholoque(s)/Paraloque(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

### Motif/Consensus Sequence/Signature

The term “motif' or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acid molecules are in solution. The hybridisation process can also occur with one of the complementary nucleic acid molecules immobilised to a matrix such as magnetic beads, Sepharose beads or any other resin. The hybridisation process can furthermore occur with one of the complementary nucleic acid molecules immobilised to a solid support such as a nitro-cellulose or nylon membrane or immobilised by e.g. photolithography to, for example, a siliceous glass support (the latter known as nucleic acid sequence arrays or microarrays or as nucleic acid sequence chips). In order to allow hybridisation to occur, the nucleic acid molecules are generally thermally or chemically denatured to melt a double strand into two single strands and/or to remove hairpins or other secondary structures from single stranded nucleic acid molecules.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acid sequences may deviate in sequence and still encode a substantially identical polypeptide, due to the degeneracy of the genetic code. Therefore medium stringency hybridisation conditions may sometimes be needed to identify such nucleic acid sequence molecules.

The Tm is the temperature under defined ionic strength and pH, at which 50% of the target sequence hybridises to a perfectly matched probe. The Tₘ, is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid sequence strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6x log₁₀[Na⁺]^{a})+0.41x%[G/C^{b}]−500x[L^{c}]⁻¹−0.61x % formamide

2) DNA-RNA or RNA-RNA hybrids:

Tₘ=79.8+18.5(log₁₀[Na⁺]^{a})+0.58 (% G/C^{b})+11.8(% G/C^{b})²−820/L^{c}

3) oligo-DNA or oligo-RNA^{d} hybrids:
For <20 nucleotides: Tₘ=2 (lₙ)
For 20-35 nucleotides: Tₘ=22+1.46 (lₙ)
^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; lₙ,=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid sequence hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acid molecules of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 pg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acid sequences or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid sequence control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, increasers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or increases expression of a nucleic acid sequence molecule in a cell, tissue or organ.

A “plant promoter” comprises regulatory elements, which mediate the expression of a coding sequence segment in plant cells. The “plant promoter” preferably originates from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid sequence molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid sequence used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a level that is in all instances below that obtained under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of plant constitutive promoters

| Gene Source | Reference |
|---|---|
| Actin | McElroy et al, Plant Cell, 2: 163-171, 1990 |
| HMGB | WO 2004/070039 |
| GOS2 | de Pater et al, Plant J Nov; 2(6): 837-44, 1992, |
| | WO 2004/065596 |
| Ubiquitin | Christensen et al, Plant Mol. Biol. 18: 675-689, |
| | 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 histone | Wu et al. Plant Mol. Biol. 11: 641-649, 1988 |
| Actin 2 | An et al, Plant J. 10(1); 107-121, 1996 |
| Rubisco small | U.S. Pat. No. 4,962,028 |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| V-ATPase | WO 01/14572 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| Rice RCc3 | Xu et al (1995) Plant Mol Biol 27(2): 237-48 |
| Arabidopsis phosphate | Kovama et al., 2005 |
| transporter PHT1 | |
| Medicago phosphate | Xiao et al., 2006 |
| transporter | |
| Arabidopsis Pyk10 | Nitz et al. (2001) Plant Sci 161(2): 337-346 |
| Tobacco root-specific | Conkling et al. (1990) Plant Phys 93(3): |
| genes RB7, RD2, RD5, | 1203-1211 |
| RH12 | |
| Barley root-specific | Lerner & Raikhel (1989) Plant Phys 91: |
| lectin | 124-129 |
| Root-specific hydroxy- | Keller & Lamb (1989) Genes & Dev 3: |
| proline rich protein | 1639-1646 |
| Arabidopsis CDC27B/ | Blilou et al. (2002) Genes & Dev 16: |
| hobbit | 2566-2575 |

A seed-specific promoter is transcriptionally active predominantly in seed tissue, but not necessarily exclusively in seed tissue (in cases of leaky expression). The seed-specific promoter may be active during seed development and/or during germination. Examples of seed-specific promoters are shown in Table 2c below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| Legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| Zein | Matzke et al Plant Mol Biol, 14(3): 323-32 1990 |
| NapA | Stalberg et al, Planta 199: 515-519, 1996. |
| Wheat LMW and HMW glutenin-1 | Mol Gen Genet 216: 81-90, 1989; NAR 17: 461-2, 1989 |
| Wheat SPA | Albani et al, Plant Cell, 9: 171-184, 1997 |
| Wheat α, β, γ-gliadins | EMBO J. 3: 1409-15, 1984 |
| Barley ltr1 promoter | Diaz et al. (1995) Mol Gen Genet 248(5): 592-8 |
| Barley B1, C, D, hordein | Theor Appl Gen 98: 1253-62, 1999; Plant J 4: 343-55, |
| | 1993; Mol Gen Genet 250: 750-60, 1996 |
| Barley DOF | Mena et al, The Plant Journal, 116(1): 53-62, 1998 |
| blz2 | EP99106056.7 |
| Synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, |
| | 1996 |
| rice α-globulin REB/OHP-1 | Nakase et al. Plant Mol. Biol. 33: 513-522, 1997 |
| rice ADP-glucose pyrophos-phorylase | Trans Res 6: 157-68, 1997 |
| Maize ESR gene family | Plant J 12: 235-46, 1997 |
| Sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | Unpublished |
| aminotransferase | |
| PRO0147, trypsin inhibitor ITR1 | Unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| Cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2d below.

TABLE 2d
Examples of green tissue-specific promoters

| Gene | Expression | Reference |
|---|---|---|
| Maize Orthophosphate dikinase | Leaf specific | Fukavama et al., 2001 |
| Maize Phosphoenolpyruvate | Leaf specific | Kausch et al., 2001 |
| carboxylase | | |
| Rice Phosphoenolpyruvate | Leaf specific | Liu et al., 2003 |
| carboxylase | | |
| Rice small subunit Rubisco | Leaf specific | Nomura et al., 2000 |
| rice beta expansin EXBP9 | Shoot specific | WO 2004/070039 |
| Pigeonpea small subunit Rubisco | Leaf specific | Panguluri et al., 2005 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of meristem-specific promoters which may be used to perform the methods of the invention are shown in Table 2e below.

TABLE 2e
Examples of meristem-specific promoters

| Gene source | Expression pattern | Reference |
|---|---|---|
| rice OSH1 | Shoot apical meristem, from | Sato et al. (1996) |
| | embryo globular stage to | Proc. Natl. Acad. |
| | seedling stage | Sci. USA, 93: |
| | | 8117-8122 |
| Rice metallothionein | Meristem specific | BAD87835.1 |
| WAK1 & WAK 2 | Shoot and root apical | Wagner & Kohorn |
| | meristems, and in expanding | (2001) Plant Cell |
| | leaves and sepals | 13(2): 303-318 |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, preferably the expression level is increased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription increasers or translation increasers. Isolated nucleic acid sequences which serve as promoter or increaser elements may be introduced in an appropriate position (typically upstream) of a non-heterologous form of a polynucleotide so as to upregulate expression of a nucleic acid sequence encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron increasement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene.

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid sequence encoding the protein of interest (target gene), or from any nucleic acid sequence capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A method for the reduction or substantial elimination of endogenous gene expression is by RNA-mediated silencing using an inverted repeat of a nucleic acid sequence or a part thereof (in this case a stretch of substantially contiguous nucleotides derived from the gene of interest, or from any nucleic acid sequence capable of encoding an orthologue, paralogue or homologue of the protein of interest), preferably capable of forming a hairpin structure. Another example of an RNA silencing method involves the introduction of nucleic acid sequences or parts thereof (in this case a stretch of substantially contiguous nucleotides derived from the gene of interest, or from any nucleic acid sequence capable of encoding an orthologue, paralogue or homologue of the protein of interest) in a sense orientation into a plant. Another example of an RNA silencing method involves the use of antisense nucleic acid sequences. Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682). Other methods, such as the use of antibodies directed to an endogenous polypeptide for inhibiting its function in planta, or interference in the signalling pathway in which a polypeptide is involved, will be well known to the skilled man. Artificial and/or natural microRNAs (miRNAs) may be used to knock out gene expression and/or mRNA translation. Endogenous miRNAs are single stranded small RNAs of typically 19-24 nucleotides long. Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs (Schwab et al., (2005) Dev Cell 8(4):517-27). Convenient tools for design and generation of amiRNAs and their precursors are also available to the public (Schwab et al., (2006) Plant Cell 18(5):1121-33).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid sequence to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid sequence construct of the invention. These marker genes enable the identification of a successful transfer of the nucleic acid sequence molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acid sequences into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid sequence molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid sequence can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acid sequences have been introduced successfully, the process according to the invention for introducing the nucleic acid sequences advantageously employs techniques which enable the removal or excision of these marker genes. One such a method is what is known as co-transformation. The co-transformation method employs two vectors simultaneously for the transformation, one vector bearing the nucleic acid sequence according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid sequence (known as the Ac/Ds technology). The transformants can be crossed with a transposase source or the transformants are transformed with a nucleic acid sequence construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acid sequences used in the method of the invention are not at their natural locus in the genome of said plant, it being possible for the nucleic acid sequences to be expressed homologously or heterologously. However, as mentioned, transgenic also means that, while the nucleic acid sequence according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acid sequences according to the invention at an unnatural locus in the genome, i.e. homologous or, preferably, heterologous expression of the nucleic acid sequences takes place. Preferred transgenic plants are mentioned herein.

### Transformation

The term “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acid sequences or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagging

T-DNA activation tagging (Hayashi et al. Science (1992) 1350-1353), involves insertion of T-DNA, usually containing a promoter (may also be a translation increaser or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### Tilling

The term “TILLING” is an abbreviation of “Targeted Induced Local Lesions In Genomes” and refers to a mutagenesis technology useful to generate and/or identify nucleic acid sequences encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei GP and Koncz C (1992) In Methods in Arabidopsis Research, Koncz C, Chua N.H., Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

### Homologous Recombination

Homologous recombination allows introduction in a genome of a selected nucleic acid sequence at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J 9(10): 3077-84) but also for crop plants, for example rice (Terada et al. (2002) Nat Biotech 20(10): 1030-4; lida and Terada (2004) Curr Opin Biotech 15(2): 132-8).

### Yield

The term “yield” in general means a measurable produce of economic value, typically related to a specified crop, to an area, and to a period of time. Individual plant parts directly contribute to yield based on their number, size and/or weight, or the actual yield is the yield per acre for a crop and year, which is determined by dividing total production (includes both harvested and appraised production) by planted acres. The term “yield” of a plant may relate to vegetative biomass, to reproductive organs, and/or to propagules (such as seeds) of that plant.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Increase

The terms “increase”, “improve” or “increase” are interchangeable and shall mean in the sense of the application at least a 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per hectare or acre; b) increased number of flowers per panicle and/or per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; f) increased number of primary panicles; (g) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in yield may also manifest itself as an increase in seed area and/or seed length and/or seed width and/or seed perimeter. Increased seed yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid sequence of interest. The term “plant” also encompasses plant cells, suspension cultures, callus tissue, embryos, meristematic regions, gametophytes, sporophytes, pollen and microspores, again wherein each of the aforementioned comprises the gene/nucleic acid sequence of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticale sp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### DETAILED DESCRIPTION OF THE INVENTION

Surprisingly, it has now been found that increasing expression in a plant of a nucleic acid sequence encoding a GRF polypeptide gives plants having increased yield-related traits relative to control plants. According to a first embodiment, the present invention provides a method for increasing yield-related traits in plants relative to control plants, comprising increasing expression in a plant of a nucleic acid sequence encoding a GRF polypeptide.

Surprisingly, it has now been found that increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide gives plants having increased yield-related traits relative to plants having increased expression of one of: (i) a nucleic acid sequence encoding a GRF polypeptide; or (ii) a nucleic acid sequence encoding a SYT polypeptide. According to a first embodiment, there is provided a method for increasing various plant yield-related traits by increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, wherein said yield-related traits are increased relative to plants having increased expression of one of: (i) a nucleic acid sequence encoding a GRF polypeptide, or (ii) a nucleic acid sequence encoding a SYT polypeptide. The increased yield-related traits comprise one or more of: increased early vigour, increased aboveground biomass, increased total seed yield per plant, increased seed filling rate, increased number of (filled) seeds, increased harvest index or increased thousand kernel weight (TKW).

### Detailed Description Relating to Growth-Regulating Factor (GRF) Polypeptides

A preferred method for increasing expression of a nucleic acid sequence encoding a GRF polypeptide is by introducing and expressing in a plant a nucleic acid sequence encoding a GRF polypeptide.

Any reference hereinafter to a “GRF protein useful in the methods of the invention” is taken to mean a GRF polypeptide as defined herein. Any reference hereinafter to a “GRF nucleic acid sequence useful in the methods of the invention” is taken to mean a nucleic acid sequence capable of encoding such a GRF polypeptide. The nucleic acid sequence to be introduced into a plant (and therefore useful in performing the methods of the invention) is any nucleic acid sequence encoding the type of polypeptide, which will now be described, hereafter also named “GRF nucleic acid sequence” or “GRF gene”.

A “GRF polypeptide” as defined herein refers to any polypeptide comprising: (i) a domain having at least 50%, 55%, 60%, 65%, 70%, 75%, 80%, 85%, 90%, 95%, 98%, 99% or more amino acid sequence identity to a QLQ domain as represented by SEQ ID NO: 115 (comprised in SEQ ID NO: 2); and (ii) a domain having at least 50%, 55%, 60%, 65%, 70%, 75%, 80%, 85%, 90%, 95%, 98%, 99% or more amino acid sequence identity to a WRC domain as represented by SEQ ID NO: 116 (comprised in SEQ ID NO: 2).

Alternatively or additionally, a “GRF polypeptide” as defined herein refers to any polypeptide comprising: (i) a QLQ domain with an InterPro accession IPR014978 (PFAM accession PF08880); (ii) a WRC domain with an InterPro accession IPR014977 (PFAM accession PF08879); and (iii) an Effector of Transcription (ET) domain comprising three Cys and one His residues in a conserved spacing (CX₉CX₁₀CX₂H).

Alternatively or additionally, a “GRF polypeptide” as defined herein refers to any polypeptide having in increasing order of preference at least 50%, 55%, 60%, 65%, 70%, 75%, 80%, 85%, 90%, 95%, 98%, 99% or more amino acid sequence identity to the GRF polypeptide as represented by SEQ ID NO: 2 or to any of the full length polypeptide sequences given in Table A.1 herein.

Alternatively or additionally, a “GRF polypeptide” interacts with GRF-interacting factor (GIF; (GIF1 to GIF3; also called synovial sarcoma translocation SYT1 to SYT3) polypeptides, such as the ones presented in Table A.2 herein, in a yeast two-hybrid interaction assay.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32: D134-D137, (2004)), or Pfam (Bateman et al., Nucleic Acids Research 30(1): 276-280 (2002). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)).

Analysis of the polypeptide sequence of SEQ ID NO: 2 is presented below in Examples 2 and 4 herein. For example, a GRF polypeptide as represented by SEQ ID NO: 2 comprises a QLQ domain with an InterPro accession IPR014978 (PFAM accession PF08880) and a WRC domain with an InterPro accession IPR014977 (PFAM accession PF08879) in the InterPro domain database. Domains may also be identified using routine techniques, such as by sequence alignment. An alignment of the QLQ domain of the polypeptides of Table A.1 herein, is shown in FIG. 2, and alignment of the WRC domain of the polypeptides of Table A.1 herein, is shown in FIG. 3. Such alignments are useful for identifying the most conserved amino acids between the GRF polypeptides, such as the QLQ and WRC amino acid residues.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., (2003) BMC Bioinformatics, 10: 29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid sequence or polypeptide sequence or over selected domains or conserved motif(s), using the programs mentioned above using the default parameters. Outside of the QLQ domain and of the WRC domain, GRF polypeptides reputedly have low amino acid sequence identity. Example 3 herein describes in Table B.1 the percentage identity between the GRF polypeptide as represented by SEQ ID NO: 2 and the GRF polypeptides listed in Table A, which can be as low as 15% amino acid sequence identity. The percentage identity can be substantially increased if the identity calculation is performed between the QLQ domain SEQ ID NO: 2 (as represented by SEQ ID NO: 115 comprised in SEQ ID NO: 2; QLQ domain of the GRF polypeptides of Table A.1 represented in FIG. 2) and the QLQ domains of the polypeptides useful in performing the invention. Similarly, the percentage identity can be substantially increased if the identity calculation is performed between the WRC domain SEQ ID NO: 2 (as represented by SEQ ID NO: 116 comprised in SEQ ID NO: 2; WRC domain of the GRF polypeptides of Table A.1 represented in FIG. 3) and the WRC domains of the polypeptides useful in performing the invention. Percentage identity over the QLQ domain amongst the polypeptide sequences useful in performing the methods of the invention ranges between 25% and 99% amino acid identity, and percentage identity over the WRC domain amongst the polypeptide sequences useful in performing the methods of the invention ranges between 60% and 99% amino acid identity. As can also be observed in FIG. 3, the WRC domain is better conserved amongst the different GRF polypeptides than the QLQ domain, as shown in FIG. 2.

The task of protein subcellular localisation prediction is important and well studied. Knowing a protein's localisation helps elucidate its function. Experimental methods for protein localization range from immunolocalization to tagging of proteins using green fluorescent protein (GFP) or beta-glucuronidase (GUS). Such methods are accurate although labor-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP and others.

Furthermore, GRF polypeptides useful in the methods of the present invention (at least in their native form) typically, but not necessarily, have transcriptional regulatory activity and capacity to interact with other proteins. Therefore, GRF polypeptides with reduced transcriptional regulatory activity, without transcriptional regulatory activity, with reduced protein-protein interaction capacity, or with no protein-protein interaction capacity, may equally be useful in the methods of the present invention. DNA-binding activity and protein-protein interactions may readily be determined in vitro or in vivo using techniques well known in the art (for example in Current Protocols in Molecular Biology, Volumes 1 and 2, Ausubel et al. (1994), Current Protocols). GRF polypeptides are capable of transcriptional activation of reporter genes in yeast cells (Kim & Kende (2004) Proc Natl Acad Sci 101(36): 13374-13379). GRF polypeptides are also capable of interacting with GRF-interacting factor polypeptides (GIF1 to GIF3; also called synovial sarcoma translocation (SYT)) in vivo in yeast cells, using a yeast two-hybrid protein-protein interaction assay (Kim & Kende, supra). In vitro binding assays are also used to show that GRF polypeptides and SYT (or GIF) polypeptides are interacting partners (Kim & Kende, supra).

The present invention is illustrated by transforming plants with the nucleic acid sequence represented by SEQ ID NO: 1, encoding the GRF polypeptide sequence of SEQ ID NO: 2. However, performance of the invention is not restricted to these sequences; the methods of the invention may advantageously be performed using any nucleic acid sequence encoding a GRF polypeptide as defined herein.

Examples of nucleic acid sequences encoding GRF polypeptides are given in Table A.1 of Example 1 herein. Such nucleic acid sequences are useful in performing the methods of the invention. The polypeptide sequences given in Table A.1 of Example 1 are example sequences of orthologues and paralogues of the GRF polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A.1 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acid sequences encoding homologues and derivatives of any one of the polypeptide sequences given in Table A.1 of Example 1, the terms “homologue” and “derivative” being as defined herein. Also useful in the methods of the invention are nucleic acid sequences encoding homologues and derivatives of orthologues or paralogues of any one of the polypeptide sequences given in Table A.1 of Example 1. Homologues and derivatives useful in the methods of the present invention have substantially the same biological and functional activity as the unmodified protein from which they are derived.

Further nucleic acid variants useful in practising the methods of the invention include portions of nucleic acid sequences encoding GRF polypeptides, nucleic acid sequences hybridising to nucleic acid sequences encoding GRF polypeptides, splice variants of nucleic acid sequences encoding GRF polypeptides, allelic variants of nucleic acid sequences encoding GRF polypeptides and variants of nucleic acid sequences encoding GRF polypeptides obtained by gene shuffling. The terms hybridising sequence, splice variant, allelic variant and gene shuffling are as described herein.

Nucleic acid sequences encoding GRF polypeptides need not be full-length nucleic acid sequences, since performance of the methods of the invention does not rely on the use of full-length nucleic acid sequences. According to the present invention, there is provided a method for increasing yield-related traits, in plants, comprising introducing and expressing in a plant a portion of any one of the nucleic acid sequences given in Table A.1 of Example 1, or a portion of a nucleic acid sequence encoding an orthologue, paralogue or homologue of any of the polypeptide sequences given in Table A.1 of Example 1.

A portion of a nucleic acid sequence may be prepared, for example, by making one or more deletions to the nucleic acid sequence. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a GRF polypeptide as defined herein, and have substantially the same biological activity as the polypeptide sequences given in Table A.1 of Example 1. Preferably, the portion is a portion of any one of the nucleic acid sequences given in Table A.1 of Example 1, or is a portion of a nucleic acid sequence encoding an orthologue or paralogue of any one of the polypeptide sequences given in Table A.1 of Example 1. Preferably the portion is, in increasing order of preference at least 400, 450, 500, 550, 600, 650, 700, 750, 800, 850, 900, 950, 1000, 1050, 1100, 1150, 1190 consecutive nucleotides in length, the consecutive nucleotides being of any one of the nucleic acid sequences given in Table A.1 of Example 1, or of a nucleic acid sequence encoding an orthologue or paralogue of any one of the polypeptide sequences given in Table A.1 of Example 1 Preferably, the portion is a portion of a nucleic sequence encoding a polypeptide sequence polypeptide comprising: (i) a domain having at least 50%, 55%, 60%, 65%, 70%, 75%, 80%, 85%, 90%, 95%, 98%, 99% or more amino acid sequence identity to a QLQ domain as represented by SEQ ID NO: 115 (comprised in SEQ ID NO: 2); and (ii) a domain having at least 50%, 55%, 60%, 65%, 70%, 75%, 80%, 85%, 90%, 95%, 98%, 99% or more amino acid sequence identity to a WRC domain as represented by SEQ ID NO: 116 (comprised in SEQ ID NO: 2). Most preferably the portion is a portion of the nucleic acid sequence of SEQ ID NO: 1.

Another nucleic acid sequence variant useful in the methods of the invention is a nucleic acid sequence capable of hybridising, under reduced stringency conditions, preferably under stringent conditions, with a nucleic acid sequence encoding a GRF polypeptide as defined herein, or with a portion as defined herein.

According to the present invention, there is provided a method for increasing yield-related traits in plants, comprising introducing and expressing in a plant a nucleic acid sequence capable of hybridizing to any one of the nucleic acid sequences given in Table A.1 of Example 1, or comprising introducing and expressing in a plant a nucleic acid sequence capable of hybridising to a nucleic acid sequence encoding an orthologue, paralogue or homologue of any of the nucleic acid sequences given in Table A.1 of Example 1.

Hybridising sequences useful in the methods of the invention encode a GRF polypeptide as defined herein, and have substantially the same biological activity as the polypeptide sequences given in Table A.1 of Example 1. Preferably, the hybridising sequence is capable of hybridising to any one of the nucleic acid sequences given in Table A.1 of Example 1, or to a portion of any of these sequences, a portion being as defined above, or wherein the hybridising sequence is capable of hybridising to a nucleic acid sequence encoding an orthologue or paralogue of any one of the polypeptide sequences given in Table A.1 of Example 1. Preferably, the hybridising sequence is capable of hybridising to a nucleic acid sequence encoding a polypeptide sequence comprising: (i) a domain having at least 50%, 55%, 60%, 65%, 70%, 75%, 80%, 85%, 90%, 95%, 98%, 99% or more amino acid sequence identity to a QLQ domain as represented by SEQ ID NO: 115 (comprised in SEQ ID NO: 2); and (ii) a domain having at least 50%, 55%, 60%, 65%, 70%, 75%, 80%, 85%, 90%, 95%, 98%, 99% or more amino acid sequence identity to a WRC domain as represented by SEQ ID NO: 116 (comprised in SEQ ID NO: 2). Most preferably, the hybridising sequence is capable of hybridising to a nucleic acid sequence as represented by SEQ ID NO: 1 or to a portion thereof.

Another nucleic acid sequence variant useful in the methods of the invention is a splice variant encoding a GRF polypeptide as defined hereinabove, a splice variant being as defined herein.

According to the present invention, there is provided a method for increasing yield-related traits, comprising introducing and expressing in a plant a splice variant of any one of the nucleic acid sequences given in Table A.1 of Example 1, or a splice variant of a nucleic acid sequence encoding an orthologue, paralogue or homologue of any of the polypeptide sequences given in Table A.1 of Example 1.

Preferred splice variants are splice variants of a nucleic acid sequence represented by SEQ ID NO: 1, or a splice variant of a nucleic acid sequence encoding an orthologue or paralogue of SEQ ID NO: 2. Preferably, the splice variant is a splice variant of a nucleic acid sequence encoding a polypeptide sequence comprising: (i) a domain having at least 50%, 55%, 60%, 65%, 70%, 75%, 80%, 85%, 90%, 95%, 98%, 99% or more amino acid sequence identity to a QLQ domain as represented by SEQ ID NO: 115 (comprised in SEQ ID NO: 2); and (ii) a domain having at least 50%, 55%, 60%, 65%, 70%, 75%, 80%, 85%, 90%, 95%, 98%, 99% or more amino acid sequence identity to a WRC domain as represented by SEQ ID NO: 116 (comprised in SEQ ID NO: 2).

Another nucleic acid sequence variant useful in performing the methods of the invention is an allelic variant of a nucleic acid sequence encoding a GRF polypeptide as defined hereinabove, an allelic variant being as defined herein.

According to the present invention, there is provided a method for increasing yield-related traits, comprising introducing and expressing in a plant an allelic variant of any one of the nucleic acid sequences given in Table A.1 of Example 1, or comprising introducing and expressing in a plant an allelic variant of a nucleic acid sequence encoding an orthologue, paralogue or homologue of any of the polypeptide sequences given in Table A.1 of Example 1.

The allelic variants useful in the methods of the present invention have substantially the same biological activity as the GRF polypeptide of SEQ ID NO: 2 and any of the polypeptide sequences depicted in Table A.1 of Example 1. Allelic variants exist in nature, and encompassed within the methods of the present invention is the use of these natural alleles. Preferably, the allelic variant is an allelic variant of SEQ ID NO: 1 or an allelic variant of a nucleic acid sequence encoding an orthologue or paralogue of SEQ ID NO: 2. Preferably, the allelic variant is an allelic variant of a polypeptide sequence comprising: (i) a domain having at least 50%, 55%, 60%, 65%, 70%, 75%, 80%, 85%, 90%, 95%, 98%, 99% or more amino acid sequence identity to a QLQ domain as represented by SEQ ID NO: 115 (comprised in SEQ ID NO: 2); and (ii) a domain having at least 50%, 55%, 60%, 65%, 70%, 75%, 80%, 85%, 90%, 95%, 98%, 99% or more amino acid sequence identity to a WRC domain as represented by SEQ ID NO: 116 (comprised in SEQ ID NO: 2).

Gene shuffling or directed evolution may also be used to generate variants of nucleic acid sequences encoding GRF polypeptides as defined above, the term “gene shuffling” being as defined herein.

According to the present invention, there is provided a method for increasing yield-related traits, comprising introducing and expressing in a plant a variant of any one of the nucleic acid sequences given in Table A.1 of Example 1, or comprising introducing and expressing in a plant a variant of a nucleic acid sequence encoding an orthologue, paralogue or homologue of any of the polypeptide sequences given in Table A.1 of Example 1, which variant nucleic acid sequence is obtained by gene shuffling.

Preferably, the variant nucleic acid sequence obtained by gene shuffling encodes a polypeptide sequence comprising: (i) a domain having at least 50%, 55%, 60%, 65%, 70%, 75%, 80%, 85%, 90%, 95%, 98%, 99% or more amino acid sequence identity to a QLQ domain as represented by SEQ ID NO: 115 (comprised in SEQ ID NO: 2); and (ii) a domain having at least 50%, 55%, 60%, 65%, 70%, 75%, 80%, 85%, 90%, 95%, 98%, 99% or more amino acid sequence identity to a WRC domain as represented by SEQ ID NO: 116 (comprised in SEQ ID NO: 2).

Furthermore, nucleic acid sequence variants may also be obtained by site-directed mutagenesis. Several methods are available to achieve site-directed mutagenesis, the most common being PCR based methods (Current Protocols in Molecular Biology. Wiley Eds.).

Nucleic acid sequences encoding GRF polypeptides may be derived from any natural or artificial source. The nucleic acid sequence may be modified from its native form in composition and/or genomic environment through deliberate human manipulation. Preferably the nucleic acid sequence encoding a GRF polypeptide is from a plant, further preferably from a dicotyledonous plant, more preferably from the family Brassicaceae, most preferably the nucleic acid sequence is from Arabidopsis thaliana.

### Detailed Description Relating to Synovial Sarcoma Translocation (SYT) Polypeptides

A preferred method for increasing expression of a nucleic acid sequence encoding a SYT polypeptide is by introducing and expressing in a plant a nucleic acid sequence encoding a SYT polypeptide.

Any reference hereinafter to a “SYT protein useful in the methods of the invention” is taken to mean a SYT polypeptide as defined herein. Any reference hereinafter to a “SYT nucleic acid sequence useful in the methods of the invention” is taken to mean a nucleic acid sequence capable of encoding such a SYT polypeptide. The nucleic acid sequence to be introduced into a plant (and therefore useful in performing the methods of the invention) is any nucleic acid sequence encoding the type of polypeptide, which will now be described, hereafter also named “SYT nucleic acid sequence” or “SYT gene”.

The term “SYT polypeptide” as defined herein refers to a polypeptide comprising from N-terminal to C-terminal: (i) an SNH domain having in increasing order of preference at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% sequence identity to the SNH domain of SEQ ID NO: 262; and (ii) a Met-rich domain; and (iii) a QG-rich domain. Preferably, the SNH domain comprises the most conserved residues as represented by SEQ ID NO: 263, and shown in black in FIG. 5.

Alternatively or additionally, a “SYT polypeptide” as defined herein refers to any polypeptide comprising a domain having in increasing order of preference at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% sequence identity to the SSXT domain with an InterPro accession IPR007726 (PFAM accession PF05030, SSXT protein (N-terminal region)) of SEQ ID NO: 264.

Alternatively or additionally, a “SYT polypeptide” as defined herein refers to any polypeptide having in increasing order of preference at least 20%, 25%, 30%, 35%, 40%, 45%, 50%, 55%, 60%, 65%, 70%, 75%, 80%, 85%, 90%, 95%, 98%, 99% or more amino acid sequence identity to the SYT polypeptide as represented by SEQ ID NO: 121 or to any of the full length polypeptide sequences given in Table A.2 herein.

Alternatively or additionally, a “SYT polypeptide” interacts with Growth-Regulating Factor (GRF) polypeptides in a yeast two-hybrid interaction assay, for examples with the GRF polypeptide sequences given in Table A.1 herein.

Analysis of the SYT polypeptide sequence of SEQ ID NO: 121 is presented below in Examples 2 and 4 herein. For example, a SYT polypeptide as represented by SEQ ID NO: 121 comprises an SSXT domain with an InterPro accession IPR007726 (PFAM accession PF05030) in the InterPro domain database. Domains may also be identified using routine techniques, such as by sequence alignment. An alignment of the SNH domain of the polypeptides of Table A.2 herein, is shown in FIG. 5. Such alignments are useful for identifying the most conserved amino acids between the SYT polypeptides, such as the most conserved residues represented in SEQ ID NO: 263.

Methods for the alignment of sequences for comparison are well known in the art, as briefly described hereinabove. The sequence identity values may be determined over the entire nucleic acid sequence or polypeptide sequence or over selected domains or conserved motif(s), using the programs mentioned above using the default parameters. Outside of the SNH domain and of the SSXT domain, SYT polypeptides reputedly have low amino acid sequence identity. Example 3 herein describes in Table B.2 the percentage identity between the SYT polypeptide as represented by SEQ ID NO: 121 and the SYT polypeptides listed in Table A.2, which can be as low as 25% amino acid sequence identity. The percentage amino acid identity can be substantially increased if the identity calculation is performed between the SNH domain as represented by SEQ ID NO: 262 (comprised in SEQ ID NO: 121) and the SNH domain of the SYT polypeptides of Table A.2 (represented in FIG. 5). Similarly, the percentage identity can be substantially increased if the identity calculation is performed between the SSXT domain as represented by SEQ ID NO: 264 (comprised in SEQ ID NO: 121) and the SSXT domains of the SYT polypeptides useful in performing the invention. The SNH domain, which is comprised in the SSXT domain, is better conserved amongst the different SYT polypeptides than the SSXT domain.

Furthermore, SYT polypeptides useful in the methods of the present invention (at least in their native form) typically, but not necessarily, have transcriptional regulatory activity and capacity to interact with other proteins. Therefore, SYT polypeptides with reduced transcriptional regulatory activity, without transcriptional regulatory activity, with reduced protein-protein interaction capacity, or with no protein-protein interaction capacity, may equally be useful in the methods of the present invention. DNA-binding activity and protein-protein interactions may readily be determined in vitro or in vivo using techniques well known in the art (for example in Current Protocols in Molecular Biology, Volumes 1 and 2, Ausubel et al. (1994), Current Protocols). SYT polypeptides are capable of interacting with GRF polypeptides in vivo in yeast cells, using a yeast two-hybrid protein-protein interaction assay (Kim & Kende, supra). In vitro binding assays are also used to show that GRF polypeptides and SYT polypeptides are interacting partners (Kim & Kende, supra).

The present invention is illustrated by transforming plants with the nucleic acid sequence represented by SEQ ID NO: 120, encoding the SYT polypeptide sequence of SEQ ID NO: 121. However, performance of the invention is not restricted to these sequences; the methods of the invention may advantageously be performed using any nucleic acid sequence encoding a SYT polypeptide as defined herein.

Examples of nucleic acid sequences encoding SYT polypeptides are given in Table A.2 of Example 1 herein. Such nucleic acid sequences are useful in performing the methods of the invention. The polypeptide sequences given in Table A.2 of Example 1 are example sequences of orthologues and paralogues of the SYT polypeptide represented by SEQ ID NO: 121, the terms “orthologues” and “paralogues” being as defined hereinabove. Further orthologues and paralogues may readily be identified by performing reciprocal blast searches (as described herein above).

Nucleic acid variants may also be useful in practising the methods of the invention. Examples of such variants include nucleic acid sequences encoding homologues and derivatives of any one of the polypeptide sequences given in Table A.2 of Example 1, the terms “homologue” and “derivative” being as defined herein. Also useful in the methods of the invention are nucleic acid sequences encoding homologues and derivatives of orthologues or paralogues of any one of the polypeptide sequences given in Table A.2 of Example 1. Homologues and derivatives useful in the methods of the present invention have substantially the same biological and functional activity as the unmodified protein from which they are derived.

Further nucleic acid variants useful in practising the methods of the invention include portions of nucleic acid sequences encoding SYT polypeptides, nucleic acid sequences hybridising to nucleic acid sequences encoding SYT polypeptides, splice variants of nucleic acid sequences encoding SYT polypeptides, allelic variants of nucleic acid sequences encoding SYT polypeptides and variants of nucleic acid sequences encoding SYT polypeptides obtained by gene shuffling. The terms hybridising sequence, splice variant, allelic variant and gene shuffling are as described herein.

Nucleic acid sequences encoding SYT polypeptides need not be full-length nucleic acid sequences, since performance of the methods of the invention does not rely on the use of full-length nucleic acid sequences. According to the present invention, there is provided a method for increasing yield-related traits, in plants, comprising introducing and expressing in a plant a portion of any one of the nucleic acid sequences given in Table A.2 of Example 1, or a portion of a nucleic acid sequence encoding an orthologue, paralogue or homologue of any of the polypeptide sequences given in Table A.2 of Example 1.

A portion of a nucleic acid sequence may be prepared, for example, by making one or more deletions to the nucleic acid sequence. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a SYT polypeptide as defined herein, and have substantially the same biological activity as the polypeptide sequences given in Table A.2 of Example 1. Preferably, the portion is a portion of any one of the nucleic acid sequences given in Table A.2 of Example 1, or is a portion of a nucleic acid sequence encoding an orthologue or paralogue of any one of the polypeptide sequences given in Table A.2 of Example 1. Preferably the portion is, in increasing order of preference at least 100, 125, 150, 175, 200 or 225 consecutive nucleotides in length, preferably at least 250, 275, 300, 325, 350, 375, 400, 425, 450 or 475 consecutive nucleotides in length, further preferably least 500, 525, 550, 575, 600, 625, 650, 675, 700 or 725 consecutive nucleotides in length, or as long as a full length SYT nucleic acid sequence, the consecutive nucleotides being of any one of the nucleic acid sequences given in Table A.2 of Example 1, or of a nucleic acid sequence encoding an orthologue or paralogue of any one of the polypeptide sequences given in Table A.2 of Example 1. Preferably, the portion is a portion of a nucleic sequence encoding a polypeptide sequence polypeptide comprising from N-terminal to C-terminal: (i) an SNH domain having in increasing order of preference at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% sequence identity to the SNH domain of SEQ ID NO: 262; and (ii) a Met-rich domain; and (iii) a QG-rich domain. Preferably, the SNH domain comprises the most conserved residues as represented by SEQ ID NO: 263, and shown in black in FIG. 5. Most preferably the portion is a portion of the nucleic acid sequence of SEQ ID NO: 120.

Another nucleic acid sequence variant useful in the methods of the invention is a nucleic acid sequence capable of hybridising, under reduced stringency conditions, preferably under stringent conditions, with a nucleic acid sequence encoding a SYT polypeptide as defined herein, or with a portion as defined herein.

According to the present invention, there is provided a method for increasing yield-related traits in plants, comprising introducing and expressing in a plant a nucleic acid sequence capable of hybridizing to any one of the nucleic acid sequences given in Table A.2 of Example 1, or comprising introducing and expressing in a plant a nucleic acid sequence capable of hybridising to a nucleic acid sequence encoding an orthologue, paralogue or homologue of any of the nucleic acid sequences given in Table A.2 of Example 1.

Hybridising sequences useful in the methods of the invention encode a SYT polypeptide as defined herein, and have substantially the same biological activity as the polypeptide sequences given in Table A.2 of Example 1. Preferably, the hybridising sequence is capable of hybridising to any one of the nucleic acid sequences given in Table A.2 of Example 1, or to a portion of any of these sequences, a portion being as defined above, or wherein the hybridising sequence is capable of hybridising to a nucleic acid sequence encoding an orthologue or paralogue of any one of the polypeptide sequences given in Table A.2 of Example 1. Preferably, the hybridising sequence is capable of hybridising to a nucleic acid sequence encoding a polypeptide sequence comprising from N-terminal to C-terminal: (i) an SNH domain having in increasing order of preference at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% sequence identity to the SNH domain of SEQ ID NO: 262; and (ii) a Met-rich domain; and (iii) a QG-rich domain. Preferably, the SNH domain comprises the most conserved residues as represented by SEQ ID NO: 263, and shown in black in FIG. 5. Most preferably, the hybridising sequence is capable of hybridising to a nucleic acid sequence as represented by SEQ ID NO: 120 or to a portion thereof.

Another nucleic acid sequence variant useful in the methods of the invention is a splice variant encoding a SYT polypeptide as defined hereinabove, a splice variant being as defined herein.

According to the present invention, there is provided a method for increasing yield-related traits, comprising introducing and expressing in a plant a splice variant of any one of the nucleic acid sequences given in Table A.2 of Example 1, or a splice variant of a nucleic acid sequence encoding an orthologue, paralogue or homologue of any of the polypeptide sequences given in Table A.2 of Example 1.

Preferred splice variants are splice variants of a nucleic acid sequence represented by SEQ ID NO: 120, or a splice variant of a nucleic acid sequence encoding an orthologue or paralogue of SEQ ID NO: 121. Preferably, the splice variant is a splice variant of a nucleic acid sequence encoding a polypeptide sequence comprising from N-terminal to C-terminal: (i) an SNH domain having in increasing order of preference at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% sequence identity to the SNH domain of SEQ ID NO: 262; and (ii) a Met-rich domain; and (iii) a QG-rich domain. Preferably, the SNH domain comprises the most conserved residues as represented by SEQ ID NO: 263, and shown in black in FIG. 5.

Another nucleic acid sequence variant useful in performing the methods of the invention is an allelic variant of a nucleic acid sequence encoding a SYT polypeptide as defined hereinabove, an allelic variant being as defined herein.

According to the present invention, there is provided a method for increasing yield-related traits, comprising introducing and expressing in a plant an allelic variant of any one of the nucleic acid sequences given in Table A.2 of Example 1, or comprising introducing and expressing in a plant an allelic variant of a nucleic acid sequence encoding an orthologue, paralogue or homologue of any of the polypeptide sequences given in Table A.2 of Example 1.

The allelic variants useful in the methods of the present invention have substantially the same biological activity as the SYT polypeptide of SEQ ID NO: 121 and any of the polypeptide sequences depicted in Table A.2 of Example 1. Allelic variants exist in nature, and encompassed within the methods of the present invention is the use of these natural alleles. Preferably, the allelic variant is an allelic variant of SEQ ID NO: 120 or an allelic variant of a nucleic acid sequence encoding an orthologue or paralogue of SEQ ID NO: 121. Preferably, the allelic variant is an allelic variant of a polypeptide sequence comprising from N-terminal to C-terminal: (i) an SNH domain having in increasing order of preference at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% sequence identity to the SNH domain of SEQ ID NO: 262; and (ii) a Met-rich domain; and (iii) a QG-rich domain. Preferably, the SNH domain comprises the most conserved residues as represented by SEQ ID NO: 263, and shown in black in FIG. 5.

Gene shuffling or directed evolution may also be used to generate variants of nucleic acid sequences encoding SYT polypeptides as defined above, the term “gene shuffling” being as defined herein.

According to the present invention, there is provided a method for increasing yield-related traits, comprising introducing and expressing in a plant a variant of any one of the nucleic acid sequences given in Table A.2 of Example 1, or comprising introducing and expressing in a plant a variant of a nucleic acid sequence encoding an orthologue, paralogue or homologue of any of the polypeptide sequences given in Table A.2 of Example 1, which variant nucleic acid sequence is obtained by gene shuffling.

Preferably, the variant nucleic acid sequence obtained by gene shuffling encodes a polypeptide sequence comprising from N-terminal to C-terminal: (i) an SNH domain having in increasing order of preference at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% sequence identity to the SNH domain of SEQ ID NO: 262; and (ii) a Met-rich domain; and (iii) a QG-rich domain. Preferably, the SNH domain comprises the most conserved residues as represented by SEQ ID NO: 263, and shown in black in FIG. 5.

Furthermore, nucleic acid sequence variants may also be obtained by site-directed mutagenesis. Several methods are available to achieve site-directed mutagenesis, the most common being PCR based methods (Current Protocols in Molecular Biology. Wiley Eds.).

Nucleic acid sequences encoding SYT polypeptides may be derived from any natural or artificial source. The nucleic acid sequence may be modified from its native form in composition and/or genomic environment through deliberate human manipulation. Preferably the nucleic acid sequence encoding a SYT polypeptide is from a plant, further preferably from a dicotyledonous plant, more preferably from the family Brassicaceae, most preferably the nucleic acid sequence is from Arabidopsis thaliana.

Performance of the methods of the invention, i.e., increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, gives plants having increased yield-related traits relative to plants having increased expression of one of: (i) a nucleic acid sequence encoding a GRF polypeptide; or (ii) a nucleic acid sequence encoding a SYT polypeptide. The terms “yield” and “seed yield” are described in more detail in the “definitions” section herein.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

Performance of the methods of the invention, i.e., increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, gives plants having increased yield-related traits relative to plants having increased expression of one of: (i) a nucleic acid sequence encoding a GRF polypeptide; or (ii) a nucleic acid sequence encoding a SYT polypeptide. Preferably said increased yield-related trait is one or more of: (i) increased early vigour; (ii) increased aboveground biomass; (iii) increased total seed yield per plant; (iv) increased seed filling rate; (v) increased number of (filled) seeds; (vi) increased harvest index; or (vii) increased thousand kernel weight (TKW).

Since the transgenic plants according to the present invention have increased yield-related traits, it is likely that these plants exhibit an increased growth rate (during at least part of their life cycle), relative to the growth rate of control plants at a corresponding stage in their life cycle.

“Control plant” may include, as specified in the “definition” section, corresponding wild type plants, or corresponding plants without the gene of interest, or corresponding plants having increased expression of one of: (i) a nucleic acid sequence encoding a GRF polypeptide; or (ii) a nucleic acid sequence encoding a SYT polypeptide. A “control plant” as used herein refers not only to whole plants, but also to plant parts, including seeds and seed parts.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect increased (early) vigour. The increase in growth rate may alter the harvest cycle of a plant allowing plants to be sown later and/or harvested sooner than would otherwise be possible (a similar effect may be obtained with earlier flowering time; delayed flowering is usually not a desirede trait in crops). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, which plants have increased growth rate relative to plants having increased expression of one of: (i) a nucleic acid sequence encoding a GRF polypeptide; or (ii) a nucleic acid sequence encoding a SYT polypeptide.

Increased yield-related traits occur whether the plant is under non-stress conditions or whether the plant is exposed to various stresses compared to control plants grown under comparable conditions. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes, and insects. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild stress conditions having increased yield-related traits, relative to control plants grown under comparable conditions. Therefore, according to the present invention, there is provided a method for increasing yield-related traits in plants grown under non-stress conditions or under mild stress conditions, which method comprises increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, which plants have increased yield-related traits relative to plants having increased expression of one of: (i) a nucleic acid sequence encoding a GRF polypeptide; or (ii) a nucleic acid sequence encoding a SYT polypeptide, grown under comparable conditions.

Performance of the methods according to the present invention results in plants grown under abiotic stress conditions having increased yield-related traits relative to control plants grown under comparable stress conditions. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. Since diverse environmental stresses activate similar pathways, the exemplification of the present invention with drought stress should not be seen as a limitation to drought stress, but more as a screen to indicate the involvement of GRF polypeptides as defined above, in increasing yield-related traits relative to control plants grown in comparable stress conditions, in abiotic stresses in general.

The term “abiotic stress” as defined herein is taken to mean any one or more of: water stress (due to drought or excess water), anaerobic stress, salt stress, temperature stress (due to hot, cold or freezing temperatures), chemical toxicity stress and oxidative stress. According to one aspect of the invention, the abiotic stress is an osmotic stress, selected from water stress, salt stress, oxidative stress and ionic stress. Preferably, the water stress is drought stress. The term salt stress is not restricted to common salt (NaCl), but may be any stress caused by one or more of: NaCl, KCl, LiCl, MgCl₂, CaCl₂, amongst others.

Performance of the methods of the invention gives plants having increased yield-related traits, under abiotic stress conditions relative to control plants grown in comparable stress conditions. Therefore, according to the present invention, there is provided a method for increasing yield-related traits in plants grown under abiotic stress conditions, which method comprises increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, which plants have increased yield-related traits relative to plants having increased expression of one of: (i) a nucleic acid sequence encoding a GRF polypeptide; or (ii) a nucleic acid sequence encoding a SYT polypeptide, grown under comparable stress conditions.

Another example of abiotic environmental stress is the reduced availability of one or more nutrients that need to be assimilated by the plants for growth and development. Because of the strong influence of nutrition utilization efficiency on plant yield and product quality, a huge amount of fertilizer is poured onto fields to optimize plant growth and quality. Productivity of plants ordinarily is limited by three primary nutrients, phosphorous, potassium and nitrogen, which is usually the rate-limiting element in plant growth of these three. Therefore the major nutritional element required for plant growth is nitrogen (N). It is a constituent of numerous important compounds found in living cells, including amino acids, proteins (enzymes), nucleic acids, and chlorophyll. 1.5% to 2% of plant dry matter is nitrogen and approximately 16% of total plant protein. Thus, nitrogen availability is a major limiting factor for crop plant growth and production (Frink et al. (1999) Proc Natl Acad Sci USA 96(4): 1175-1180), and has as well a major impact on protein accumulation and amino acid composition. Therefore, of great interest are crop plants with increased yield-related traits, when grown under nitrogen-limiting conditions.

Performance of the methods of the invention gives plants grown under conditions of reduced nutrient availability, particularly under conditions of reduced nitrogen availablity, having increased yield-related traits relative to control plants grown under comparable stress conditions. Therefore, according to the present invention, there is provided a method for increasing yield-related traits in plants grown under conditions of reduced nutrient availablity, preferably reduced nitrogen availability, which method comprises increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, which plants have increased yield-related traits relative to plants having increased expression of one of: (i) a nucleic acid sequence encoding a GRF polypeptide; or (ii) a nucleic acid sequence encoding a SYT polypeptide, grown under comparable stress conditions. Reduced nutrient availability may result from a deficiency or excess of nutrients such as nitrogen, phosphates and other phosphorous-containing compounds, potassium, calcium, cadmium, magnesium, manganese, iron and boron, amongst others. Preferably, reduced nutrient availablity is reduced nitrogen availability.

The present invention encompasses plants or parts thereof (including seeds) or cells thereof obtainable by the methods according to the present invention. The plants or parts thereof or cells thereof comprise (i) an isolated nucleic acid transgene encoding a Growth-Regulating Factor (GRF) polypeptide; and (ii) an isolated nucleic acid transgene encoding a synovial sarcoma translocation (SYT) polypeptide.

As mentioned above, a preferred method for increasing expression of: (i) a nucleic acid sequence encoding a GRF polypeptide; and (ii) a nucleic acid sequence encoding a SYT polypeptide, is by introducing and expressing in a plant: (i) a nucleic acid sequence encoding a GRF polypeptide; and (ii) a nucleic acid sequence encoding a SYT polypeptide. Therefore, according to the present invention, there is provided a method for increasing yield-related traits in plants, which method comprises introducing and expressing in a plant: (i) a nucleic acid sequence encoding a GRF polypeptide; and (ii) a nucleic acid sequence encoding a SYT polypeptide, which plants have increased yield-related traits relative to plants having increased expression of one of: (i) a nucleic acid sequence encoding a GRF polypeptide; or (ii) a nucleic acid sequence encoding a SYT polypeptide.

Methods for introducing and expressing two or more transgenes (also called gene stacking) in transgenic plants are well known in the art (see for example, a review by Halpin (2005) Plant Biotech J (3): 141-155. Gene stacking can proceed by interative steps, where two or more transgenes can be sequentially introduced into a plant by crossing a plant containing one transgene with individuals harbouring other transgenes or, alternatively, by re-transforming (or super-transforming) a plant containing one transgene with new genes.

According to the present invention, there is provided a method for increasing yield-related traits in plants, which method comprises sequentially introducing and expressing in a plant: (i) a nucleic acid sequence encoding a GRF polypeptide; and (ii) a nucleic acid sequence encoding a SYT polypeptide, which plants have increased yield-related traits relative to plants having increased expression of one of: (i) a nucleic acid sequence encoding a GRF polypeptide; or (ii) a nucleic acid sequence encoding a SYT polypeptide.

Preferably, the nucleic acid sequences of (i) and (ii) are sequentially introduced and expressed by crossing. A crossing is performed between a female parent plant comprising an introduced and expressed isolated nucleic acid sequence encoding a GRF polypeptide, and a male parent plant comprising an introduced and expressed isolated nucleic acid sequence encoding a SYT polypeptide, or reciprocally, and by selecting in the progeny for the presence and expression of both transgenes. Therefore, according to the present invention, there is provided a method for increasing yield-related traits in plants, by crossing a female parent plant comprising an introduced and expressed isolated nucleic acid sequence encoding a GRF polypeptide, and a male parent plant comprising an introduced and expressed isolated nucleic acid sequence encoding a SYT polypeptide, or reciprocally, and by selecting in the progeny for the presence and expression of both transgenes, wherein said plants have increased yield-related traits relative to the parent plants, or to any other control plants as defined herein.

Alternatively the nucleic acid sequences of (i) and (ii) are sequentially introduced and expressed by re-transformation. Re-transformation is performed by introducing and expressing a nucleic acid sequence encoding GRF polypeptide into a plant, plant part, or plant cell comprising an introduced and expressed nucleic acid sequence encoding a SYT polypeptide, or reciprocally, and by selecting in the progeny for the presence and expression of both transgenes. Therefore, according to the present invention, there is provided a method for increasing yield-related traits in plants, by re-transformation performed by introducing and expressing a nucleic acid sequence encoding GRF polypeptide into a plant, plant part, or plant cell comprising an introduced and expressed nucleic acid sequence encoding a SYT polypeptide, or reciprocally, and by selecting in the progeny for the presence and expression of both transgenes, wherein said plants have increased yield-related traits relative to the plants having increased expression of one of: (i) a nucleic acid sequence encoding a GRF polypeptide; or (ii) a nucleic acid sequence encoding a SYT polypeptide, or to any other control plants as defined herein.

Alternatively, gene stacking can occur via simultaneous transformation, or co-transformation, which is faster and can be used in a whole range of transformation techniques, as described in the “definition” section herein.

When using Agrobacterium transformation for example, the transgenes (at least two) can be present in a number of conformations that are well known in the art, some of which are recited below:
(i) the nucleic acid encoding sequences are fused to form a single polypeptide when translated, and placed under the control of a single promoter;
(ii) the nucleic acid encoding sequences are sequentially placed downstream of a single promoter, separated by nucleic acid signals that influence mRNA synthesis (internal ribosome entry sites IRES, 2A stuttering signals, etc.), or polypeptide synthesis (polyproteins separated by protease substrate sites, etc.);
(iii) the nucleic acid encoding sequences are independently driven by separate promoters, and the promoter-nucleic acid encoding sequences combinations are located within one T-DNA;
(iv) the nucleic acid encoding sequences are independently driven by separate promoters, and the promoter-nucleic acid encoding sequences combinations are located in different T-DNAs on one plasmid;
(v) the nucleic acid encoding sequences are independently driven by separate promoters, and the promoter-coding sequence combinations are located in different T-DNAs on different plasmids hosted in one or in separate Agrobacterium strains.

When direct genetic transformation is considered, using physical or chemical delivery systems (e.g., microprojectile bombardment, PEG, electroporation, liposome, glass needles, etc.), the transgenes (at least two) can also be present in a number of conformations, but essentially do not need to be comprised in a vector capable of being replicated in Agrobacteria or viruses, intermediates of the genetic transformation. The two transgenes can be comprised in one or more nucleic acid molecules, but simultaneously used for the genetic transformation process.

According to the present invention, there is provided a method for increasing yield-related traits in plants, which method comprises simultaneously introducing and expressing in a plant: (i) a nucleic acid sequence encoding a GRF polypeptide; and (ii) a nucleic acid sequence encoding a SYT polypeptide, which plants have increased yield-related traits relative to plants having increased expression of one of: (i) a nucleic acid sequence encoding a GRF polypeptide; or (ii) a nucleic acid sequence encoding a SYT polypeptide.

The nucleic acid sequences of (i) and (ii) that are simultaneously introduced and expressed, are comprised in one or more nucleic acid molecules. Therefore, according to the present invention is provided increasing yield-related traits in plants, which method comprises simultaneously introducing and expressing in a plant: (i) a nucleic acid sequence encoding a GRF polypeptide; and (ii) a nucleic acid sequence encoding a SYT polypeptide, comprised in one or more nucleic acid molecules, which plants have increased yield-related traits relative to plants having increased expression of one of: (i) a nucleic acid sequence encoding a GRF polypeptide; or (ii) a nucleic acid sequence encoding a SYT polypeptide.

The invention also provides genetic constructs and vectors to facilitate introduction and/or increased expression in plants of nucleic acid sequences encoding GRF polypeptides. The gene constructs may be inserted into vectors, which may be commercially available, suitable for transforming into plants and for expression of the gene of interest in the transformed cells.

The invention also provides use of a gene construct as defined herein in the methods of the invention.

More specifically, the present invention provides a construct comprising:
(a) a nucleic acid sequence encoding a GRF polypeptide as defined above;
(b) a nucleic acid sequence encoding a SYT polypeptide as defined above;
(c) one or more control sequences capable of increasing expression of the nucleic acid sequence of (a) and of (b); and optionally
(d) a transcription termination sequence.

The nucleic acid sequences of (a) and (b) can be comprised in one nucleic acid molecule as represented by SEQ ID NO: 267 or by SEQ ID NO: 268, which nucleic acid molecule encodes a polypeptide sequence as represented by SEQ ID NO: 269 or by SEQ ID NO: 270.

The term “control sequence” and “termination sequence” are as defined herein. Preferably, one of the control sequences of a construct is a constitutive promoter. An example of a constitutive promoter is a GOS2 promoter, preferably a rice GOS2 promoter, more preferably a GOS2 promoter as represented by SEQ ID NO: 117.

In one construct, a single control sequence is used to drive the expression of both nucleic acid sequences of (a) and (b) comprised in one nucleic acid molecule as represented by SEQ ID NO: 267 or by SEQ ID NO: 268, which nucleic acid molecule encodes a polypeptide sequence as represented by SEQ ID NO: 269 or by SEQ ID NO: 270.

The present invention also provides for a mixture of constructs useful for example, for simultaneous introduction and expression in plants of (a) a nucleic acid sequence encoding a GRF polypeptide as defined above; and of (b) a nucleic acid sequence encoding a SYT polypeptide as defined above, wherein at least one construct comprises:
(a) a nucleic acid sequence encoding a GRF polypeptide as defined above;
(b) one or more control sequences capable of driving expression of the nucleic acid sequence of (a); and optionally
(c) a transcription termination sequence, and wherein at least one other construct comprises:
(d) a nucleic acid sequence encoding a SYT polypeptide as defined above;
(e) one or more control sequences capable of driving expression of the nucleic acid sequence of (d); and optionally
(f) a transcription termination sequence.

Preferably, one of the control sequences of a construct is a constitutive promoter. An example of a constitutive promoter is a GOS2 promoter, preferably a rice GOS2 promoter, more preferably a GOS2 promoter as represented by SEQ ID NO: 117.

The invention also provides for the use of a construct comprising: (a) a nucleic acid sequence encoding a GRF polypeptide as defined above; and (b) a nucleic acid sequence encoding a SYT polypeptide as defined above, or of a mixture of constructs comprising (a) and (b) as defined above, in a method for making plants having increased yield-related traits relative to plants having increased expression of one of: (a) a nucleic acid sequence encoding a GRF polypeptide, or (b) a nucleic acid sequence encoding a SYT polypeptide, which increased yield-related traits are one or more of: (i) increased early vigour; (ii) increased aboveground biomass; (iii) increased total seed yield per plant; (iv) increased seed filling rate; (v) increased number of (filled) seeds; (vi) increased harvest index; or (vii) increased thousand kernel weight (TKW).

The invention also provides for plants, plant parts or plant cells transformed with a construct comprising: (a) a nucleic acid sequence encoding a GRF polypeptide as defined above; and (b) a nucleic acid sequence encoding a SYT polypeptide as defined above, or with a mixture of constructs comprising (a) and (b) as defined above.

Plants are transformed with one or more vectors comprising any of the nucleic acid sequences described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to increase expression of the nucleic acid sequence. A constitutive promoter is particularly useful in the methods.

Other organ-specific promoters, for example for preferred expression in leaves, stems, tubers, meristems, seeds (embryo and/or endosperm), are useful in performing the methods of the invention. See the “Definitions” section herein for definitions of the various promoter types.

It should be clear that the applicability of the present invention is not restricted to: (i) a nucleic acid sequence encoding the GRF polypeptide, as represented by SEQ ID NO: 1, with expression driven by a constitutive promoter; or (ii) a nucleic acid sequence encoding the SYT polypeptide, as represented by SEQ ID NO: 120, with expression driven by a constitutive promoter.

Optionally, one or more terminator sequences may be used in the construct introduced into a plant. Additional regulatory elements may include transcriptional as well as translational increasers. Those skilled in the art will be aware of terminator and increaser sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, increaser, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acid sequences, it is advantageous to use marker genes (or reporter genes). Therefore, the genetic construct may optionally comprise a selectable marker gene. Selectable markers are described in more detail in the “definitions” section herein.

It is known that upon stable or transient integration of nucleic acid sequences into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid sequence molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid sequence can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having increased yield-related traits, comprising introduction and expression in a plant of: (i) any nucleic acid sequence encoding a GRF polypeptide as defined hereinabove; and (ii) any nucleic acid sequence encoding a SYT polypeptide as defined hereinabove, which plants have increased yield-related traits relative to plants having increased expression of: (i) any nucleic acid sequence encoding a GRF polypeptide as defined hereinabove; or (ii) any nucleic acid sequence encoding a SYT polypeptide as defined hereinabove.

More specifically, the present invention provides a method for the production of transgenic plants having increased yield-related traits relative to plants having increased expression of one of: (i) a nucleic acid sequence encoding a GRF polypeptide, or (ii) a nucleic acid sequence encoding a SYT polypeptide, comprising:
a. introducing and expressing in a plant, plant part, or plant cell, a nucleic acid sequence encoding a GRF polypeptide as defined above, under the control of a constitutive promoter; and
b. introducing and expressing in a plant, plant part, or plant cell, a nucleic acid sequence encoding a SYT polypeptide as defined above, under the control of a constitutive promoter; and
c. cultivating the plant cell, plant part, or plant under conditions promoting plant growth and development.

The nucleic acid sequence of (i) may be any of the nucleic acid sequences capable of encoding a GRF polypeptide as defined herein, and the nucleic acid sequence of (ii) may be any of the nucleic acid sequences capable of encoding a SYT polypeptide as defined herein.

The nucleic acid sequence may be introduced directly into a plant cell or into the plant itself (including introduction into a tissue, organ or any other part of a plant). According to a preferred feature of the present invention, the nucleic acid sequence is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing (i) an isolated nucleic acid sequence encoding a GRF polypeptide as defined hereinabove, operably linked to a constitutive promoter; and (ii) an isolated nucleic acid sequence encoding a SYT polypeptide as defined hereinabove, operably linked to a constitutive promoter. Preferred host cells according to the invention are plant cells. Host plants for the nucleic acid sequences or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants, which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant comprising: (i) an isolated nucleic acid sequence encoding a GRF (as defined hereinabove); and (ii) an isolated nucleic acid sequence encoding a SYT (as defined hereinabove), such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

Methods for increasing expression of nucleic acid sequences or genes, or gene products, are well documented in the art and examples are provided in the definitions section.

As mentioned above, a preferred method for increasing expression of (i) a nucleic acid sequence encoding a GRF polypeptide; and (ii) a nucleic acid sequence encoding a SYT polypeptide, is by introducing and expressing in a plant (i) a nucleic acid sequence encoding a GRF polypeptide; and (ii) a nucleic acid sequence encoding a SYT polypeptide; however the effects of performing the method, i.e. increasing yield-related traits, may also be achieved using other well known techniques, including but not limited to T-DNA activation tagging, TILLING, homologous recombination. A description of these techniques is provided in the definitions section.

The present invention also encompasses use of (i) nucleic acid sequences encoding GRF polypeptides as described herein; and nucleic acid sequences encoding SYT polypeptides as described herein, and use of these GRF polypeptides and SYT polypeptides in increasing any of the aforementioned yield-related traits in plants, under normal growth conditions, under abiotic stress growth (preferably osmotic stress growth conditions) conditions, and under growth conditions of reduced nutrient availability, preferably under conditions of reduced nitrogen availability.

Nucleic acid sequences encoding GRF polypeptides and SYT polypeptides described herein, or the polypeptides themselves, may find use in breeding programmes in which a DNA marker is identified that may be genetically linked to a polypeptide-encoding gene. The genes/nucleic acid sequences, or the GRF polypeptides and SYT polypeptides themselves may be used to define a molecular marker. This DNA or protein marker may then be used in breeding programmes to select plants having increased yield-related traits, as defined hereinabove in the methods of the invention.

Allelic variants of a gene/nucleic acid sequence encoding a GRF polypeptide and SYT polypeptide may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield-related traits. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acid sequences encoding GRF polypeptides and SYT polypeptides may also be used as probes for genetically and physically mapping the genes that they are a part of, and as markers for traits linked to those genes. Such information may be useful in plant breeding in order to develop lines with desired phenotypes. Such use of nucleic acid sequences encoding a GRF polypeptide and/or a SYT polypeptide requires only a nucleic acid sequence of at least 15 nucleotides in length. The nucleic acid sequences encoding a GRF polypeptide and/or a SYT polypeptide may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acid sequences encoding a GRF polypeptide. The resulting banding patterns may then be subjected to genetic analyses using computer programs such as MapMaker (Lander et al. (1987) Genomics 1: 174-181) in order to construct a genetic map. In addition, the nucleic acid sequences may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid sequence encoding a specific polypeptide in the genetic map previously obtained using this population (Botstein et al. (1980) Am. J. Hum. Genet. 32: 314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid sequence probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid sequence probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid sequence amplification-based methods for genetic and physical mapping may be carried out using the nucleic acid sequences. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic acid sequence Res. 18:3671), Radiation Hybrid Mapping (Walter et al. (1997) Nat. Genet. 7:22-28) and Happy Mapping (Dear and Cook (1989) Nucleic acid sequence Res. 17:6795-6807). For these methods, the sequence of a nucleic acid sequence is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having increased yield-related traits, as described hereinbefore. These traits may also be combined with other economically advantageous traits, such as further yield-increasing traits, tolerance to abiotic and biotic stresses, tolerance to herbicides, insectides, traits modifying various architectural features and/or biochemical and/or physiological features.

### DESCRIPTION OF FIGURES

The present invention will now be described with reference to the following figures in which:

FIG. 1 represents a cartoon of a GRF polypeptide as represented by SEQ ID NO: 2, which comprises the following features: (i) a QLQ domain with an InterPro accession IPR014978 (PFAM accession PF08880); (ii) a WRC domain with an InterPro accession IPR014977 (PFAM accession PF08879); and (iii) an Effector of Transcription (ET) domain comprising three Cys and one His residues in a conserved spacing (CX₉CX₁₀CX₂H), and located within the WRC domain.

FIG. 2 shows an AlignX (from Vector NTI 10.3, Invitrogen Corporation) multiple sequence alignment of the QLQ domain of GRF polypeptides from Table A.1 (as represented by SEQ ID NO: 115 for SEQ ID NO: 2). The conserved QLQ amino acid residues are located on the top of the multiple alignment. Two other very conserved residues (boxed in black) are E (Glu) and P (Pro).

FIG. 3 shows an AlignX (from Vector NTI 10.3, Invitrogen Corporation) multiple sequence alignment of the WRC domain of GRF polypeptides from Table A.1 (as represented by SEQ ID NO: 116 for SEQ ID NO: 2). The conserved WRC amino acid residues are in bold in the consensus sequence. The three Cys and one His residues in a conserved spacing (CX₉CX₁₀CX₂H), designated as the Effector of Transcription (ET) domain, are boxed vertically across the alignement, and also identified at the bottom of the alignment. The putative nuclear localisation signal (NLS) comprised in the WRC domain, is double-underlined.

FIG. 4 shows the typical domain structure of SYT polypeptides from plants and mammals. The conserved SNH domain is located at the N-terminal end of the polypeptide. The C-terminal remainder of the polypeptide consists of a QG-rich domain in plant SYT polypeptides, and of a QPGY-rich domain in mammalian SYT polypeptides. A Met-rich domain is typically comprised within the first half of the QG-rich (from the N-term to the C-term) in plants or QPGY-rich in mammals. A second Met-rich domain may precede the SNH domain in plant SYT polypeptides

FIG. 5 shows a multiple alignment of the N-terminal end of several SYT polypeptides, using VNTI AlignX multiple alignment program, based on a modified ClustalW algorithm (InforMax, Bethesda, Md., http://www.informaxinc.com), with default settings for gap opening penalty of 10 and a gap extension of 0.05). The SNH domain is boxed across the plant and human SYT polypeptides. The last line in the alignment consists of a consensus sequence derived from the aligned sequences.

FIG. 6 shows a multiple alignment of several plant SYT polypeptides, using VNTI AlignX multiple alignment program, based on a modified ClustalW algorithm (InforMax, Bethesda, Md., http://www.informaxinc.com), with default settings for gap opening penalty of 10 and a gap extension of 0.05). The two main domains, from N-terminal to C-terminal, are boxed and identified as SNH domain and the Met-rich/QG-rich domain. Additionally, the N-terminal Met-rich domain is also boxed, and the positions of SEQ ID NO: 90 and SEQ ID NO 91 are underlined in bold.

FIG. 7 shows on the left a panicle from a rice plant (Oryza sativa ssp. Japonica cv. Nipponbare) transformed with a control vector, and on the right a panicle from a rice plant (Oryza sativa ssp. Japonica cv. Nipponbare) transformed with two constructs: (1) a nucleic acid sequence encoding a GRF polypeptide under the control of a GOS2 promoter (pGOS2) from rice; and (2) a nucleic acid sequence encoding a SYT polypeptide under the control of a GOS2 promoter (pGOS2) from rice;

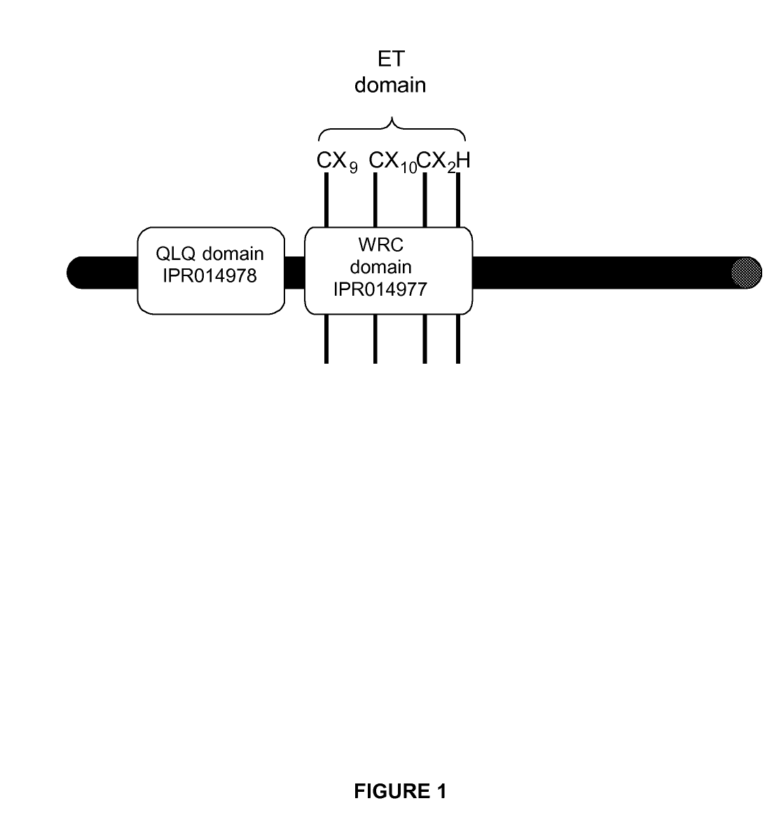

FIG. 8 shows on the top row, from left to right, 30 mature rice seeds (Oryza sativa ssp. Japonica cv. Nipponbare) from:
a. plants transformed with one construct comprising a nucleic acid sequence encoding a SYT polypeptide under the control of a GOS2 promoter (pGOS2) from rice;
b. plants transformed with two constructs: (1) a nucleic acid sequence encoding a GRF polypeptide under the control of a GOS2 promoter (pGOS2) from rice; and (2) a nucleic acid sequence encoding a SYT polypeptide under the control of a GOS2 promoter (pGOS2) from rice;
c. plants transformed with one construct comprising a nucleic acid sequence encoding a GRF polypeptide under the control of a GOS2 promoter (pGOS2) from rice;
d. nullizygote plants (control plants) from a;
e. nullizygote plants (control plants) from c;

FIG. 9 shows the binary vector for increased expression in Oryza sativa of a nucleic acid sequence encoding a GRF polypeptide under the control of a GOS2 promoter (pGOS2) from rice, or alternatively for increased expression in Oryza sativa of a nucleic acid sequence encoding a SYT polypeptide under the control of a GOS2 promoter (pGOS2) from rice.

FIG. 10 details examples of sequences useful in performing the methods according to the present invention.

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid sequence or polypeptide sequences to sequence databases and by calculating the statistical significance of matches. For example, the polypeptide encoded by the nucleic acid sequence of the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid sequence (or polypeptide) sequences over a particular length. In some instances, the default parameters may be adjusted to modify the stringency of the search. For example the E-value may be increased to show less stringent matches. This way, short nearly exact matches may be identified.

Table A.1 provides a list of nucleic acid sequences related encoding GRF polypeptides useful in performing the methods of the present invention. Table A.2 provides a list of nucleic acid sequences related encoding SYT polypeptides useful in performing the methods of the present invention.

TABLE A.1
Examples of GRF polypeptide sequences, and encoding nucleic acid sequences:

| | | | | Database |
|---|---|---|---|---|
| | Source | Nucleic acid | Polypeptide | accession |
| Name | organism | SEQ ID NO: | SEQ ID NO: | number |
| Arath_GRF_At3G13960.1 | Arabidopsis | 1 | 2 | AT3G13960.1 |
| | thaliana | | | |
| Arath_GRF_At2G06200.1 | Arabidopsis | 3 | 4 | At2G06200.1 |
| | thaliana | | | |
| Arath_GRF_At2G22840.1 | Arabidopsis | 5 | 6 | At2G22840.1 |
| | thaliana | | | |
| Arath_GRF_At2G36400.1 | Arabidopsis | 7 | 8 | At2G36400.1 |
| | thaliana | | | |
| Arath_GRF_At2G45480.1 | Arabidopsis | 9 | 10 | At2G45480.1 |
| | thaliana | | | |
| Arath_GRF_At3G52910.1 | Arabidopsis | 11 | 12 | At3G52910.1 |
| | thaliana | | | |
| Arath_GRF_At4G24150.1 | Arabidopsis | 13 | 14 | At4G24150.1 |
| | thaliana | | | |
| Arath_GRF_At4G37740.1 | Arabidopsis | 15 | 16 | At4G37740.1 |
| | thaliana | | | |
| Arath_GRF_At5G53660.1 | Arabidopsis | 17 | 18 | At5G53660.1 |
| | thaliana | | | |
| Aqufo_GRF | Aquilegia | 19 | 20 | DT756681.1 |
| | formosa x | | | DR946716.1 |
| | Aquilegia | | | |
| | pubescens | | | |
| Brana_GRF | Brassica | 21 | 22 | CN730217.1 |
| | napus | | | ES922527 |
| Horvu_GRF | Hordeum | 23 | 24 | AK250947.1 |
| | vulgare | | | |
| Lyces_GRF | Lycopersicon | 25 | 26 | BT013977.1 |
| | esculentum | | | |
| Medtr_GRF | Medicago | 27 | 28 | AC144645.17 |
| | truncatula | | | |
| Medtr_GRF like | Medicago | 29 | 30 | AC174350.4 |
| | truncatula | | | |
| Orysa_GRF_Os02g47280.2 | Oryza sativa | 31 | 32 | Os02g47280.2 |
| Orysa_GRF_Os02g53690.1 | Oryza sativa | 33 | 34 | Os02g53690.1 |
| Orysa_GRF_Os03g51970.1 | Oryza sativa | 35 | 36 | Os03g51970.1 |
| Orysa_GRF_Os04g48510.1 | Oryza sativa | 37 | 38 | Os04g48510.1 |
| Orysa_GRF_Os04g51190.1 | Oryza sativa | 39 | 40 | Os04g51190.1 |
| Orysa_GRF_Os06g02560.1 | Oryza sativa | 41 | 42 | Os06g02560.1 |
| Orysa_GRF_Os11g35030.1 | Oryza sativa | 43 | 44 | Os11g35030.1 |
| Orysa_GRF_Os12g29980.1 | Oryza sativa | 45 | 46 | Os12g29980.1 |
| Oyrsa_GRF_Os03g47140.1 | Oryza sativa | 47 | 48 | Os03g47140.1 |
| Orysa_GRF_gi_115447910_ref_NM_001054270.1 | Oryza sativa | 49 | 50 | NM_001054270.1 |
| Orysa_GRF_gi_115460325_ref_NM_001060298.1 | Oryza sativa | 51 | 52 | NM_001060298.1 |
| Orysa_GRF_gi_115471984_ref_NM_001066126.1 | Oryza sativa | 53 | 54 | NM_001066126.1 |
| Poptr_GRF_lcl_scaff_XIV.39 | Populus | 55 | 56 | lcl_scaff_XIV.39 |
| | tremuloides | | | |
| Poptr_GRF_lcl_scaff_II.1070 | Populus | 57 | 58 | lcl_scaff_II.1070 |
| | tremuloides | | | |
| Poptr_GRF_lcl_scaff_I.1018 | Populus | 59 | 60 | lcl_scaff_I.1018 |
| | tremuloides | | | |
| Poptr_GRF_lcl_scaff_28.10 | Populus | 61 | 62 | lcl_scaff_28.10 |
| | tremuloides | | | |
| Poptr_GRF_lcl_scaff_I.995 | Populus | 63 | 64 | lcl_scaff_I.995 |
| | tremuloides | | | |
| Poptr_GRF_lcl_scaff_III.741 | Populus | 65 | 66 | lcl_scaff_III.741 |
| | tremuloides | | | |
| Poptr_GRF_lcl_scaff_VII.1274 | Populus | 67 | 68 | lcl_scaff_VII.1274 |
| | tremuloides | | | |
| Poptr_GRF_lcl_scaff_XII.277 | Populus | 69 | 70 | lcl_scaff_XII.277 |
| | tremuloides | | | |
| Poptr_GRF_lcl_scaff_XIII.769 | Populus | 71 | 72 | lcl_scaff_XIII.769 |
| | tremuloides | | | |
| Poptr_GRF_lcl_scaff_XIV.174 | Populus | 73 | 74 | lcl_scaff_XIV.174 |
| | tremuloides | | | |
| Poptr_GRF_lcl_scaff_XIV.51 | Populus | 75 | 76 | lcl_scaff_XIV.51 |
| | tremuloides | | | |
| Poptr_GRF_lcl_scaff_XIX.480 | Populus | 77 | 78 | lcl_scaff_XIX.480 |
| | tremuloides | | | |
| Poptr_GRF_lcl_scaff_28.309 | Populus | 79 | 80 | lcl_scaff_28.309 |
| | tremuloides | | | |
| Poptr_GRF_lcl_scaff_I.688 | Populus | 81 | 82 | lcl_scaff_I.688 |
| | tremuloides | | | |
| Sacof_GRF | Saccharum | 83 | 84 | CA084837.1 |
| | officinarum | | | CA238919.1 |
| | | | | CA122516.1 |
| Vitvi_GRF | Vitis vinifera | 85 | 86 | AM468035 |
| Zeama_GRF10_gi_146008494_gb_EF515849.1 | Zea mays | 87 | 88 | EF515849.1 |
| Zeama_GRF11_gi_146008515_gb_EF515850.1 | Zea mays | 89 | 90 | EF515850.1 |
| Zeama_GRF12_gi_146008534_gb_EF515851.1 | Zea mays | 91 | 92 | EF515851.1 |
| Zeama_GRF13_gi_146008539_gb_EF515852.1 | Zea mays | 93 | 94 | EF515852.1 |
| Zeama_GRF14_gi_146008560_gb_EF515853.1 | Zea mays | 95 | 96 | EF515853.1 |
| Zeama_GRF1_gi_146008330_gb_EF515840.1 | Zea mays | 97 | 98 | EF515840.1 |
| Zeama_GRF2_gi_146008352_gb_EF515841.1 | Zea mays | 99 | 100 | EF515841.1 |
| Zeama_GRF3_gi_146008368_gb_EF515842.1 | Zea mays | 101 | 102 | EF515842.1 |
| Zeama_GRF4_gi_146008393_gb_EF515843.1 | Zea mays | 103 | 104 | EF515843.1 |
| Zeama_GRF5_gi_146008412_gb_EF515844.1 | Zea mays | 105 | 106 | EF515844.1 |
| Zeama_GRF6_gi_146008429_gb_EF515845.1 | Zea mays | 107 | 108 | EF515845.1 |
| Zeama_GRF7_gi_146008440_gb_EF515846.1 | Zea mays | 109 | 110 | EF515846.1 |
| Zeama_GRF8_gi_146008461_gb_EF515847.1 | Zea mays | 111 | 112 | EF515847.1 |
| Zeama_GRF9_gi_146008475_gb_EF515848.1 | Zea mays | 113 | 114 | EF515848.1 |

TABLE A.2
Examples of SYT polypeptide sequences, and encoding nucleic acid sequences:

| | | | Translated | |
|---|---|---|---|---|
| | | Nucleic acid | polypeptide | Database |
| | | sequence | sequence | accession |
| Name | Source organism | SEQ ID NO | SEQ ID NO | number |
| Arath_SYT1 | Arabidopsis thaliana | 120 | 121 | AY102639.1 |
| Arath_SYT2 | Arabidopsis thaliana | 122 | 123 | AY102640.1 |
| Arath_SYT3 | Arabidopsis thaliana | 124 | 125 | AY102641.1 |
| Allce_SYT2 | Allium cepa | 126 | 127 | CF437485 |
| Aqufo_SYT1 | Aquilegia formosa x | 128 | 129 | DT758802.1 |
| | Aquilegia pubescens | | | |
| Aqufo_SYT2 | Aquilegia formosa x | 130 | 131 | TA15831_338618 |
| | Aquilegia pubescens | | | T25K16.15 |
| Aspof_SYT1 | Aspergilus officinalis | 132 | 133 | CV287542 |
| Betvu_SYT2 | Beta vulgaris | 134 | 135 | BQ594749.1 |
| | | | | BQ594658.1 |
| Bradi_SYT3 | Brachypodium | 136 | 137 | DV480064.1 |
| | distachyon | | | |
| Brana_SYT1 | Brassica napus | 138 | 139 | CD823592 |
| Brana_SYT2 | Brassica napa | 140 | 141 | CN732814 |
| Chlre_SYT | Chlamydomonas | 142 | 143 | BQ814858, |
| | reinhardtii | | | jgi_Chlre3_194013_estExt_fgenesh2_pg.C_510025 |
| Citsi_SYT1 | Citrus sinensis | 144 | 145 | CB290588 |
| Citsi_SYT2 | Citrus sinensis | 146 | 147 | CV717501 |
| Cryja_SYT1 | Cryptomeria japonica | 148 | 149 | TA3001_3369_2 |
| Curlo_SYT2 | Curcuma longa | 150 | 151 | TA2676_136217 |
| Eupes_SYT2 | Euphorbia esula | 152 | 153 | DV144834 |
| Frave_SYT2 | Fragaria vesca | 154 | 155 | DY668312 |
| Glyma_SYT1.1 | Glycine max | 156 | 157 | TA55102_3847 |
| Glyma_SYT1.2 | Glycine max | 158 | 159 | TA51451_3847 |
| Glyma_SYT2.1 | Glycine max | 160 | 161 | BQ612648 |
| Glyma_SYT2.2 | Glycine max | 162 | 163 | TA48452_3847 |
| Glyso_SYT2 | Glycine soya | 164 | 165 | CA799921 |
| Gosar_SYT1 | Gossypium arboreum | 166 | 167 | BM359324 |
| Goshi_SYT1 | Gossypium hirsutum | 168 | 169 | DT558852 |
| Goshi_SYT2 | Gossypium hirsutum | 170 | 171 | DT563805 |
| Helan_SYT1 | Helianthus annuus | 172 | 173 | TA12738_4232 |
| Horvu_SYT2 | Hordeum vulgare | 174 | 175 | CA032350 |
| Lacse_SYT2 | Lactuca serriola | 176 | 177 | DW110765 |
| Lyces_SYT1 | Lycopersicon | 178 | 179 | AW934450.1 |
| | esculentum | | | BP893155.1 |
| Maldo_SYT2 | Malus domestica | 180 | 181 | CV084230 |
| | | | | DR997566 |
| Medtr_SYT1 | Medicago trunculata | 182 | 183 | CA858507.1 |
| Medtr_SYT2 | Medicago trunculata | 184 | 185 | CA858743 |
| | | | | BI310799.1 |
| | | | | AL382135.1 |
| Orysa_SYT1 | Oryza sativa | 186 | 187 | AK058575 |
| Orysa_SYT2 | Oryza sativa | 188 | 189 | AK105366 |
| Orysa_SYT3 | Oryza sativa | 190 | 191 | BP185008 |
| Panvi_SYT3 | Panicum virgatum | 192 | 193 | DN152517 |
| Phypa_SYT1.1 | Physcomitrella patens | 194 | 195 | TA28566_3218 |
| Phypa_SYT1.2 | Physcomitrella patens | 196 | 197 | TA21282_3218 |
| Phypa_SYT1.3 | Physcomitrella patens | 198 | 199 | TA20922_3218 |
| Phypa_SYT1.4 | Physcomitrella patens | 200 | 201 | TA29452_3218 |
| Picsi_SYT1 | Picea sitchensis | 202 | 203 | DR484100 |
| | | | | DR478464.1 |
| Pinta_SYT1 | Pinus taeda | 204 | 205 | DT625916 |
| Poptr_SYT1 | Populus trichocarpa | 206 | 207 | DT476906 |
| Poptr_SYT2 | Populus trichocarpa | 208 | 209 | scaff_XIV.493 |
| Poptr_SYT1.2 | Populus trichocarpa | 210 | 211 | CV257942.1 |
| Prupe_SYT2 | Prunus | 212 | 213 | DT454880.1 |
| | persica | | | DT455286.1 |
| Sacof_SYT1 | Saccharum officinarum | 214 | 215 | CA078249.1 |
| | | | | CA078630 |
| | | | | CA082679 |
| | | | | CA234526 |
| | | | | CA239244 |
| | | | | CA083312 |
| Sacof_SYT2 | Saccharum officinarum | 216 | 217 | CA110367 |
| Sacof_SYT3 | Saccharum officinarum | 218 | 219 | CA161933.1 |
| | | | | CA265085 |
| Soltu_SYT1.1 | Solanum tuberosum | 220 | 221 | CK265597 |
| Soltu_SYT1.2 | Solanum tuberosum | 222 | 223 | BG590990 |
| Soltu_SYT3 | Solanum tuberosum | 224 | 225 | CK272804 |
| Sorbi_SYT1 | Sorghum bicolor | 226 | 227 | TA40712_4558 |
| Sorbi_SYT2 | Sorghum bicolor | 228 | 229 | CF482417 |
| | | | | CW376917 |
| Sorbi_SYT3 | Sorghum bicolor | 230 | 231 | CX611128 |
| Taxof_SYT2 | Taraxacum officinale | 232 | 233 | TA1299_50225 |
| Taxof_SYT3 | Taraxacum officinale | 234 | 235 | TA5000_50225 |
| Triae_SYT1 | Triticum aestivum | 236 | 237 | TA105893_4565 |
| Triae_SYT2 | Triticum aestivum | 238 | 239 | CD901951 |
| Triae_SYT3 | Triticum aestivum | 240 | 241 | BJ246754 |
| | | | | BJ252709 |
| Vitvi_SYT1.1 | Vitis vinifera | 242 | 243 | DV219834 |
| Vitvi_SYT1.2 | Vitis vinifera | 244 | 245 | EE108079 |
| Vitvi_SYT2.1 | Vitis vinifera | 246 | 247 | EC939550 |
| Vitvi_SYT2.2 | Vitis vinifera | 248 | 249 | EE094148.1 |
| | | | | EC964169.1 |
| Volca_SYT | Volvox carteri | 250 | 251 | JGI_CBHO11121.fwdJGI_CBHO11121.rev |
| Welmi_SYT | Welwitschia mirabilis | 252 | 253 | DT598761 |
| Zeama_SYT1 | Zea mays | 254 | 255 | BG874129.1 |
| | | | | CA409022.1 |
| Zeama_SYT2 | Zea mays | 256 | 257 | AY106697 |
| Zeama_SYT3 | Zea mays | 258 | 259 | CO468901 |
| Homsa_SYT | Homo sapiens | 260 | 261 | CR542103 |

In some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid sequence or polypeptide sequence of interest. On other instances, special nucleic acid sequence databases have been created for particular organisms, such as by the Joint Genome Institute, for example for poplar and Ostreococcus tauri.

### Example 2

### Alignment of Polypeptide Sequences Useful in Performing the Methods of the Invention

### Alignment of GRF Polypeptide Sequences

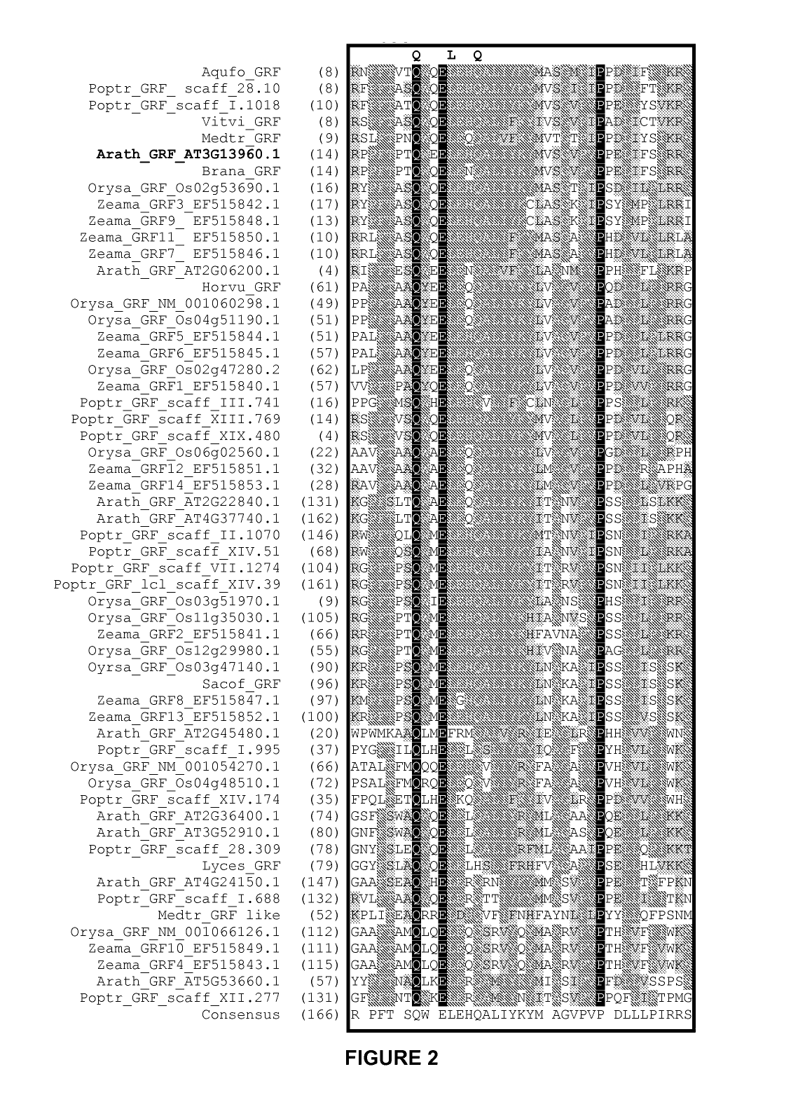

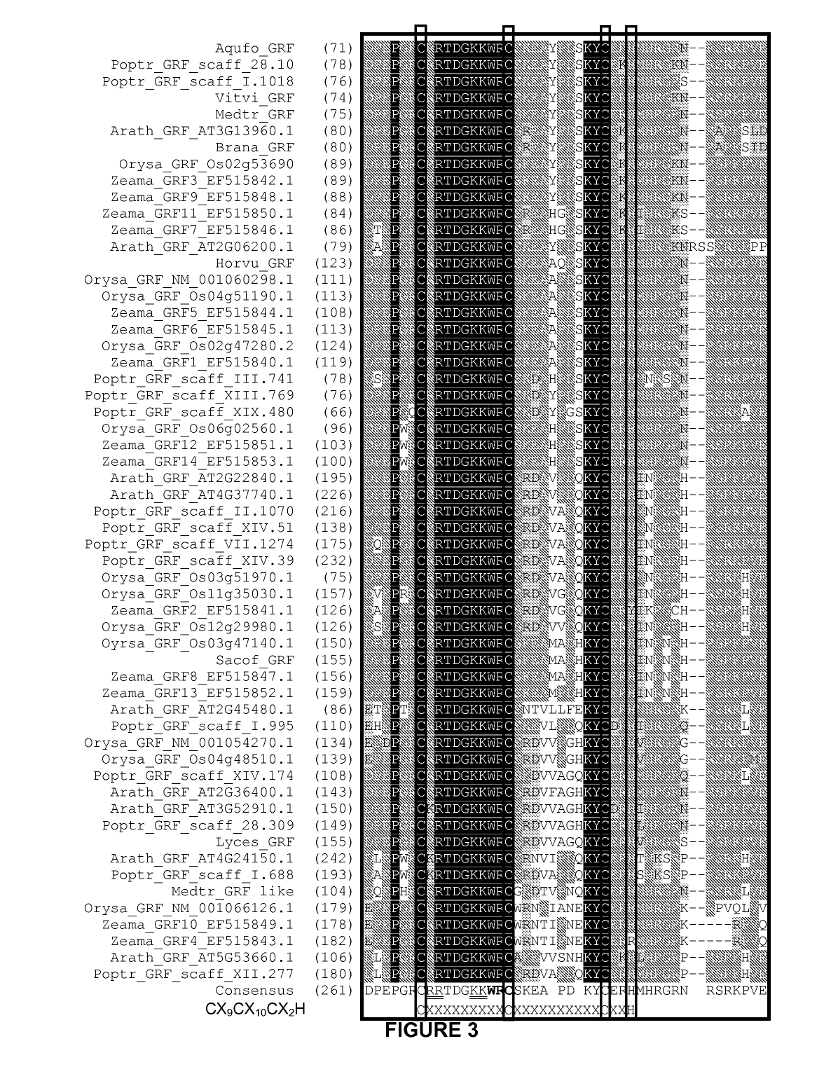

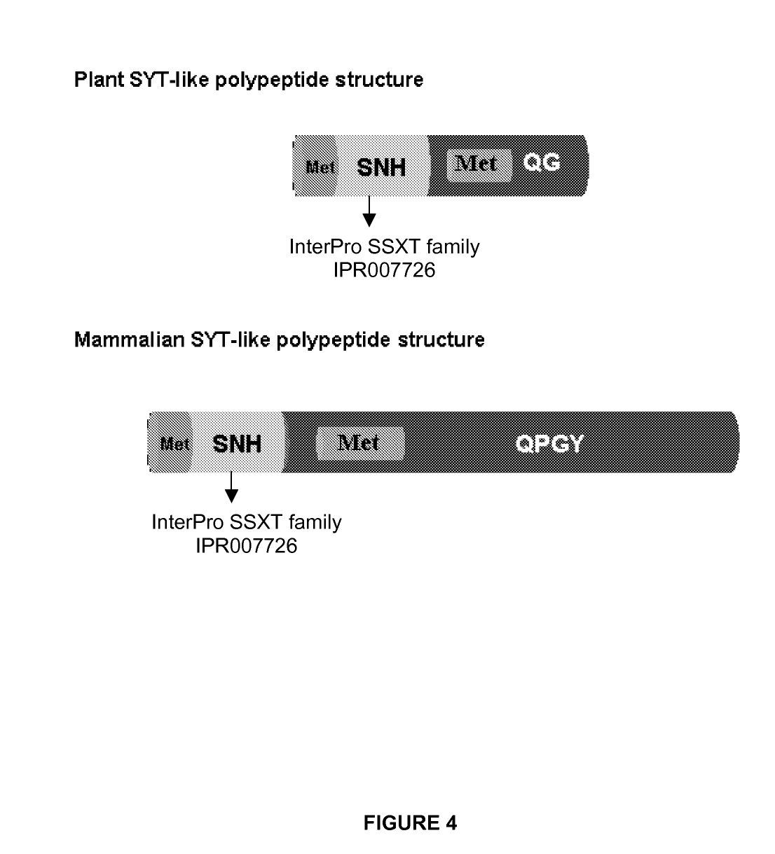

Multiple sequence alignment of all the GRF polypeptide sequences in Table A.1 was performed using the AlignX algorithm (from Vector NTI 10.3, Invitrogen Corporation). Results of the alignment for the QLQ domain of GRF polypeptides from Table A.1 (as represented by SEQ ID NO: 115 for SEQ ID NO: 2) are shown in FIG. 2 of the present application. The conserved QLQ amino acid residues are located on the top of the multiple alignement. Two other very conserved residues (boxed in black) are E (Glu) and P (Pro). Results of the alignment for the WRC domain of the GRF polypeptides from Table A.1 (as represented by SEQ ID NO: 116 for SEQ ID NO: 2) are shown in FIG. 3 of the present application. The conserved WRC amino acid residues are in bold in the consensus sequence. The Effector of Transcription (ET) domain, comprising three Cys and one His residues in a conserved spacing (CX₉CX₁₀CX₂H), is identified at the bottom of the alignment.

### Alignment of SYT Polypeptide Sequences

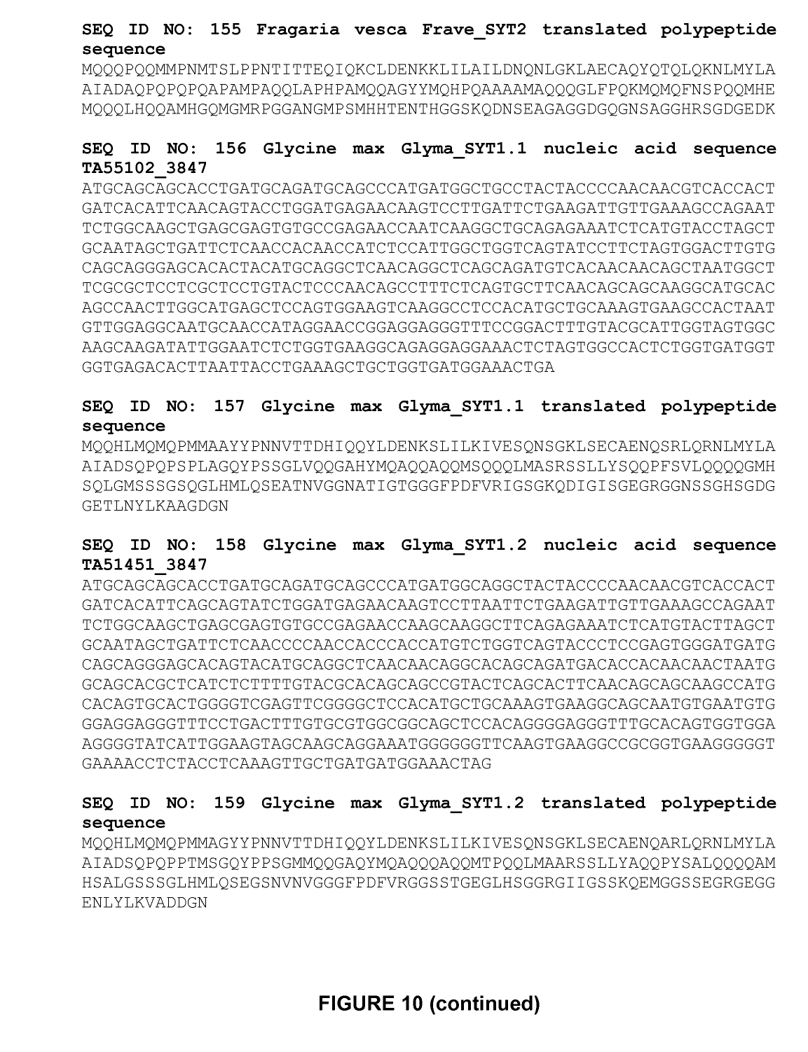

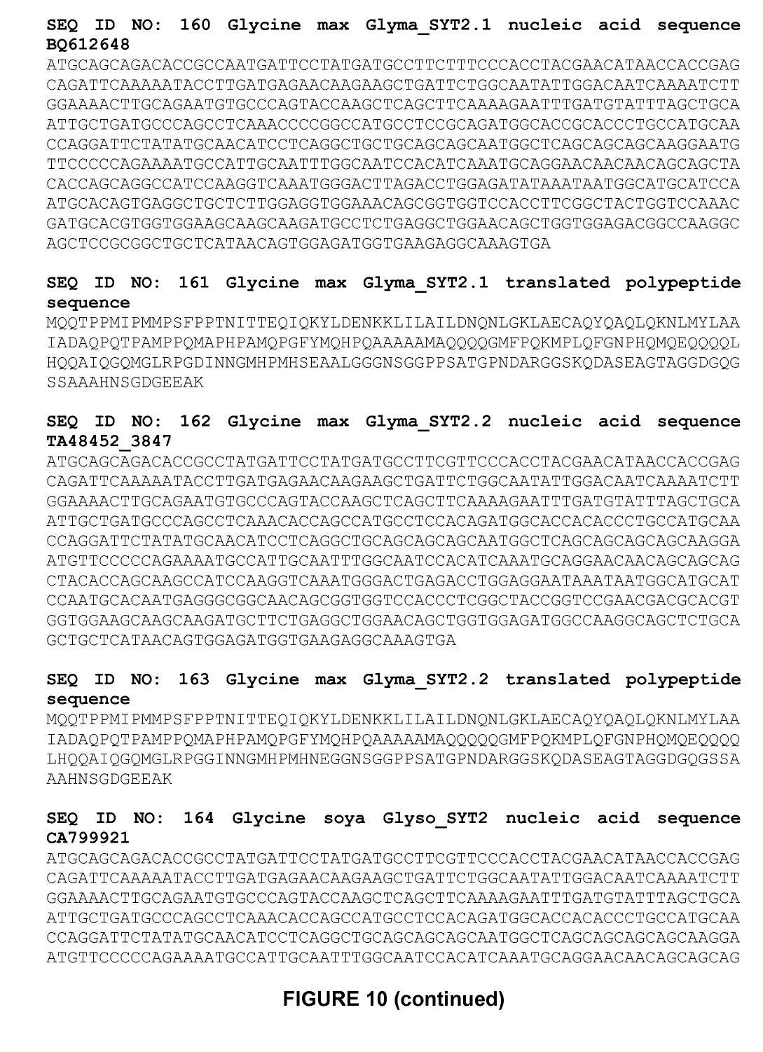

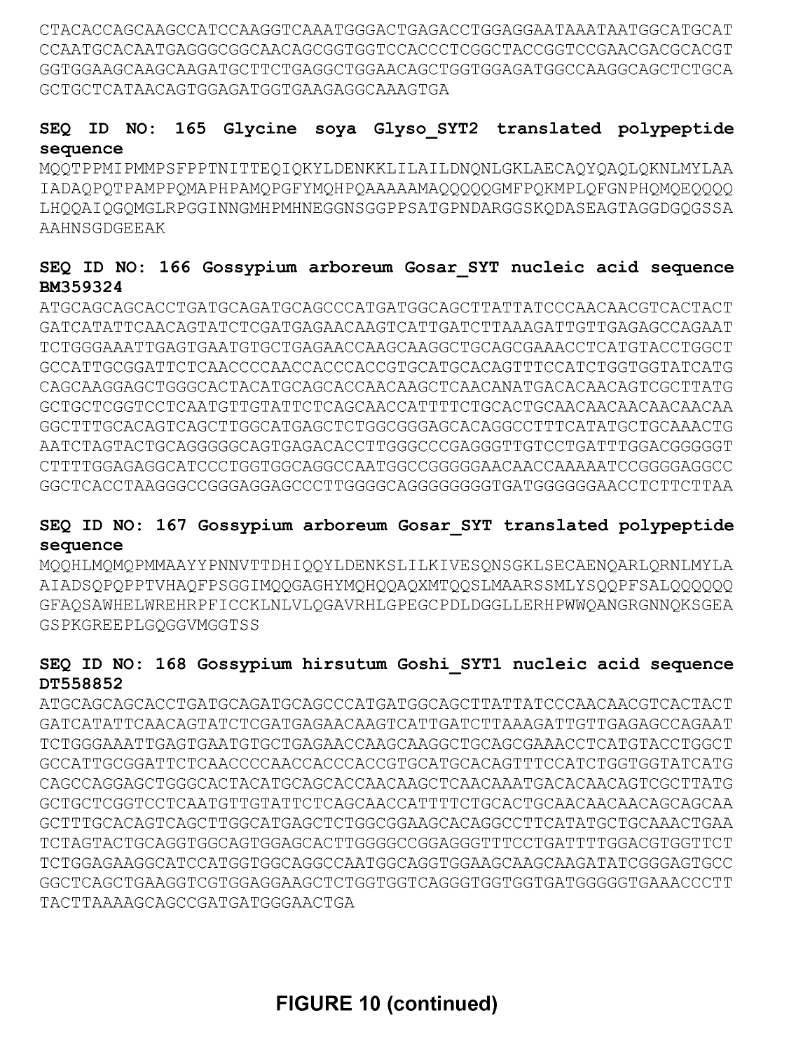

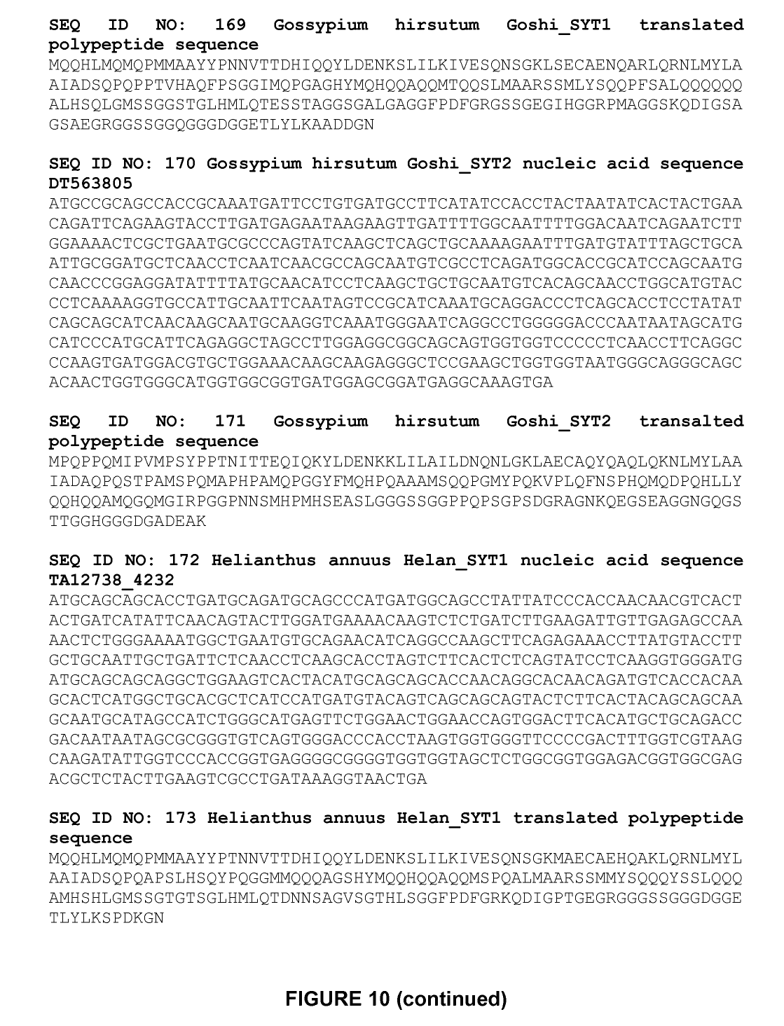

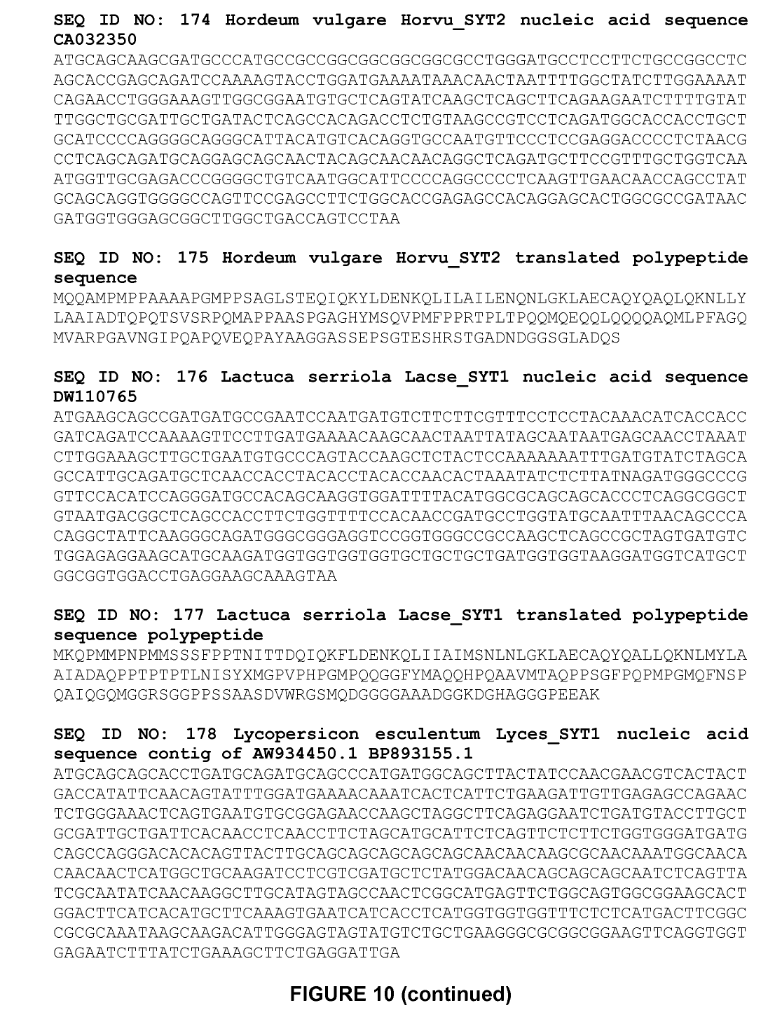

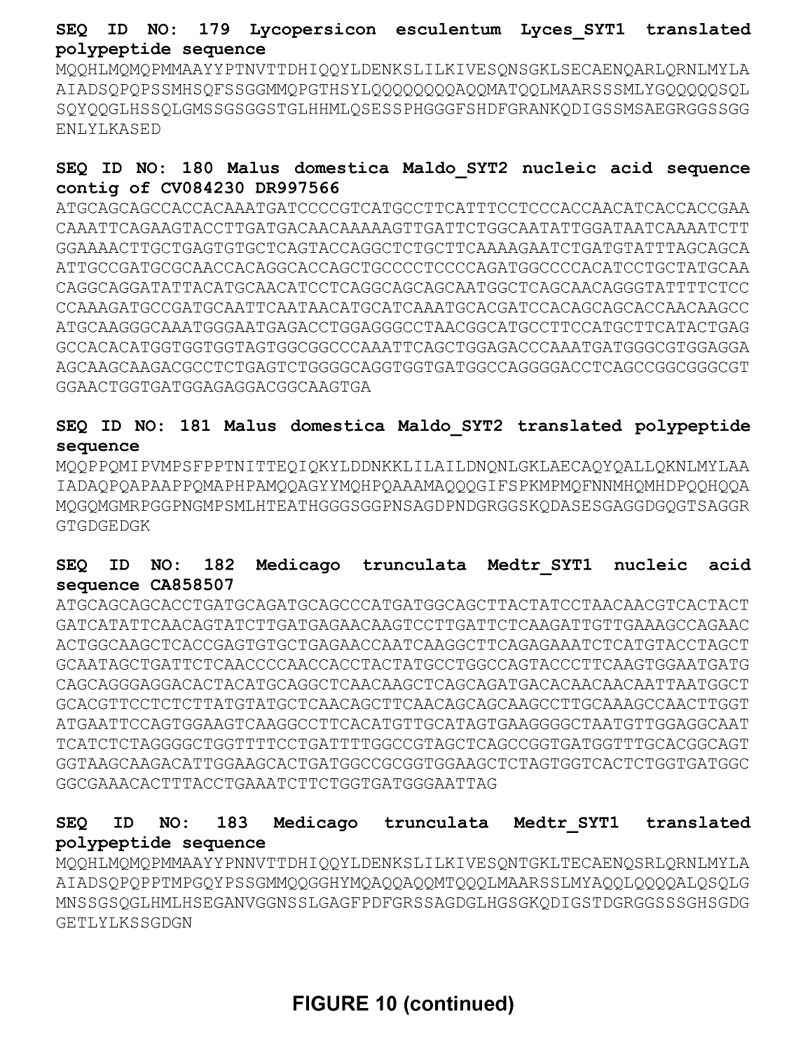

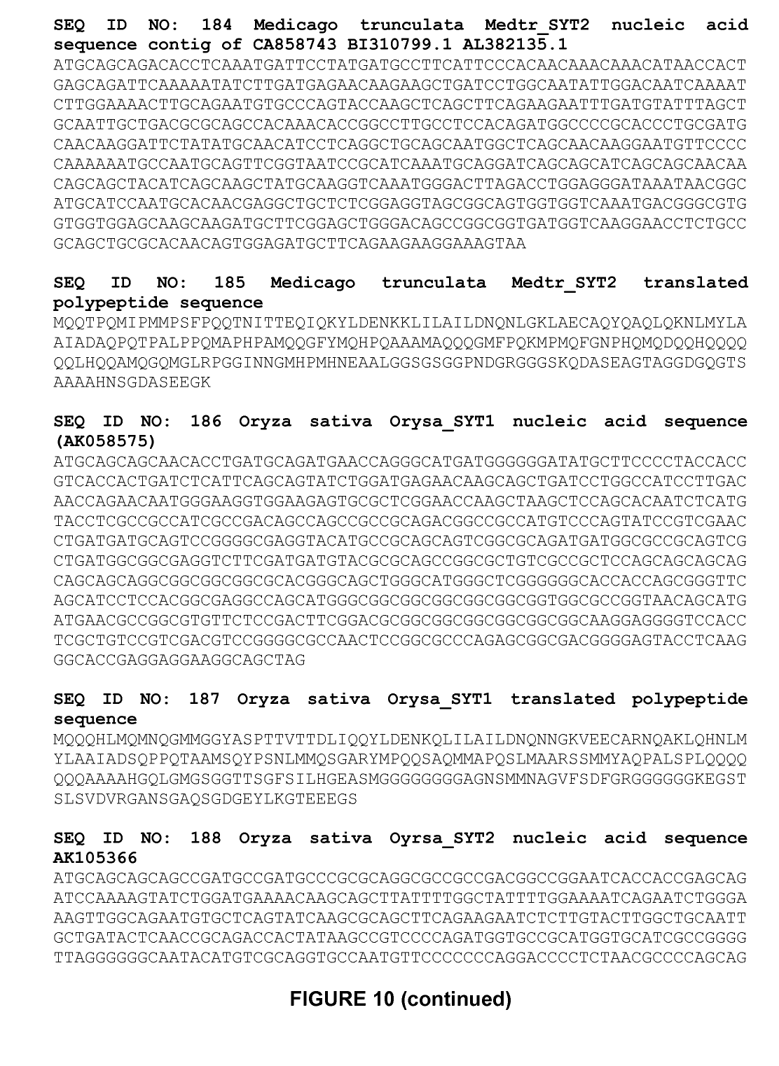

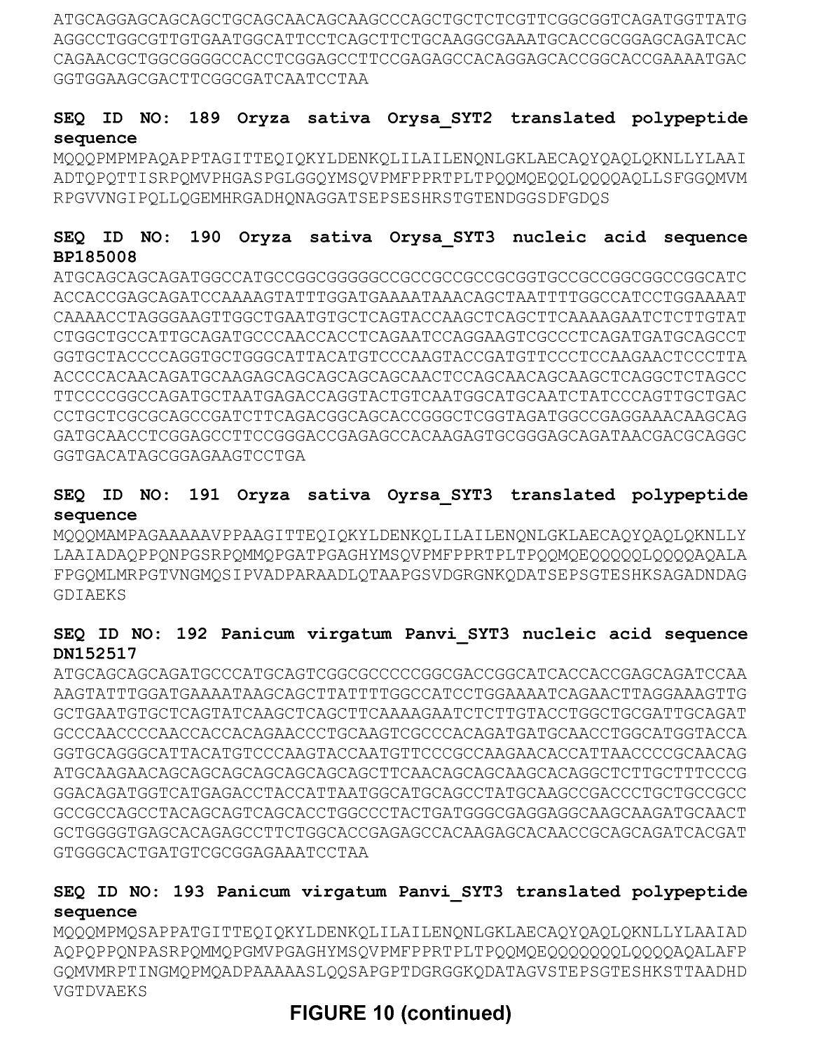

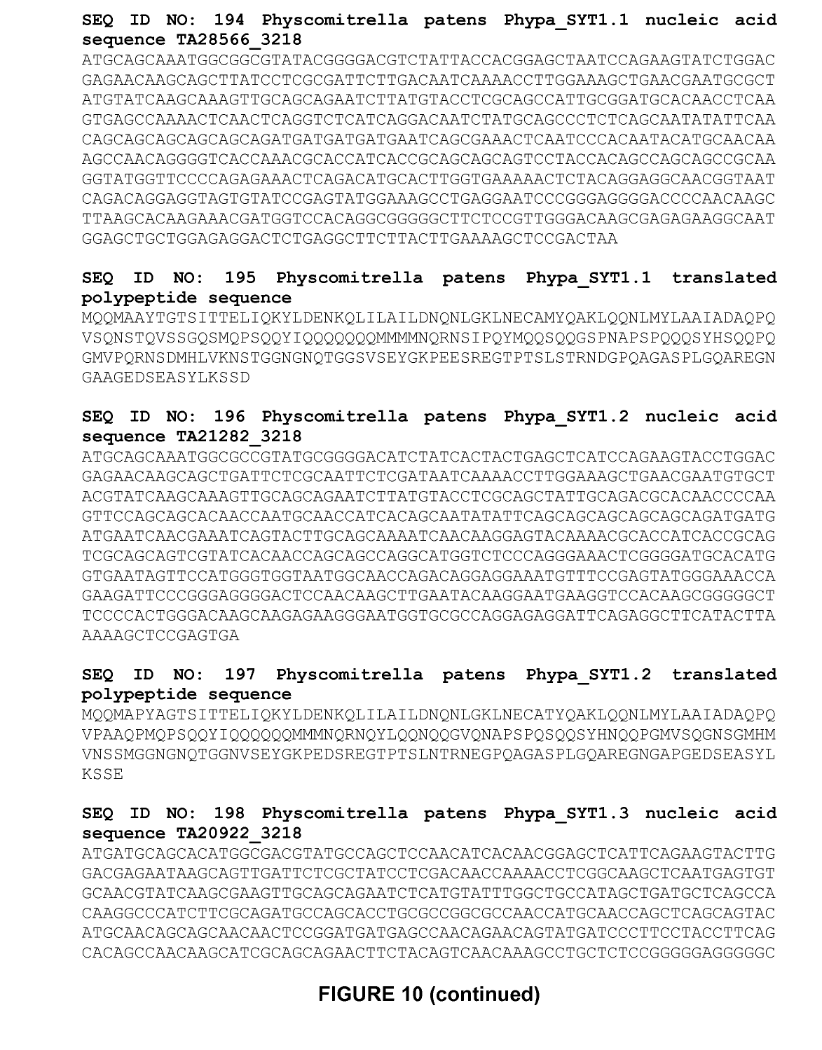

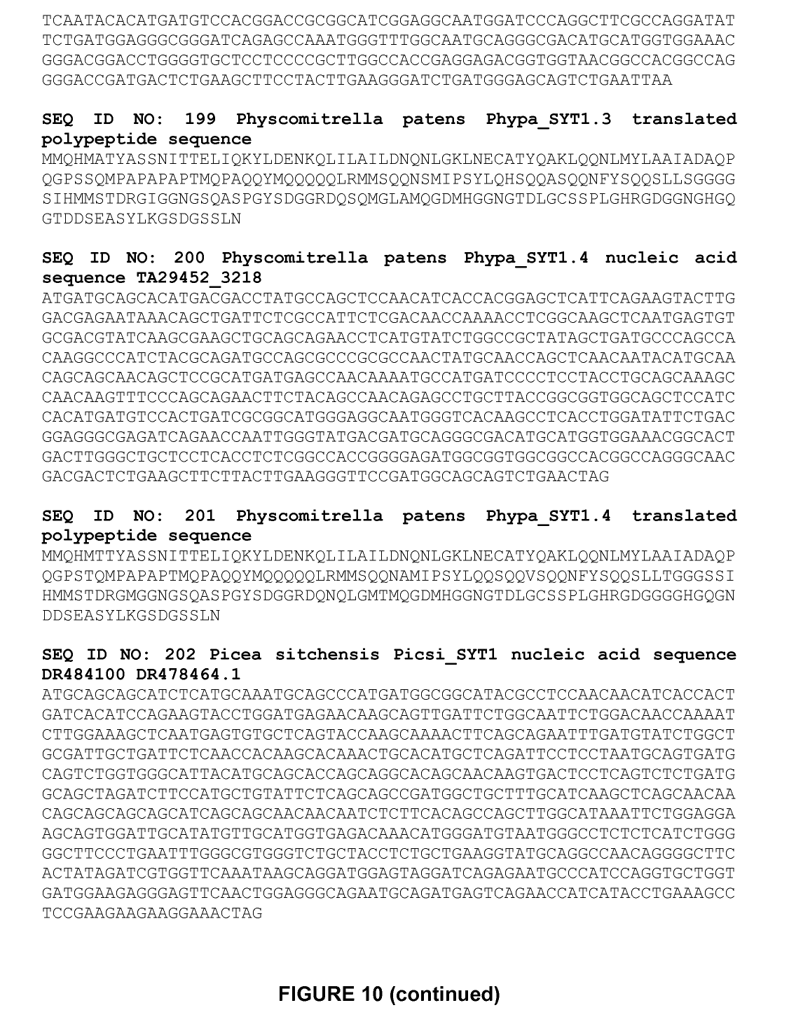

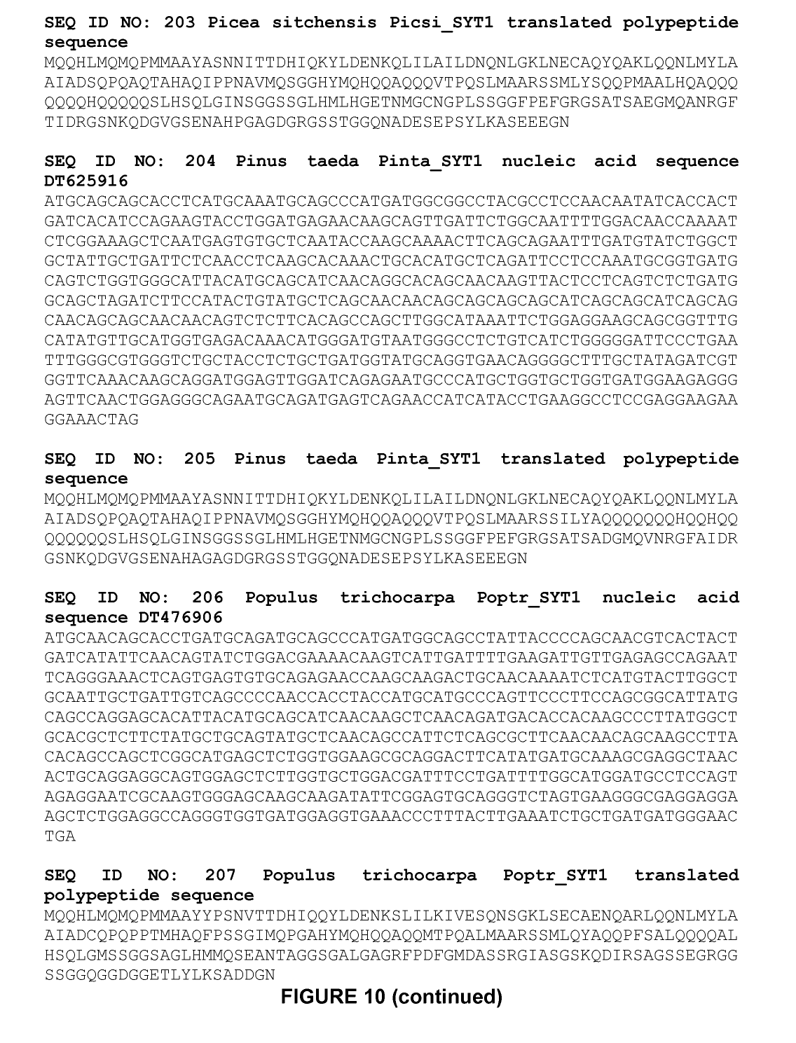

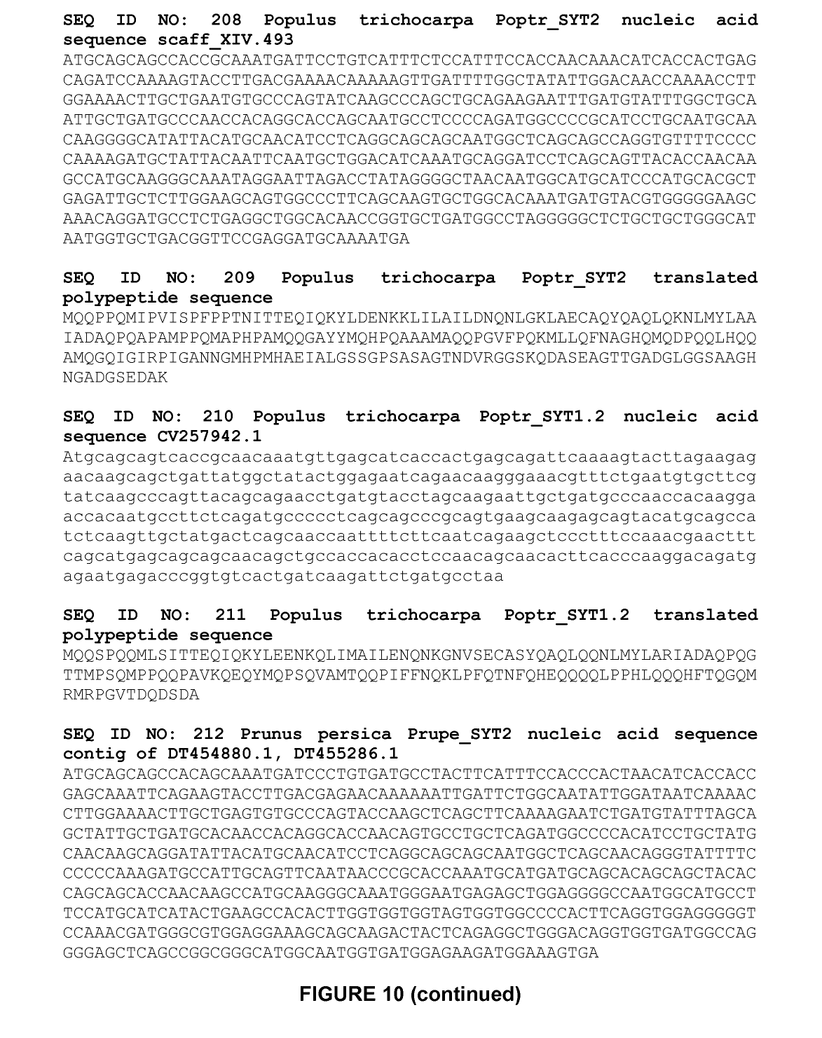

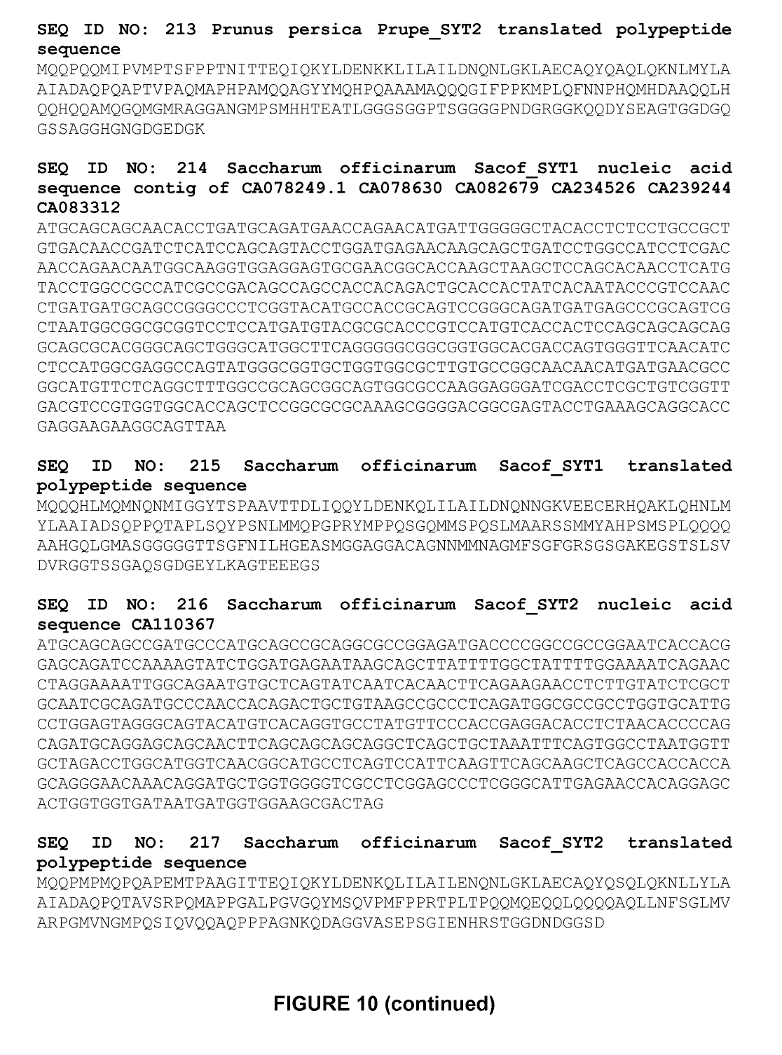

Multiple sequence alignment of all the SYT polypeptide sequences in Table A.2 was performed using the AlignX algorithm (based on a modified ClustalW algorithm; from Vector NTI 10.3, Invitrogen Corporation) with default settings for gap opening penalty of 10 and a gap extension of 0.05), and is shown in FIG. 6, The two main domains, from N-terminal to C-terminal, are boxed and identified as SNH domain and the Met-rich/QG-rich domain. Additionally, the N-terminal Met-rich domain is also boxed.

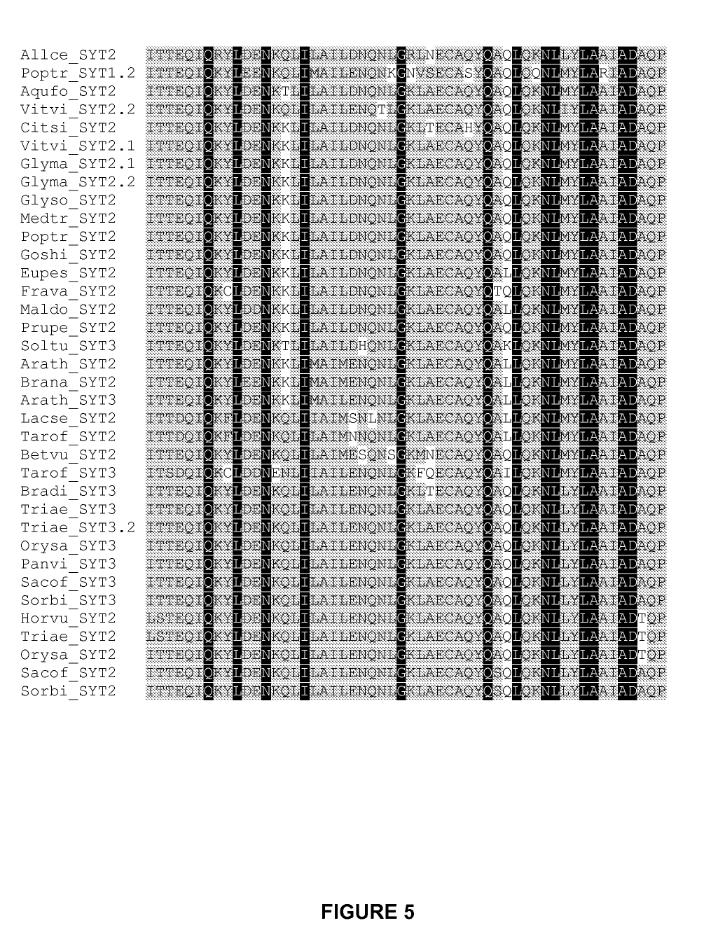

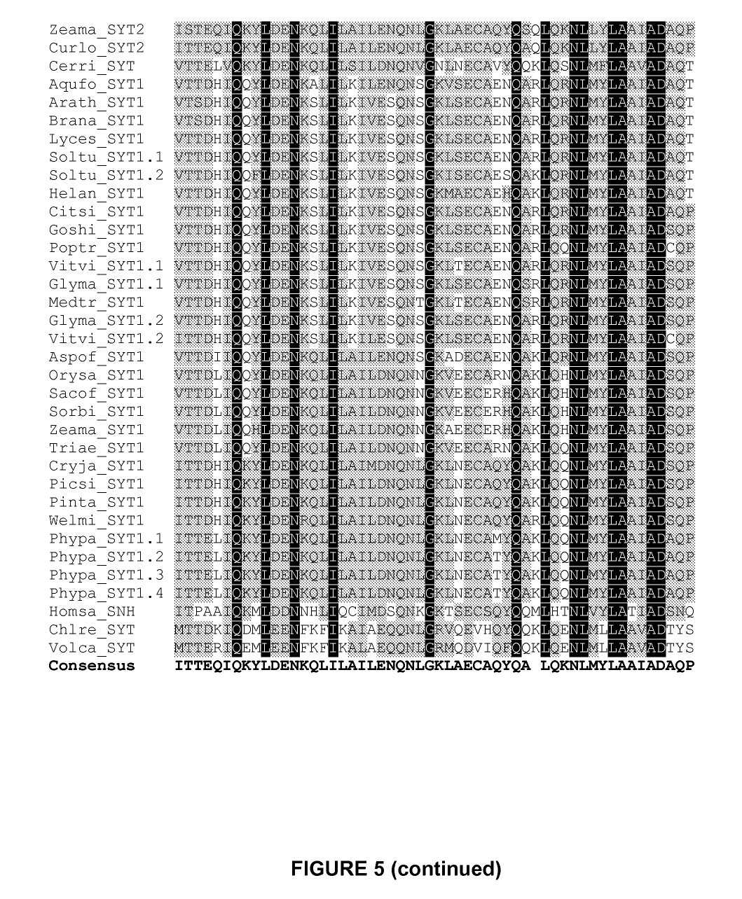

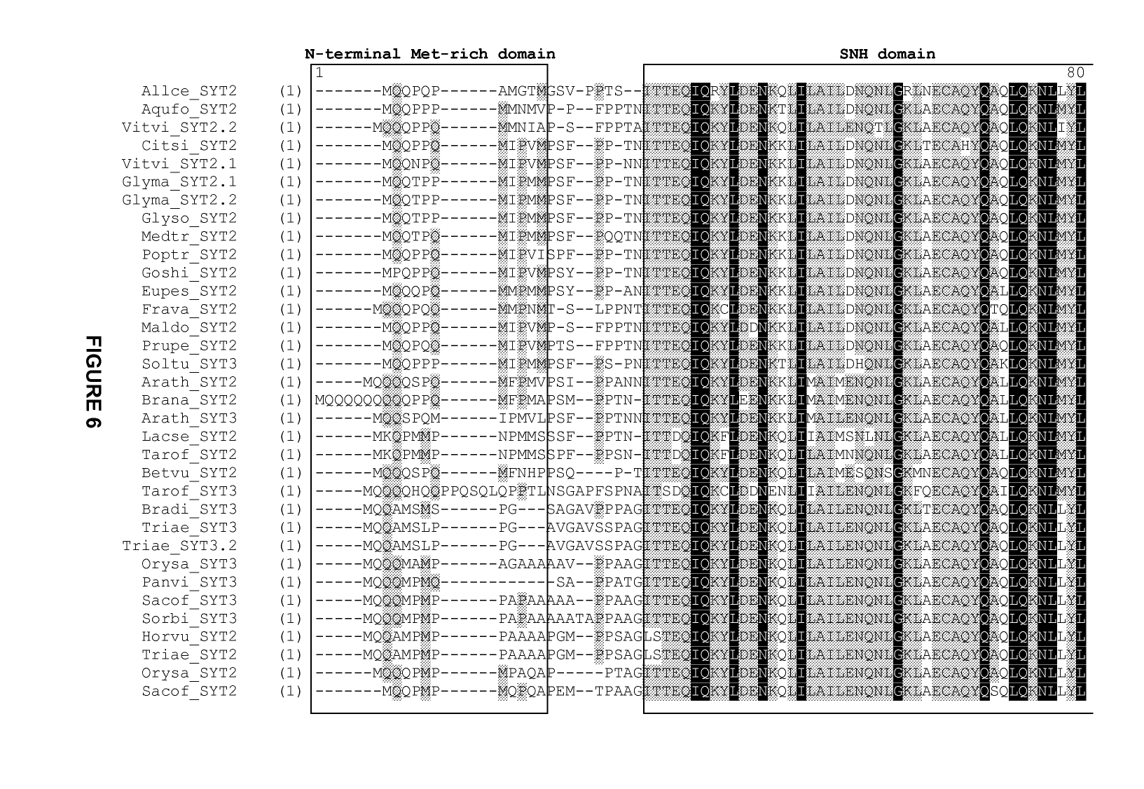

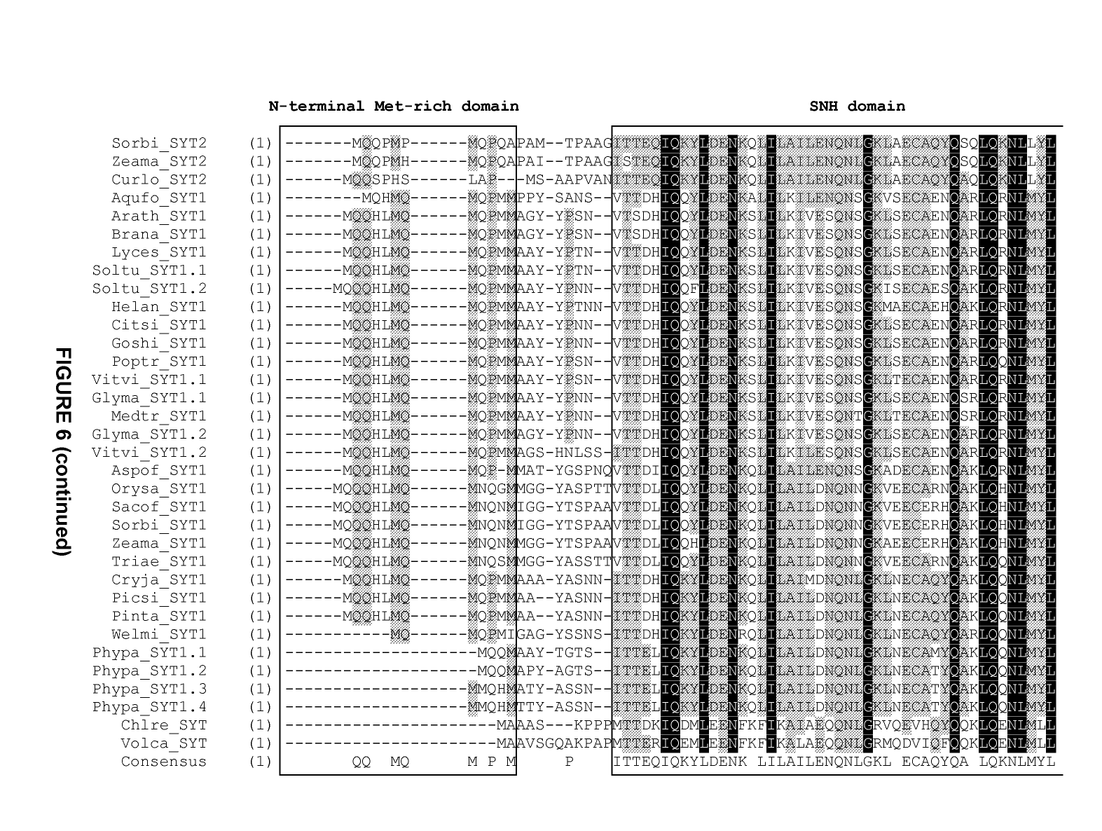

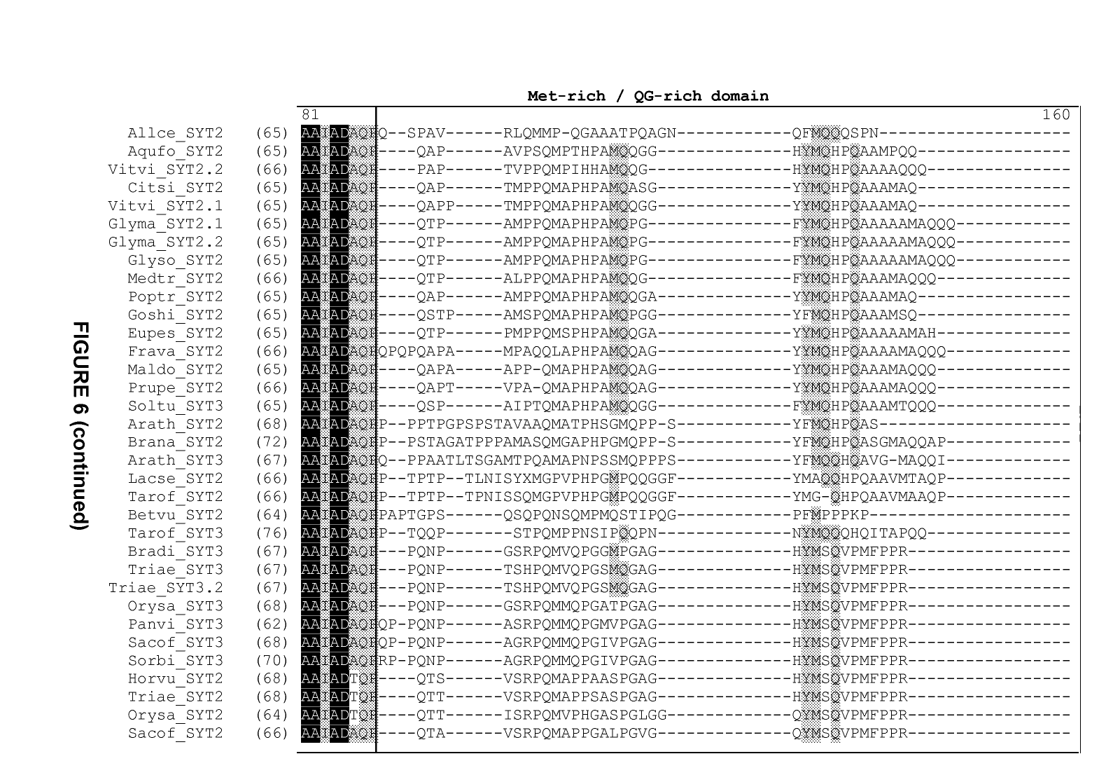

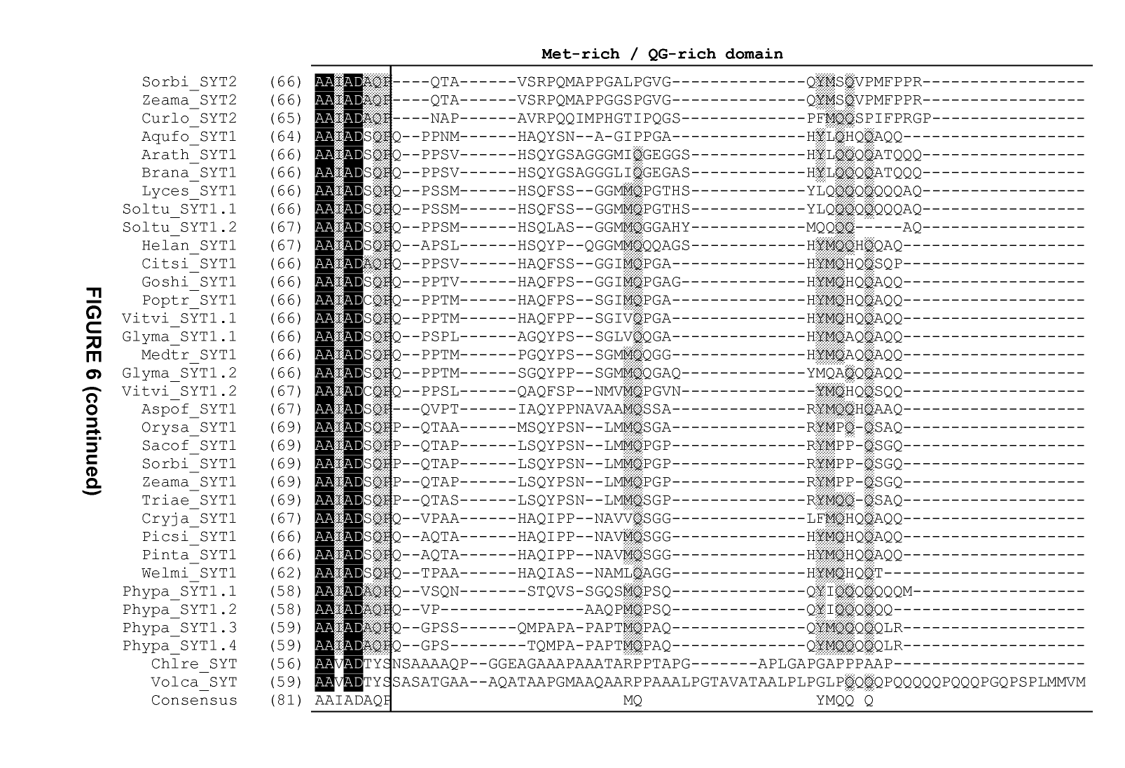

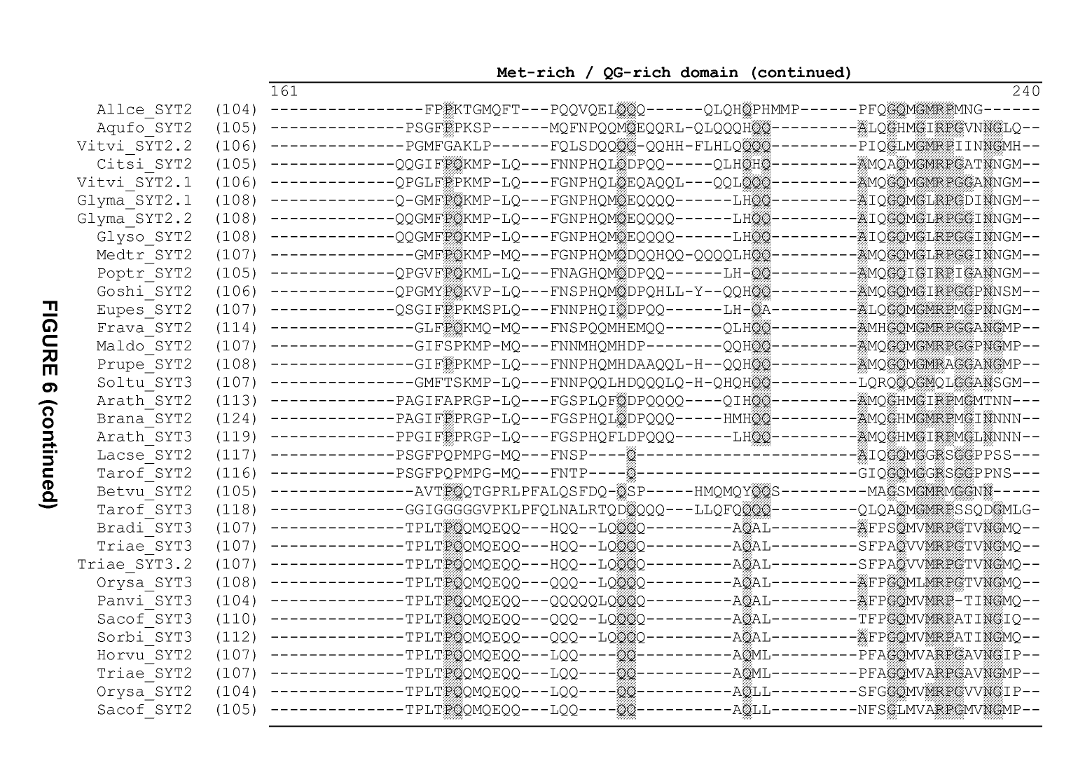

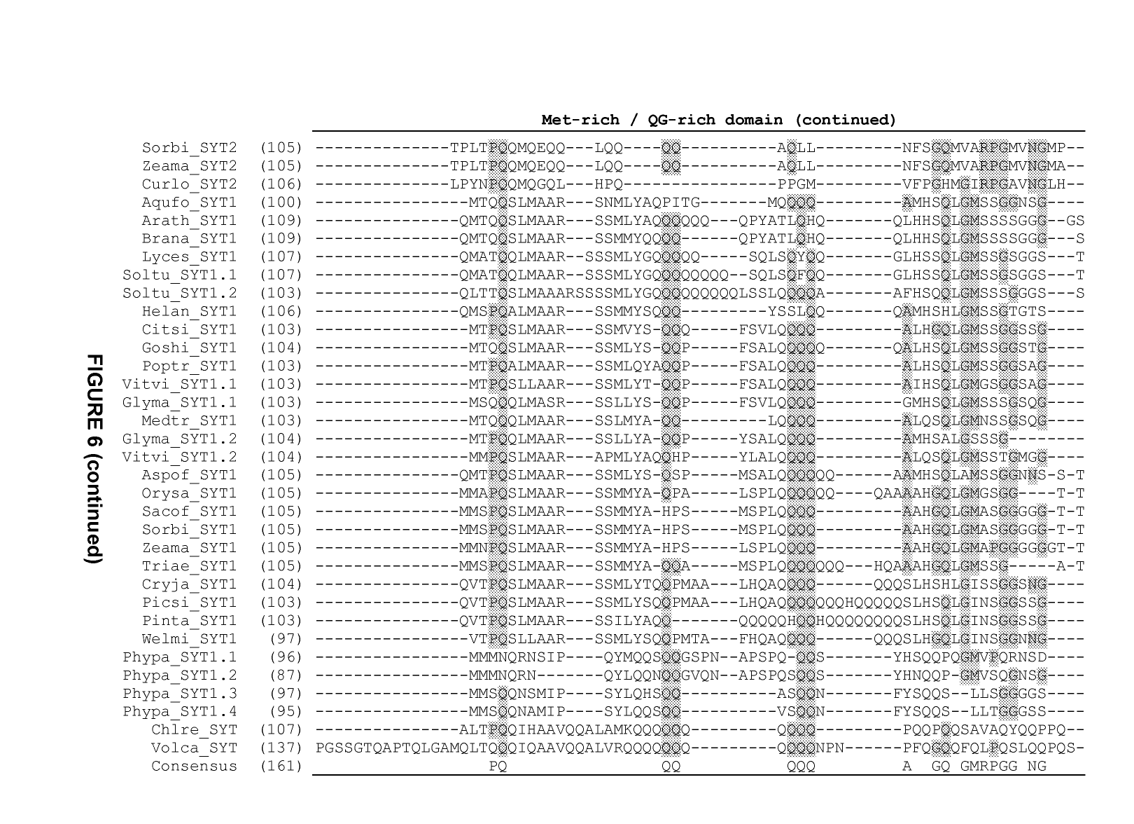

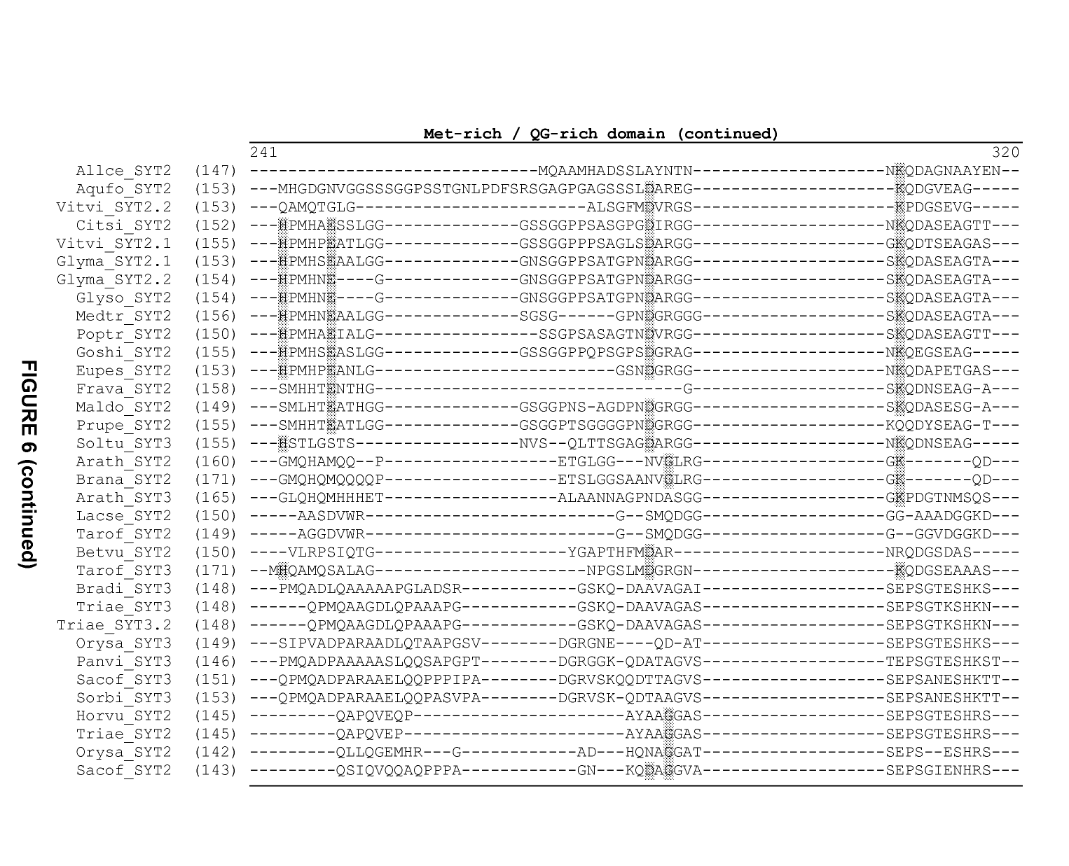

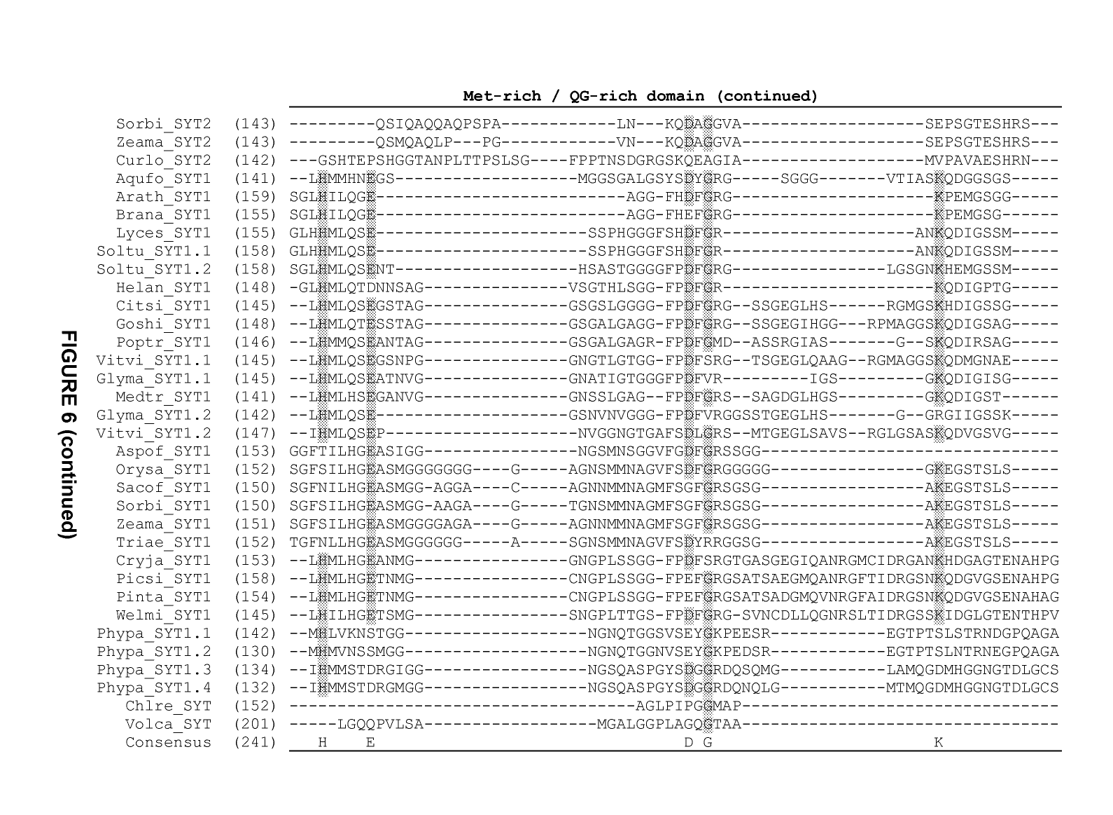

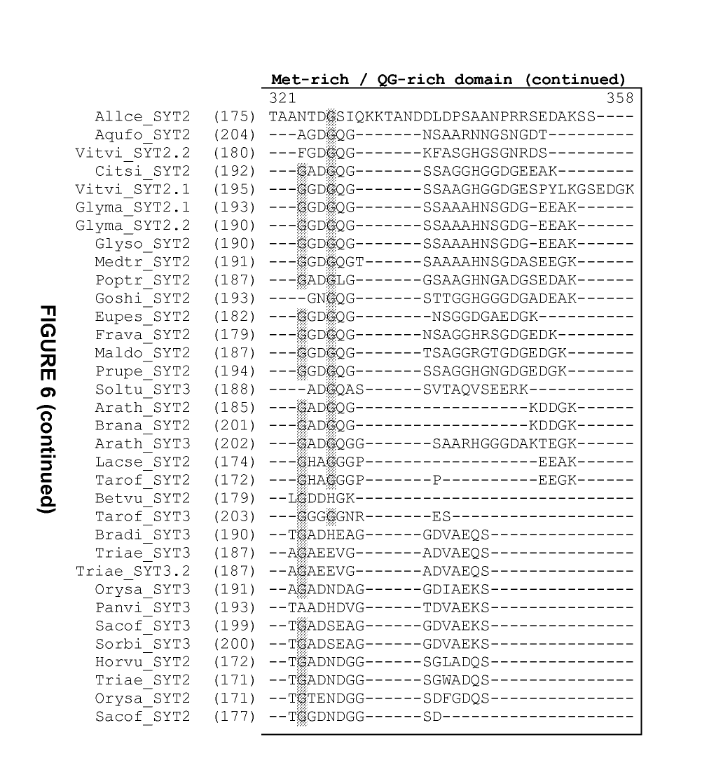

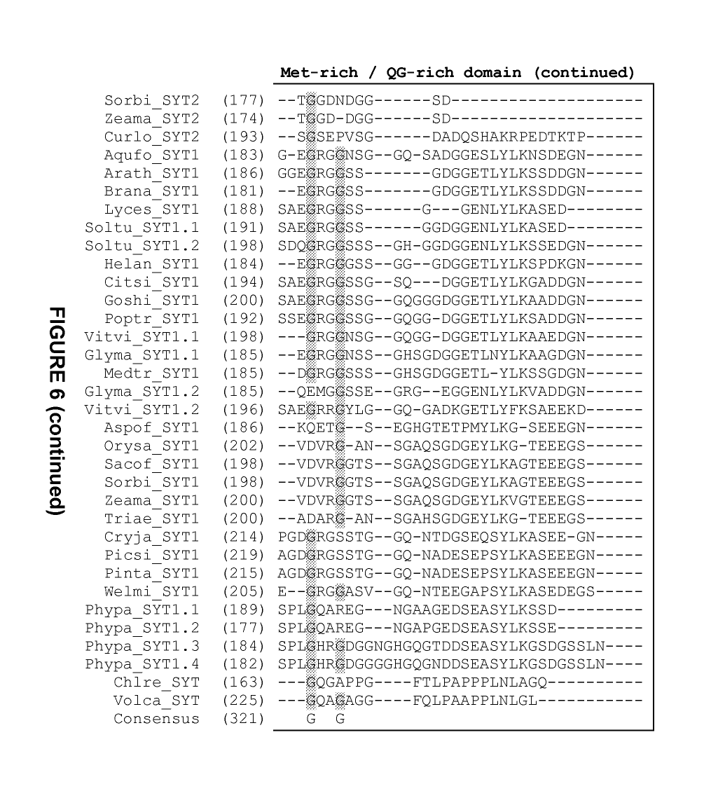

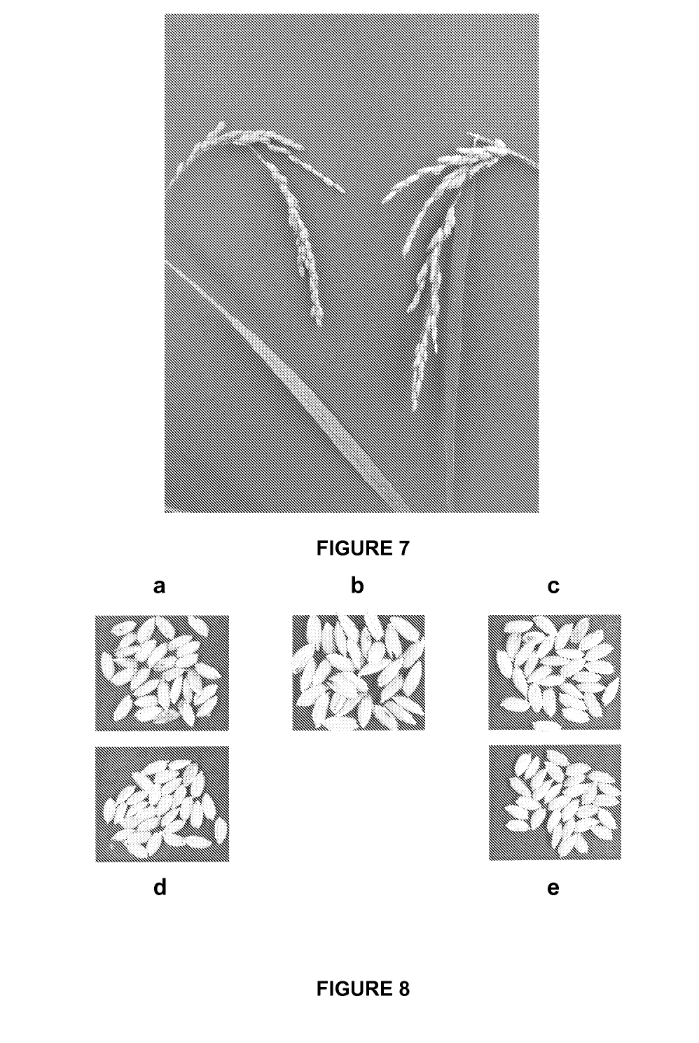

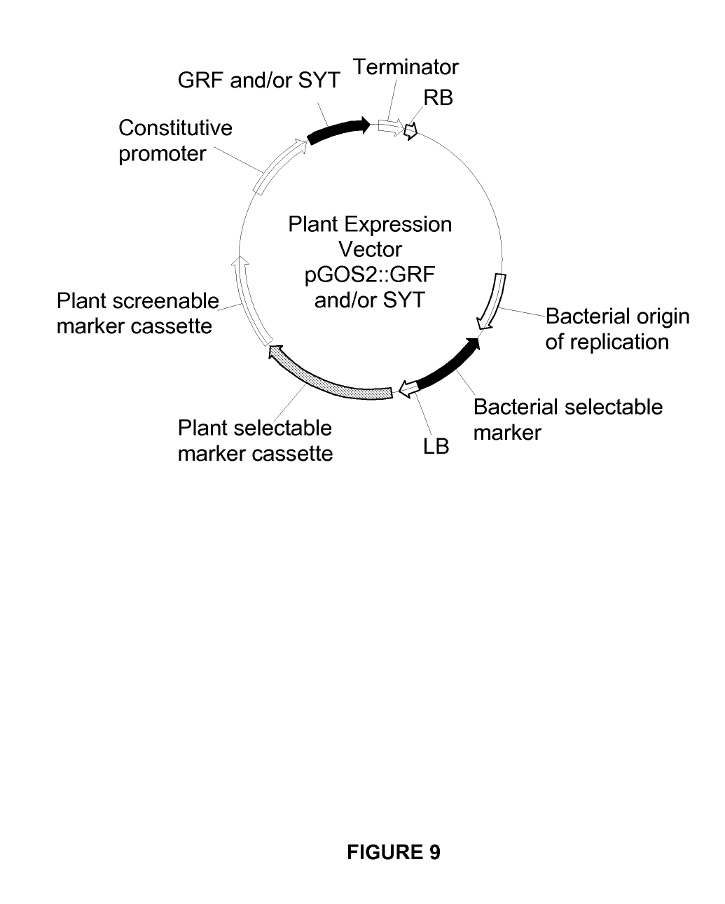

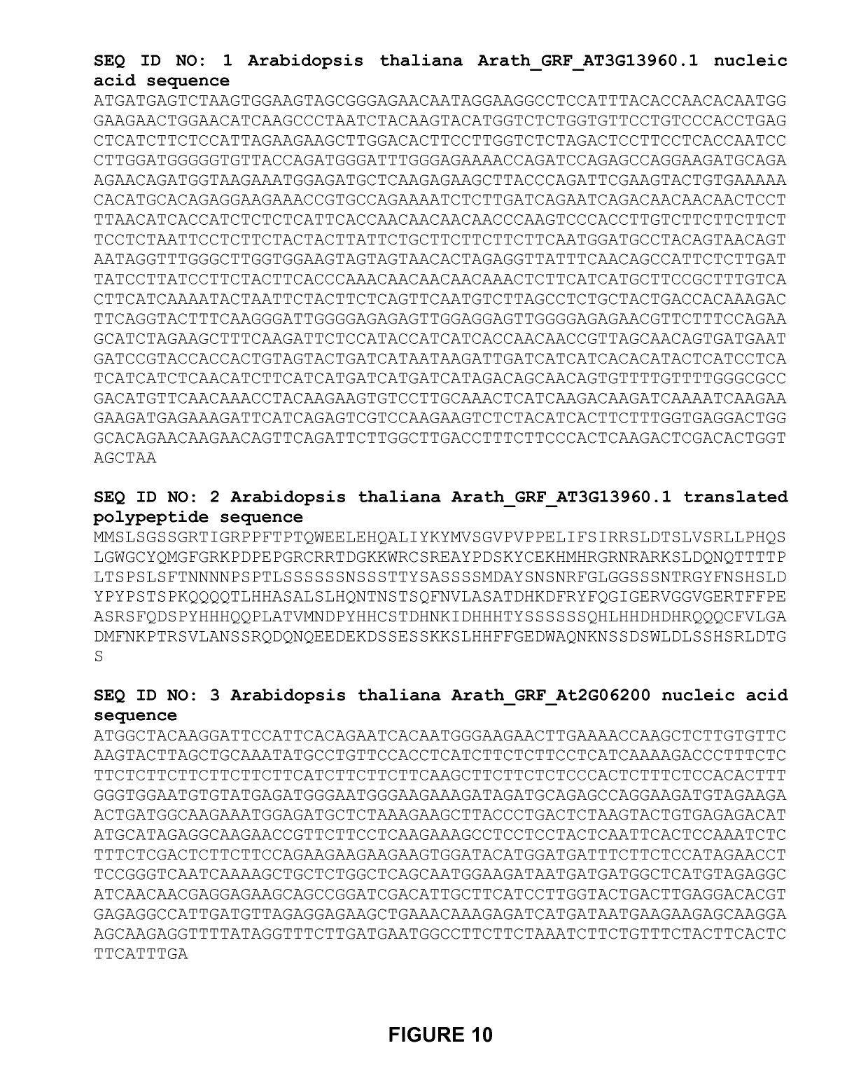

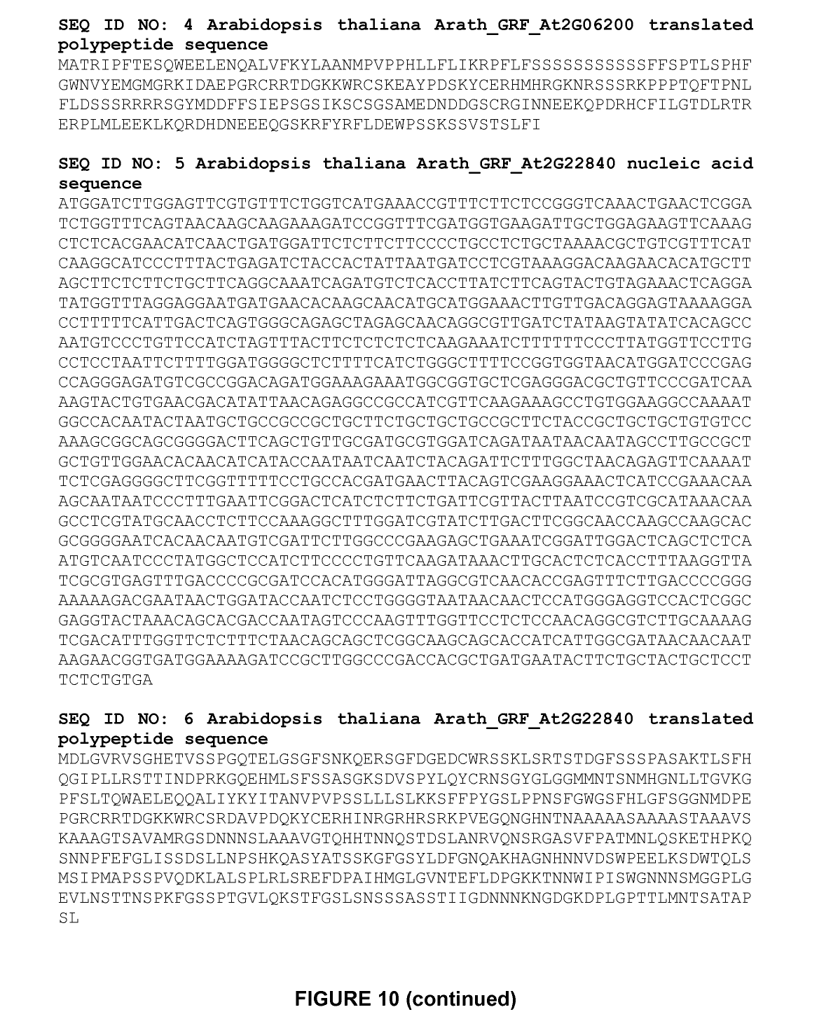

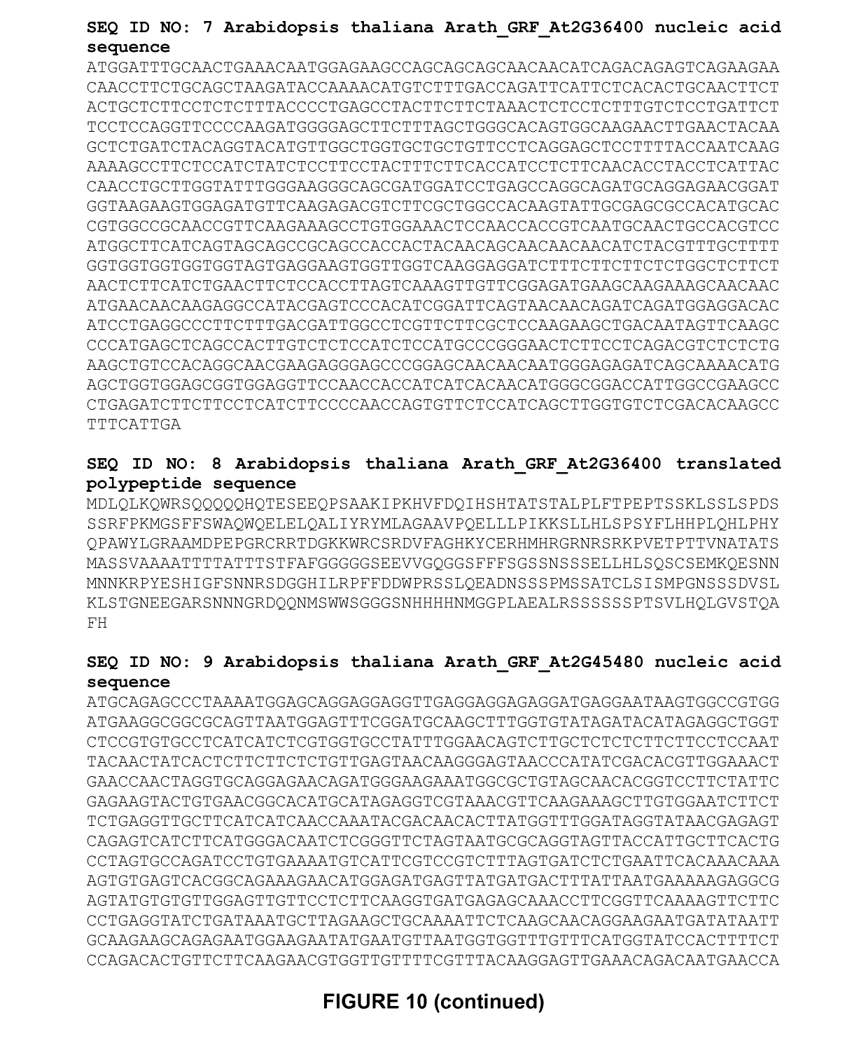

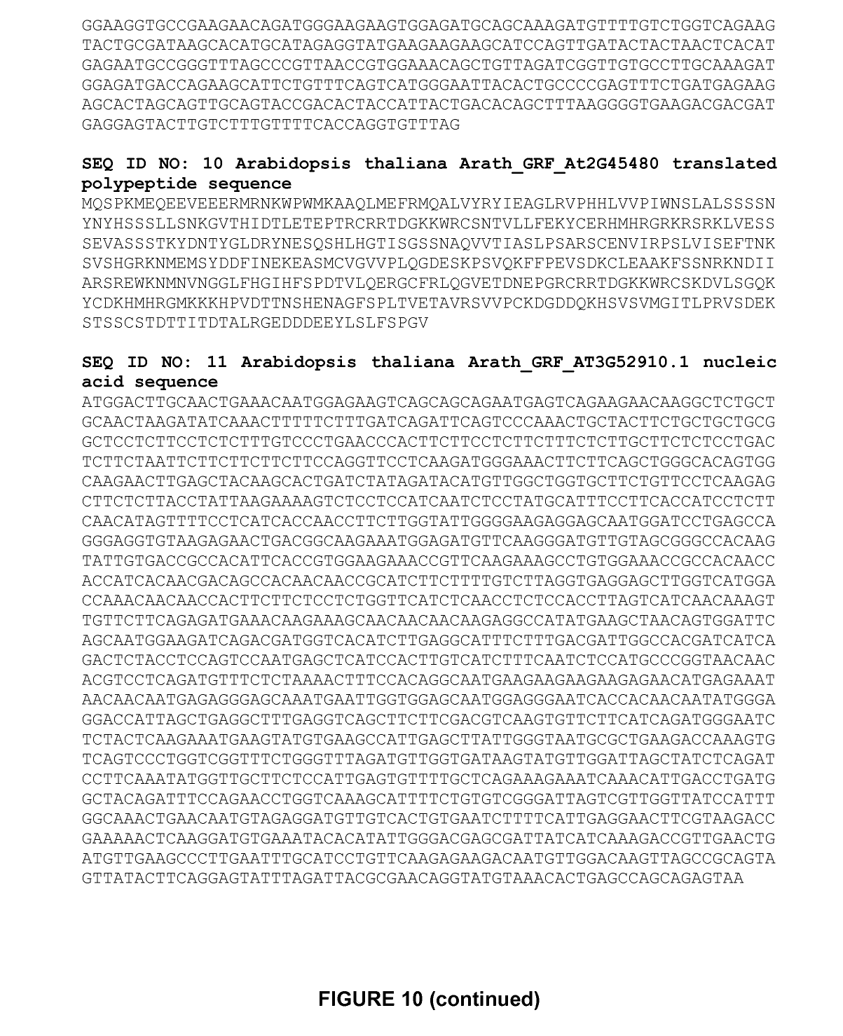

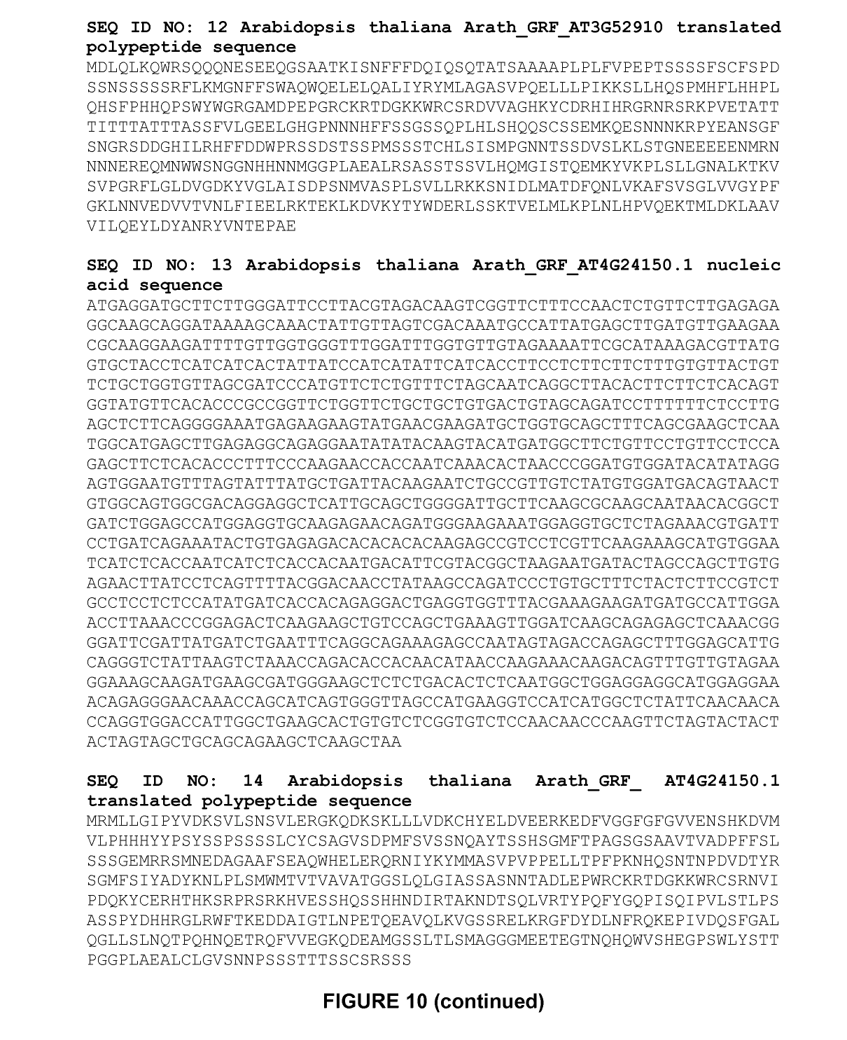

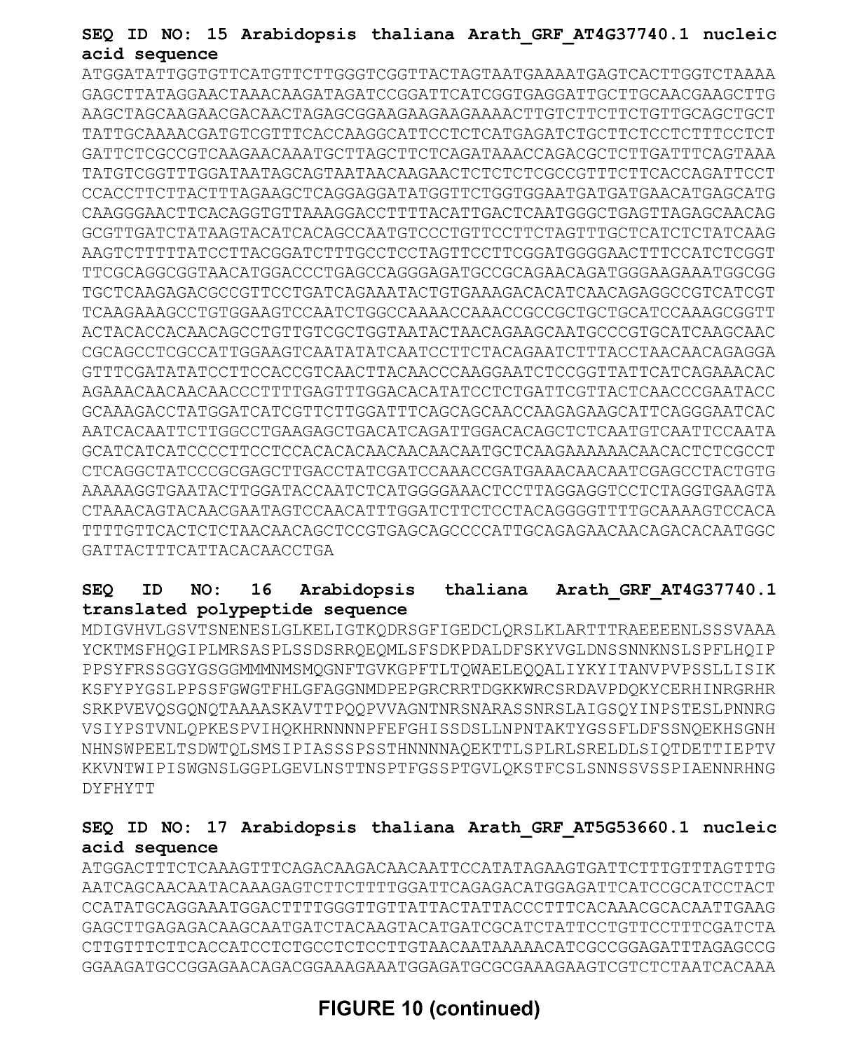

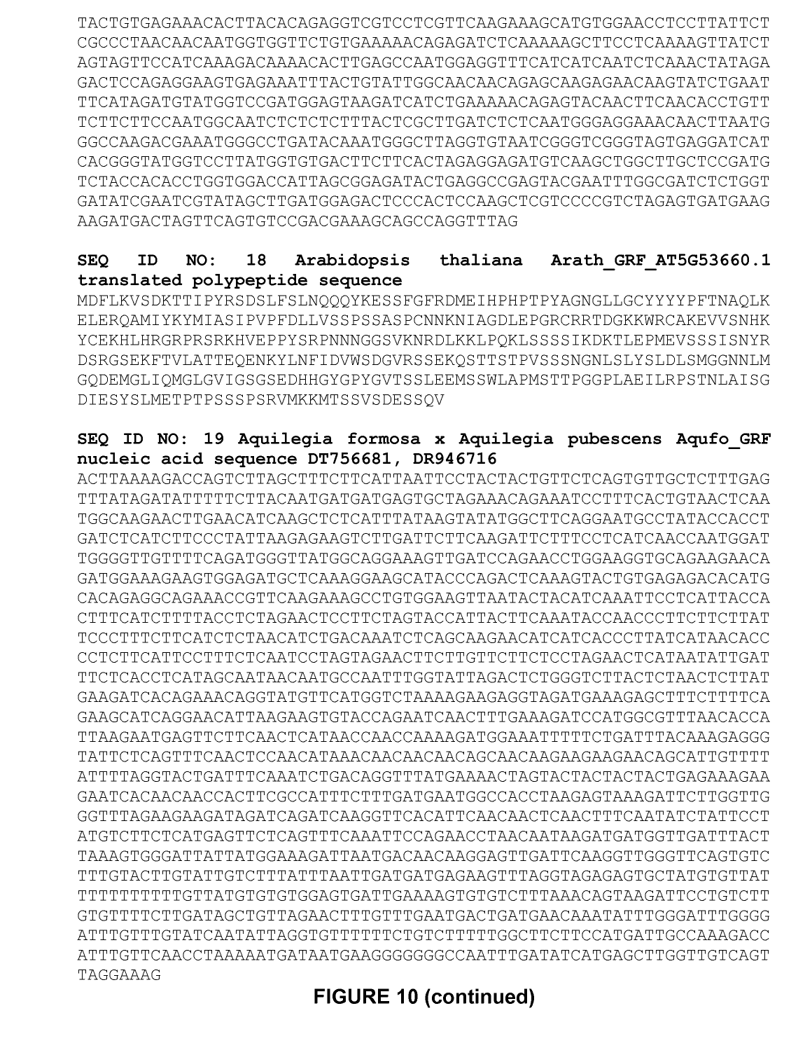

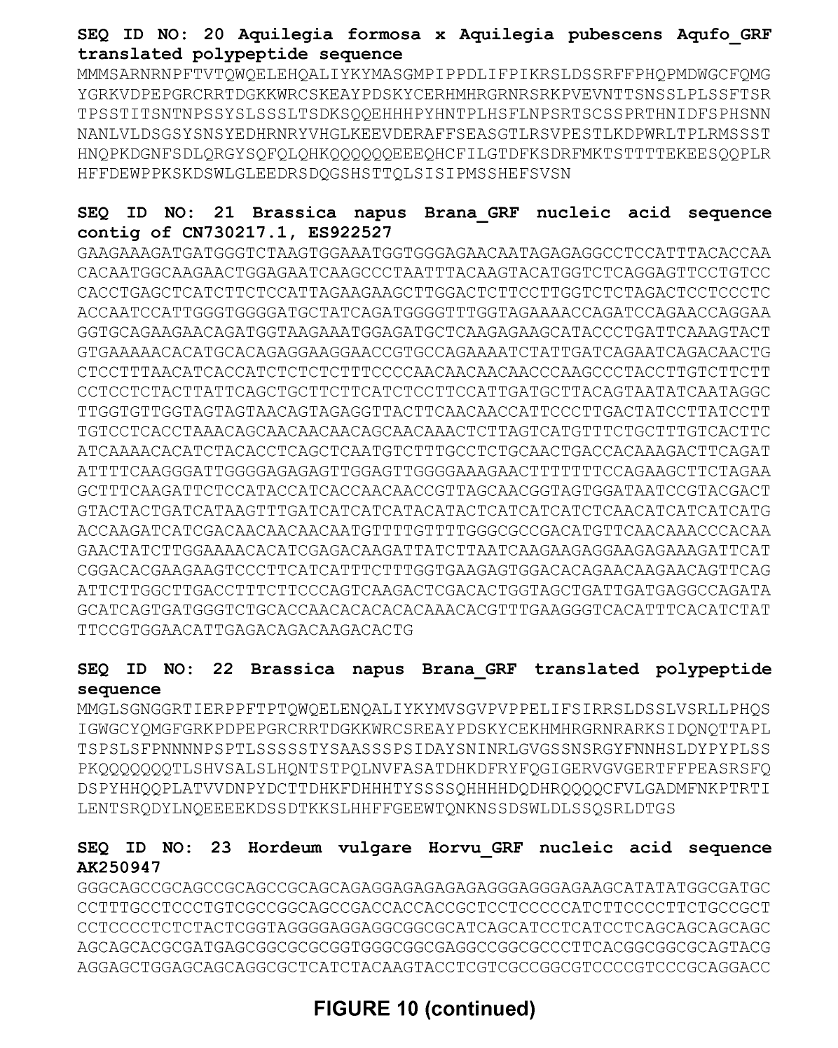

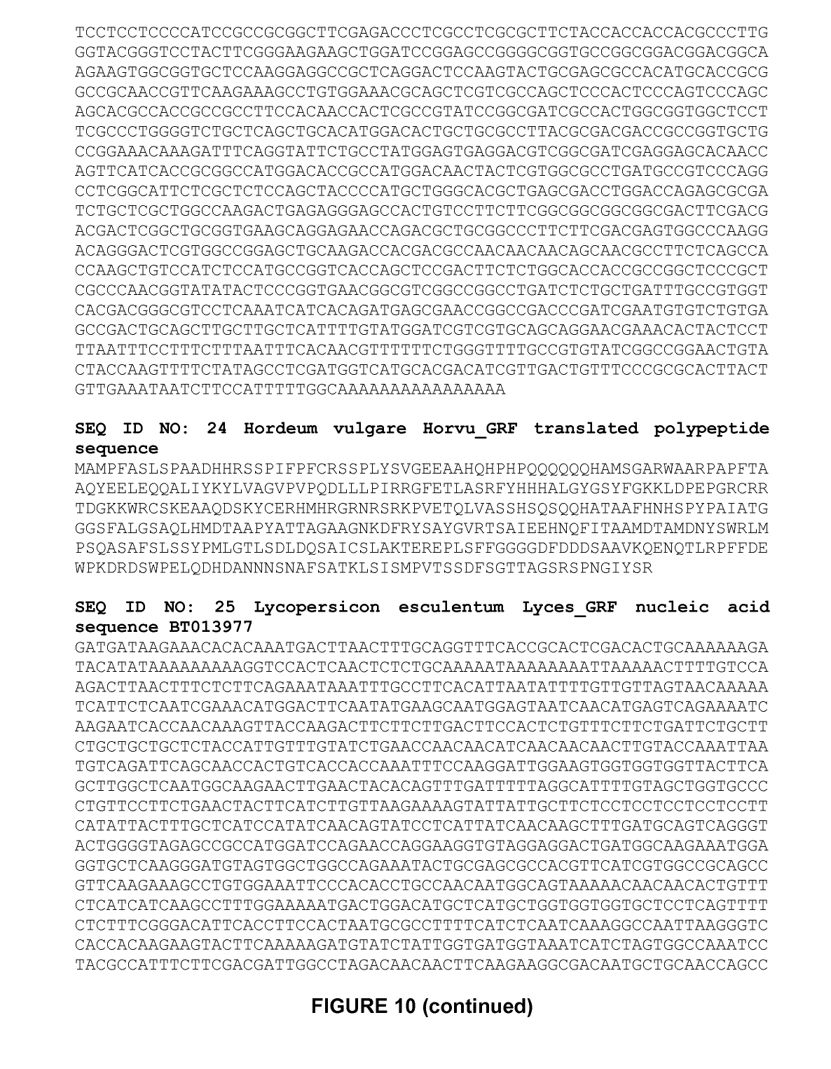

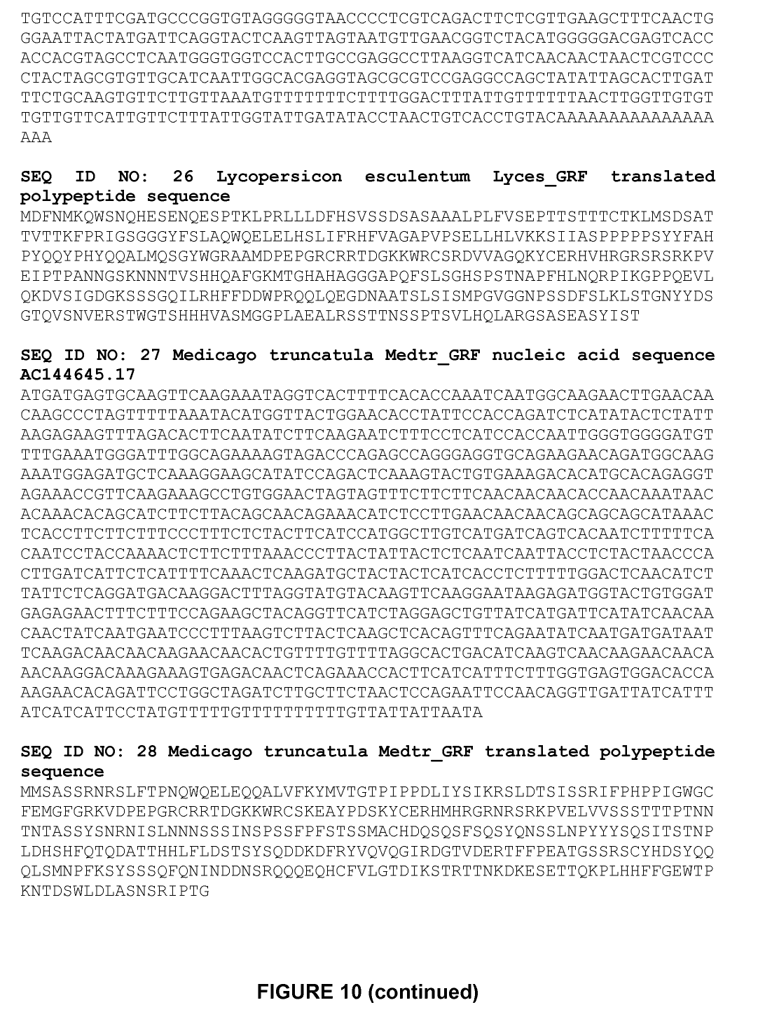

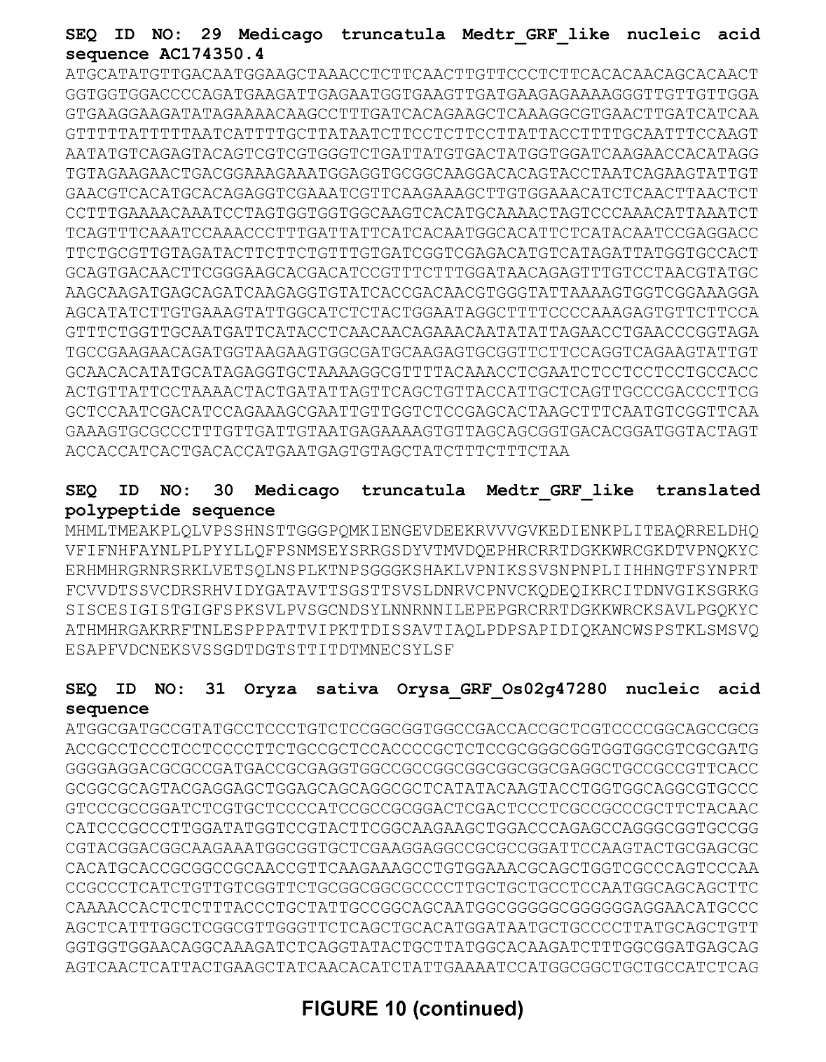

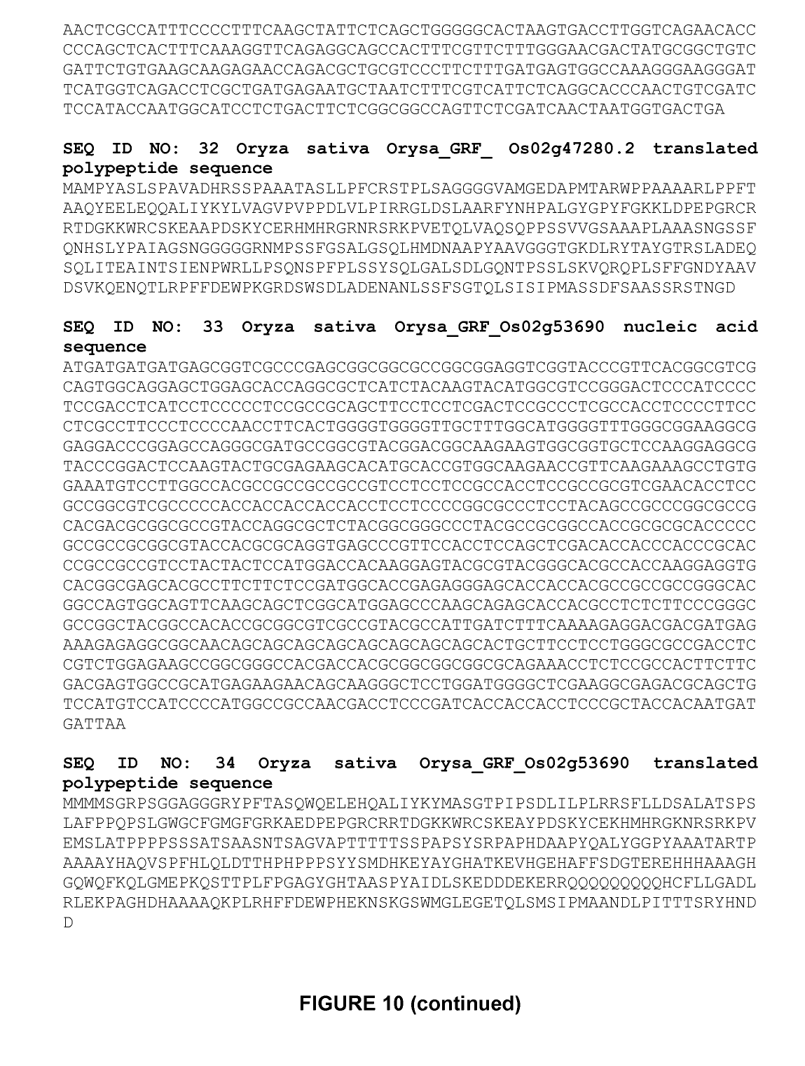

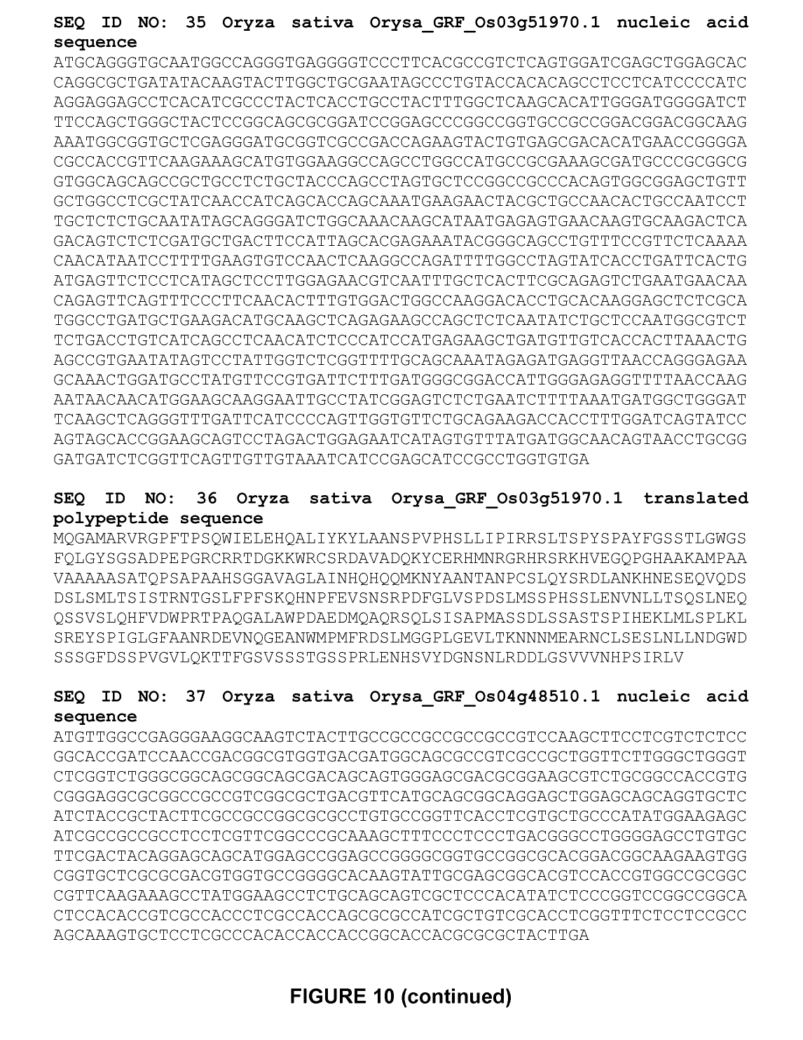

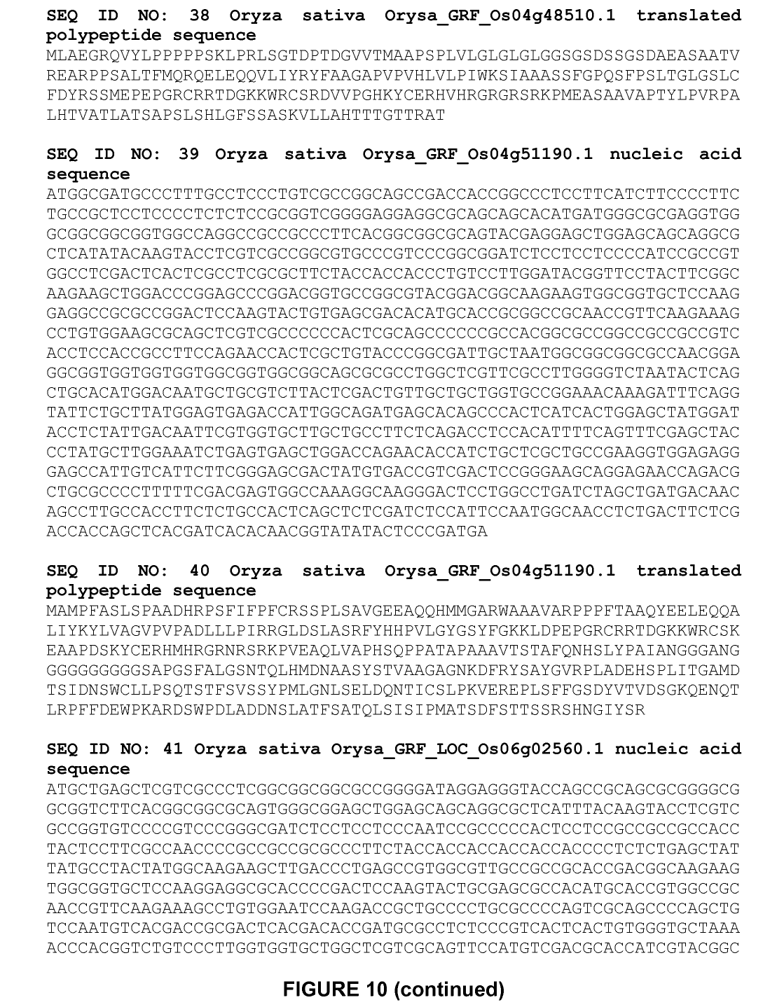

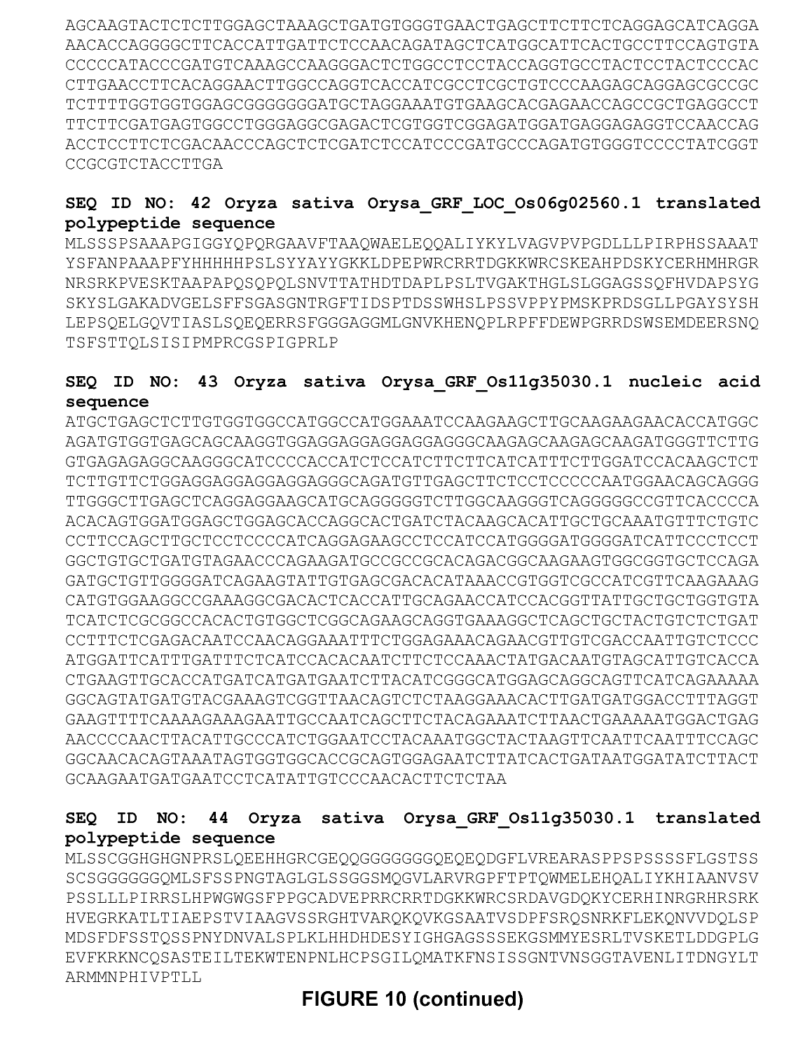

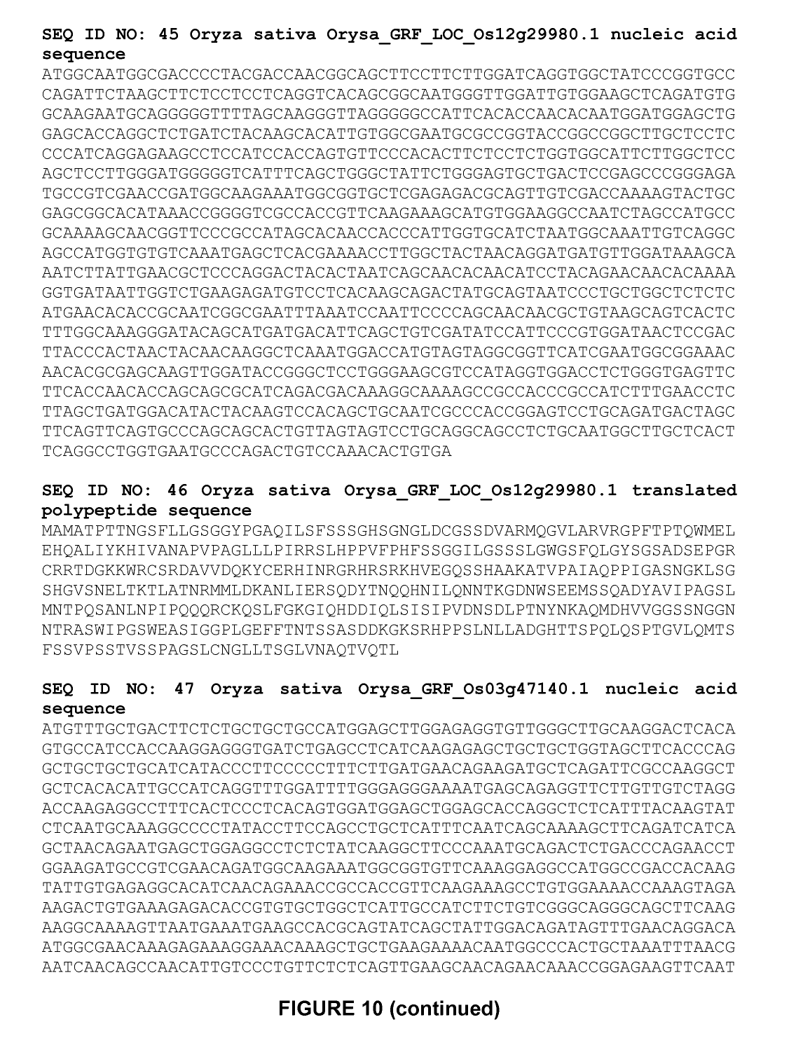

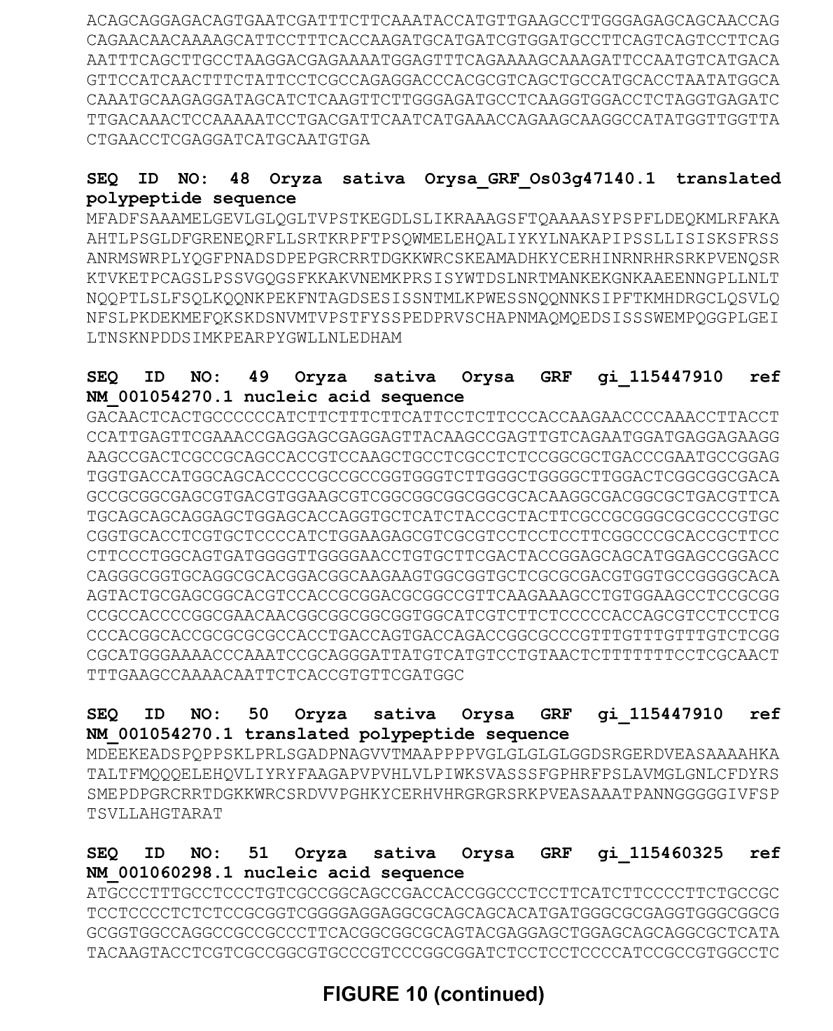

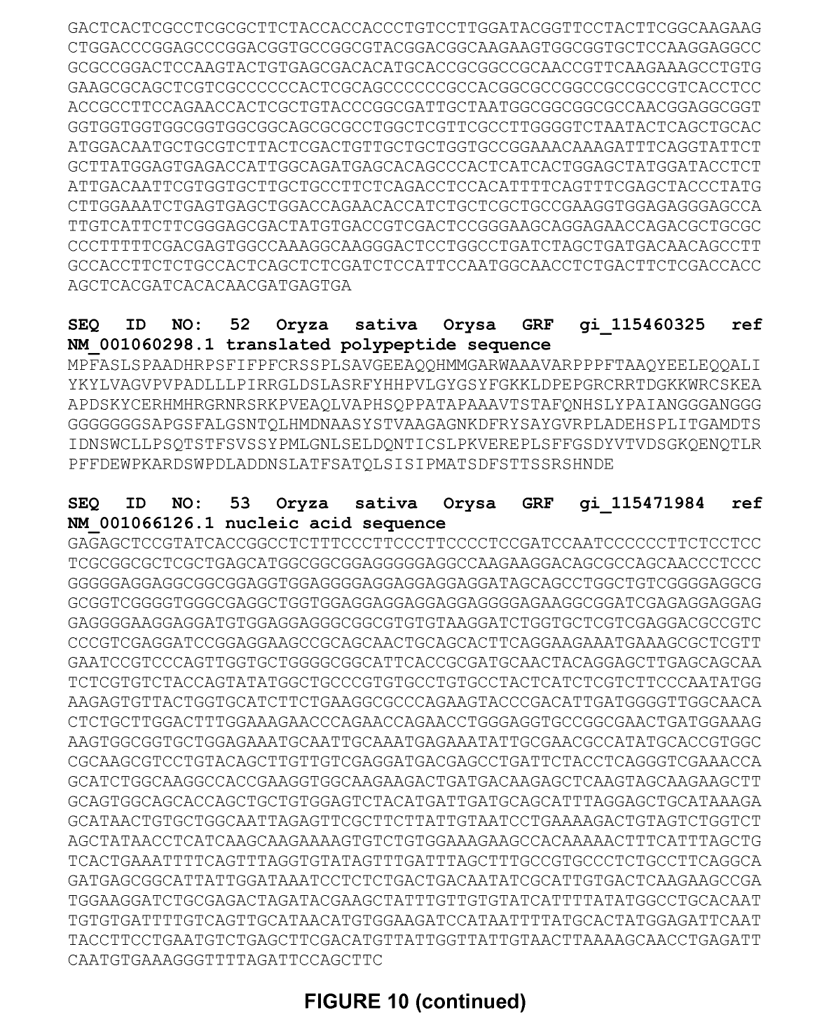

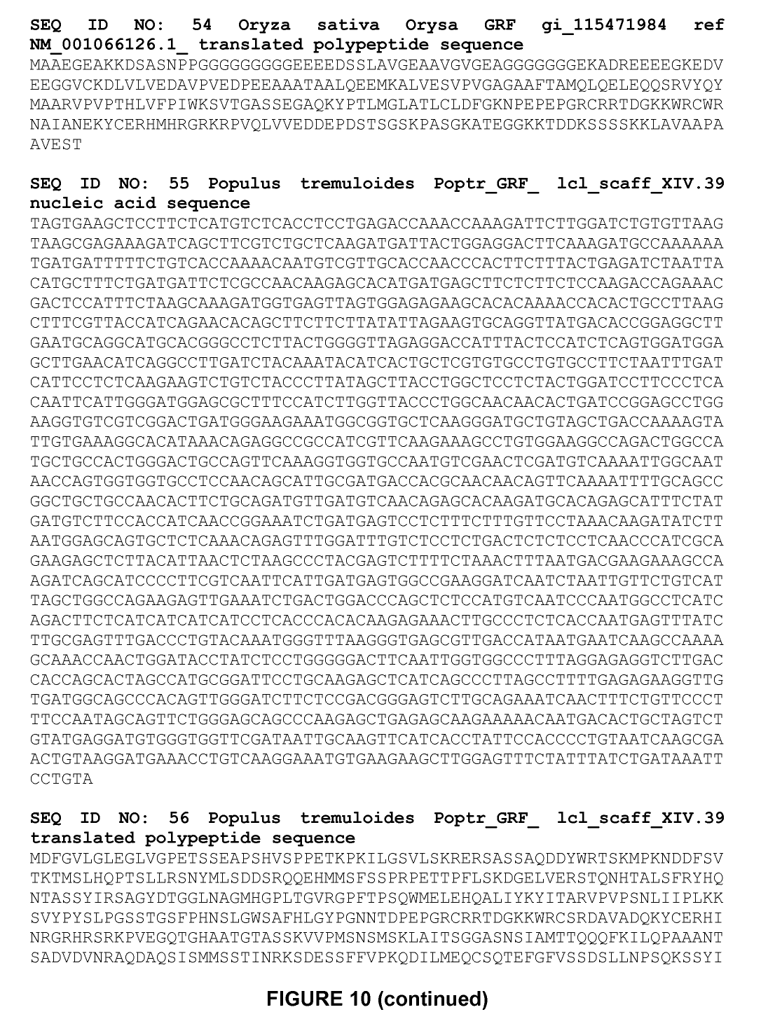

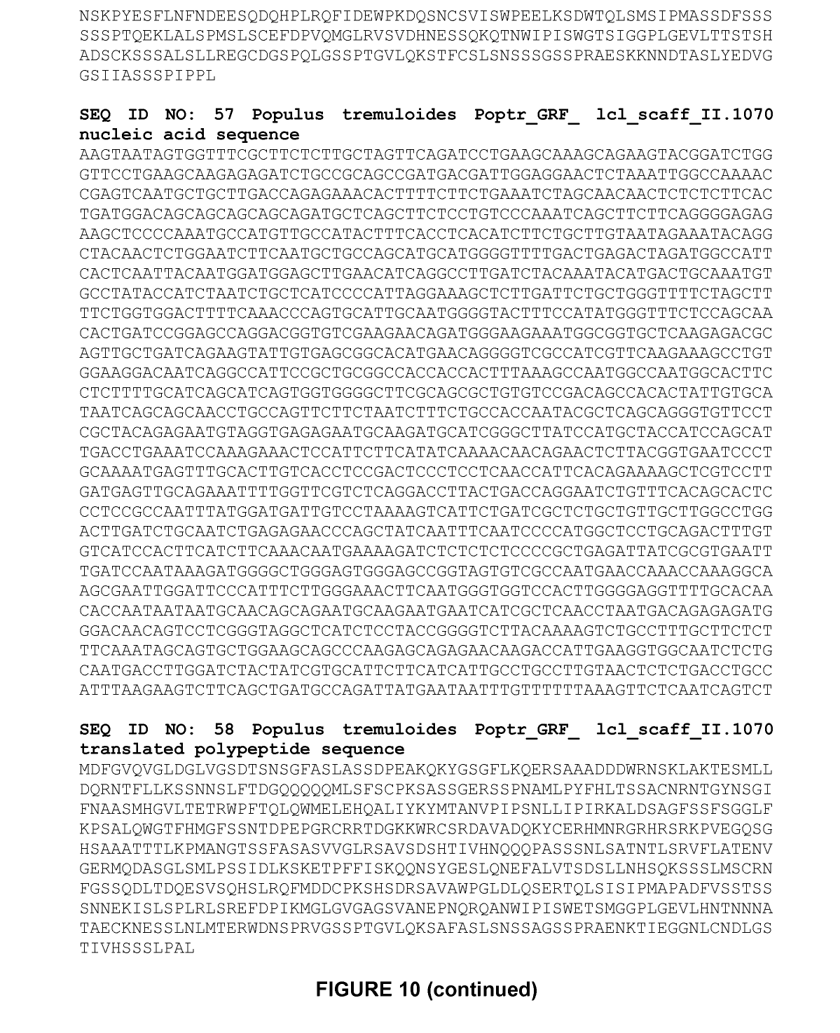

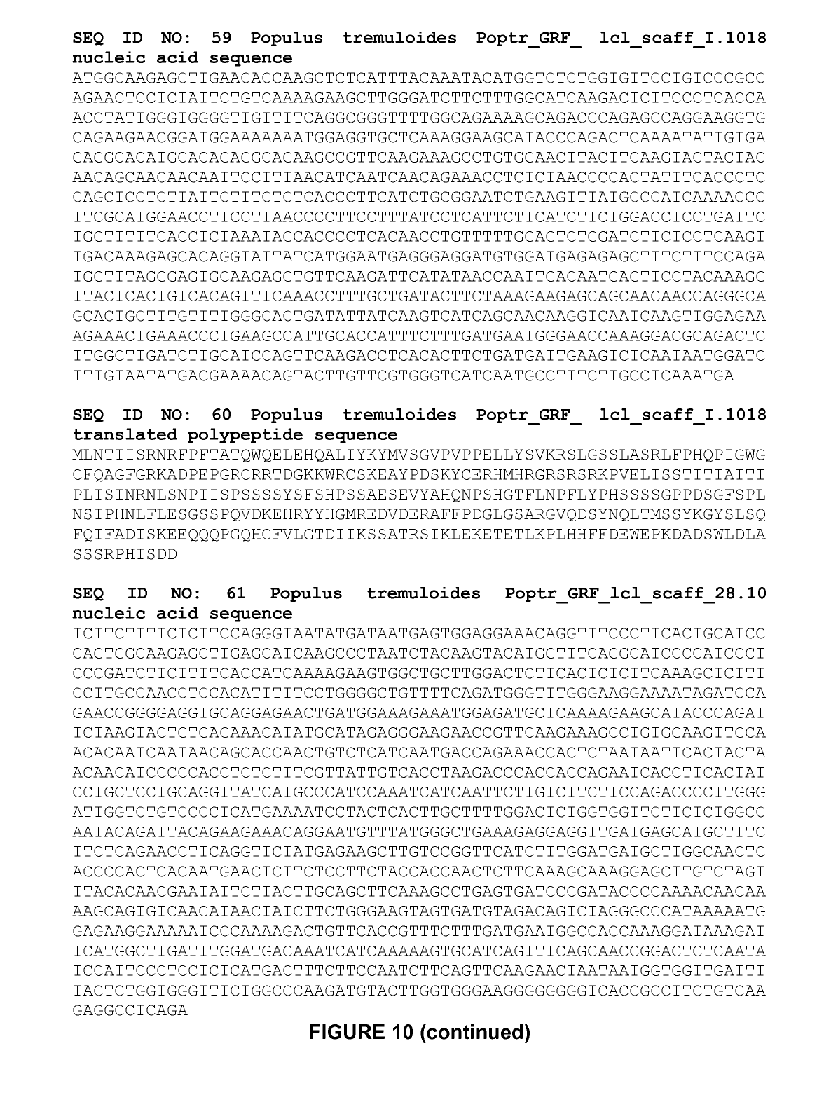

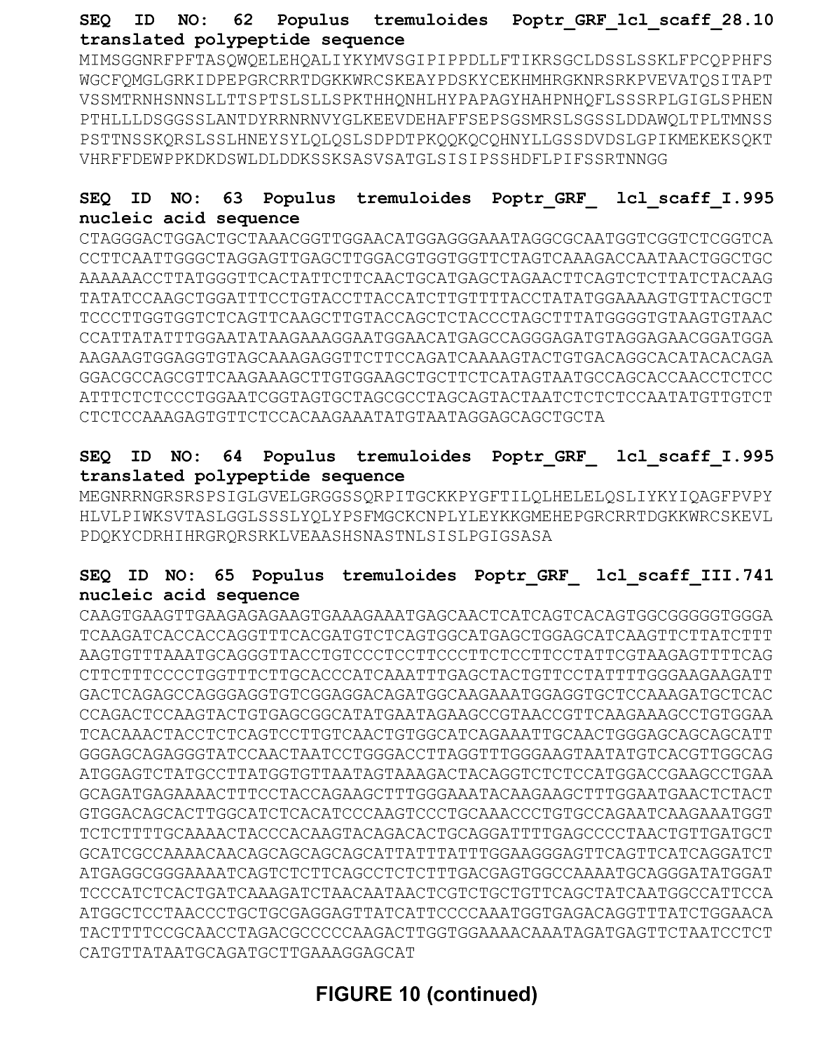

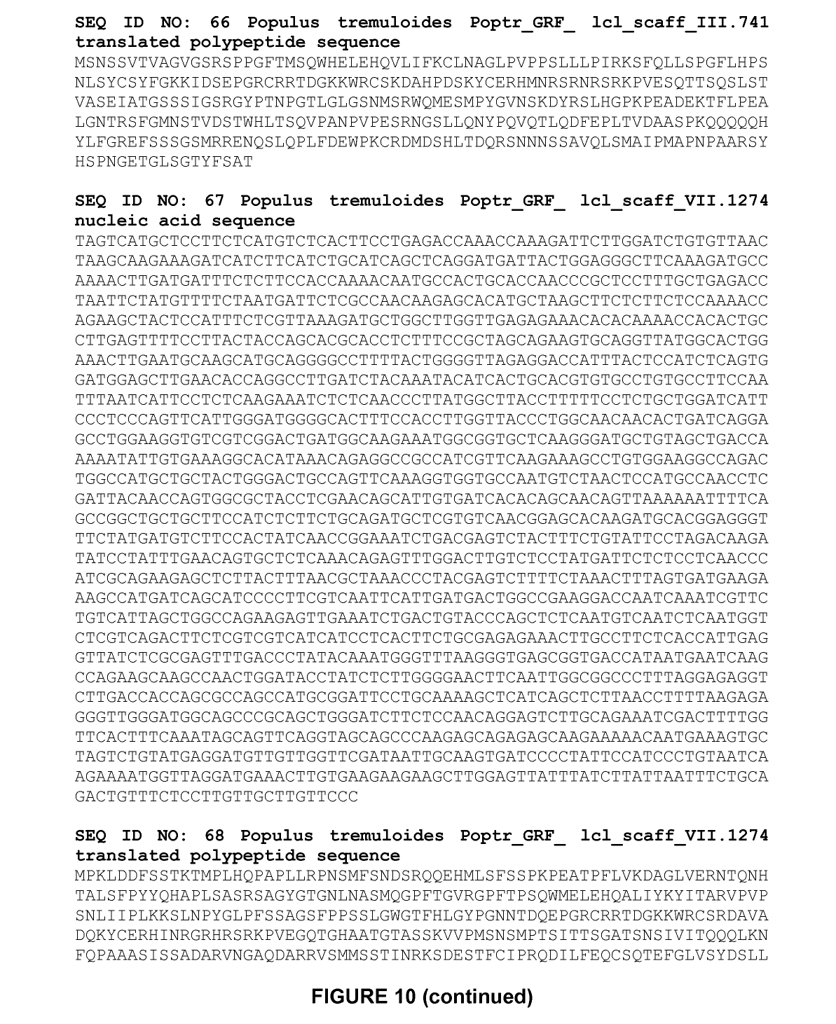

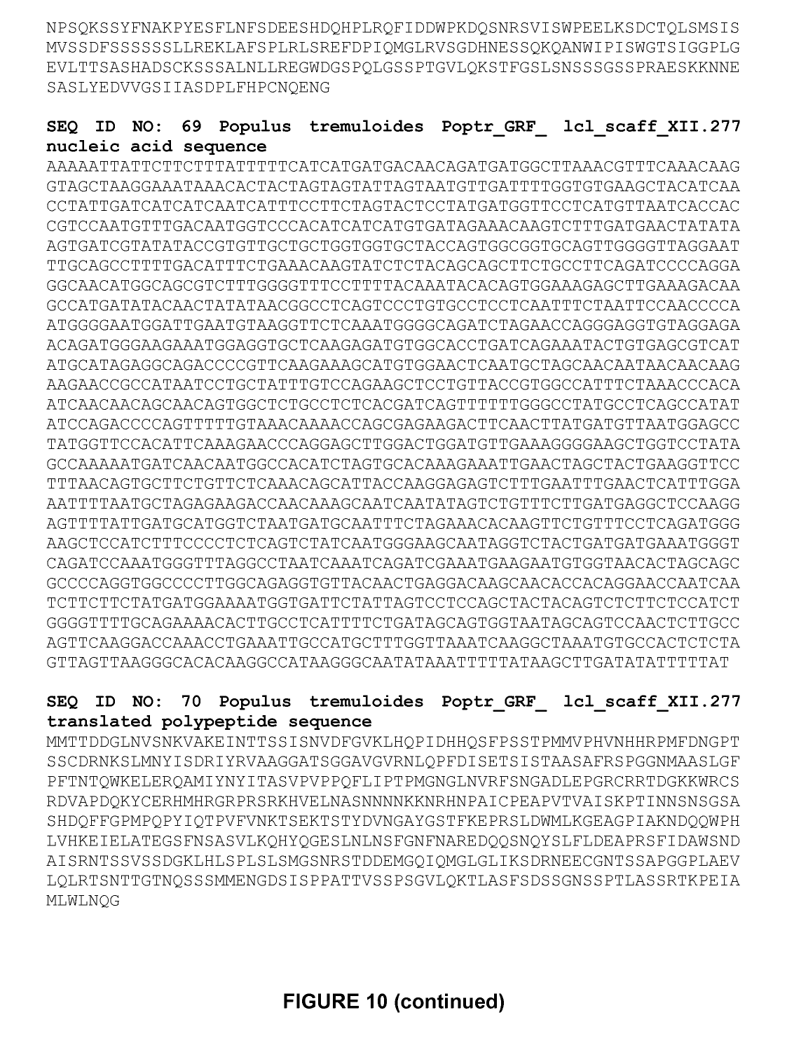

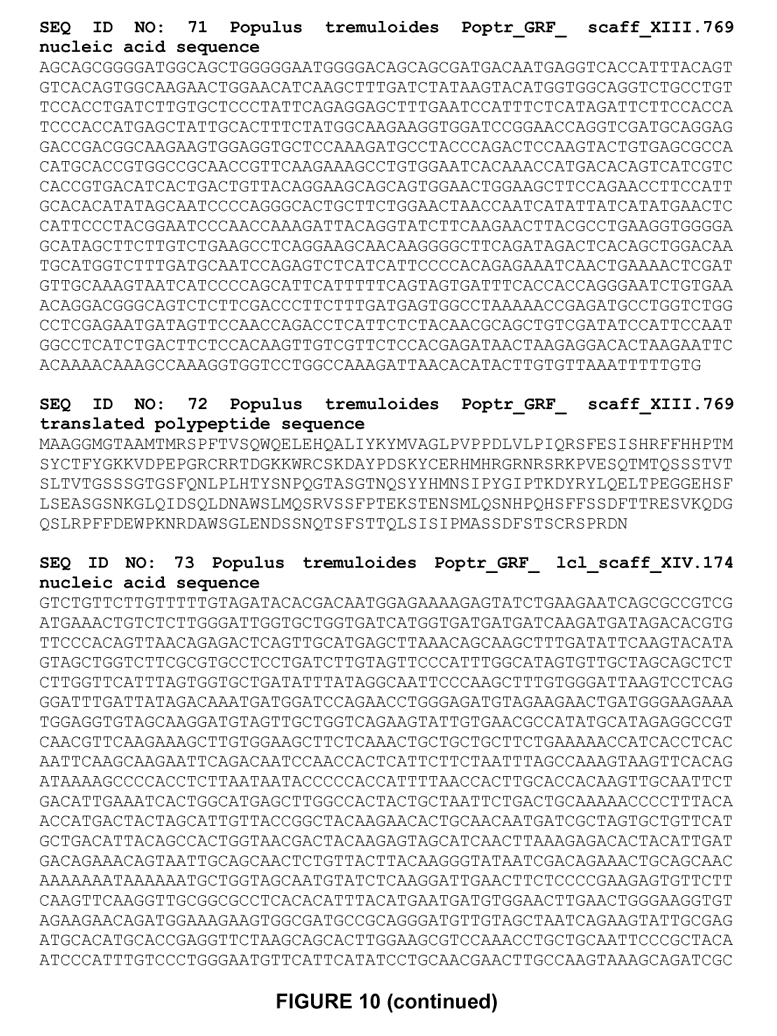

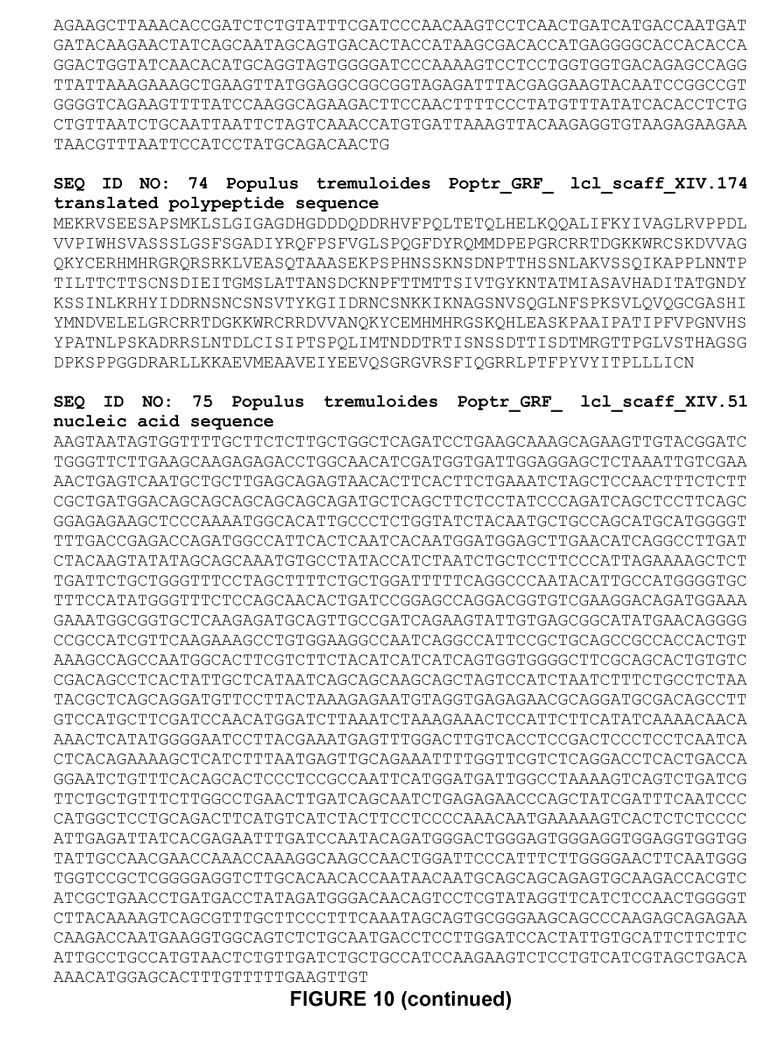

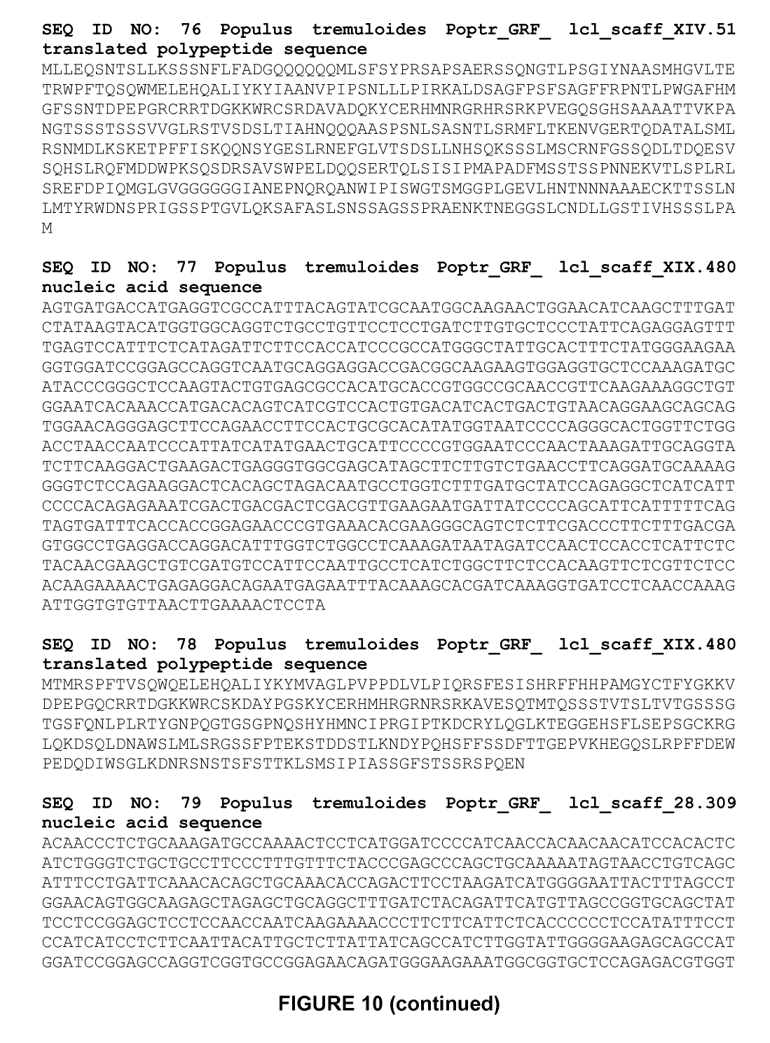

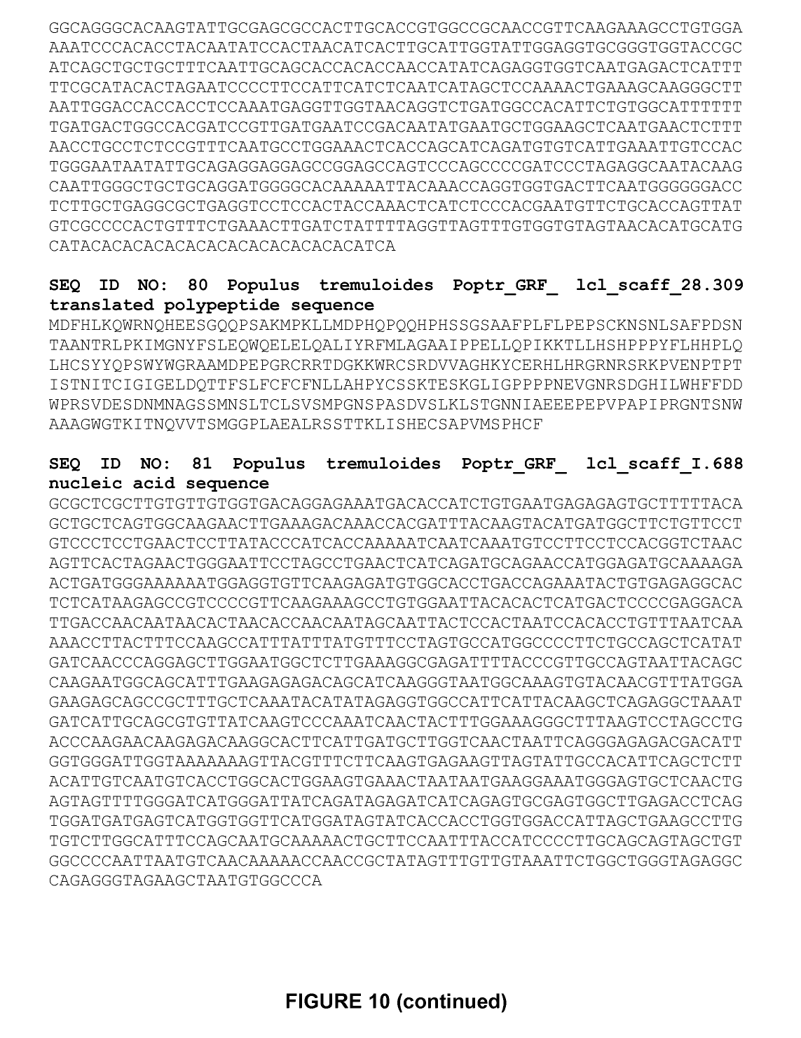

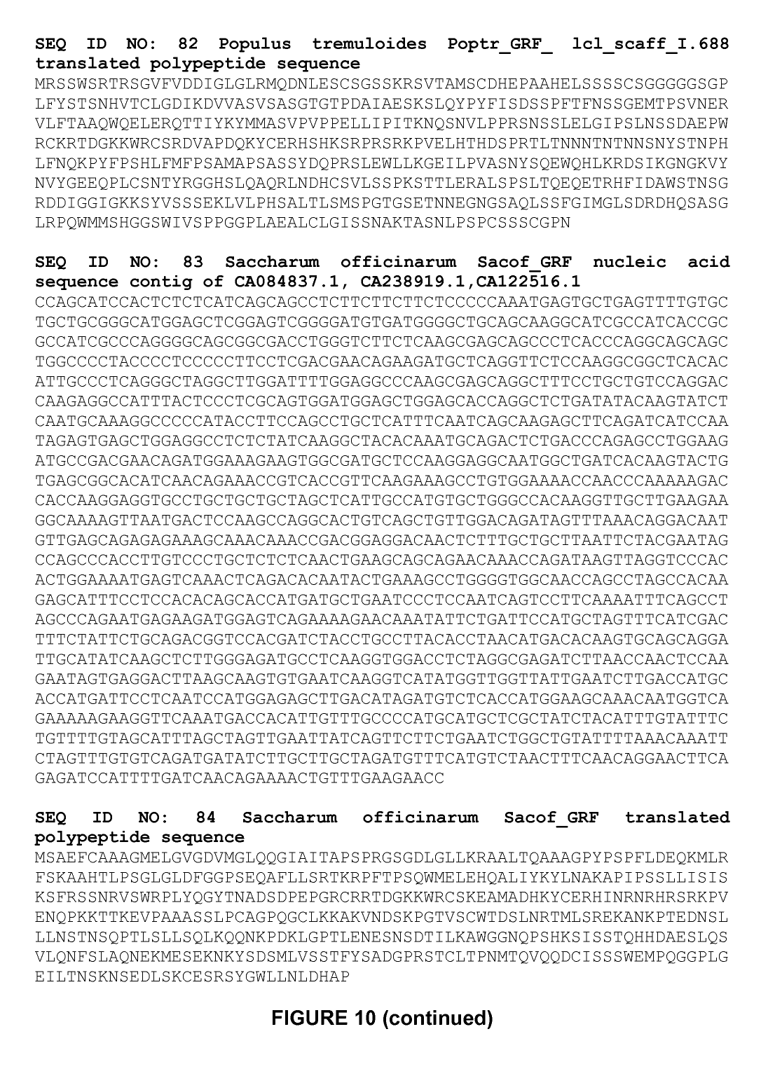

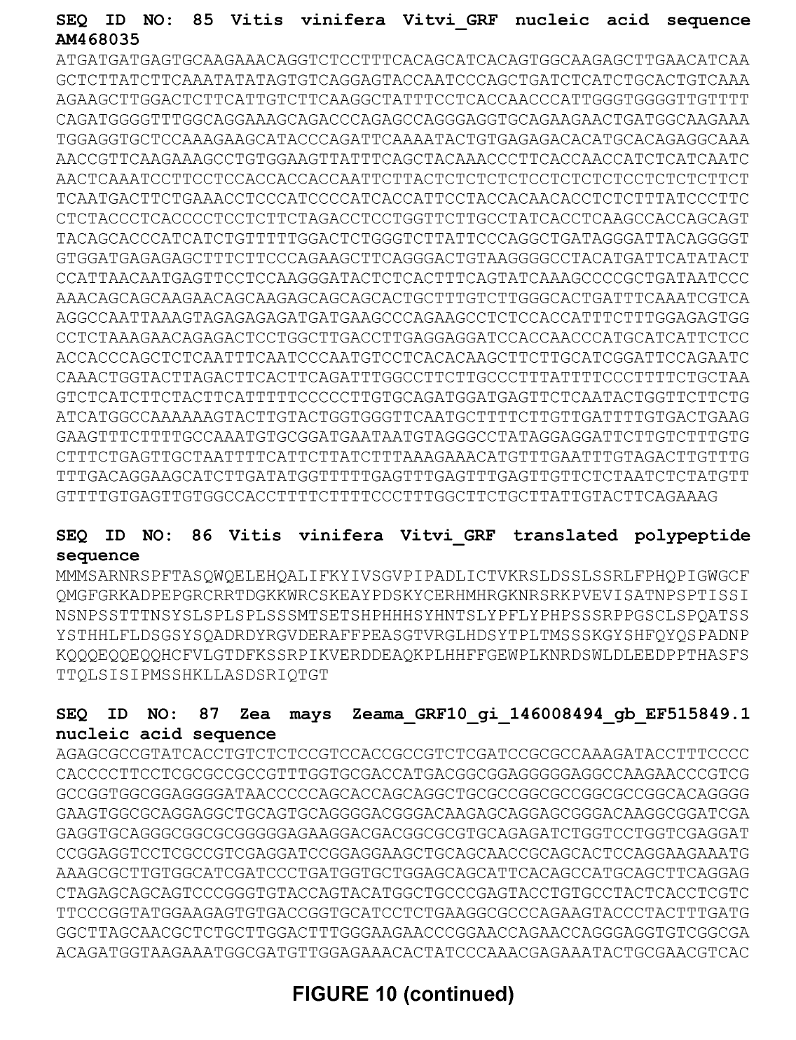

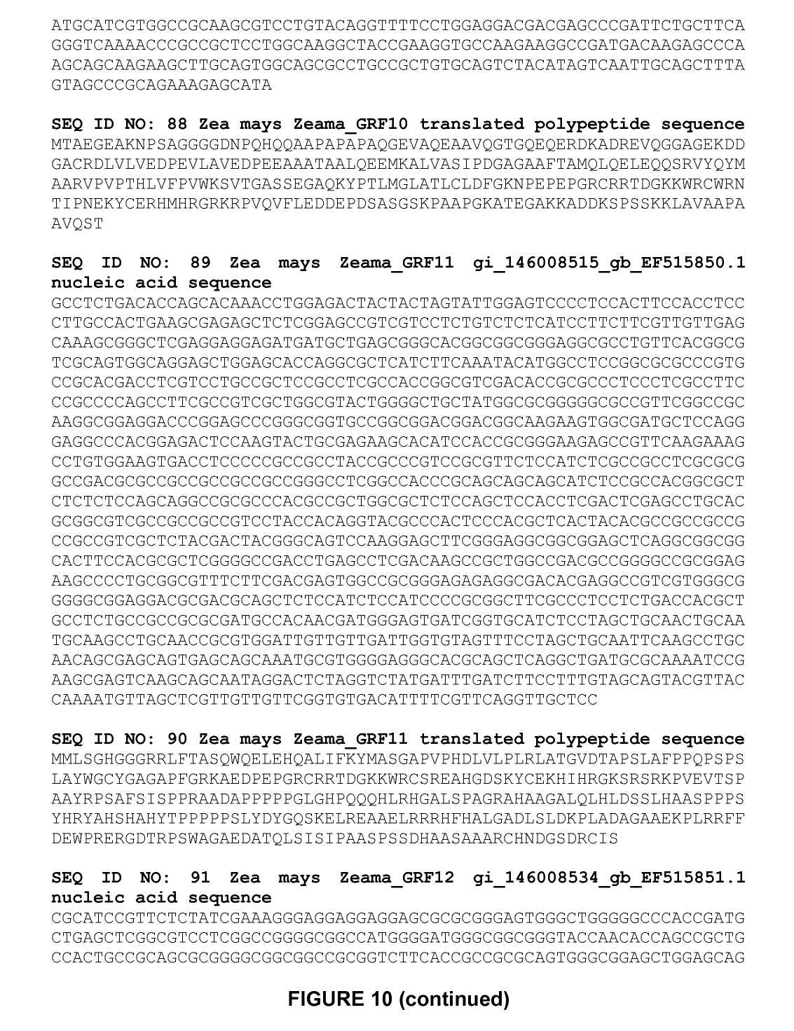

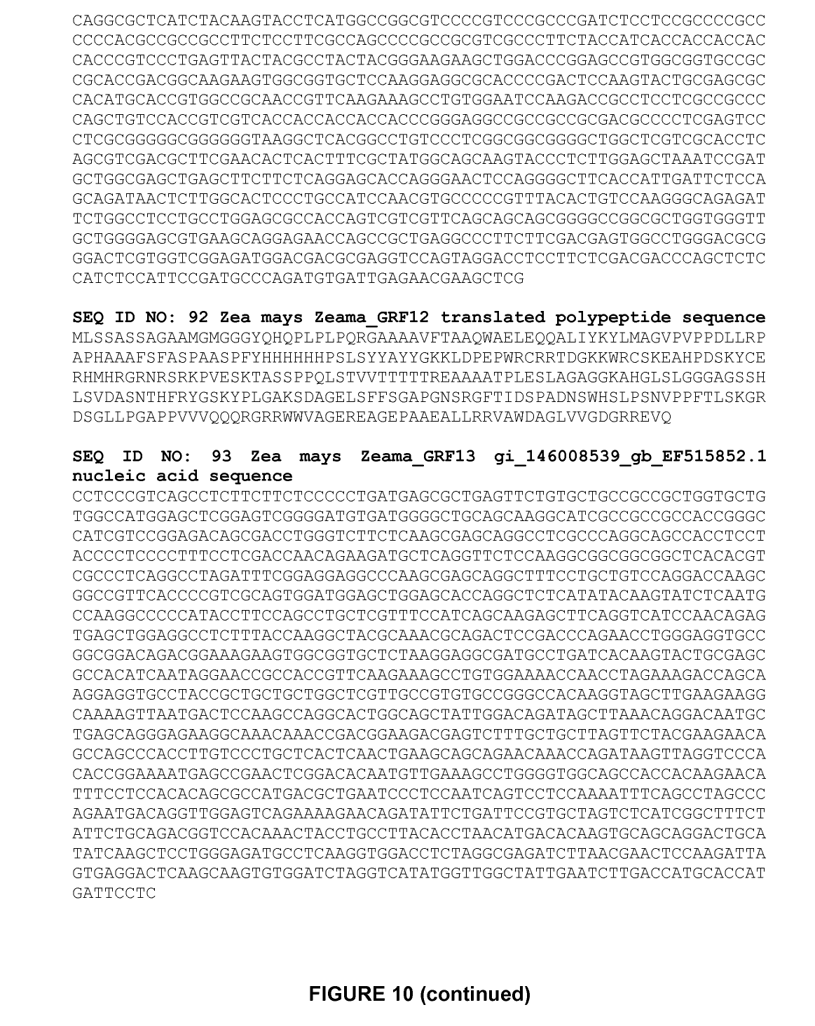

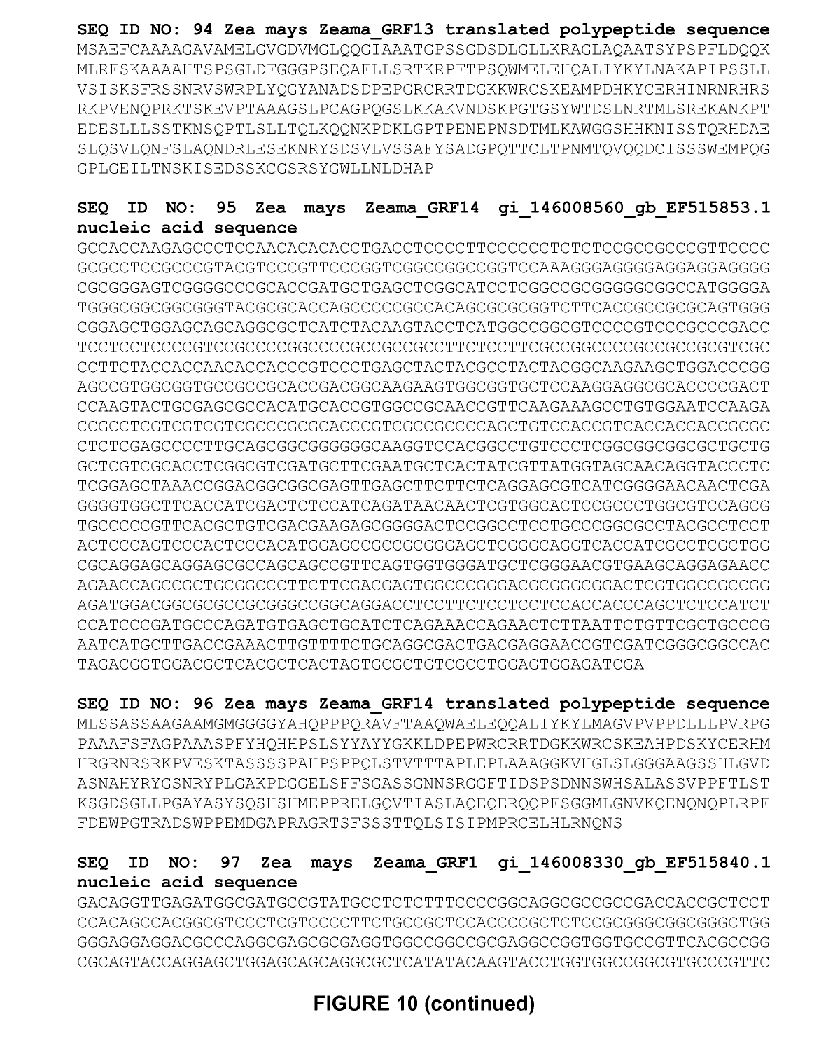

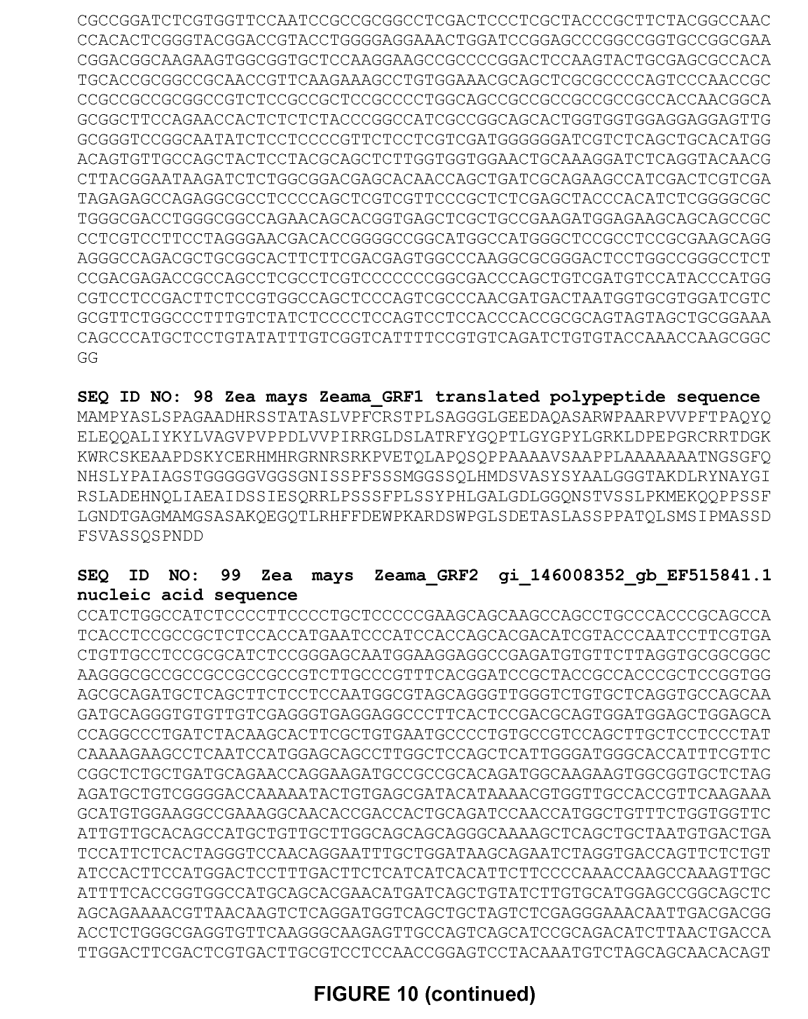

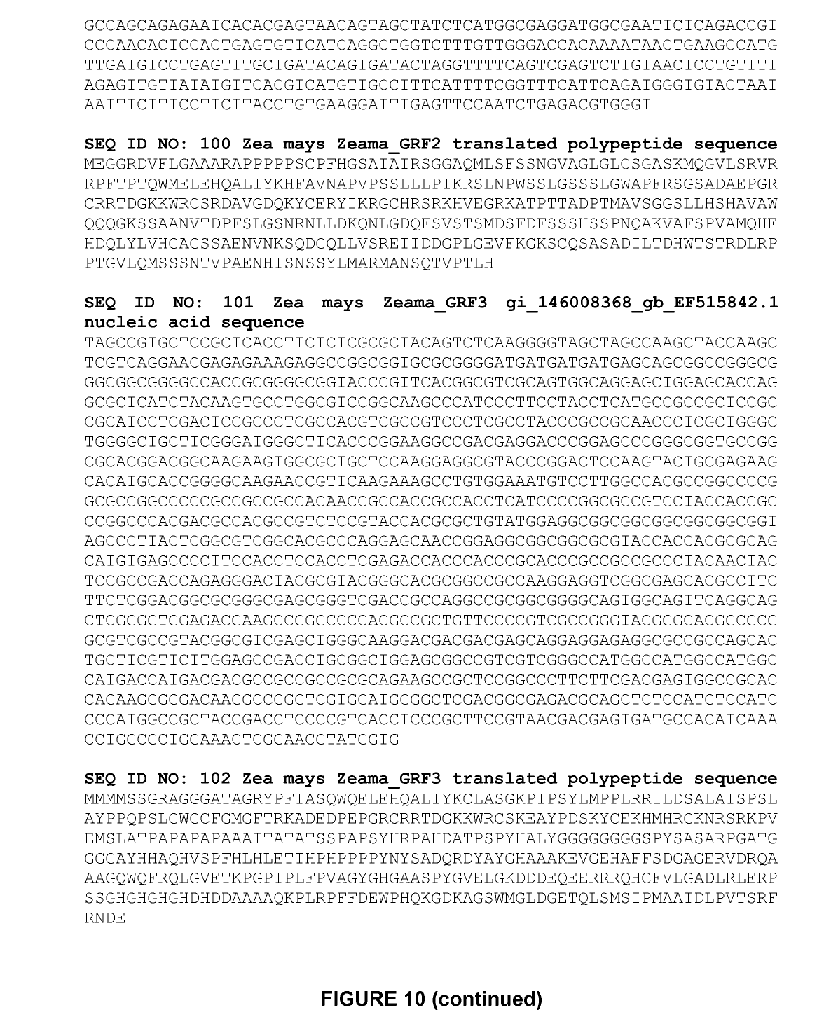

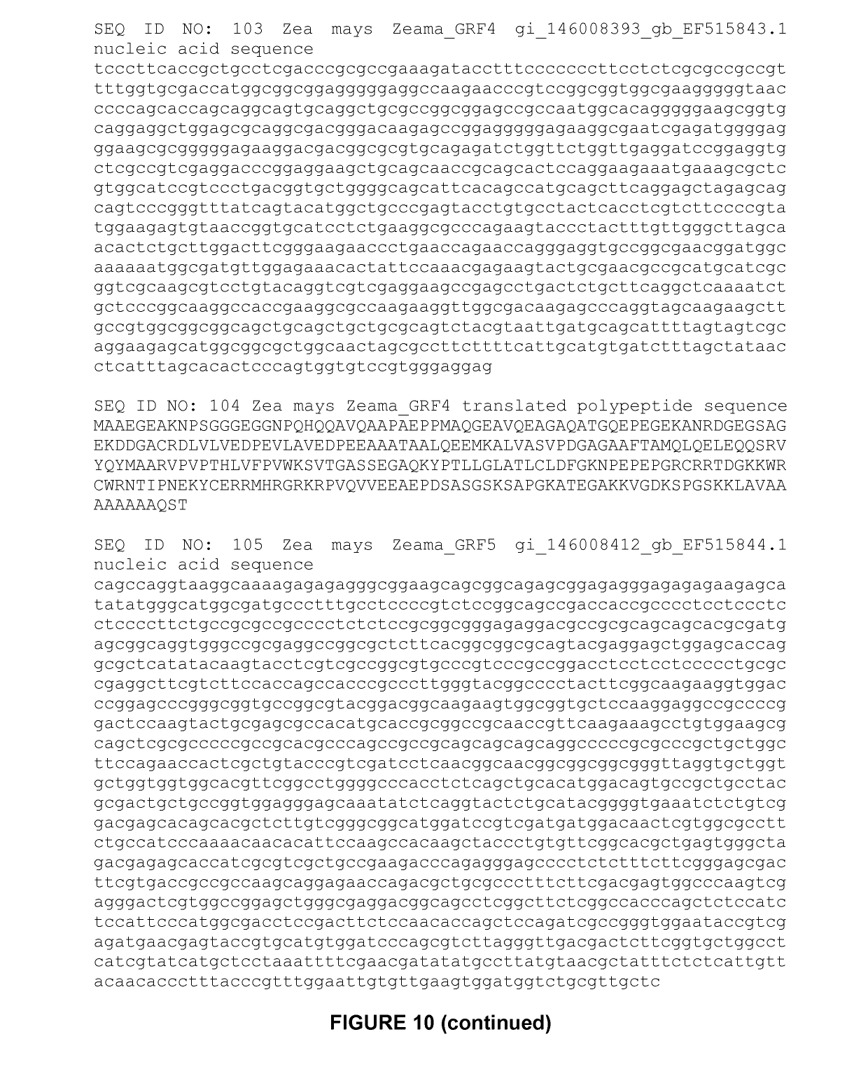

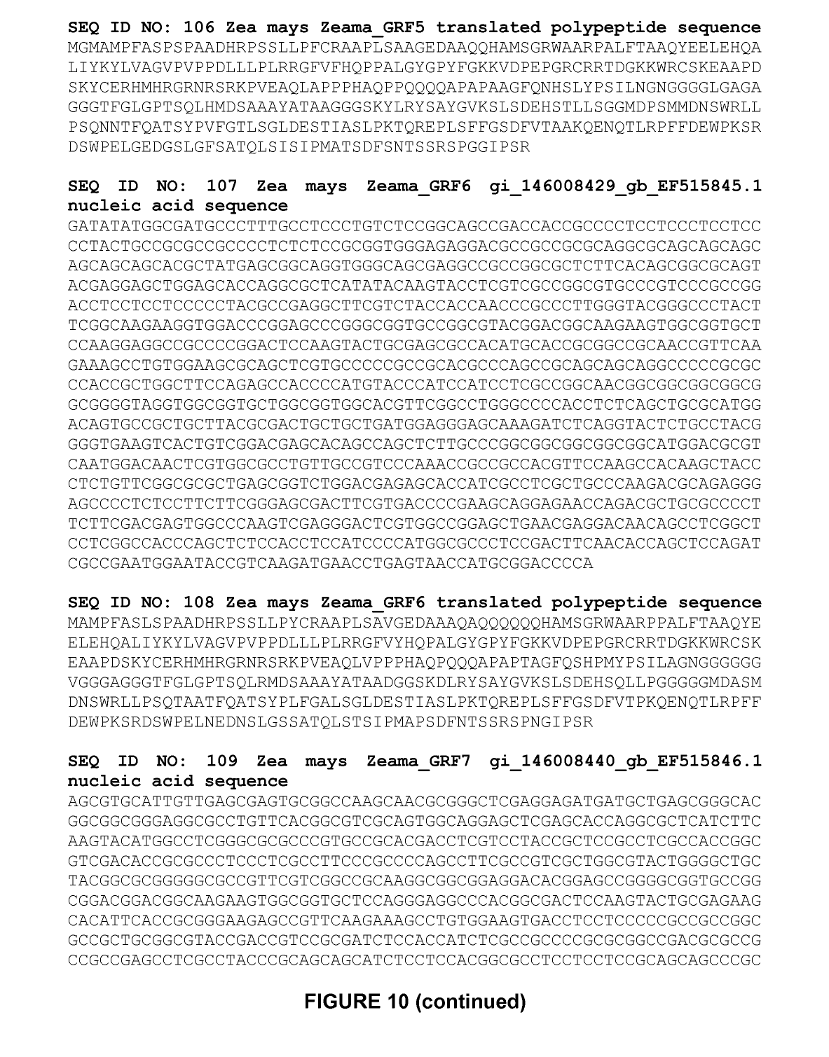

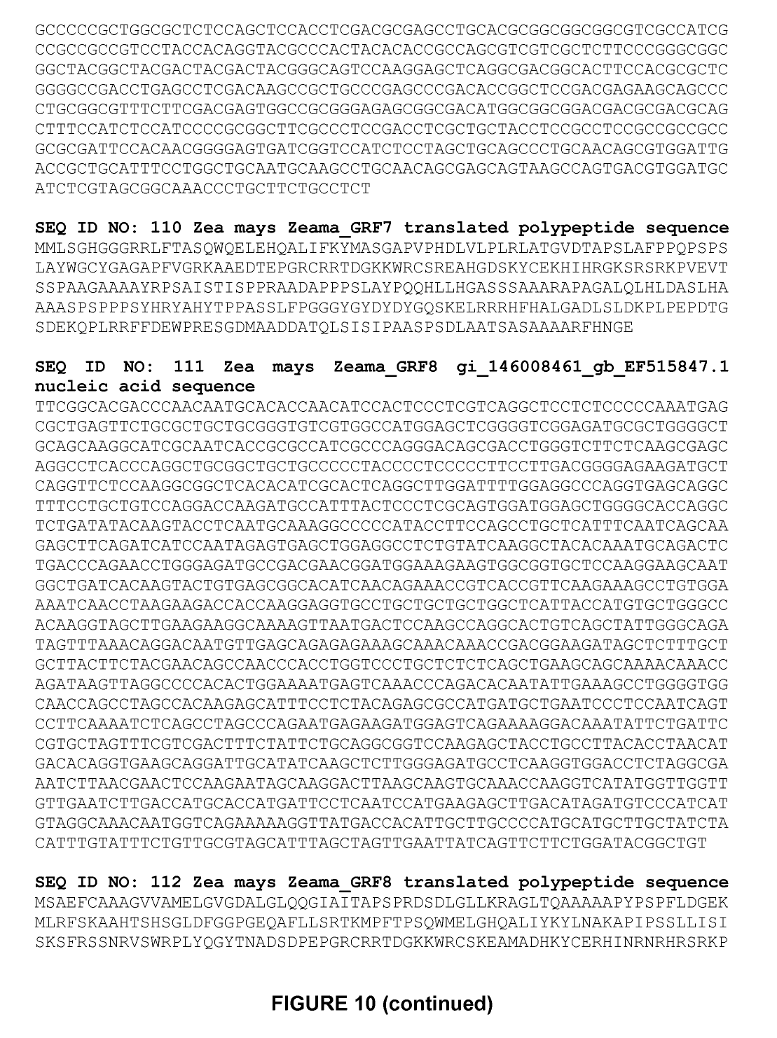

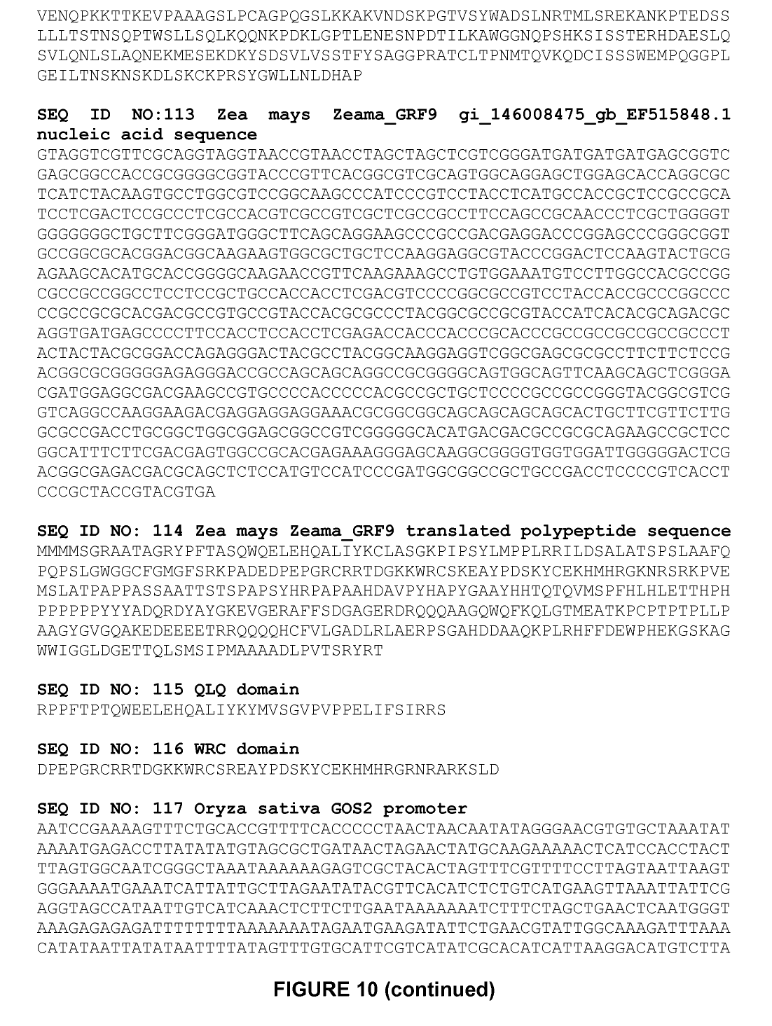

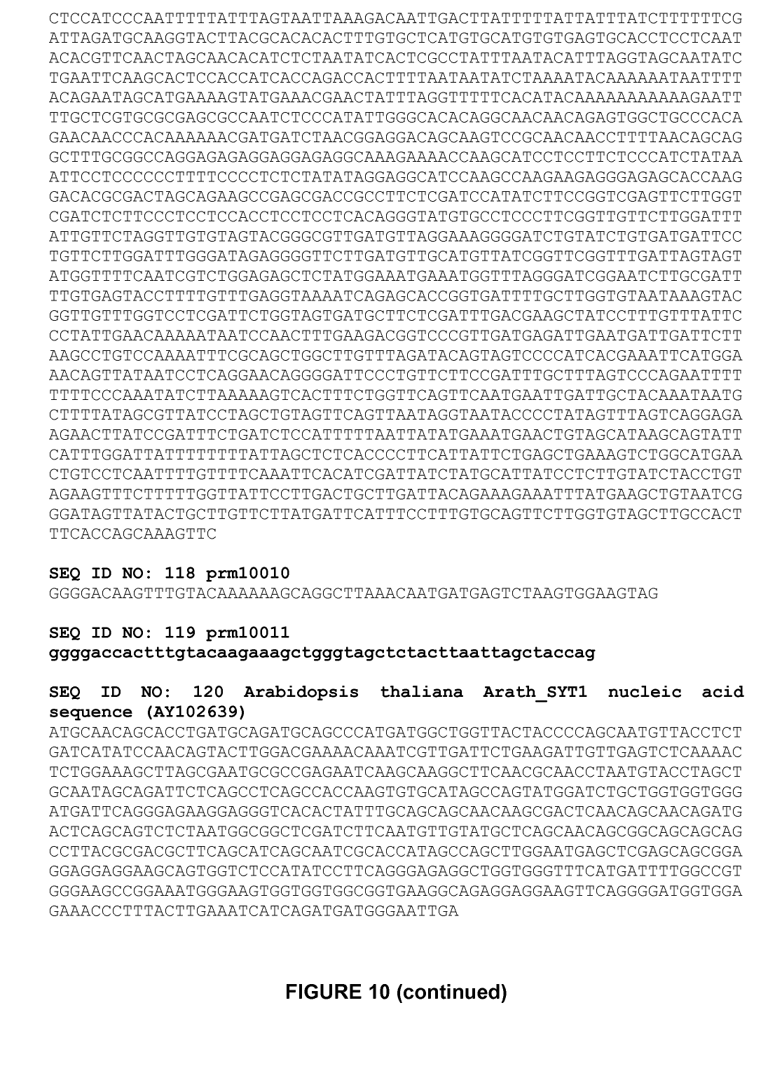

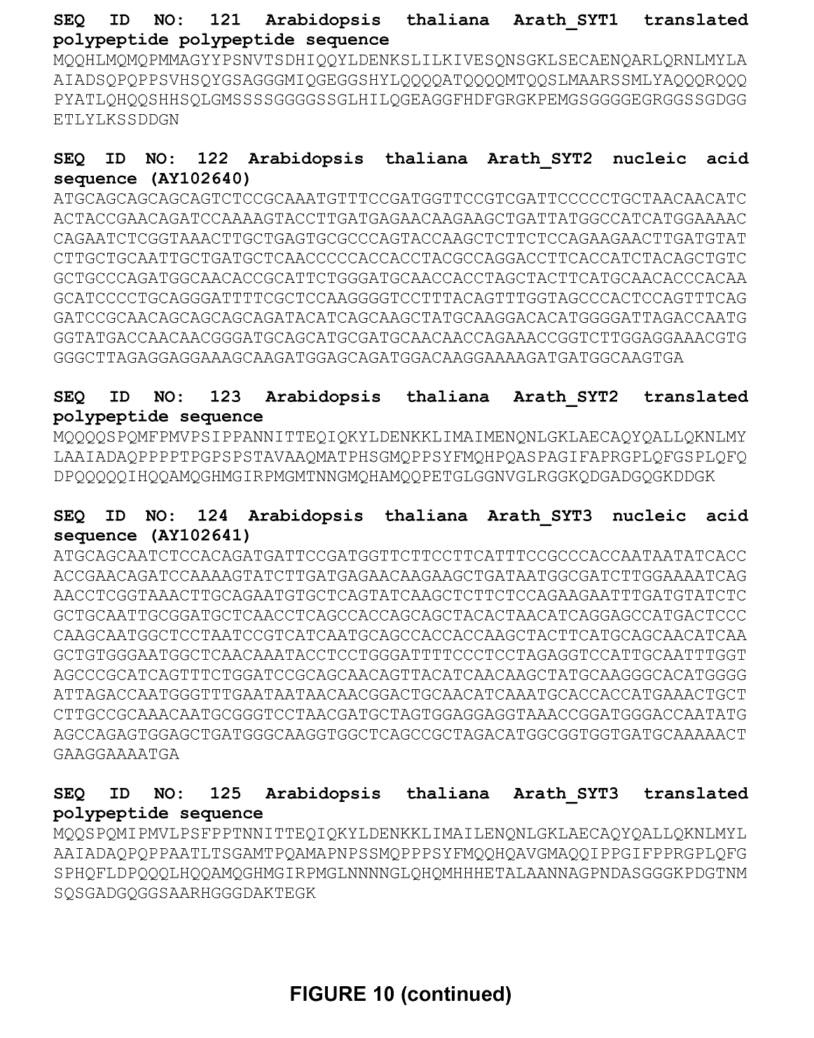

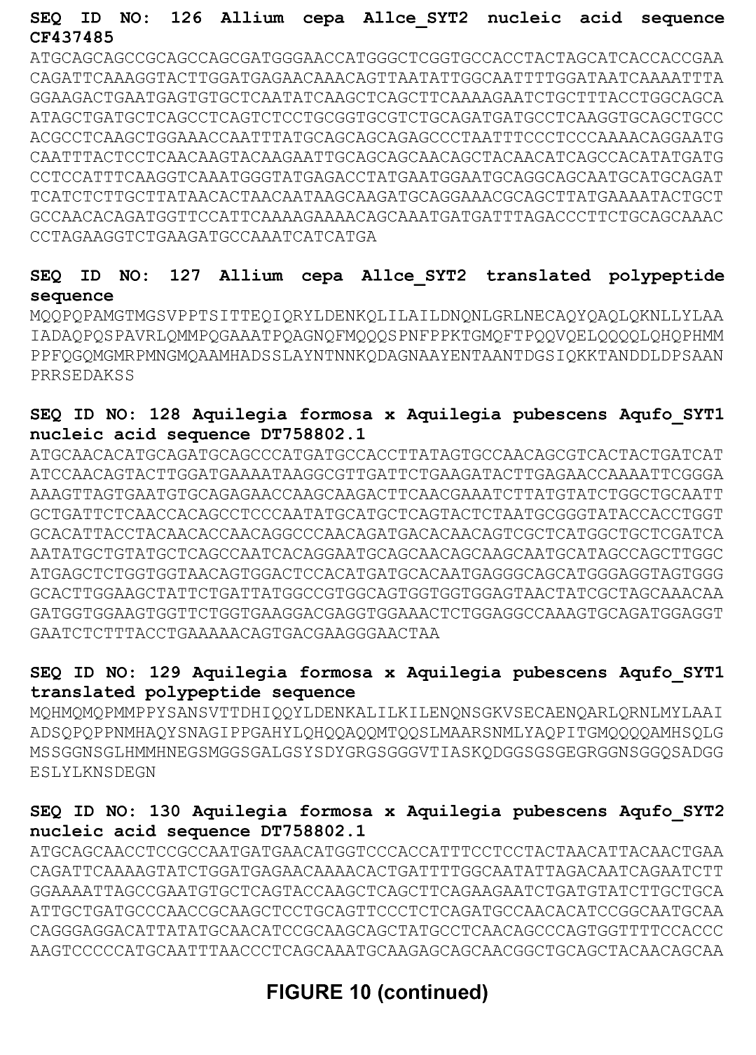

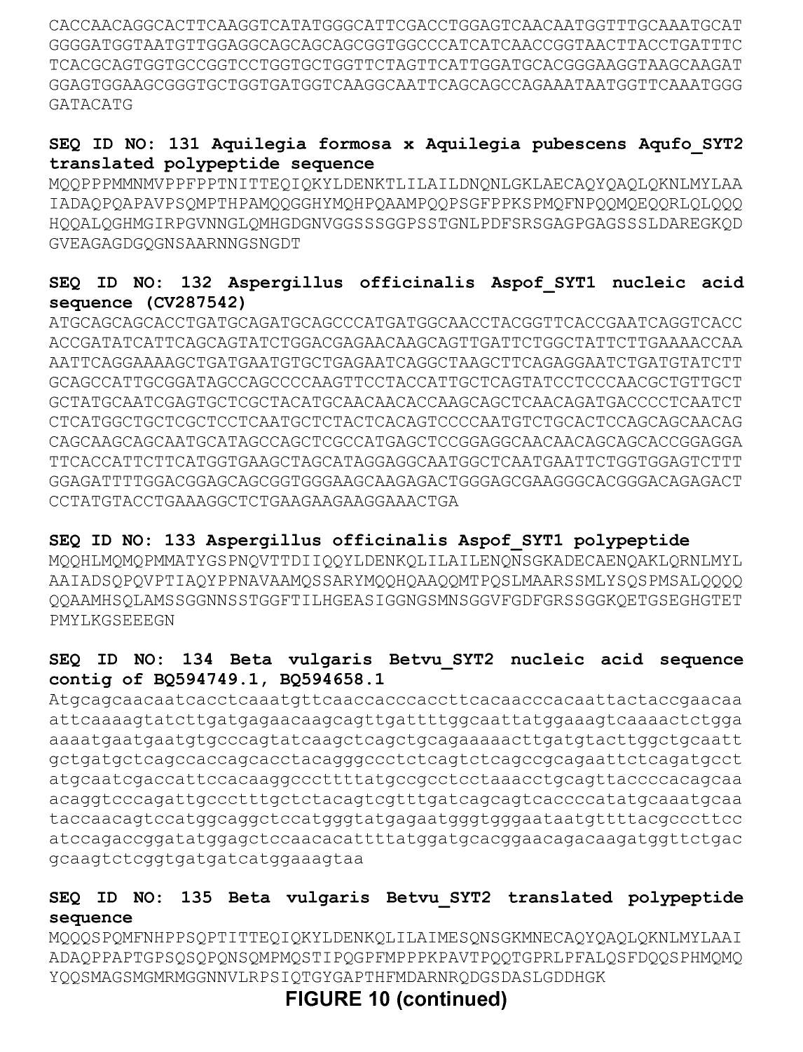

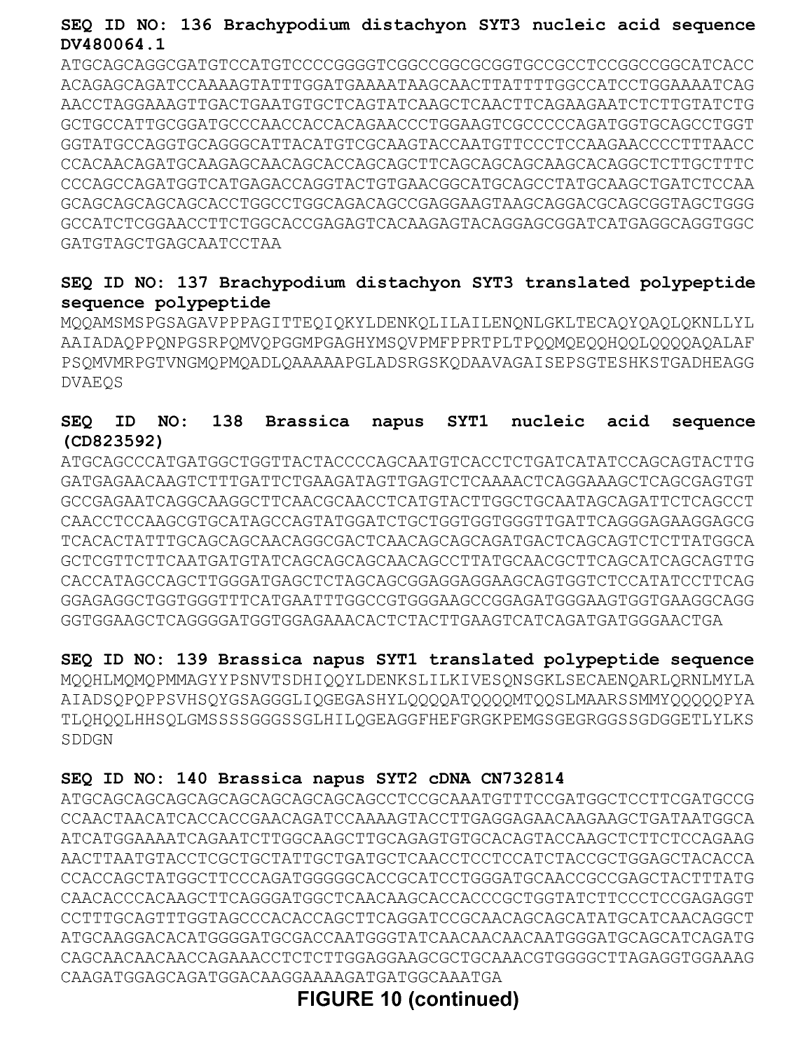

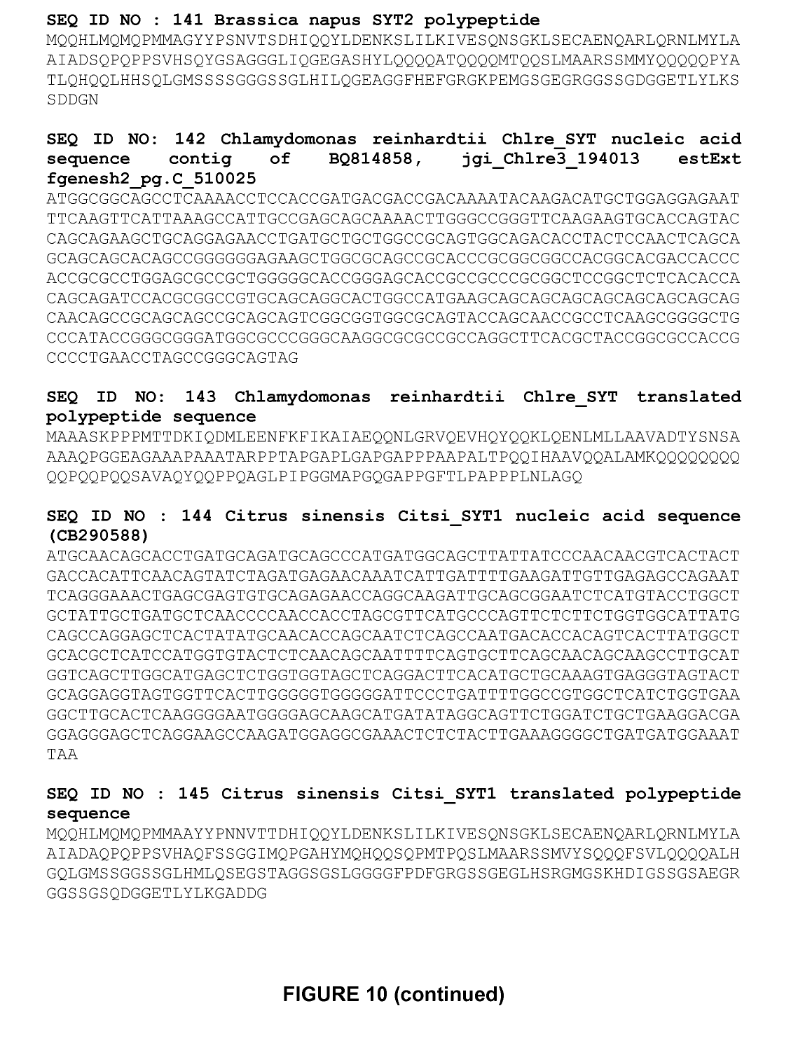

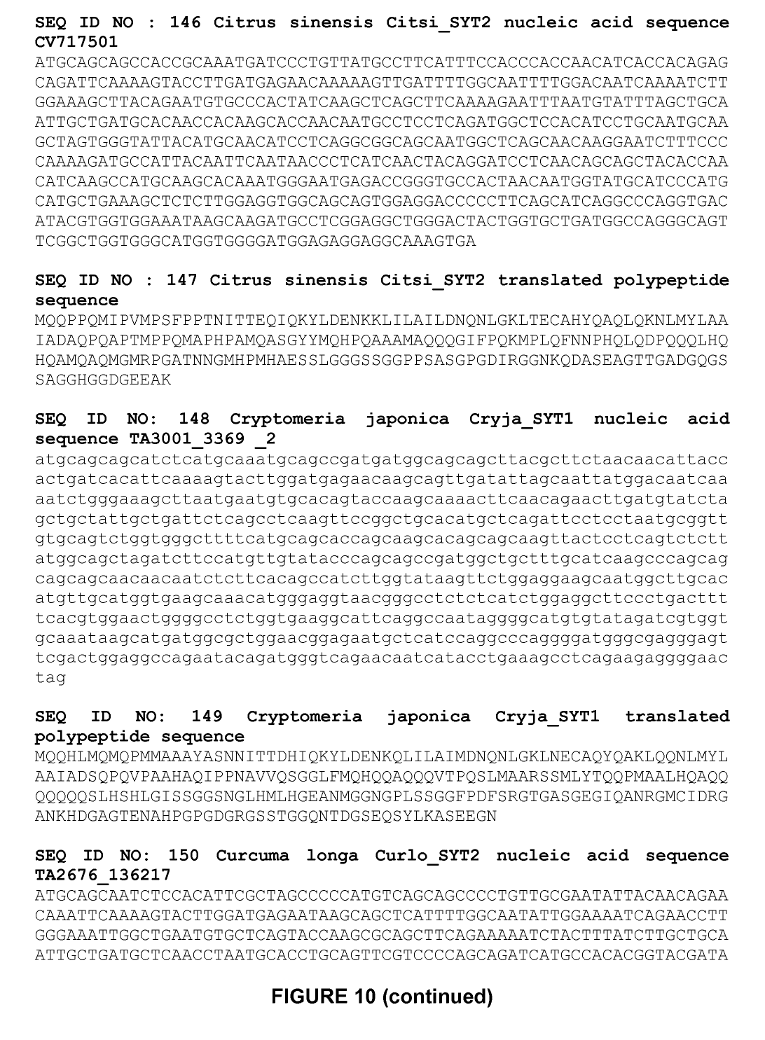

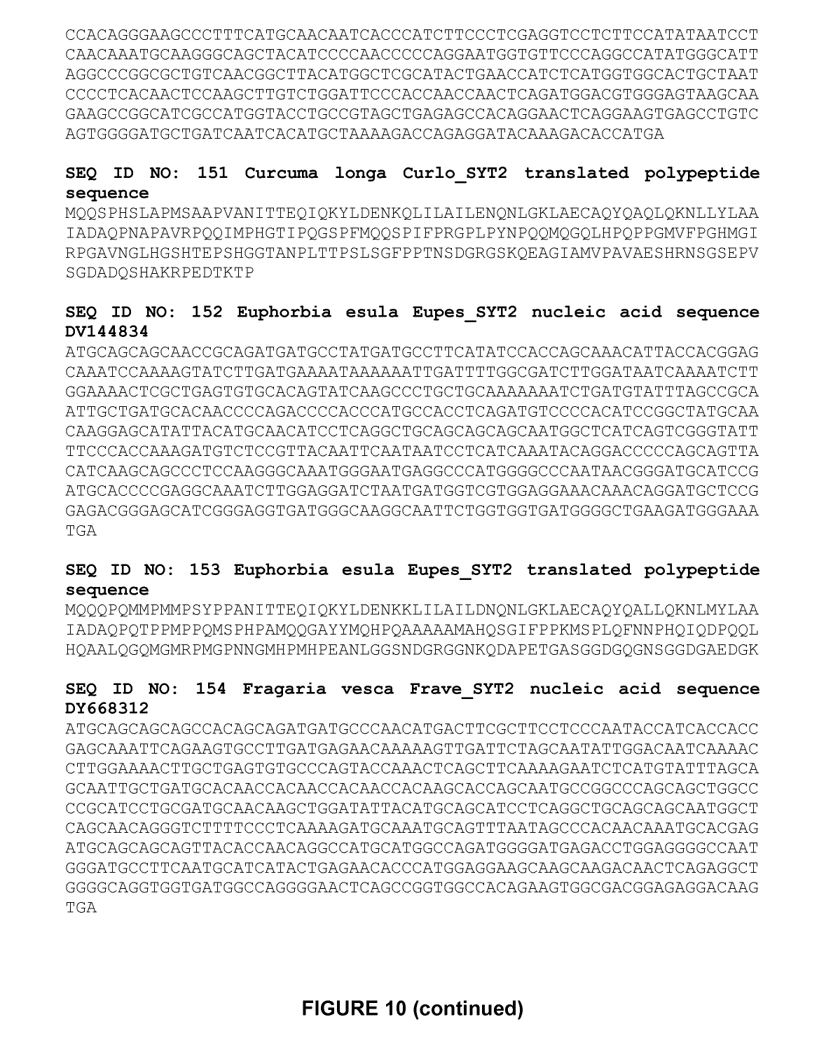

Results of the alignment for the SNH domain of SYT polypeptides from Table A.2 (as represented by SEQ ID NO: 115 for SEQ ID NO: 2) are shown in FIG. 5 of the present application. The most conserved amino acid residues within the SNH domain, as represented by SEQ ID NO: 263, are boxed in black.

### Example 3

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B.1 for the global similarity and identity over the full length of the GRF polypeptide sequences (excluding the partial polypeptide sequences), and in Table B.2 for the global similarity and identity over the full length of the SYT polypeptide sequences.

The percentage identity between the full length GRF polypeptide sequences useful in performing the methods of the invention can be as low as 15% amino acid identity compared to SEQ ID NO: 2.

The percentage identity can be substantially increased if the identity calculation is performed between the QLQ domain SEQ ID NO: 2 (as represented by SEQ ID NO: 115 comprised in SEQ ID NO: 2; QLQ domain of the GRF polypeptides of Table A.1 represented in FIG. 2) and the QLQ domains of the polypeptides useful in performing the invention. Similarly, the percentage identity can be substantially increased if the identity calculation is performed between the WRC domain SEQ ID NO: 2 (as represented by SEQ ID NO: 116 comprised in SEQ ID NO: 2; WRC domain of the GRF polypeptides of Table A.1 represented in FIG. 3) and the WRC domains of the polypeptides useful in performing the invention. Percentage identity over the QLQ domain amongst the polypeptide sequences useful in performing the methods of the invention ranges between 25% and 99% amino acid identity, and percentage identity over the WRC domain amongst the polypeptide sequences useful in performing the methods of the invention ranges between 60% and 99% amino acid identity. As can also be observed in FIG. 3, the WRC domain is better conserved amongst the different GRF polypeptides than the QLQ domain, as shown in FIG. 2

The percentages in amino acid identity between the QLQ domains, and the percentage identity between the WRC domains are significantly higher than the percentage amino acid identity calculated between the full length GRF polypeptide sequences.

TABLE B.1
MatGAT results for global similarity and identity over the full length of the GRF polypeptide sequences.

| | 1 | 2 | 3 | 4 | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|---|---|
| 1. Aqufo_GRF | | 31 | 22 | 25 | 23 | 38 | 22 | 19 | 22 | 23 | 39 | 31 | 21 | 46 | 23 | 18 | 34 | 15 | 33 |
| 2. Arath_GRF_AT2G06200.1 | 43 | | 18 | 23 | 20 | 28 | 18 | 17 | 18 | 21 | 27 | 26 | 21 | 32 | 19 | 21 | 25 | 21 | 26 |
| 3. Arath_GRF_AT2G22840.1 | 36 | 25 | | 26 | 19 | 22 | 22 | 23 | 57 | 21 | 21 | 24 | 27 | 22 | 20 | 16 | 24 | 15 | 26 |
| 4. Arath_GRF_AT2G36400.1 | 43 | 31 | 38 | | 23 | 27 | 48 | 23 | 26 | 26 | 24 | 26 | 47 | 25 | 20 | 24 | 28 | 19 | 28 |
| 5. Arath_GRF_AT2G45480.1 | 38 | 30 | 33 | 39 | | 21 | 21 | 16 | 17 | 23 | 22 | 23 | 21 | 24 | 29 | 16 | 23 | 16 | 21 |
| 6. Arath_GRF_AT3G13960.1 | 53 | 38 | 34 | 44 | 34 | | 23 | 18 | 21 | 22 | 83 | 29 | 22 | 45 | 21 | 16 | 29 | 16 | 29 |
| 7. Arath_GRF_AT3G52910.1 | 34 | 26 | 40 | 56 | 36 | 36 | | 20 | 24 | 22 | 23 | 22 | 31 | 22 | 19 | 17 | 22 | 15 | 23 |
| 8. Arath_GRF_AT4G24150.1 | 31 | 25 | 38 | 36 | 32 | 30 | 35 | | 23 | 25 | 19 | 21 | 25 | 18 | 16 | 17 | 21 | 18 | 23 |
| 9. Arath_GRF_AT4G37740.1 | 35 | 24 | 72 | 38 | 33 | 31 | 40 | 39 | | 21 | 23 | 23 | 26 | 23 | 20 | 18 | 23 | 17 | 24 |
| 10. Arath_GRF_AT5G53660.1 | 37 | 30 | 35 | 40 | 35 | 37 | 34 | 36 | 33 | | 24 | 27 | 25 | 23 | 23 | 19 | 24 | 14 | 25 |
| 11. Brana_GRF | 54 | 39 | 33 | 41 | 35 | 90 | 33 | 33 | 34 | 39 | | 28 | 21 | 47 | 21 | 16 | 29 | 15 | 31 |
| 12. Horvu_GRF | 49 | 34 | 35 | 42 | 39 | 44 | 35 | 32 | 35 | 41 | 47 | | 25 | 25 | 23 | 21 | 68 | 20 | 62 |
| 13. Lyces_GRF | 42 | 30 | 38 | 64 | 37 | 41 | 43 | 38 | 36 | 41 | 40 | 42 | | 24 | 21 | 25 | 25 | 18 | 27 |
| 14. Medtr_GRF | 61 | 44 | 34 | 38 | 36 | 65 | 34 | 31 | 36 | 38 | 63 | 44 | 40 | | 22 | 17 | 31 | 14 | 31 |
| 15. Medtr_GRF\like | 37 | 27 | 32 | 33 | 46 | 37 | 33 | 31 | 34 | 37 | 37 | 37 | 34 | 36 | | 16 | 22 | 20 | 24 |
| 16. Orysa_GRF_NM_001054270.1 | 27 | 37 | 23 | 31 | 25 | 24 | 23 | 24 | 24 | 28 | 25 | 29 | 32 | 27 | 24 | | 22 | 35 | 22 |
| 17. Orysa_GRF_NM_001060298.1 | 53 | 35 | 36 | 44 | 35 | 46 | 35 | 32 | 35 | 42 | 46 | 78 | 41 | 48 | 36 | 30 | | 20 | 70 |
| 18. Orysa_GRF_NM_001066126.1 | 27 | 38 | 24 | 29 | 28 | 29 | 23 | 26 | 25 | 26 | 28 | 30 | 31 | 29 | 29 | 46 | 32 | | 20 |
| 19. Orysa_GRF_Os02g47280.2 | 51 | 38 | 36 | 47 | 36 | 46 | 36 | 35 | 35 | 39 | 49 | 73 | 44 | 47 | 37 | 30 | 78 | 29 | |
| 20. Orysa_GRF_Os02g53690.1 | 57 | 38 | 36 | 43 | 36 | 52 | 33 | 32 | 34 | 36 | 52 | 49 | 40 | 52 | 33 | 26 | 49 | 26 | 50 |
| 21. Orysa_GRF_Os03g51970.1 | 40 | 31 | 49 | 40 | 39 | 40 | 37 | 29 | 45 | 40 | 40 | 38 | 38 | 40 | 38 | 23 | 38 | 24 | 40 |
| 22. Orysa_GRF_Os04g48510.1 | 29 | 41 | 26 | 30 | 28 | 26 | 24 | 27 | 26 | 31 | 28 | 31 | 33 | 30 | 28 | 71 | 32 | 47 | 32 |
| 23. Orysa_GRF_Os04g51190.1 | 52 | 35 | 35 | 44 | 34 | 45 | 36 | 32 | 34 | 41 | 46 | 79 | 41 | 44 | 35 | 29 | 98 | 31 | 78 |
| 24. Orysa_GRF_Os06g02560.1 | 52 | 38 | 32 | 38 | 37 | 41 | 33 | 29 | 33 | 40 | 45 | 56 | 40 | 46 | 34 | 28 | 54 | 24 | 55 |
| 25. Orysa_GRF_Os11g35030.1 | 38 | 32 | 43 | 37 | 36 | 38 | 35 | 34 | 40 | 38 | 38 | 38 | 39 | 35 | 37 | 27 | 37 | 29 | 40 |
| 26. Orysa_GRF_Os12g29980.1 | 39 | 33 | 46 | 40 | 37 | 40 | 35 | 37 | 43 | 39 | 40 | 41 | 40 | 38 | 39 | 27 | 41 | 28 | 42 |
| 27. Oyrsa_GRF_Os03g47140.1 | 37 | 30 | 39 | 40 | 40 | 37 | 35 | 34 | 36 | 40 | 42 | 39 | 38 | 32 | 38 | 26 | 36 | 28 | 37 |
| 28. Poptr_GRF_lcl_scaff_28.10 | 67 | 43 | 34 | 40 | 39 | 52 | 37 | 31 | 34 | 37 | 55 | 53 | 42 | 60 | 37 | 24 | 55 | 27 | 53 |
| 29. Poptr_GRF_lcl_scaff_28.309 | 40 | 32 | 36 | 65 | 33 | 38 | 46 | 33 | 35 | 42 | 38 | 42 | 59 | 41 | 33 | 30 | 40 | 32 | 41 |
| 30. Poptr_GRF_lcl_scaff_I.1018 | 62 | 43 | 32 | 39 | 35 | 58 | 35 | 29 | 32 | 42 | 59 | 45 | 39 | 71 | 33 | 27 | 50 | 28 | 48 |
| 31. Poptr_GRF_lcl_scaff_I.688 | 32 | 25 | 39 | 36 | 32 | 31 | 37 | 46 | 38 | 35 | 32 | 35 | 38 | 33 | 33 | 22 | 34 | 26 | 36 |
| 32. Poptr_GRF_lcl_scaff_I.995 | 26 | 34 | 22 | 27 | 24 | 24 | 20 | 22 | 22 | 28 | 25 | 26 | 28 | 28 | 22 | 51 | 26 | 42 | 27 |
| 33. Poptr_GRF_lcl_scaff_II.1070 | 31 | 24 | 59 | 36 | 32 | 33 | 39 | 35 | 54 | 34 | 31 | 34 | 33 | 32 | 33 | 21 | 33 | 24 | 34 |
| 34. Poptr_GRF_lcl_scaff_III.741 | 52 | 38 | 34 | 38 | 36 | 45 | 32 | 31 | 33 | 36 | 45 | 53 | 38 | 48 | 38 | 28 | 53 | 27 | 50 |
| 35. Poptr_GRF_lcl_scaff_VII.1274 | 38 | 25 | 57 | 37 | 34 | 35 | 41 | 37 | 58 | 37 | 34 | 35 | 36 | 34 | 33 | 22 | 36 | 25 | 36 |
| 36. Poptr_GRF_lcl_scaff_XII.277 | 34 | 25 | 40 | 37 | 33 | 33 | 37 | 42 | 44 | 38 | 33 | 33 | 34 | 32 | 33 | 22 | 32 | 24 | 35 |
| 37. Poptr_GRF_lcl_scaff_XIII.769 | 57 | 42 | 32 | 42 | 38 | 46 | 32 | 30 | 34 | 34 | 46 | 53 | 39 | 53 | 35 | 31 | 57 | 26 | 57 |
| 38. Poptr_GRF_lcl_scaff_XIV.174 | 33 | 25 | 36 | 35 | 43 | 35 | 39 | 34 | 36 | 35 | 35 | 35 | 35 | 35 | 42 | 23 | 31 | 25 | 36 |
| 39. Poptr_GRF_lcl_scaff_XIV.39 | 34 | 22 | 59 | 36 | 33 | 32 | 38 | 34 | 55 | 35 | 30 | 34 | 36 | 32 | 32 | 21 | 32 | 24 | 33 |
| 40. Poptr_GRF_lcl_scaff_XIV.51 | 37 | 27 | 60 | 41 | 35 | 35 | 42 | 40 | 54 | 37 | 36 | 37 | 36 | 37 | 37 | 22 | 36 | 23 | 35 |
| 41. Poptr_GRF_lcl_scaff_XIX.480 | 54 | 42 | 32 | 40 | 35 | 44 | 31 | 28 | 32 | 36 | 47 | 51 | 38 | 49 | 32 | 33 | 54 | 28 | 52 |
| 42. Sacof_GRF | 37 | 28 | 41 | 39 | 37 | 39 | 37 | 35 | 39 | 36 | 41 | 37 | 40 | 35 | 37 | 27 | 37 | 28 | 38 |
| 43. Vitvi_GRF | 70 | 43 | 35 | 41 | 35 | 56 | 33 | 32 | 33 | 37 | 58 | 48 | 39 | 69 | 34 | 26 | 51 | 24 | 50 |
| 44. Zeama_GRF10_EF515849.1 | 26 | 36 | 23 | 29 | 27 | 27 | 23 | 26 | 25 | 26 | 26 | 31 | 32 | 26 | 30 | 44 | 32 | 81 | 32 |
| 45. Zeama_GRF11_EF515850.1 | 50 | 41 | 29 | 41 | 33 | 42 | 28 | 25 | 30 | 35 | 42 | 44 | 35 | 45 | 33 | 31 | 46 | 30 | 45 |
| 46. Zeama_GRF12_EF515851.1 | 44 | 38 | 31 | 40 | 32 | 41 | 30 | 30 | 30 | 39 | 44 | 46 | 38 | 42 | 31 | 32 | 46 | 33 | 45 |
| 47. Zeama_GRF13_EF515852.1 | 37 | 29 | 39 | 40 | 37 | 40 | 36 | 37 | 39 | 36 | 38 | 40 | 37 | 35 | 38 | 26 | 39 | 29 | 41 |
| 48. Zeama_GRF14_EF515853.1 | 49 | 36 | 33 | 45 | 36 | 43 | 35 | 30 | 33 | 42 | 42 | 53 | 42 | 43 | 34 | 28 | 55 | 27 | 54 |
| 49. Zeama_GRF1_EF515840.1 | 50 | 35 | 38 | 47 | 37 | 47 | 36 | 34 | 34 | 39 | 45 | 67 | 41 | 43 | 38 | 29 | 74 | 29 | 79 |
| 50. Zeama_GRF2_EF515841.1 | 42 | 35 | 38 | 41 | 36 | 41 | 30 | 31 | 37 | 45 | 41 | 40 | 38 | 41 | 39 | 29 | 43 | 31 | 40 |
| 51. Zeama_GRF3_EF515842.1 | 51 | 36 | 33 | 41 | 38 | 49 | 34 | 27 | 31 | 36 | 49 | 45 | 40 | 46 | 36 | 27 | 48 | 25 | 50 |
| 52. Zeama_GRF4_EF515843.1 | 24 | 36 | 24 | 30 | 27 | 28 | 21 | 25 | 26 | 25 | 28 | 31 | 31 | 26 | 28 | 45 | 31 | 80 | 32 |
| 53. Zeama_GRF5_EF515844.1 | 50 | 35 | 35 | 42 | 35 | 42 | 34 | 32 | 34 | 40 | 43 | 75 | 40 | 41 | 36 | 31 | 80 | 31 | 72 |
| 54. Zeama_GRF6_EF515845.1 | 50 | 36 | 35 | 40 | 35 | 44 | 33 | 30 | 36 | 39 | 45 | 76 | 40 | 42 | 37 | 30 | 80 | 32 | 71 |
| 55. Zeama_GRF7_EF515846.1 | 48 | 41 | 31 | 39 | 34 | 44 | 29 | 27 | 31 | 37 | 45 | 42 | 35 | 47 | 32 | 32 | 46 | 31 | 45 |
| 56. Zeama_GRF8_EF515847.1 | 38 | 29 | 39 | 38 | 36 | 38 | 34 | 37 | 40 | 37 | 38 | 37 | 39 | 35 | 38 | 27 | 37 | 30 | 38 |
| 57. Zeama_GRF9_EF515848.1 | 57 | 42 | 31 | 37 | 31 | 49 | 32 | 29 | 31 | 32 | 50 | 45 | 40 | 52 | 35 | 31 | 45 | 27 | 45 |
| | 20 | 21 | 22 | 23 | 24 | 25 | 26 | 27 | 28 | 29 | 30 | 31 | 32 | 33 | 34 | 35 | 36 | 37 | 38 |
| 1. Aqufo_GRF | 41 | 29 | 18 | 34 | 35 | 23 | 23 | 23 | 54 | 23 | 47 | 21 | 18 | 20 | 34 | 25 | 21 | 44 | 23 |
| 2. Arath_GRF_AT2G06200.1 | 28 | 22 | 21 | 25 | 27 | 20 | 22 | 20 | 34 | 22 | 32 | 16 | 24 | 16 | 27 | 17 | 15 | 30 | 19 |
| 3. Arath_GRF_AT2G22840.1 | 23 | 32 | 21 | 24 | 22 | 30 | 31 | 28 | 22 | 26 | 22 | 24 | 17 | 42 | 22 | 40 | 27 | 21 | 20 |
| 4. Arath_GRF_AT2G36400.1 | 25 | 25 | 23 | 28 | 27 | 24 | 25 | 25 | 23 | 51 | 27 | 26 | 20 | 25 | 25 | 27 | 23 | 27 | 22 |
| 5. Arath_GRF_AT2G45480.1 | 22 | 23 | 17 | 22 | 24 | 18 | 22 | 21 | 22 | 19 | 23 | 18 | 17 | 17 | 23 | 21 | 19 | 24 | 28 |
| 6. Arath_GRF_AT3G13960.1 | 35 | 26 | 17 | 28 | 27 | 23 | 25 | 22 | 36 | 21 | 41 | 20 | 17 | 22 | 27 | 23 | 21 | 31 | 22 |
| 7. Arath_GRF_AT3G52910.1 | 21 | 23 | 18 | 22 | 20 | 23 | 22 | 22 | 24 | 37 | 22 | 22 | 14 | 23 | 22 | 22 | 21 | 23 | 23 |
| 8. Arath_GRF_AT4G24150.1 | 19 | 17 | 18 | 22 | 20 | 22 | 23 | 20 | 19 | 22 | 20 | 32 | 15 | 21 | 19 | 20 | 25 | 20 | 17 |
| 9. Arath_GRF_AT4G37740.1 | 21 | 27 | 19 | 24 | 24 | 29 | 29 | 26 | 21 | 24 | 23 | 24 | 17 | 38 | 21 | 42 | 27 | 23 | 19 |
| 10. Arath_GRF_AT5G53660.1 | 22 | 22 | 18 | 24 | 25 | 23 | 24 | 25 | 22 | 28 | 26 | 24 | 18 | 22 | 22 | 25 | 27 | 23 | 21 |
| 11. Brana_GRF | 35 | 26 | 18 | 29 | 28 | 21 | 24 | 24 | 37 | 21 | 44 | 19 | 17 | 20 | 30 | 22 | 21 | 31 | 23 |
| 12. Horvu_GRF | 29 | 24 | 22 | 70 | 42 | 25 | 28 | 25 | 32 | 25 | 30 | 21 | 21 | 23 | 35 | 23 | 23 | 39 | 21 |
| 13. Lyces_GRF | 22 | 24 | 25 | 25 | 25 | 23 | 26 | 24 | 22 | 45 | 26 | 25 | 21 | 23 | 24 | 25 | 23 | 25 | 22 |
| 14. Medtr_GRF | 38 | 26 | 17 | 28 | 30 | 22 | 24 | 22 | 43 | 24 | 56 | 22 | 18 | 22 | 29 | 22 | 21 | 36 | 23 |
| 15. Medtr_GRF\like | 20 | 20 | 18 | 22 | 21 | 24 | 23 | 22 | 22 | 19 | 20 | 21 | 16 | 19 | 25 | 20 | 21 | 23 | 29 |
| 16. Orysa_GRF_NM_001054270.1 | 18 | 16 | 66 | 21 | 20 | 21 | 21 | 19 | 16 | 23 | 18 | 18 | 38 | 16 | 19 | 16 | 16 | 21 | 18 |
| 17. Orysa_GRF_NM_001060298.1 | 33 | 24 | 23 | 98 | 42 | 25 | 27 | 26 | 36 | 25 | 32 | 22 | 20 | 22 | 36 | 25 | 24 | 43 | 19 |
| 18. Orysa_GRF_NM_001066126.1 | 14 | 16 | 34 | 20 | 14 | 20 | 18 | 18 | 12 | 19 | 15 | 18 | 29 | 16 | 16 | 15 | 16 | 14 | 16 |
| 19. Orysa_GRF_Os02g47280.2 | 34 | 25 | 24 | 70 | 41 | 25 | 29 | 24 | 33 | 27 | 32 | 23 | 20 | 23 | 35 | 25 | 25 | 44 | 23 |
| 20. Orysa_GRF_Os02g53690.1 | | 27 | 20 | 33 | 34 | 23 | 25 | 22 | 41 | 23 | 38 | 19 | 19 | 22 | 30 | 23 | 20 | 35 | 23 |
| 21. Orysa_GRF_Os03g51970.1 | 40 | | 18 | 24 | 25 | 28 | 38 | 25 | 27 | 21 | 28 | 22 | 15 | 37 | 25 | 38 | 24 | 25 | 22 |
| 22. Orysa_GRF_Os04g48510.1 | 29 | 24 | | 23 | 22 | 23 | 22 | 19 | 18 | 24 | 19 | 18 | 38 | 18 | 22 | 17 | 17 | 23 | 20 |
| 23. Orysa_GRF_Os04g51190.1 | 50 | 37 | 32 | | 42 | 24 | 27 | 26 | 36 | 24 | 31 | 22 | 21 | 22 | 36 | 25 | 24 | 42 | 22 |
| 24. Orysa_GRF_Os06g02560.1 | 47 | 35 | 32 | 54 | | 24 | 25 | 23 | 36 | 27 | 30 | 21 | 21 | 22 | 39 | 24 | 21 | 42 | 24 |
| 25. Orysa_GRF_Os11g35030.1 | 39 | 45 | 29 | 39 | 36 | | 37 | 28 | 21 | 24 | 25 | 25 | 19 | 29 | 23 | 29 | 23 | 23 | 20 |
| 26. Orysa_GRF_Os12g29980.1 | 37 | 55 | 29 | 41 | 37 | 54 | | 28 | 24 | 26 | 26 | 22 | 17 | 32 | 26 | 34 | 24 | 27 | 24 |
| 27. Oyrsa_GRF_Os03g47140.1 | 35 | 44 | 27 | 40 | 34 | 43 | 48 | | 22 | 26 | 25 | 23 | 18 | 27 | 23 | 27 | 23 | 26 | 21 |
| 28. Poptr_GRF_lcl_scaff_28.10 | 55 | 40 | 27 | 55 | 48 | 38 | 38 | 38 | | 25 | 44 | 21 | 19 | 23 | 35 | 24 | 22 | 41 | 22 |
| 29. Poptr_GRF_lcl_scaff_28.309 | 39 | 34 | 33 | 41 | 42 | 39 | 40 | 39 | 42 | | 26 | 24 | 20 | 24 | 24 | 24 | 22 | 26 | 21 |
| 30. Poptr_GRF_lcl_scaff_I.1018 | 52 | 40 | 31 | 48 | 48 | 38 | 37 | 35 | 59 | 39 | | 22 | 20 | 19 | 32 | 23 | 22 | 36 | 21 |
| 31. Poptr_GRF_lcl_scaff_I.688 | 33 | 36 | 24 | 34 | 33 | 34 | 37 | 37 | 36 | 33 | 31 | | 16 | 25 | 20 | 25 | 29 | 20 | 21 |
| 32. Poptr_GRF_lcl_scaff_I.995 | 27 | 21 | 50 | 26 | 28 | 25 | 24 | 24 | 25 | 29 | 28 | 21 | | 14 | 19 | 16 | 13 | 22 | 17 |
| 33. Poptr_GRF_lcl_scaff_II.1070 | 33 | 50 | 24 | 33 | 31 | 40 | 46 | 39 | 34 | 32 | 31 | 39 | 19 | | 22 | 47 | 28 | 22 | 21 |
| 34. Poptr_GRF_lcl_scaff_III.741 | 43 | 38 | 32 | 53 | 58 | 38 | 40 | 36 | 51 | 41 | 50 | 32 | 28 | 31 | | 23 | 20 | 47 | 22 |
| 35. Poptr_GRF_lcl_scaff_VII.1274 | 34 | 52 | 25 | 37 | 32 | 41 | 46 | 41 | 35 | 35 | 33 | 39 | 20 | 62 | 34 | | 28 | 23 | 23 |
| 36. Poptr_GRF_lcl_scaff_XII.277 | 32 | 37 | 22 | 33 | 31 | 36 | 37 | 35 | 35 | 34 | 33 | 46 | 18 | 43 | 31 | 41 | | 22 | 22 |
| 37. Poptr_GRF_lcl_scaff_XIII.769 | 48 | 37 | 35 | 56 | 57 | 38 | 39 | 38 | 54 | 41 | 54 | 29 | 31 | 30 | 63 | 32 | 31 | | 23 |
| 38. Poptr_GRF_lcl_scaff_XIV.174 | 34 | 37 | 25 | 32 | 34 | 33 | 35 | 35 | 34 | 35 | 35 | 37 | 23 | 37 | 33 | 39 | 39 | 33 | |
| 39. Poptr_GRF_lcl_scaff_XIV.39 | 33 | 47 | 22 | 32 | 30 | 38 | 42 | 38 | 33 | 31 | 30 | 36 | 19 | 68 | 30 | 78 | 40 | 30 | 37 |
| 40. Poptr_GRF_lcl_scaff_XIV.51 | 40 | 57 | 24 | 38 | 36 | 43 | 52 | 40 | 38 | 34 | 34 | 41 | 20 | 79 | 35 | 66 | 45 | 34 | 41 |
| 41. Poptr_GRF_lcl_scaff_XIX.480 | 47 | 35 | 34 | 53 | 53 | 35 | 37 | 35 | 52 | 39 | 53 | 30 | 31 | 30 | 59 | 31 | 29 | 88 | 31 |
| 42. Sacof_GRF | 35 | 43 | 30 | 40 | 35 | 45 | 46 | 82 | 38 | 39 | 35 | 38 | 24 | 40 | 40 | 40 | 38 | 37 | 33 |
| 43. Vitvi_GRF | 58 | 40 | 29 | 51 | 50 | 38 | 40 | 36 | 65 | 40 | 70 | 31 | 27 | 30 | 51 | 34 | 32 | 54 | 34 |
| 44. Zeama_GRF10_EF515849.1 | 22 | 24 | 44 | 32 | 28 | 30 | 28 | 29 | 25 | 34 | 25 | 24 | 41 | 23 | 25 | 23 | 24 | 28 | 24 |
| 45. Zeama_GRF11_EF515850.1 | 53 | 35 | 35 | 46 | 47 | 35 | 36 | 33 | 49 | 38 | 46 | 29 | 29 | 28 | 46 | 31 | 29 | 50 | 30 |
| 46. Zeama_GRF12_EF515851.1 | 40 | 34 | 32 | 45 | 67 | 36 | 35 | 34 | 45 | 39 | 45 | 30 | 32 | 27 | 50 | 31 | 30 | 52 | 32 |
| 47. Zeama_GRF13_EF515852.1 | 35 | 44 | 28 | 40 | 36 | 43 | 47 | 78 | 37 | 37 | 34 | 37 | 23 | 41 | 38 | 39 | 38 | 37 | 34 |
| 48. Zeama_GRF14_EF515853.1 | 47 | 40 | 30 | 54 | 77 | 39 | 36 | 39 | 50 | 41 | 45 | 35 | 28 | 32 | 52 | 33 | 32 | 52 | 35 |
| 49. Zeama_GRF1_EF515840.1 | 51 | 42 | 30 | 74 | 50 | 40 | 41 | 43 | 49 | 42 | 45 | 37 | 25 | 37 | 46 | 38 | 37 | 52 | 36 |
| 50. Zeama_GRF2_EF515841.1 | 40 | 46 | 33 | 43 | 42 | 66 | 54 | 42 | 40 | 36 | 43 | 33 | 29 | 39 | 41 | 40 | 35 | 42 | 33 |
| 51. Zeama_GRF3_EF515842.1 | 80 | 38 | 29 | 48 | 46 | 37 | 39 | 33 | 53 | 39 | 49 | 33 | 25 | 30 | 41 | 33 | 31 | 45 | 34 |
| 52. Zeama_GRF4_EF515843.1 | 27 | 24 | 44 | 32 | 26 | 30 | 27 | 29 | 25 | 32 | 28 | 26 | 41 | 24 | 27 | 23 | 24 | 28 | 25 |
| 53. Zeama_GRF5_EF515844.1 | 48 | 38 | 32 | 80 | 54 | 38 | 41 | 39 | 52 | 41 | 45 | 34 | 26 | 33 | 49 | 35 | 33 | 52 | 34 |
| 54. Zeama_GRF6_EF515845.1 | 46 | 38 | 31 | 81 | 51 | 38 | 39 | 36 | 51 | 41 | 43 | 33 | 26 | 33 | 51 | 35 | 33 | 53 | 33 |
| 55. Zeama_GRF7_EF515846.1 | 54 | 35 | 34 | 45 | 48 | 34 | 36 | 35 | 47 | 36 | 49 | 31 | 30 | 28 | 48 | 31 | 29 | 49 | 29 |
| 56. Zeama_GRF8_EF515847.1 | 35 | 43 | 28 | 39 | 34 | 46 | 44 | 79 | 38 | 38 | 36 | 37 | 23 | 39 | 37 | 40 | 37 | 35 | 33 |
| 57. Zeama_GRF9_EF515848.1 | 73 | 40 | 31 | 45 | 46 | 35 | 39 | 33 | 52 | 39 | 52 | 30 | 27 | 31 | 45 | 32 | 29 | 47 | 32 |
| | 39 | 40 | 41 | 42 | 43 | 44 | 45 | 46 | 47 | 48 | 49 | 50 | 51 | 52 | 53 | 54 | 55 | 56 | 57 |
| 1. Aqufo_GRF | 21 | 24 | 43 | 22 | 57 | 15 | 34 | 28 | 23 | 33 | 34 | 25 | 37 | 13 | 32 | 32 | 33 | 22 | 38 |
| 2. Arath_GRF_AT2G06200.1 | 15 | 18 | 30 | 20 | 32 | 20 | 29 | 27 | 20 | 26 | 24 | 23 | 27 | 20 | 27 | 25 | 28 | 20 | 30 |
| 3. Arath_GRF_AT2G22840.1 | 44 | 39 | 21 | 29 | 23 | 16 | 21 | 22 | 29 | 23 | 25 | 29 | 21 | 15 | 25 | 24 | 21 | 30 | 22 |
| 4. Arath_GRF_AT2G36400.1 | 25 | 27 | 27 | 27 | 24 | 18 | 26 | 30 | 25 | 29 | 31 | 27 | 26 | 19 | 26 | 25 | 24 | 25 | 24 |
| 5. Arath_GRF_AT2G45480.1 | 20 | 21 | 23 | 19 | 23 | 17 | 21 | 21 | 18 | 25 | 22 | 21 | 21 | 17 | 21 | 20 | 22 | 19 | 19 |
| 6. Arath_GRF_AT3G13960.1 | 22 | 25 | 30 | 24 | 40 | 15 | 29 | 26 | 23 | 28 | 29 | 25 | 33 | 15 | 28 | 29 | 31 | 23 | 33 |
| 7. Arath_GRF_AT3G52910.1 | 22 | 25 | 21 | 22 | 21 | 14 | 19 | 21 | 22 | 22 | 24 | 22 | 21 | 14 | 21 | 20 | 20 | 23 | 20 |
| 8. Arath_GRF_AT4G24150.1 | 20 | 22 | 18 | 21 | 19 | 19 | 17 | 22 | 22 | 21 | 22 | 20 | 17 | 17 | 22 | 22 | 18 | 22 | 18 |
| 9. Arath_GRF_AT4G37740.1 | 42 | 35 | 23 | 27 | 22 | 15 | 20 | 21 | 29 | 23 | 24 | 28 | 20 | 16 | 23 | 22 | 21 | 28 | 20 |
| 10. Arath_GRF_AT5G53660.1 | 23 | 24 | 23 | 25 | 22 | 14 | 22 | 24 | 24 | 25 | 24 | 24 | 21 | 13 | 26 | 23 | 22 | 24 | 21 |
| 11. Brana_GRF | 21 | 24 | 31 | 24 | 41 | 13 | 29 | 27 | 25 | 26 | 30 | 24 | 34 | 13 | 28 | 28 | 30 | 23 | 34 |
| 12. Horvu_GRF | 22 | 26 | 38 | 24 | 31 | 20 | 31 | 35 | 25 | 38 | 54 | 26 | 30 | 19 | 63 | 64 | 30 | 23 | 29 |
| 13. Lyces_GRF | 25 | 24 | 25 | 24 | 20 | 20 | 23 | 27 | 25 | 25 | 23 | 25 | 23 | 19 | 25 | 24 | 23 | 24 | 24 |
| 14. Medtr_GRF | 21 | 23 | 35 | 23 | 53 | 12 | 30 | 26 | 22 | 27 | 29 | 25 | 33 | 13 | 28 | 27 | 32 | 22 | 35 |
| 15. Medtr_GRF\like | 19 | 22 | 22 | 21 | 21 | 19 | 20 | 21 | 21 | 22 | 22 | 24 | 22 | 20 | 25 | 25 | 20 | 21 | 23 |
| 16. Orysa_GRF_NM_001054270.1 | 16 | 17 | 21 | 20 | 17 | 36 | 21 | 23 | 21 | 20 | 23 | 22 | 18 | 34 | 23 | 21 | 22 | 20 | 20 |
| 17. Orysa_GRF_NM_001060298.1 | 22 | 25 | 39 | 25 | 34 | 19 | 34 | 37 | 26 | 41 | 63 | 28 | 35 | 18 | 69 | 68 | 35 | 26 | 32 |
| 18. Orysa_GRF_NM_001066126.1 | 16 | 14 | 15 | 19 | 15 | 73 | 17 | 20 | 19 | 15 | 19 | 19 | 15 | 73 | 19 | 20 | 20 | 18 | 19 |
| 19. Orysa_GRF_Os02g47280.2 | 23 | 23 | 42 | 25 | 34 | 20 | 32 | 35 | 28 | 41 | 72 | 26 | 32 | 20 | 61 | 59 | 31 | 25 | 31 |
| 20. Orysa_GRF_Os02g53690.1 | 21 | 25 | 35 | 21 | 42 | 15 | 43 | 30 | 22 | 32 | 36 | 26 | 69 | 16 | 33 | 33 | 43 | 21 | 64 |
| 21. Orysa_GRF_Os03g51970.1 | 34 | 43 | 26 | 26 | 27 | 14 | 25 | 25 | 26 | 27 | 27 | 30 | 26 | 14 | 25 | 25 | 27 | 26 | 28 |
| 22. Orysa_GRF_Os04g48510.1 | 16 | 18 | 21 | 22 | 19 | 34 | 21 | 23 | 21 | 23 | 24 | 26 | 19 | 32 | 23 | 21 | 22 | 20 | 20 |
| 23. Orysa_GRF_Os04g51190.1 | 22 | 24 | 39 | 26 | 34 | 19 | 34 | 36 | 26 | 41 | 62 | 28 | 34 | 19 | 71 | 69 | 35 | 26 | 32 |
| 24. Orysa_GRF_Os06g02560.1 | 22 | 26 | 41 | 22 | 33 | 15 | 35 | 57 | 24 | 68 | 39 | 27 | 34 | 14 | 42 | 41 | 36 | 23 | 33 |
| 25. Orysa_GRF_Os11g35030.1 | 27 | 29 | 22 | 29 | 23 | 18 | 23 | 23 | 29 | 25 | 24 | 55 | 22 | 18 | 24 | 24 | 22 | 29 | 21 |
| 26. Orysa_GRF_Os12g29980.1 | 31 | 37 | 27 | 26 | 27 | 16 | 25 | 24 | 28 | 24 | 27 | 42 | 25 | 16 | 28 | 26 | 25 | 26 | 25 |
| 27. Oyrsa_GRF_Os03g47140.1 | 27 | 25 | 24 | 71 | 22 | 15 | 24 | 24 | 65 | 25 | 26 | 28 | 22 | 17 | 25 | 25 | 23 | 67 | 22 |
| 28. Popt_ GRF_lcl_scaff_28.10 | 21 | 25 | 39 | 22 | 52 | 14 | 35 | 28 | 25 | 35 | 32 | 25 | 37 | 14 | 33 | 31 | 33 | 23 | 38 |
| 29. Poptr_GRF_lcl_scaff_28.309 | 23 | 24 | 25 | 25 | 25 | 19 | 24 | 27 | 25 | 25 | 25 | 23 | 23 | 19 | 25 | 24 | 24 | 25 | 24 |
| 30. Poptr_GRFscaff_I.1018 | 21 | 23 | 36 | 23 | 56 | 14 | 34 | 31 | 24 | 31 | 33 | 29 | 36 | 14 | 30 | 30 | 34 | 24 | 37 |
| 31. Poptr_GRFscaff_I.688 | 24 | 24 | 21 | 25 | 21 | 16 | 20 | 21 | 25 | 23 | 25 | 22 | 20 | 16 | 25 | 21 | 21 | 24 | 19 |
| 32. Poptr_GRFscaff_I.995 | 14 | 15 | 21 | 18 | 19 | 30 | 21 | 23 | 17 | 20 | 19 | 21 | 18 | 29 | 20 | 20 | 22 | 17 | 18 |
| 33. Poptr_GRFscaff_II.1070 | 50 | 75 | 21 | 27 | 21 | 15 | 20 | 20 | 27 | 22 | 24 | 28 | 20 | 16 | 23 | 22 | 19 | 25 | 20 |
| 34. Poptr_GRFscaff_III.741 | 20 | 25 | 44 | 24 | 33 | 15 | 30 | 34 | 25 | 36 | 32 | 25 | 29 | 16 | 35 | 36 | 30 | 24 | 32 |
| 35. Poptr_GRFscaff_VII.1274 | 74 | 50 | 21 | 26 | 25 | 15 | 22 | 22 | 25 | 23 | 26 | 31 | 21 | 16 | 25 | 25 | 22 | 25 | 21 |
| 36. Poptr_GRFscaff_XII.277 | 28 | 27 | 22 | 25 | 22 | 17 | 19 | 20 | 24 | 22 | 23 | 22 | 20 | 16 | 24 | 24 | 19 | 25 | 18 |
| 37. Poptr_GRFscaff_XIII.769 | 22 | 25 | 81 | 26 | 41 | 13 | 33 | 35 | 25 | 40 | 39 | 26 | 33 | 13 | 41 | 40 | 32 | 25 | 34 |
| 38. Poptr_GRFscaff_XIV.174 | 23 | 24 | 25 | 20 | 22 | 18 | 20 | 23 | 21 | 23 | 23 | 21 | 21 | 18 | 22 | 23 | 19 | 19 | 21 |
| 39. Poptr_GRFscaff_XIV.39 | | 45 | 21 | 25 | 21 | 14 | 19 | 21 | 25 | 22 | 23 | 28 | 19 | 14 | 24 | 23 | 19 | 26 | 19 |
| 40. Poptr_GRFscaff_XIV.51 | 61 | | 22 | 26 | 24 | 16 | 23 | 23 | 25 | 24 | 26 | 30 | 24 | 14 | 25 | 25 | 22 | 25 | 23 |
| 41. Poptr_GRFscaff_XIX.480 | 30 | 32 | | 22 | 39 | 15 | 30 | 33 | 24 | 37 | 37 | 26 | 32 | 17 | 40 | 40 | 32 | 22 | 32 |
| 42. Sacof_GRF | 37 | 42 | 35 | | 22 | 18 | 22 | 25 | 86 | 24 | 25 | 30 | 22 | 17 | 27 | 26 | 23 | 91 | 21 |
| 43. Vitvi_GRF | 31 | 37 | 53 | 36 | | 14 | 34 | 29 | 22 | 33 | 33 | 26 | 40 | 13 | 31 | 32 | 36 | 23 | 42 |
| 44. Zeama_GRF10_EF515849 | 22 | 24 | 26 | 28 | 27 | | 17 | 21 | 17 | 14 | 19 | 18 | 15 | 86 | 19 | 19 | 19 | 18 | 16 |
| 45. Zeama_GRF11_EF515850 | 27 | 31 | 48 | 34 | 46 | 28 | | 32 | 23 | 34 | 33 | 26 | 41 | 18 | 33 | 31 | 75 | 22 | 41 |
| 46. Zeama_GRF12_EF515851 | 28 | 33 | 48 | 36 | 45 | 33 | 46 | | 27 | 61 | 33 | 24 | 29 | 20 | 34 | 34 | 32 | 24 | 31 |
| 47. Zeama_GRF13_EF515852 | 38 | 42 | 35 | 90 | 35 | 26 | 35 | 35 | | 24 | 26 | 29 | 22 | 18 | 27 | 26 | 23 | 86 | 22 |
| 48. Zeama_GRF14_EF515853 | 31 | 36 | 48 | 38 | 48 | 25 | 45 | 67 | 38 | | 38 | 26 | 33 | 15 | 40 | 39 | 34 | 24 | 34 |
| 49. Zeama_GRF1_EF515840 | 34 | 41 | 50 | 42 | 48 | 30 | 46 | 43 | 42 | 52 | | 29 | 35 | 18 | 57 | 57 | 32 | 25 | 34 |
| 50. Zeama_GRF2_EF515841 | 38 | 41 | 38 | 40 | 42 | 30 | 39 | 36 | 41 | 42 | 42 | | 23 | 19 | 28 | 26 | 27 | 30 | 24 |
| 51. Zeama_GRF3_EF515842 | 29 | 33 | 43 | 35 | 54 | 24 | 52 | 42 | 33 | 50 | 52 | 38 | | 15 | 33 | 34 | 41 | 22 | 72 |
| 52. Zeama_GRF4_EF515843 | 23 | 24 | 30 | 27 | 28 | 90 | 31 | 33 | 28 | 26 | 31 | 33 | 25 | | 19 | 20 | 20 | 18 | 16 |
| 53. Zeama_GRF5_EF515844.1 | 33 | 35 | 52 | 38 | 47 | 32 | 46 | 44 | 38 | 53 | 68 | 43 | 47 | 31 | | 87 | 33 | 27 | 31 |
| 54. Zeama_GRF6_EF515845.1 | 32 | 36 | 51 | 37 | 46 | 31 | 44 | 43 | 38 | 53 | 71 | 38 | 47 | 31 | 90 | | 33 | 25 | 32 |
| 55. Zeama_GRF7_EF515846.1 | 27 | 32 | 47 | 35 | 51 | 32 | 83 | 46 | 35 | 47 | 44 | 38 | 52 | 31 | 47 | 43 | | 23 | 40 |
| 56. Zeama_GRF8_EF515847.1 | 38 | 41 | 35 | 94 | 37 | 27 | 33 | 34 | 91 | 38 | 40 | 43 | 36 | 29 | 39 | 35 | 34 | | 21 |
| 57. Zeama_GRF9_EF515848.1 | 28 | 36 | 45 | 35 | 59 | 25 | 54 | 43 | 35 | 48 | 44 | 39 | 79 | 27 | 45 | 44 | 54 | 33 | |

TABLE B.2
MatGAT results for global similarity and identity over the full length of the SYT polypeptide sequences.

| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|
| 1. Allce_SYT2 | | 34 | 49 | 31 | 46 | 46 | 34 | 39 | 50 | 32 | 40 | 38 | 29 | 32 | 47 | 36 | 46 | 49 | 47 | 29 | 34 | 48 | 48 | 48 |
| 2. Aqufo_SYT1 | 53 | | 39 | 60 | 37 | 38 | 58 | 35 | 36 | 60 | 35 | 34 | 26 | 69 | 41 | 55 | 36 | 44 | 40 | 64 | 62 | 39 | 41 | 41 |
| 3. Aqufo_SYT2 | 61 | 56 | | 38 | 50 | 52 | 38 | 45 | 47 | 38 | 50 | 34 | 29 | 42 | 61 | 46 | 47 | 59 | 56 | 39 | 40 | 65 | 64 | 64 |
| 4. Arath_SYT1 | 49 | 78 | 52 | | 36 | 36 | 56 | 35 | 35 | 95 | 36 | 31 | 27 | 67 | 37 | 51 | 32 | 44 | 37 | 65 | 65 | 38 | 38 | 38 |
| 5. Arath_SYT2 | 59 | 54 | 62 | 50 | | 64 | 38 | 52 | 45 | 36 | 78 | 34 | 30 | 39 | 56 | 39 | 43 | 61 | 53 | 36 | 38 | 55 | 54 | 54 |
| 6. Arath_SYT3 | 59 | 53 | 67 | 52 | 68 | | 38 | 41 | 43 | 35 | 65 | 33 | 31 | 37 | 60 | 40 | 44 | 60 | 54 | 37 | 36 | 61 | 62 | 62 |
| 7. Aspof_SYT1 | 55 | 74 | 51 | 67 | 54 | 54 | | 36 | 35 | 56 | 38 | 36 | 27 | 58 | 42 | 56 | 32 | 44 | 42 | 59 | 59 | 41 | 39 | 39 |
| 8. Betvu_SYT2 | 47 | 45 | 55 | 47 | 61 | 52 | 47 | | 41 | 35 | 48 | 38 | 32 | 35 | 46 | 35 | 40 | 44 | 46 | 33 | 36 | 42 | 44 | 44 |
| 9. Bradi_SYT3 | 63 | 51 | 56 | 50 | 56 | 53 | 46 | 51 | | 34 | 40 | 33 | 32 | 36 | 50 | 37 | 48 | 46 | 45 | 35 | 35 | 50 | 51 | 51 |
| 10. Brana_SYT1 | 49 | 77 | 52 | 96 | 52 | 50 | 68 | 50 | 50 | | 34 | 30 | 25 | 66 | 37 | 50 | 34 | 42 | 37 | 67 | 64 | 38 | 37 | 37 |
| 11. Brana_SYT2 | 60 | 53 | 65 | 54 | 83 | 73 | 55 | 56 | 51 | 53 | | 37 | 32 | 36 | 57 | 36 | 40 | 62 | 52 | 35 | 36 | 54 | 54 | 54 |
| 12. Cerri_SYT\partial | 54 | 49 | 46 | 44 | 48 | 47 | 51 | 50 | 46 | 46 | 48 | | 26 | 33 | 36 | 33 | 34 | 39 | 38 | 32 | 32 | 38 | 38 | 38 |
| 13. Chlre_SYT | 39 | 35 | 39 | 36 | 42 | 38 | 37 | 45 | 46 | 33 | 40 | 37 | | 24 | 29 | 27 | 25 | 28 | 30 | 24 | 24 | 28 | 31 | 31 |
| 14. Citsi_SYT1 | 47 | 83 | 59 | 80 | 55 | 55 | 71 | 46 | 49 | 77 | 53 | 47 | 32 | | 42 | 58 | 34 | 43 | 39 | 71 | 72 | 41 | 42 | 42 |
| 15. Citsi_SYT2 | 61 | 58 | 72 | 54 | 66 | 69 | 58 | 57 | 61 | 55 | 68 | 51 | 40 | 61 | | 44 | 45 | 73 | 67 | 40 | 40 | 82 | 81 | 81 |
| 16. Cryja_SYT | 48 | 70 | 57 | 64 | 51 | 53 | 68 | 42 | 48 | 64 | 48 | 46 | 36 | 73 | 57 | | 41 | 43 | 39 | 53 | 54 | 43 | 44 | 44 |
| 17. Curlo_SYT | 62 | 49 | 59 | 46 | 54 | 57 | 44 | 47 | 61 | 46 | 55 | 46 | 35 | 50 | 58 | 53 | | 46 | 41 | 37 | 34 | 47 | 47 | 47 |
| 18. Eupes_SYT2 | 62 | 59 | 68 | 59 | 73 | 67 | 58 | 54 | 56 | 59 | 74 | 55 | 39 | 57 | 80 | 54 | 59 | | 67 | 42 | 43 | 73 | 74 | 74 |
| 19. Frava_SYT2 | 61 | 57 | 64 | 53 | 64 | 62 | 55 | 56 | 54 | 52 | 59 | 50 | 41 | 56 | 75 | 51 | 56 | 76 | | 39 | 37 | 68 | 69 | 69 |
| 20. Glyma_SYT1.1 | 49 | 79 | 55 | 79 | 51 | 56 | 71 | 47 | 50 | 81 | 51 | 45 | 33 | 79 | 60 | 67 | 53 | 58 | 57 | | 73 | 38 | 39 | 39 |
| 21. Glyma_SYT1.2 | 50 | 74 | 53 | 77 | 53 | 50 | 71 | 51 | 49 | 75 | 53 | 50 | 33 | 79 | 56 | 67 | 48 | 55 | 50 | 83 | | 39 | 41 | 41 |
| 22. Glyma_SYT2.1 | 61 | 59 | 75 | 54 | 67 | 72 | 54 | 52 | 61 | 55 | 67 | 50 | 35 | 61 | 89 | 55 | 63 | 80 | 75 | 56 | 53 | | 97 | 97 |
| 23. Glyma_SYT2.2 | 59 | 61 | 74 | 52 | 68 | 72 | 53 | 55 | 61 | 53 | 67 | 51 | 42 | 61 | 87 | 56 | 63 | 81 | 77 | 58 | 54 | 98 | | ## |
| 24. Glyso_SYT2 | 59 | 61 | 74 | 52 | 68 | 72 | 53 | 55 | 61 | 53 | 67 | 51 | 42 | 61 | 87 | 56 | 63 | 81 | 77 | 58 | 54 | 98 | ## | |
| 25. Goshi_SYT1 | 45 | 81 | 57 | 78 | 49 | 55 | 70 | 46 | 50 | 76 | 49 | 45 | 31 | 90 | 61 | 75 | 48 | 55 | 53 | 81 | 79 | 59 | 56 | 56 |
| 26. Goshi_SYT2 | 59 | 61 | 73 | 55 | 65 | 73 | 57 | 57 | 54 | 52 | 69 | 47 | 39 | 61 | 87 | 58 | 57 | 78 | 72 | 57 | 56 | 86 | 85 | 85 |
| 27. Helan_SYT1 | 54 | 77 | 58 | 81 | 52 | 53 | 71 | 52 | 47 | 81 | 49 | 45 | 35 | 82 | 58 | 68 | 53 | 56 | 54 | 82 | 79 | 53 | 53 | 53 |
| 28. Horvu_SYT2 | 61 | 51 | 51 | 47 | 53 | 53 | 48 | 50 | 74 | 48 | 56 | 48 | 47 | 46 | 54 | 44 | 55 | 58 | 56 | 45 | 45 | 52 | 57 | 57 |
| 29. Lacse_SYT2 | 50 | 51 | 54 | 47 | 59 | 52 | 46 | 52 | 51 | 50 | 61 | 45 | 39 | 49 | 60 | 45 | 49 | 63 | 58 | 47 | 47 | 59 | 60 | 60 |
| 30. Lyces_SYT1 | 48 | 75 | 53 | 81 | 51 | 56 | 65 | 49 | 51 | 80 | 51 | 46 | 36 | 77 | 57 | 64 | 46 | 53 | 56 | 78 | 74 | 56 | 57 | 57 |
| 31. Maldo_SYT2 | 55 | 60 | 70 | 56 | 66 | 70 | 59 | 56 | 58 | 53 | 68 | 49 | 36 | 61 | 84 | 56 | 59 | 80 | 79 | 59 | 53 | 82 | 82 | 82 |
| 32. Medtr_SYT1 | 53 | 83 | 62 | 78 | 54 | 56 | 72 | 48 | 51 | 78 | 53 | 44 | 39 | 83 | 65 | 68 | 51 | 52 | 54 | 90 | 83 | 57 | 58 | 58 |
| 33. Medtr_SYT2 | 59 | 60 | 73 | 55 | 69 | 70 | 56 | 56 | 58 | 54 | 70 | 50 | 39 | 61 | 84 | 58 | 62 | 81 | 75 | 58 | 56 | 90 | 89 | 89 |
| 34. Orysa_SYT1 | 49 | 65 | 55 | 61 | 48 | 51 | 71 | 47 | 46 | 60 | 47 | 49 | 34 | 67 | 52 | 65 | 48 | 52 | 51 | 62 | 60 | 54 | 52 | 52 |
| 35. Orysa_SYT2 | 62 | 48 | 53 | 47 | 55 | 50 | 51 | 50 | 72 | 49 | 53 | 49 | 43 | 47 | 52 | 48 | 56 | 58 | 55 | 48 | 48 | 55 | 56 | 56 |
| 36. Orysa_SYT3 | 63 | 51 | 58 | 48 | 58 | 50 | 45 | 49 | 87 | 49 | 54 | 51 | 37 | 46 | 58 | 44 | 60 | 58 | 56 | 48 | 48 | 57 | 60 | 60 |
| 37. Panvi_SYT3 | 63 | 51 | 56 | 49 | 53 | 56 | 51 | 53 | 84 | 52 | 55 | 45 | 44 | 54 | 59 | 47 | 60 | 58 | 56 | 46 | 46 | 62 | 62 | 62 |
| 38. Phypa_SYT1.1 | 53 | 57 | 49 | 58 | 45 | 51 | 51 | 44 | 48 | 56 | 49 | 50 | 38 | 57 | 54 | 54 | 52 | 50 | 48 | 60 | 55 | 57 | 57 | 57 |
| 39. Phypa_SYT1.2 | 51 | 60 | 51 | 52 | 46 | 48 | 55 | 50 | 51 | 56 | 51 | 49 | 38 | 56 | 54 | 55 | 53 | 50 | 49 | 53 | 52 | 54 | 54 | 54 |
| 40. Phypa_SYT1.3 | 51 | 59 | 52 | 56 | 48 | 49 | 57 | 47 | 49 | 54 | 50 | 48 | 35 | 58 | 55 | 59 | 47 | 50 | 50 | 57 | 57 | 54 | 50 | 50 |
| 41. Phypa_SYT1.4 | 51 | 59 | 49 | 56 | 50 | 50 | 56 | 46 | 53 | 52 | 50 | 47 | 34 | 58 | 54 | 57 | 47 | 51 | 52 | 56 | 56 | 56 | 53 | 53 |
| 42. Picsi_SYT1 | 46 | 71 | 57 | 64 | 49 | 48 | 67 | 42 | 47 | 63 | 48 | 45 | 38 | 70 | 59 | 90 | 51 | 54 | 52 | 67 | 67 | 55 | 55 | 55 |
| 43. Pinta_SYT1 | 48 | 71 | 59 | 65 | 49 | 50 | 67 | 43 | 49 | 62 | 48 | 44 | 39 | 71 | 58 | 87 | 53 | 55 | 53 | 67 | 66 | 56 | 54 | 54 |
| 44. Poptr_SYT1 | 50 | 83 | 58 | 80 | 54 | 53 | 70 | 46 | 48 | 79 | 51 | 46 | 35 | 90 | 61 | 72 | 47 | 56 | 54 | 82 | 80 | 57 | 56 | 56 |
| 45. Poptr_SYT2 | 61 | 60 | 72 | 54 | 64 | 71 | 56 | 54 | 57 | 56 | 71 | 43 | 39 | 60 | 86 | 56 | 63 | 81 | 75 | 61 | 55 | 83 | 83 | 83 |
| 46. Poptr_SYT3 | 45 | 40 | 46 | 43 | 49 | 42 | 41 | 50 | 43 | 45 | 44 | 49 | 39 | 37 | 46 | 38 | 41 | 47 | 52 | 41 | 41 | 47 | 48 | 48 |
| 47. Prupe_SYT2 | 56 | 59 | 72 | 56 | 63 | 67 | 62 | 55 | 58 | 54 | 67 | 48 | 36 | 58 | 82 | 57 | 60 | 76 | 80 | 58 | 53 | 80 | 82 | 82 |
| 48. Sacof_SYT1 | 49 | 64 | 53 | 58 | 47 | 50 | 68 | 48 | 50 | 58 | 48 | 52 | 33 | 64 | 50 | 61 | 50 | 52 | 51 | 63 | 58 | 52 | 50 | 50 |
| 49. Sacof_SYT2 | 59 | 49 | 51 | 50 | 55 | 49 | 48 | 53 | 72 | 50 | 54 | 50 | 44 | 46 | 54 | 46 | 57 | 58 | 55 | 48 | 51 | 54 | 55 | 55 |
| 50. Sacof_SYT3 | 60 | 49 | 56 | 48 | 53 | 56 | 47 | 47 | 80 | 50 | 57 | 44 | 46 | 51 | 55 | 48 | 61 | 53 | 53 | 47 | 49 | 58 | 58 | 58 |
| 51. Soltu_SYT1 | 51 | 75 | 55 | 80 | 49 | 56 | 62 | 48 | 50 | 79 | 48 | 46 | 36 | 78 | 58 | 64 | 50 | 54 | 52 | 79 | 70 | 56 | 55 | 55 |
| 52. Soltu_SYT2 | 47 | 74 | 56 | 80 | 54 | 56 | 65 | 47 | 50 | 78 | 53 | 45 | 34 | 81 | 58 | 65 | 51 | 57 | 54 | 80 | 75 | 51 | 56 | 56 |
| 53. Soltu_SYT3 | 58 | 57 | 70 | 54 | 61 | 59 | 52 | 53 | 55 | 56 | 64 | 49 | 38 | 52 | 75 | 53 | 53 | 74 | 72 | 53 | 53 | 75 | 74 | 74 |
| 54. Sorbi_SYT1 | 49 | 63 | 54 | 60 | 46 | 50 | 68 | 44 | 49 | 58 | 48 | 51 | 33 | 64 | 50 | 62 | 52 | 52 | 51 | 64 | 56 | 52 | 50 | 50 |
| 55. Sorbi_SYT2 | 61 | 50 | 52 | 47 | 57 | 54 | 48 | 53 | 73 | 50 | 53 | 49 | 43 | 47 | 55 | 46 | 58 | 60 | 57 | 48 | 51 | 56 | 54 | 54 |
| 56. Sorbi_SYT3 | 62 | 50 | 55 | 48 | 53 | 55 | 48 | 46 | 82 | 50 | 56 | 48 | 38 | 51 | 53 | 48 | 60 | 54 | 52 | 46 | 45 | 58 | 58 | 58 |
| 57. Tarof_SYT2 | 47 | 52 | 55 | 51 | 61 | 51 | 52 | 51 | 48 | 52 | 60 | 47 | 40 | 50 | 62 | 49 | 51 | 65 | 60 | 51 | 49 | 60 | 61 | 61 |
| 58. Tarof_SYT3 | 50 | 51 | 57 | 50 | 54 | 54 | 50 | 56 | 57 | 49 | 56 | 45 | 37 | 51 | 60 | 47 | 48 | 55 | 52 | 52 | 49 | 57 | 56 | 56 |
| 59. Triae_SYT1 | 51 | 65 | 57 | 64 | 48 | 50 | 72 | 48 | 47 | 61 | 48 | 48 | 39 | 68 | 54 | 67 | 49 | 50 | 51 | 65 | 63 | 56 | 54 | 54 |
| 60. Triae_SYT2 | 61 | 50 | 51 | 46 | 55 | 53 | 49 | 51 | 74 | 48 | 55 | 48 | 47 | 46 | 53 | 45 | 54 | 58 | 54 | 46 | 47 | 52 | 56 | 56 |
| 61. Triae_SYT3 | 60 | 49 | 57 | 49 | 58 | 52 | 46 | 50 | 90 | 48 | 56 | 47 | 44 | 48 | 60 | 44 | 60 | 59 | 56 | 49 | 49 | 59 | 60 | 60 |
| 62. Triae_SYT3.2 | 60 | 49 | 57 | 49 | 58 | 52 | 46 | 50 | 90 | 48 | 56 | 47 | 44 | 48 | 60 | 44 | 60 | 59 | 56 | 49 | 49 | 59 | 60 | 60 |
| 63. Vitvi_SYT1.1 | 50 | 81 | 57 | 76 | 53 | 54 | 72 | 46 | 48 | 77 | 51 | 45 | 35 | 90 | 59 | 76 | 50 | 55 | 52 | 82 | 83 | 56 | 59 | 59 |
| 64. Vitvi_SYT1.2 | 44 | 76 | 55 | 70 | 51 | 54 | 65 | 50 | 47 | 67 | 53 | 49 | 32 | 80 | 56 | 67 | 51 | 55 | 50 | 73 | 76 | 56 | 56 | 56 |
| 65. Vitvi_SYT2.1 | 59 | 61 | 75 | 57 | 67 | 73 | 57 | 54 | 60 | 54 | 68 | 47 | 37 | 65 | 83 | 60 | 60 | 79 | 70 | 64 | 55 | 83 | 82 | 82 |
| 66. Vitvi_SYT2.2 | 56 | 49 | 64 | 53 | 67 | 61 | 49 | 64 | 55 | 55 | 63 | 50 | 41 | 54 | 68 | 49 | 54 | 67 | 65 | 51 | 49 | 70 | 70 | 70 |
| 67. Volca_SYT | 39 | 34 | 41 | 38 | 39 | 39 | 38 | 37 | 42 | 36 | 42 | 37 | 54 | 38 | 37 | 39 | 38 | 34 | 39 | 38 | 39 | 37 | 38 | 38 |
| 68. Welmi_SYT1 | 54 | 71 | 60 | 64 | 53 | 53 | 66 | 47 | 47 | 61 | 48 | 49 | 36 | 71 | 54 | 83 | 50 | 56 | 51 | 65 | 64 | 58 | 57 | 57 |
| 69. Zeama_SYT1 | 49 | 62 | 53 | 59 | 45 | 50 | 68 | 43 | 45 | 58 | 48 | 48 | 32 | 63 | 50 | 57 | 49 | 51 | 51 | 60 | 60 | 52 | 50 | 50 |
| 70. Zeama_SYT2 | 59 | 50 | 48 | 46 | 54 | 48 | 49 | 52 | 74 | 50 | 51 | 51 | 45 | 50 | 52 | 45 | 55 | 60 | 57 | 50 | 47 | 55 | 55 | 55 |
| 71. Zeama_SYT3 | 58 | 49 | 55 | 47 | 50 | 54 | 46 | 46 | 80 | 49 | 52 | 42 | 41 | 51 | 53 | 50 | 59 | 52 | 54 | 49 | 46 | 56 | 56 | 56 |
| | 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 |
| 1. Allce_SYT2 | 32 | 46 | 35 | 48 | 39 | 31 | 43 | 34 | 48 | 35 | 50 | 51 | 51 | 37 | 39 | 38 | 38 | 32 | 34 | 34 | 48 | 36 | 47 | 32 |
| 2. Aqufo_SYT1 | 70 | 43 | 60 | 36 | 38 | 60 | 42 | 64 | 43 | 54 | 36 | 34 | 36 | 40 | 43 | 42 | 41 | 56 | 55 | 69 | 40 | 29 | 40 | 50 |
| 3. Aqufo_SYT2 | 42 | 60 | 44 | 45 | 45 | 39 | 60 | 44 | 64 | 42 | 45 | 48 | 47 | 37 | 40 | 41 | 40 | 45 | 47 | 42 | 61 | 39 | 62 | 39 |
| 4. Arath_SYT1 | 66 | 38 | 67 | 35 | 35 | 71 | 40 | 65 | 40 | 50 | 36 | 34 | 35 | 37 | 36 | 40 | 40 | 50 | 52 | 66 | 37 | 31 | 38 | 45 |
| 5. Arath_SYT2 | 36 | 55 | 38 | 43 | 49 | 37 | 54 | 39 | 56 | 33 | 43 | 46 | 45 | 33 | 32 | 36 | 37 | 37 | 37 | 39 | 53 | 38 | 54 | 36 |
| 6. Arath_SYT3 | 39 | 60 | 37 | 43 | 45 | 37 | 61 | 40 | 60 | 35 | 41 | 40 | 45 | 37 | 38 | 37 | 39 | 35 | 36 | 38 | 62 | 35 | 57 | 34 |
| 7. Aspof_SYT1 | 59 | 45 | 58 | 36 | 36 | 53 | 45 | 57 | 42 | 61 | 39 | 34 | 39 | 36 | 41 | 44 | 43 | 56 | 56 | 57 | 39 | 33 | 46 | 56 |
| 8. Betvu_SYT2 | 36 | 46 | 38 | 39 | 43 | 38 | 44 | 34 | 44 | 33 | 40 | 40 | 43 | 35 | 37 | 36 | 37 | 34 | 34 | 35 | 42 | 41 | 44 | 33 |
| 9. Bradi_SYT3 | 38 | 47 | 35 | 68 | 41 | 36 | 47 | 37 | 50 | 36 | 66 | 80 | 78 | 37 | 37 | 38 | 38 | 36 | 38 | 38 | 47 | 35 | 48 | 36 |
| 10. Brana_SYT1 | 66 | 36 | 67 | 35 | 34 | 70 | 38 | 65 | 39 | 50 | 37 | 34 | 34 | 37 | 35 | 40 | 38 | 49 | 50 | 67 | 36 | 32 | 36 | 46 |
| 11. Brana_SYT2 | 34 | 55 | 36 | 43 | 48 | 35 | 56 | 36 | 56 | 35 | 41 | 41 | 43 | 35 | 38 | 36 | 36 | 37 | 36 | 36 | 56 | 37 | 55 | 34 |
| 12. Cerri_SYT\partial | 32 | 36 | 31 | 35 | 31 | 32 | 36 | 30 | 37 | 34 | 33 | 36 | 34 | 38 | 39 | 35 | 37 | 35 | 33 | 31 | 34 | 36 | 37 | 37 |
| 13. Chlre_SYT | 23 | 29 | 27 | 33 | 28 | 25 | 27 | 26 | 29 | 25 | 29 | 27 | 32 | 26 | 26 | 26 | 24 | 29 | 29 | 24 | 27 | 30 | 28 | 22 |
| 14. Citsi_SYT1 | 87 | 43 | 70 | 35 | 35 | 68 | 43 | 73 | 43 | 54 | 37 | 35 | 39 | 39 | 40 | 44 | 42 | 57 | 57 | 82 | 41 | 27 | 41 | 50 |
| 15. Citsi_SYT2 | 45 | 78 | 40 | 44 | 50 | 40 | 78 | 46 | 76 | 40 | 44 | 47 | 49 | 41 | 42 | 42 | 41 | 45 | 46 | 44 | 79 | 40 | 79 | 37 |
| 16. Cryja_SYT | 63 | 44 | 53 | 34 | 36 | 50 | 43 | 56 | 45 | 52 | 37 | 33 | 34 | 41 | 44 | 46 | 45 | 85 | 82 | 59 | 42 | 29 | 44 | 47 |
| 17. Curlo_SYT | 37 | 44 | 37 | 46 | 39 | 34 | 47 | 36 | 46 | 35 | 48 | 48 | 47 | 37 | 38 | 33 | 34 | 38 | 40 | 32 | 49 | 31 | 47 | 36 |
| 18. Eupes_SYT2 | 42 | 69 | 40 | 48 | 50 | 40 | 73 | 42 | 73 | 43 | 50 | 48 | 48 | 39 | 40 | 39 | 40 | 44 | 44 | 44 | 73 | 40 | 70 | 40 |
| 19. Frava_SYT2 | 39 | 62 | 39 | 44 | 51 | 38 | 72 | 41 | 68 | 40 | 46 | 46 | 46 | 38 | 38 | 40 | 42 | 39 | 40 | 40 | 66 | 43 | 73 | 39 |
| 20. Glyma_SYT1.1 | 73 | 39 | 71 | 34 | 34 | 66 | 43 | 79 | 41 | 51 | 36 | 32 | 33 | 40 | 36 | 41 | 40 | 53 | 52 | 73 | 41 | 29 | 40 | 50 |
| 21. Glyma_SYT1.2 | 71 | 44 | 65 | 35 | 34 | 62 | 39 | 74 | 39 | 50 | 38 | 37 | 33 | 39 | 39 | 41 | 41 | 54 | 54 | 72 | 40 | 31 | 39 | 47 |
| 22. Glyma_SYT2.1 | 42 | 75 | 38 | 46 | 50 | 40 | 75 | 42 | 84 | 41 | 47 | 48 | 51 | 42 | 42 | 42 | 42 | 43 | 45 | 42 | 77 | 38 | 73 | 37 |
| 23. Glyma_SYT2.2 | 41 | 73 | 37 | 48 | 51 | 41 | 75 | 41 | 84 | 40 | 47 | 49 | 51 | 42 | 40 | 40 | 41 | 44 | 45 | 41 | 77 | 39 | 75 | 35 |
| 24. Glyso_SYT2 | 41 | 73 | 37 | 48 | 51 | 41 | 75 | 41 | 84 | 40 | 47 | 49 | 51 | 42 | 40 | 40 | 41 | 44 | 45 | 41 | 77 | 39 | 75 | 35 |
| 25. Goshi_SYT1 | | 44 | 71 | 35 | 34 | 68 | 45 | 73 | 42 | 53 | 36 | 35 | 38 | 38 | 37 | 42 | 42 | 62 | 62 | 85 | 42 | 28 | 45 | 48 |
| 26. Goshi_SYT2 | 60 | | 40 | 46 | 50 | 39 | 73 | 42 | 73 | 41 | 45 | 46 | 46 | 40 | 41 | 43 | 43 | 44 | 45 | 44 | 72 | 38 | 73 | 36 |
| 27. Helan_SYT1 | 82 | 57 | | 33 | 37 | 66 | 45 | 68 | 40 | 50 | 35 | 37 | 37 | 38 | 39 | 43 | 42 | 53 | 53 | 70 | 41 | 30 | 42 | 50 |
| 28. Horvu_SYT2 | 45 | 54 | 44 | | 39 | 35 | 45 | 37 | 45 | 33 | 78 | 69 | 63 | 34 | 36 | 38 | 36 | 35 | 36 | 35 | 45 | 35 | 46 | 31 |
| 29. Lacse_SYT2 | 47 | 62 | 52 | 51 | | 35 | 48 | 38 | 48 | 36 | 41 | 43 | 40 | 32 | 37 | 35 | 36 | 33 | 36 | 33 | 51 | 36 | 49 | 32 |
| 30. Lyces_SYT1 | 75 | 54 | 79 | 49 | 49 | | 40 | 64 | 41 | 47 | 39 | 36 | 35 | 41 | 37 | 38 | 36 | 51 | 51 | 65 | 41 | 31 | 37 | 45 |
| 31. Maldo_SYT2 | 60 | 83 | 61 | 57 | 59 | 58 | | 45 | 75 | 41 | 46 | 45 | 46 | 39 | 41 | 42 | 40 | 44 | 45 | 44 | 74 | 38 | 85 | 40 |
| 32. Medtr_SYT1 | 82 | 60 | 80 | 51 | 51 | 77 | 58 | | 43 | 53 | 39 | 35 | 37 | 40 | 40 | 42 | 41 | 56 | 58 | 74 | 43 | 31 | 44 | 51 |
| 33. Medtr_SYT2 | 58 | 85 | 56 | 53 | 57 | 60 | 82 | 60 | | 40 | 49 | 49 | 51 | 40 | 41 | 41 | 40 | 44 | 46 | 44 | 77 | 41 | 75 | 36 |
| 34. Orysa_SYT1 | 66 | 54 | 62 | 43 | 43 | 60 | 53 | 61 | 52 | | 35 | 36 | 37 | 36 | 37 | 38 | 37 | 52 | 52 | 53 | 37 | 28 | 41 | 82 |
| 35. Orysa_SYT2 | 45 | 55 | 46 | 84 | 53 | 50 | 55 | 50 | 58 | 46 | | 68 | 62 | 38 | 35 | 38 | 37 | 37 | 37 | 36 | 45 | 38 | 48 | 35 |
| 36. Orysa_SYT3 | 47 | 55 | 48 | 76 | 51 | 49 | 54 | 51 | 59 | 47 | 74 | | 78 | 39 | 39 | 38 | 39 | 36 | 38 | 35 | 46 | 34 | 47 | 40 |
| 37. Panvi_SYT3 | 51 | 59 | 51 | 72 | 51 | 50 | 57 | 52 | 62 | 52 | 70 | 85 | | 39 | 36 | 39 | 39 | 37 | 37 | 36 | 49 | 38 | 48 | 36 |
| 38. Phypa_SYT1.1 | 53 | 53 | 54 | 49 | 44 | 58 | 52 | 56 | 55 | 48 | 46 | 53 | 50 | | 82 | 50 | 51 | 42 | 44 | 42 | 39 | 35 | 39 | 34 |
| 39. Phypa_SYT1.2 | 53 | 52 | 56 | 47 | 49 | 54 | 54 | 54 | 52 | 48 | 45 | 54 | 47 | 87 | | 51 | 53 | 43 | 44 | 40 | 41 | 36 | 42 | 37 |
| 40. Phypa_SYT1.3 | 57 | 57 | 57 | 47 | 43 | 50 | 53 | 56 | 52 | 49 | 45 | 51 | 51 | 65 | 65 | | 93 | 45 | 46 | 43 | 41 | 35 | 40 | 38 |
| 41. Phypa_SYT1.4 | 57 | 55 | 58 | 47 | 46 | 48 | 49 | 55 | 54 | 50 | 47 | 51 | 51 | 67 | 68 | 96 | | 46 | 46 | 42 | 40 | 36 | 42 | 36 |
| 42. Picsi_SYT1 | 75 | 59 | 67 | 45 | 42 | 64 | 57 | 67 | 57 | 65 | 48 | 47 | 49 | 52 | 53 | 56 | 56 | | 94 | 59 | 42 | 29 | 43 | 47 |
| 43. Pinta_SYT1 | 76 | 59 | 67 | 45 | 43 | 65 | 58 | 68 | 59 | 63 | 46 | 48 | 49 | 55 | 55 | 57 | 57 | 95 | | 58 | 42 | 30 | 44 | 47 |
| 44. Poptr_SYT1 | 91 | 61 | 83 | 45 | 47 | 76 | 59 | 83 | 61 | 66 | 46 | 46 | 49 | 59 | 56 | 58 | 56 | 72 | 71 | | 43 | 29 | 44 | 49 |
| 45. Poptr_SYT2 | 57 | 83 | 61 | 55 | 62 | 58 | 80 | 60 | 83 | 51 | 56 | 56 | 61 | 51 | 56 | 50 | 55 | 55 | 54 | 60 | | 40 | 74 | 38 |
| 46. Poptr_SYT3 | 39 | 46 | 41 | 46 | 45 | 42 | 46 | 41 | 49 | 37 | 44 | 42 | 45 | 46 | 44 | 42 | 43 | 38 | 39 | 36 | 48 | | 39 | 26 |
| 47. Prupe_SYT2 | 60 | 82 | 58 | 53 | 59 | 51 | 87 | 60 | 83 | 54 | 56 | 56 | 57 | 51 | 53 | 50 | 50 | 56 | 56 | 59 | 78 | 47 | | 39 |
| 48. Sacof_SYT1 | 62 | 52 | 59 | 41 | 43 | 60 | 51 | 61 | 52 | 88 | 44 | 51 | 47 | 48 | 50 | 50 | 48 | 60 | 62 | 63 | 52 | 37 | 52 | |
| 49. Sacof_SYT2 | 46 | 55 | 46 | 86 | 56 | 53 | 53 | 46 | 53 | 45 | 84 | 72 | 73 | 45 | 49 | 50 | 47 | 45 | 46 | 47 | 53 | 44 | 54 | 44 |
| 50. Sacof_SYT3 | 51 | 58 | 48 | 71 | 46 | 52 | 54 | 48 | 56 | 45 | 70 | 84 | 87 | 51 | 47 | 52 | 52 | 49 | 50 | 48 | 52 | 44 | 55 | 48 |
| 51. Soltu_SYT1 | 76 | 56 | 77 | 49 | 47 | 97 | 54 | 77 | 58 | 60 | 49 | 49 | 53 | 59 | 53 | 51 | 53 | 65 | 63 | 77 | 58 | 40 | 53 | 59 |
| 52. Soltu_SYT2 | 81 | 56 | 78 | 46 | 49 | 77 | 55 | 74 | 56 | 64 | 47 | 49 | 53 | 59 | 56 | 50 | 48 | 65 | 67 | 82 | 58 | 39 | 56 | 63 |
| 53. Soltu_SYT3 | 50 | 75 | 55 | 54 | 58 | 58 | 76 | 56 | 75 | 47 | 53 | 57 | 57 | 51 | 50 | 50 | 52 | 53 | 53 | 53 | 74 | 49 | 74 | 50 |
| 54. Sorbi_SYT1 | 60 | 52 | 59 | 42 | 42 | 59 | 51 | 61 | 52 | 88 | 44 | 51 | 47 | 48 | 48 | 51 | 47 | 62 | 62 | 63 | 51 | 37 | 52 | 99 |
| 55. Sorbi_SYT2 | 45 | 57 | 46 | 87 | 52 | 52 | 56 | 48 | 56 | 45 | 86 | 71 | 74 | 46 | 50 | 50 | 48 | 45 | 46 | 48 | 53 | 46 | 54 | 46 |
| 56. Sorbi_SYT3 | 50 | 57 | 48 | 72 | 48 | 52 | 54 | 50 | 59 | 45 | 71 | 84 | 86 | 48 | 48 | 53 | 52 | 49 | 48 | 48 | 54 | 44 | 54 | 48 |
| 57. Tarof_SYT2 | 50 | 63 | 54 | 50 | 92 | 46 | 62 | 52 | 58 | 47 | 52 | 49 | 49 | 44 | 50 | 47 | 47 | 46 | 45 | 50 | 62 | 50 | 61 | 46 |
| 58. Tarof_SYT3 | 50 | 57 | 53 | 51 | 46 | 52 | 58 | 54 | 57 | 46 | 47 | 57 | 54 | 47 | 47 | 46 | 47 | 46 | 47 | 50 | 58 | 44 | 54 | 46 |
| 59. Triae_SYT1 | 67 | 56 | 63 | 44 | 43 | 65 | 54 | 64 | 54 | 92 | 47 | 47 | 51 | 51 | 53 | 54 | 52 | 67 | 67 | 67 | 52 | 38 | 56 | 87 |
| 60. Triae_SYT2 | 44 | 54 | 46 | 99 | 50 | 50 | 53 | 48 | 55 | 43 | 84 | 75 | 72 | 47 | 44 | 48 | 47 | 46 | 46 | 45 | 55 | 45 | 53 | 44 |
| 61. Triae_SYT3 | 51 | 61 | 49 | 75 | 50 | 51 | 59 | 51 | 60 | 46 | 74 | 82 | 80 | 47 | 50 | 46 | 51 | 47 | 50 | 49 | 58 | 44 | 57 | 50 |
| 62. Triae_SYT3.2 | 51 | 61 | 49 | 75 | 50 | 51 | 59 | 51 | 60 | 46 | 74 | 82 | 80 | 47 | 50 | 46 | 51 | 47 | 50 | 49 | 58 | 44 | 57 | 50 |
| 63. Vitvi_SYT1.1 | 91 | 58 | 82 | 47 | 48 | 74 | 60 | 83 | 52 | 65 | 46 | 45 | 49 | 56 | 53 | 59 | 58 | 74 | 73 | 91 | 54 | 39 | 59 | 61 |
| 64. Vitvi_SYT1.2 | 78 | 56 | 73 | 44 | 49 | 69 | 56 | 75 | 59 | 62 | 47 | 47 | 50 | 52 | 51 | 58 | 58 | 69 | 68 | 80 | 56 | 41 | 55 | 65 |
| 65. Vitvi_SYT2.1 | 63 | 82 | 58 | 55 | 59 | 56 | 78 | 65 | 84 | 57 | 55 | 58 | 60 | 53 | 52 | 51 | 54 | 60 | 61 | 65 | 79 | 47 | 81 | 53 |
| 66. Vitvi_SYT2.2 | 49 | 68 | 53 | 56 | 51 | 50 | 68 | 56 | 69 | 47 | 53 | 53 | 53 | 50 | 53 | 47 | 46 | 48 | 51 | 52 | 70 | 52 | 67 | 47 |
| 67. Volca_SYT | 37 | 37 | 36 | 42 | 35 | 39 | 40 | 36 | 39 | 35 | 38 | 40 | 38 | 37 | 35 | 37 | 36 | 41 | 41 | 38 | 39 | 31 | 37 | 37 |
| 68. Welmi_SYT1 | 74 | 57 | 66 | 45 | 45 | 63 | 59 | 66 | 60 | 64 | 48 | 47 | 50 | 53 | 56 | 60 | 57 | 83 | 82 | 69 | 59 | 36 | 57 | 63 |
| 69. Zeama_SYT1 | 62 | 52 | 59 | 43 | 42 | 58 | 52 | 61 | 52 | 88 | 44 | 50 | 49 | 46 | 45 | 48 | 49 | 60 | 59 | 63 | 50 | 36 | 52 | 97 |
| 70. Zeama_SYT2 | 47 | 55 | 48 | 83 | 53 | 51 | 53 | 48 | 53 | 45 | 84 | 72 | 71 | 44 | 47 | 51 | 49 | 45 | 45 | 46 | 52 | 46 | 52 | 46 |
| 71. Zeama_SYT3 | 52 | 57 | 49 | 69 | 49 | 48 | 54 | 51 | 57 | 48 | 68 | 84 | 84 | 47 | 46 | 51 | 51 | 49 | 55 | 52 | 55 | 42 | 54 | 45 |

| | 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 |
|---|---|---|---|---|---|---|---|---|---|---|---|---|---|---|---|---|---|---|---|---|---|---|---|
| 1. Allce_SYT2 | 49 | 49 | 34 | 31 | 46 | 31 | 50 | 50 | 37 | 36 | 35 | 47 | 48 | 48 | 35 | 32 | 47 | 45 | 28 | 36 | 35 | 49 | 48 |
| 2. Aqufo_SYT1 | 36 | 34 | 60 | 59 | 39 | 50 | 36 | 34 | 39 | 33 | 53 | 35 | 33 | 33 | 68 | 62 | 44 | 36 | 25 | 56 | 50 | 36 | 35 |
| 3. Aqufo_SYT2 | 46 | 46 | 40 | 40 | 62 | 39 | 46 | 44 | 46 | 45 | 43 | 44 | 47 | 47 | 41 | 39 | 63 | 58 | 29 | 44 | 39 | 43 | 44 |
| 4. Arath_SYT1 | 35 | 32 | 72 | 70 | 39 | 46 | 33 | 32 | 37 | 35 | 50 | 34 | 34 | 34 | 62 | 56 | 40 | 39 | 25 | 47 | 46 | 34 | 32 |
| 5. Arath_SYT2 | 45 | 43 | 36 | 36 | 49 | 36 | 46 | 45 | 50 | 44 | 35 | 45 | 46 | 46 | 39 | 36 | 56 | 53 | 29 | 36 | 36 | 46 | 42 |
| 6. Arath_SYT3 | 41 | 43 | 36 | 37 | 50 | 35 | 44 | 43 | 43 | 43 | 35 | 43 | 42 | 42 | 37 | 36 | 60 | 50 | 28 | 35 | 35 | 40 | 44 |
| 7. Aspof_SYT1 | 36 | 34 | 53 | 56 | 39 | 56 | 36 | 34 | 40 | 37 | 60 | 36 | 33 | 33 | 58 | 54 | 44 | 37 | 27 | 52 | 55 | 37 | 35 |
| 8. Betvu_SYT2 | 41 | 37 | 37 | 32 | 42 | 33 | 41 | 37 | 43 | 45 | 32 | 39 | 40 | 40 | 36 | 35 | 43 | 52 | 27 | 32 | 32 | 40 | 38 |
| 9. Bradi_SYT3 | 67 | 77 | 36 | 35 | 43 | 36 | 69 | 78 | 42 | 43 | 37 | 68 | 85 | 85 | 36 | 36 | 49 | 49 | 28 | 36 | 35 | 70 | 77 |
| 10. Brana_SYT1 | 35 | 32 | 70 | 67 | 39 | 47 | 33 | 32 | 37 | 34 | 48 | 34 | 32 | 32 | 63 | 54 | 39 | 38 | 24 | 44 | 47 | 35 | 33 |
| 11. Brana_SYT2 | 43 | 43 | 34 | 35 | 51 | 34 | 43 | 43 | 50 | 43 | 35 | 44 | 43 | 43 | 36 | 35 | 55 | 51 | 31 | 31 | 34 | 42 | 42 |
| 12. Cerri_SYT\partial | 36 | 35 | 32 | 31 | 39 | 35 | 35 | 33 | 32 | 32 | 34 | 34 | 34 | 34 | 32 | 32 | 35 | 36 | 25 | 35 | 34 | 35 | 33 |
| 13. Chlre_SYT | 32 | 32 | 26 | 25 | 29 | 22 | 31 | 28 | 30 | 28 | 26 | 33 | 32 | 32 | 24 | 23 | 28 | 30 | 48 | 28 | 21 | 31 | 31 |
| 14. Citsi_SYT1 | 33 | 37 | 68 | 70 | 37 | 50 | 37 | 36 | 37 | 36 | 52 | 35 | 36 | 36 | 80 | 68 | 46 | 40 | 24 | 53 | 50 | 38 | 36 |
| 15. Citsi_SYT2 | 44 | 46 | 42 | 41 | 68 | 38 | 44 | 43 | 51 | 46 | 41 | 44 | 48 | 48 | 43 | 40 | 79 | 59 | 27 | 39 | 37 | 45 | 44 |
| 16. Cryja_SYT | 37 | 35 | 51 | 52 | 44 | 48 | 37 | 36 | 38 | 33 | 52 | 35 | 35 | 35 | 65 | 54 | 47 | 37 | 23 | 70 | 44 | 36 | 37 |
| 17. Curlo_SYT | 48 | 49 | 34 | 34 | 40 | 37 | 49 | 48 | 41 | 34 | 37 | 45 | 48 | 48 | 36 | 33 | 47 | 44 | 24 | 39 | 35 | 46 | 47 |
| 18. Eupes_SYT2 | 47 | 45 | 41 | 43 | 63 | 40 | 49 | 45 | 54 | 47 | 41 | 48 | 46 | 46 | 43 | 42 | 73 | 58 | 25 | 41 | 40 | 50 | 44 |
| 19. Frava_SYT2 | 45 | 44 | 39 | 40 | 61 | 39 | 46 | 44 | 51 | 42 | 39 | 43 | 46 | 46 | 39 | 37 | 65 | 58 | 28 | 37 | 39 | 46 | 44 |
| 20. Glyma_SYT1.1 | 35 | 32 | 66 | 68 | 38 | 50 | 36 | 32 | 36 | 35 | 51 | 35 | 33 | 33 | 74 | 61 | 46 | 36 | 25 | 48 | 48 | 38 | 36 |
| 21. Glyma_SYT1.2 | 39 | 32 | 62 | 65 | 39 | 48 | 39 | 33 | 37 | 33 | 51 | 37 | 35 | 35 | 74 | 63 | 43 | 38 | 26 | 49 | 48 | 37 | 32 |
| 22. Glyma_SYT2.1 | 47 | 49 | 41 | 37 | 66 | 37 | 48 | 47 | 51 | 44 | 42 | 45 | 49 | 49 | 42 | 41 | 77 | 61 | 27 | 41 | 38 | 46 | 47 |
| 23. Glyma_SYT2.2 | 48 | 49 | 41 | 38 | 67 | 35 | 46 | 48 | 52 | 44 | 42 | 47 | 50 | 50 | 42 | 41 | 76 | 61 | 27 | 41 | 37 | 47 | 47 |
| 24. Glyso_SYT2 | 48 | 49 | 41 | 38 | 67 | 35 | 46 | 48 | 52 | 44 | 42 | 47 | 50 | 50 | 42 | 41 | 76 | 61 | 27 | 41 | 37 | 47 | 47 |
| 25. Goshi_SYT1 | 34 | 37 | 69 | 67 | 39 | 47 | 35 | 36 | 36 | 34 | 54 | 33 | 38 | 38 | 84 | 66 | 46 | 38 | 26 | 57 | 48 | 36 | 38 |
| 26. Goshi_SYT2 | 46 | 47 | 40 | 40 | 63 | 36 | 47 | 46 | 50 | 43 | 40 | 45 | 47 | 47 | 42 | 39 | 73 | 58 | 27 | 41 | 37 | 47 | 45 |
| 27. Helan_SYT1 | 35 | 35 | 65 | 67 | 37 | 50 | 35 | 35 | 40 | 36 | 51 | 32 | 35 | 35 | 67 | 58 | 43 | 39 | 25 | 47 | 49 | 36 | 36 |
| 28. Horvu_SYT2 | 76 | 65 | 35 | 34 | 44 | 31 | 77 | 65 | 40 | 40 | 34 | 97 | 67 | 67 | 37 | 34 | 46 | 48 | 28 | 34 | 32 | 76 | 65 |
| 29. Lacse_SYT2 | 42 | 38 | 35 | 35 | 47 | 32 | 42 | 39 | 89 | 38 | 34 | 39 | 40 | 40 | 35 | 35 | 49 | 42 | 25 | 33 | 34 | 42 | 40 |
| 30. Lyces_SYT1 | 38 | 34 | 97 | 70 | 41 | 45 | 38 | 35 | 35 | 36 | 49 | 36 | 36 | 36 | 64 | 58 | 43 | 36 | 26 | 47 | 45 | 37 | 34 |
| 31. Maldo_SYT2 | 45 | 44 | 40 | 40 | 65 | 40 | 46 | 45 | 52 | 46 | 41 | 43 | 47 | 47 | 43 | 42 | 74 | 58 | 30 | 41 | 39 | 43 | 46 |
| 32. Medtr_SYT1 | 35 | 34 | 64 | 67 | 44 | 52 | 36 | 35 | 39 | 36 | 53 | 36 | 36 | 36 | 73 | 61 | 51 | 41 | 24 | 51 | 51 | 39 | 36 |
| 33. Medtr_SYT2 | 47 | 47 | 41 | 41 | 67 | 37 | 48 | 48 | 50 | 45 | 41 | 47 | 50 | 50 | 41 | 41 | 76 | 61 | 28 | 42 | 37 | 46 | 48 |
| 34. Orysa_SYT1 | 32 | 34 | 48 | 53 | 38 | 82 | 32 | 35 | 37 | 36 | 88 | 33 | 36 | 36 | 53 | 49 | 43 | 36 | 24 | 48 | 83 | 31 | 37 |
| 35. Orysa_SYT2 | 75 | 63 | 38 | 35 | 43 | 35 | 76 | 63 | 43 | 39 | 36 | 77 | 65 | 65 | 36 | 36 | 47 | 47 | 26 | 37 | 34 | 75 | 62 |
| 36. Orysa_SYT3 | 68 | 79 | 36 | 37 | 44 | 39 | 67 | 79 | 43 | 43 | 36 | 70 | 75 | 75 | 34 | 37 | 48 | 47 | 26 | 36 | 38 | 67 | 80 |
| 37. Panvi_SYT3 | 66 | 83 | 37 | 36 | 45 | 36 | 68 | 83 | 42 | 40 | 36 | 64 | 71 | 71 | 35 | 39 | 50 | 47 | 28 | 36 | 37 | 66 | 82 |
| 38. Phypa_SYT1.1 | 35 | 38 | 42 | 40 | 40 | 34 | 36 | 36 | 34 | 33 | 37 | 33 | 36 | 36 | 39 | 38 | 41 | 38 | 24 | 42 | 35 | 36 | 39 |
| 39. Phypa_SYT1.2 | 36 | 37 | 39 | 40 | 38 | 36 | 36 | 38 | 38 | 32 | 41 | 33 | 36 | 36 | 40 | 42 | 42 | 39 | 26 | 44 | 35 | 36 | 36 |
| 40. Phypa_SYT1.3 | 39 | 41 | 39 | 38 | 39 | 39 | 38 | 39 | 39 | 33 | 42 | 38 | 34 | 34 | 43 | 42 | 42 | 36 | 27 | 43 | 37 | 39 | 39 |
| 41. Phypa_SYT1.4 | 35 | 40 | 39 | 36 | 41 | 35 | 38 | 40 | 38 | 34 | 38 | 36 | 36 | 36 | 42 | 42 | 43 | 36 | 27 | 41 | 35 | 36 | 39 |
| 42. Picsi_SYT1 | 37 | 36 | 52 | 53 | 44 | 48 | 37 | 35 | 37 | 35 | 53 | 35 | 37 | 37 | 61 | 54 | 47 | 38 | 25 | 71 | 47 | 35 | 36 |
| 43. Pinta_SYT1 | 38 | 37 | 51 | 54 | 44 | 48 | 37 | 35 | 36 | 36 | 52 | 36 | 40 | 40 | 60 | 54 | 48 | 40 | 26 | 69 | 46 | 36 | 39 |
| 44. Poptr_SYT1 | 34 | 34 | 66 | 69 | 39 | 49 | 37 | 34 | 37 | 35 | 53 | 33 | 37 | 37 | 82 | 69 | 49 | 39 | 24 | 52 | 50 | 35 | 38 |
| 45. Poptr_SYT2 | 45 | 43 | 41 | 40 | 63 | 37 | 45 | 43 | 51 | 43 | 39 | 46 | 47 | 47 | 40 | 40 | 76 | 60 | 28 | 42 | 37 | 45 | 44 |
| 46. Poptr_SYT3 | 37 | 35 | 29 | 28 | 40 | 26 | 39 | 34 | 40 | 37 | 28 | 35 | 34 | 34 | 30 | 30 | 40 | 44 | 25 | 28 | 2.1 | 39 | 34 |
| 47. Prupe_SYT2 | 46 | 46 | 37 | 40 | 67 | 38 | 47 | 46 | 51 | 45 | 42 | 46 | 46 | 46 | 42 | 40 | 77 | 60 | 27 | 40 | 38 | 46 | 46 |
| 48. Sacof_SYT1 | 35 | 35 | 45 | 50 | 38 | 98 | 34 | 37 | 36 | 33 | 79 | 33 | 34 | 34 | 48 | 48 | 39 | 35 | 23 | 47 | 95 | 34 | 35 |
| 49. Sacof_SYT2 | | 64 | 38 | 36 | 43 | 32 | 96 | 65 | 44 | 41 | 32 | 76 | 64 | 64 | 34 | 37 | 48 | 44 | 27 | 34 | 32 | 90 | 64 |
| 50. Sacof_SYT3 | 70 | | 33 | 35 | 44 | 35 | 66 | 95 | 38 | 40 | 34 | 65 | 73 | 73 | 33 | 37 | 48 | 45 | 29 | 36 | 35 | 64 | 90 |
| 51. Soltu_SYT1 | 51 | 48 | | 73 | 39 | 45 | 39 | 36 | 35 | 36 | 49 | 36 | 36 | 36 | 64 | 57 | 44 | 36 | 27 | 47 | 43 | 37 | 35 |
| 52. Soltu_SYT2 | 48 | 50 | 80 | | 39 | 50 | 33 | 34 | 36 | 36 | 52 | 34 | 34 | 34 | 65 | 56 | 47 | 37 | 24 | 49 | 48 | 33 | 36 |
| 53. Soltu_SYT3 | 54 | 55 | 54 | 54 | | 38 | 43 | 43 | 49 | 44 | 37 | 44 | 44 | 44 | 39 | 37 | 65 | 55 | 26 | 41 | 35 | 44 | 43 |
| 54. Sorbi_SYT1 | 45 | 48 | 58 | 62 | 50 | | 34 | 36 | 35 | 33 | 79 | 33 | 35 | 35 | 50 | 48 | 39 | 35 | 23 | 47 | 95 | 33 | 33 |
| 55. Sorbi_SYT2 | 97 | 71 | 51 | 46 | 53 | 46 | | 67 | 44 | 41 | 32 | 78 | 66 | 66 | 34 | 36 | 48 | 44 | 25 | 35 | 34 | 92 | 65 |
| 56. Sorbi_SYT3 | 70 | 97 | 52 | 50 | 55 | 48 | 73 | | 40 | 42 | 34 | 66 | 74 | 74 | 34 | 39 | 47 | 46 | 28 | 37 | 35 | 65 | 91 |
| 57. Tarof_SYT2 | 54 | 44 | 45 | 48 | 60 | 45 | 52 | 46 | | 40 | 34 | 40 | 41 | 41 | 36 | 36 | 50 | 46 | 25 | 37 | 35 | 43 | 39 |
| 58. Tarof_SYT3 | 53 | 54 | 51 | 50 | 55 | 46 | 52 | 57 | 47 | | 37 | 40 | 41 | 41 | 35 | 37 | 45 | 51 | 29 | 34 | 35 | 41 | 40 |
| 59. Triae_SYT1 | 47 | 46 | 64 | 66 | 49 | 86 | 43 | 47 | 44 | 52 | | 34 | 36 | 36 | 52 | 50 | 43 | 36 | 22 | 49 | 77 | 31 | 37 |
| 60. Triae_SYT2 | 84 | 71 | 50 | 45 | 54 | 44 | 86 | 71 | 49 | 49 | 44 | | 67 | 67 | 36 | 33 | 47 | 49 | 27 | 33 | 34 | 76 | 65 |
| 61. Triae_SYT3 | 72 | 79 | 50 | 49 | 59 | 49 | 74 | 81 | 50 | 53 | 49 | 75 | | 99 | 37 | 35 | 49 | 48 | 27 | 38 | 35 | 67 | 73 |
| 62. Triae_SYT3.2 | 72 | 79 | 50 | 49 | 59 | 49 | 74 | 81 | 50 | 53 | 49 | 75 | 99 | | 37 | 35 | 49 | 48 | 27 | 38 | 35 | 67 | 73 |
| 63. Vitvi_SYT1.1 | 45 | 45 | 74 | 81 | 52 | 62 | 45 | 47 | 44 | 52 | 65 | 46 | 49 | 49 | | 69 | 45 | 38 | 25 | 56 | 49 | 34 | 36 |
| 64. Vitvi_SYT1.2 | 50 | 48 | 68 | 72 | 51 | 64 | 50 | 50 | 47 | 51 | 64 | 45 | 48 | 48 | 81 | | 43 | 39 | 24 | 48 | 48 | 37 | 39 |
| 65. Vitvi_SYT2.1 | 55 | 56 | 59 | 64 | 73 | 53 | 56 | 56 | 59 | 56 | 60 | 55 | 58 | 58 | 63 | 59 | | 60 | 32 | 45 | 39 | 46 | 47 |
| 66. Vitvi_SYT2.2 | 51 | 49 | 51 | 50 | 64 | 47 | 51 | 51 | 56 | 61 | 47 | 56 | 57 | 57 | 52 | 52 | 67 | | 26 | 37 | 34 | 48 | 47 |
| 67. Volca_SYT | 38 | 41 | 39 | 39 | 38 | 37 | 37 | 42 | 34 | 41 | 37 | 42 | 39 | 39 | 38 | 39 | 42 | 37 | | 23 | 23 | 25 | 27 |
| 68. Welmi_SYT1 | 45 | 48 | 62 | 67 | 52 | 63 | 47 | 50 | 49 | 48 | 66 | 44 | 50 | 50 | 69 | 66 | 59 | 49 | 39 | | 45 | 35 | 37 |
| 69. Zeama_SYT1 | 42 | 45 | 56 | 59 | 48 | 97 | 46 | 46 | 44 | 47 | 86 | 44 | 48 | 48 | 60 | 63 | 54 | 45 | 37 | 61 | | 33 | 35 |
| 70. Zeama_SYT2 | 93 | 68 | 50 | 45 | 54 | 44 | 94 | 69 | 53 | 51 | 44 | 83 | 74 | 74 | 44 | 48 | 52 | 56 | 36 | 44 | 45 | | 61 |
| 71. Zeama_SYT3 | 69 | 92 | 49 | 50 | 53 | 46 | 69 | 92 | 46 | 53 | 49 | 69 | 80 | 80 | 49 | 51 | 57 | 51 | 39 | 50 | 49 | 64 | |

The percentage identity between the full length SYT polypeptide sequences useful in performing the methods of the invention can be as low as 25% amino acid identity compared to the polypeptide sequence of SEQ ID NO: 121 (see Table B.2 and FIG. 6).

The percentage identity can be substantially increased if the identity calculation is performed between the SNH domain as represented by SEQ ID NO: 262 (comprised in SEQ ID NO: 121) and the SNH domains of the polypeptides useful in performing the invention. Percentage identity over the SNH domain amongst the polypeptide sequences useful in performing the methods of the invention ranges between 30% and 99% amino acid identity.

The percentages in amino acid identity between the SNH domain of the polypeptides of Table A.2 are significantly higher than the percentage amino acid identity calculated between the full length SYT polypeptide sequences.

### Example 4

### Identification of Domains Comprised in Polypeptide Sequences Useful In Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Interpro is hosted at the European Bioinformatics Institute in the United Kingdom.

The results of the InterPro scan of the polypeptide sequence as represented by SEQ ID NO: 2 are presented in Table C.1.

TABLE C.1
InterPro scan results of the polypeptide sequence as represented
by SEQ ID NO: 2

| | | Integrated | Integrated |
|---|---|---|---|
| | Integrated | database | database |
| InterPro accession | database | accession | accession |
| number and name | name | number | name |
| IPR014977 | PFAM | PF08879 | WRC |
| WRC domain | | | |
| IPR014978 | PFAM | PF08880 | QLQ |
| QLQ domain | | | |

The results of the InterPro scan of the polypeptide sequence as represented by SEQ ID NO: 121 are presented in Table C.2.

TABLE C.2
InterPro scan results of the polypeptide sequence as represented
by SEQ ID NO: 2

| | | Integrated | Integrated |
|---|---|---|---|
| | Integrated | database | database |
| InterPro accession | database | accession | accession |
| number and name | name | number | name |
| IPR007726 | PFAM | PF05030 | SSXT protein (N- |
| SSXT domain/family | | | terminal region) |
| IPR007726 | Panther | PTHR23107 | SYNOVIAL |
| SSXT domain/family | | | SARCOMA |
| | | | ASSOCIATED SS18 |
| | | | PROTEIN |

Furthermore, the presence of a Met-rich domain or a QG-rich domain in the SYT polypeptide sequences may also readily be identified. As shown in FIG. 6, the Met-rich domain and QG-rich domain follows the SNH domain. The QG-rich domain may be taken to be substantially the C-terminal remainder of the polypeptide (minus the SHN domain); the Met-rich domain is typically comprised within the first half of the QG-rich (from the N-term to the C-term) domain. Primary amino acid composition (in %) to determine if a polypeptide domain is rich in specific amino acids may be calculated using software programs from the ExPASy server (Gasteiger E et al. (2003) ExPASy: the proteomics server for in-depth protein knowledge and analysis. Nucleic Acids Res 31:3784-3788), in particular the ProtParam tool. The composition of the polypeptide of interest may then be compared to the average amino acid composition (in %) in the Swiss-Prot Protein Sequence data bank (Table C.3). Within this databank, the average Met (M) content is of 2.37%, the average Gln (Q) content is of 3.93% and the average Gly (G) content is of 6.93% (Table C.3). As defined herein, a Met-rich domain or a QG-rich domain has Met content (in %) or a Gln and Gly content (in %) above the average amino acid composition (in %) in the Swiss-Prot Protein Sequence data bank. For example in SEQ ID NO: 121, the Met-rich domain at the N-terminal preceding the SNH domain (from amino acid positions 1 to 24) has Met content of 20.8% and a QG-rich domain (from amino acid positions 71 to 200) has a Gln (Q) content of 18.6% and a Gly (G) content of 21.4%. Preferably, the Met domain as defined herein has a Met content (in %) that is at least 1.25, 1.5, 1.75, 2.0, 2.25, 2.5, 2.75, 3.0, 3.25, 3.5, 3.75, 4.0, 4.25, 4.5, 4.75, 5.0, 5.25, 5.0, 5.75, 6.0, 6.25, 6.5, 6.75, 7.0, 7.25, 7.5, 7.75, 8.0, 8.25, 8.5, 8.75, 9.0, 9.25, 9.5, 9.75, 10 or more as much as the average amino acid composition (in %) of said kind of protein sequences, which are included in the Swiss-Prot Protein Sequence data bank. Preferably, the QG-rich domain as defined herein has a Gln (Q) content and/or a Gly (G) content that is at least 1.25, 1.5, 1.75, 2.0, 2.25, 2.5, 2.75, 3.0, 3.25, 3.5, 3.75, 4.0, 4.25, 4.5, 4.75, 5.0, 5.25, 5.0, 5.75, 6.0, 6.25, 6.5, 6.75, 7.0, 7.25, 7.5, 7.75, 8.0, 8.25, 8.5, 8.75, 9.0, 9.25, 9.5, 9.75, 10 or more as much as the average amino acid composition (in %) of said kind of protein sequences, which are included in the Swiss-Prot Protein Sequence data bank.

TABLE C.3
Mean amino acid composition (%) of proteins in
SWISS PROT Protein Sequence data bank
(July 2004):

| Residue | % |
|---|---|
| A = Ala | 7.80 |
| C = Cys | 1.57 |
| D = Asp | 5.30 |
| E = Glu | 6.59 |
| F = Phe | 4.02 |
| G = Gly | 6.93 |
| H = His | 2.27 |
| I = Ile | 5.91 |
| K = Lys | 5.93 |
| L = Leu | 9.62 |
| M = Met | 2.37 |
| N = Asn | 4.22 |
| P = Pro | 4.85 |
| Q = Gln | 3.93 |
| R = Arg | 5.29 |
| S = Ser | 6.89 |
| T = Thr | 5.46 |
| V = Val | 6.69 |
| W = Trp | 1.16 |
| Y = Tyr | 3.09 |

### Example 5

### Subcellular Localisation Prediction of the GRF Polypeptide Sequences Useful in Performing the Methods of the Invention

Experimental methods for protein localization range from immunolocalization to tagging of proteins using green fluorescent protein (GFP) or beta-glucuronidase (GUS). For example, a GRF polypeptide fused to a GUS reporter gene was used to transform transiently onion epidermal cells (van der Knapp et al. (2000) Plant Phys 122: 695-704). The nucleus was identified as the subcellular compartment of the GRF polypeptide. Such methods to identify subcellular compartmentalisation of GRF polypeptides are well known in the art.

A predicted nuclear localisation signal (NLS) was found by multiple sequence alignment, followed by eye inspection, in the WRC domain (CRRTDGKKWRC) of the GRF polypeptide of Table A. An NLS is one or more short sequences of positively charged lysines or arginines.

Computational prediction of protein localisation from sequence data was performed. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP and others. LOCtree is an algorithm that can predict the subcellular localization and DNA-binding propensity of non-membrane proteins in non-plant and plant eukaryotes as well as prokaryotes. LOCtree classifies eukaryotic animal proteins into one of five subcellular classes, while plant proteins are classified into one of six classes and prokaryotic proteins are classified into one of three classes. Table D below shows the output of LOCtree using the polypeptide sequence information of SEQ ID NO: 2. High confidence predictions have reliability index values greater than 5.

TABLE D
Output of LOCtree using the polypeptide sequence
information of SEQ ID NO: 2.

| | | Intermediate localization prediction | |
|---|---|---|---|
| Predicted | Reliability | (output of different SVMs in | Reliability |
| Localization | index | hierarchical tree) | index |
| DNA | 6 | Not secreted, Nuclear, | 8, 6, 9 |
| binding | | DNA-binding | |

The predicted subcellular compartment of the GRF polypeptide as represented by SEQ ID NO: 2 using the LOCTree algorithm is the nucleus.

### Example 6

### Assay Related to the Polypeptide Sequences Useful in Performing the Methods of the Invention

GRF polypeptides and SYT polypeptids useful in the methods of the present invention (at least in their native form) typically, but not necessarily, have transcriptional regulatory activity and capacity to interact with other proteins. DNA-binding activity and protein-protein interactions may readily be determined in vitro or in vivo using techniques well known in the art (for example in Current Protocols in Molecular Biology, Volumes 1 and 2, Ausubel et al. (1994), Current Protocols). GRF polypeptides are capable of transcriptional activation of reporter genes in yeast cells (Kim & Kende (2004) Proc Natl Acad Sci 101(36): 13374-13379). GRF polypeptides are also capable of interacting with SYT polypeptides (also called GRF interacting factor or GIF) in vivo in yeast cells, using a yeast two-hybrid protein-protein interaction assay (Kim & Kende, supra). In vitro binding assays are also used to show that GRF polypeptides and SYT polypeptides are interacting partners (Kim & Kende, supra). The experiments described in this publication are useful in characterizing GRF polypeptides and SYT polypeptides, and are well known in the art.

### Example 7

### Cloning of Nucleic Acid Sequences Useful in Performing the Methods Of the Invention

Unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Cloning of a Nucleic Acid Sequence as Represented by SEQ ID NO: 1

The Arabidopsis thaliana cDNA encoding the GRF polypeptide sequence as represented by SEQ ID NO: 2 was amplified by PCR using as template an Arabidopsis cDNA bank synthesized from mRNA extracted from mixed plant tissues. primer prm08136 SEQ ID NO: 42: 5′-ggggaccactttgtacaagaaagctgggttaaaaaccattttaacgcacg), The following primers, which include the AttB sites for Gateway recombination, were used for PCR amplification:

1) Prm 10010 (SEQ ID NO: 118, sense):
5′-GGGGACAAGTTTGTACAAAAAAGCAGGCTTAAACAATGATGAGTCTA
AGTGGAAGTAG-3′
2) Prm 10011 (SEQ ID NO: 119, reverse,
complementary):
5′-GGGGACCACTTTGTACAAGAAAGCTGGGTAGCTCTACTTAATTAGCT
ACCAG-3′

### Cloning of a Nucleic Acid Sequence as Represented by SEQ ID NO: 120

The Arabidopsis thaliana cDNA encoding the SYT polypeptide sequence as represented by SEQ ID NO: 121 was amplified by PCR using as template an Arabidopsis cDNA bank synthesized from mRNA extracted from mixed plant tissues. The following primers, which include the AttB sites for Gateway recombination, were used for PCR amplification:

1) Prm06681 (SEQ ID NO: 265, sense):
5′-GGGGACAAGTTTGTACAAAAAAGCAGGCTTAAACAATGCAACAGCAC
CTGATG-3′
2) Prm 06682 (SEQ ID NO: 266, reverse,
complementary):
5′-GGGGACCACTTTGTACAAGAAAGCTGGGTCATCATTAAGATTCCTTG
TGC-3′

PCR reactions were independently performed for SEQ ID NO: 1 and SEQ ID NO: 120, using Hifi Taq DNA polymerase in standard conditions. A PCR fragment of the expected length (including attB sites) was amplified and purified also using standard methods. The first step of the Gateway procedure, the BP reaction, was then performed, during which the PCR fragment recombined in vivo with the pDONR201 plasmid to produce, according to the Gateway terminology, an “entry clone”. Plasmid pDONR201 was purchased from Invitrogen, as part of the Gateway® technology.

### Example 8

### Expression Vector Construction Using the Nucleic Acid Sequences as Represented by SEQ ID NO: 1 and by SEQ ID NO: 120

The entry clones independently comprising SEQ ID NO: 1 and SEQ ID NO: 120 were subsequently used independently in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 117) for constitutive expression was located upstream of this Gateway cassette.

After the LR recombination step, the resulting expression vectors pGOS2::GRF and pGOS2:: SYT (FIG. 9) were independently transformed into Agrobacterium strain LBA4044 according to methods well known in the art.

### Example 9

### Plant Transformation

### Rice Transformation

The Agrobacterium containing the expression vector pGOS2:: SYT was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing each individual expression vector was used independently for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for each construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Rice Re-Transformation

By rice re-transformation is meant herein the transformation of rice plants already transgenic for another construct.

In particular, seeds harvested from transgenic homozygous plants expressing the nucleic acid sequence coding for a SYT polypeptide were re-transformed with the expression vector of Example 7. Except for this difference in initial plant source material, and the use of a different selectable marker for the re-transformation compared to the selectable marker for the initial transformation, the rest of the procedure was as described above.

### Example 10

### Phenotypic Evaluation Procedure

### 10.1 Evaluation Setup

Approximately 35 independent T0 rice re-transformants were generated. These plants were further transferred from a tissue culture chamber to a greenhouse for growing and harvest of T1 seed. Greenhouse conditions were of shorts days (12 hours light), 28° C. in the light and 22° C. in the dark, and a relative humidity of 70%.

PCR checks were performed to check for the presence of (i) the isolated nucleic acid transfene encoding a GRF polypeptide as represented by SEQ ID NO: 2; and of (ii) the isolated nucleic acid transfene encoding a SYT polypeptide as represented by SEQ ID NO: 121. PCR checks were also done for the presence and copy number of promoters, terminators and plant selectable markers. Selected transgenic plants were further grown until homozygous for both transgene loci.

### 10.2 Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### 10.3 Parameters Measured

### Seed-Related Parameter Measurements

Individual seed parameters (including width, length, area) were measured using a custom-made device consisting of two main components, a weighing and imaging device, coupled to software for image analysis.

### Example 11

### Results of Seed Size Measurements from Seeds Harvested from Re-Transformed Rice Plants

Homozygous transgenic rice plants expressing the nucleic acid sequence coding for a SYT polypeptide as represented by SEQ ID NO: 121 under the control of a constitutive promoter were re-transformed with the expression vector of Example 7 hereinabove, comprising the nucleic acid sequence coding for the GRF polypeptide of SEQ ID NO: 2, also under the control of a constitutive promoter. The re-transformed rice plants were further grown until homozygous for both loci.

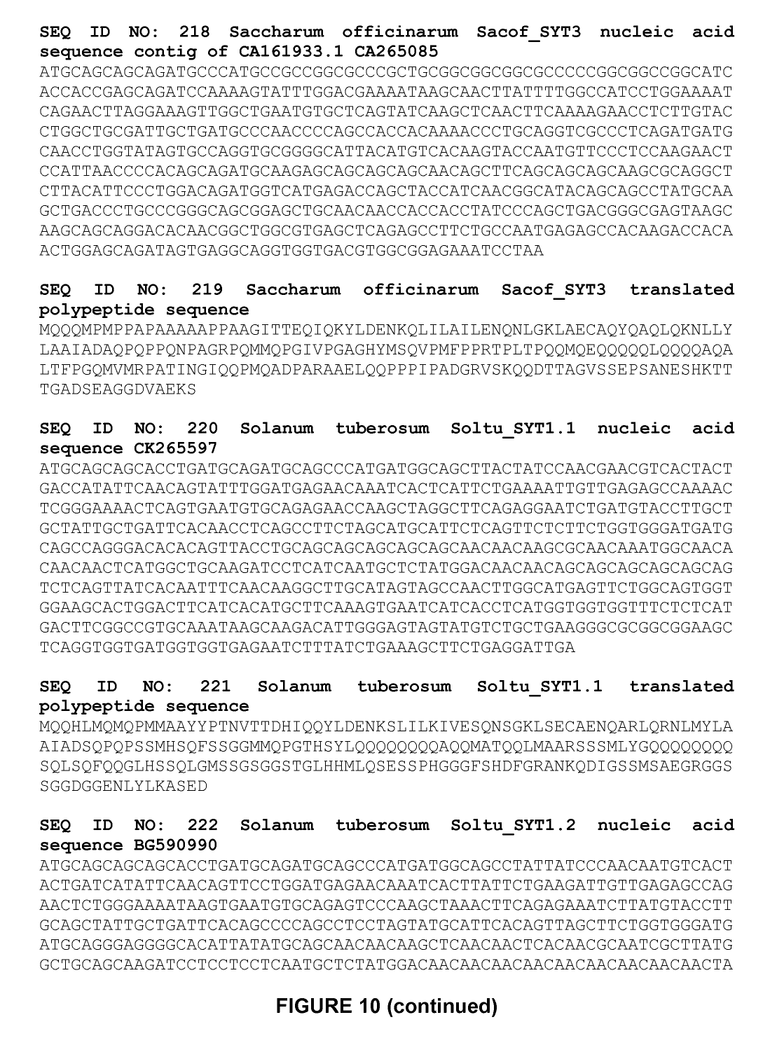

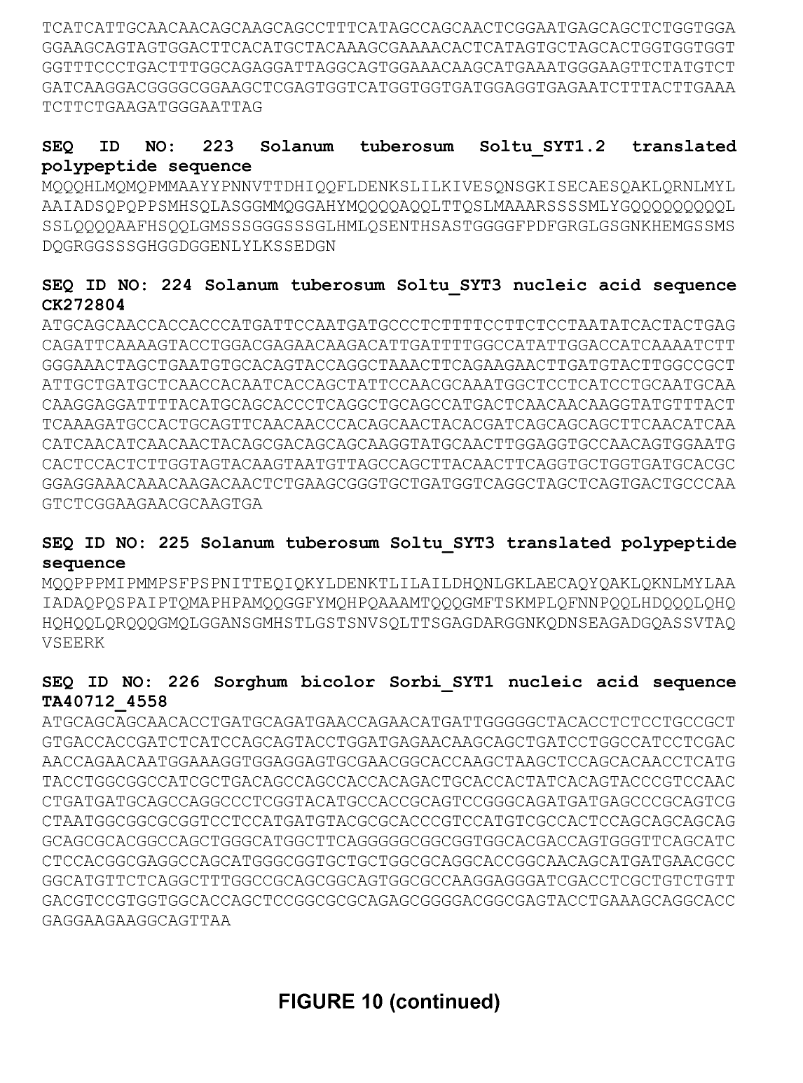

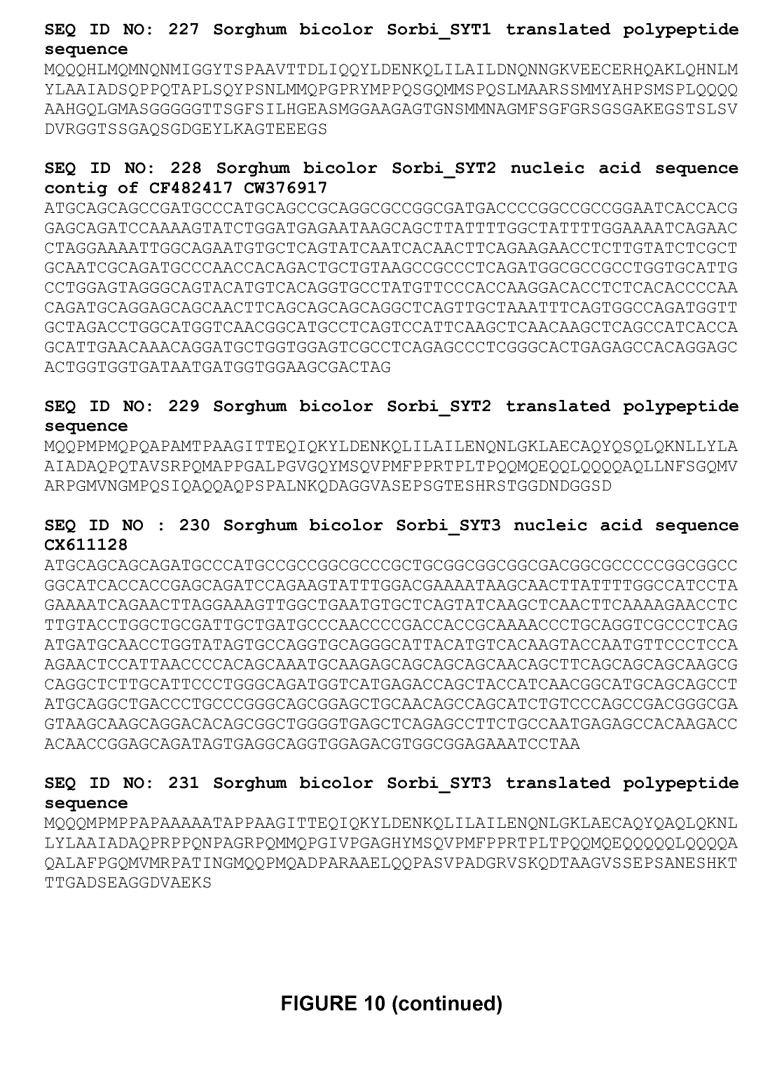

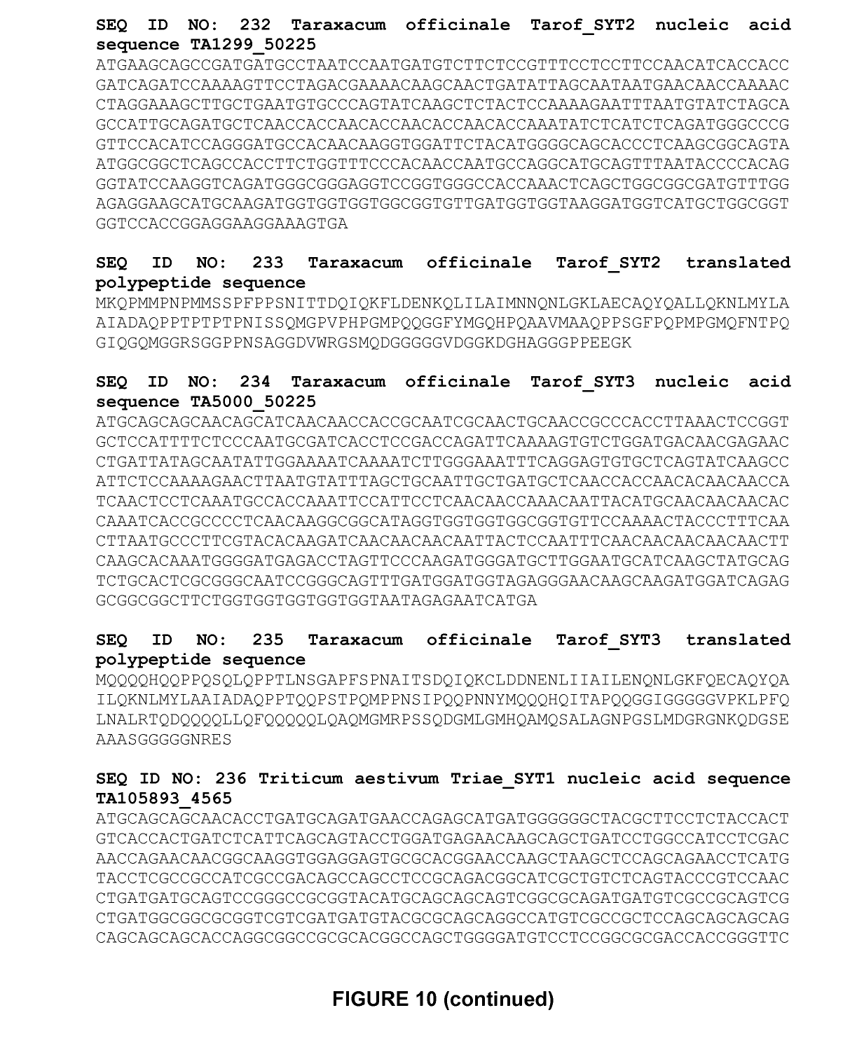

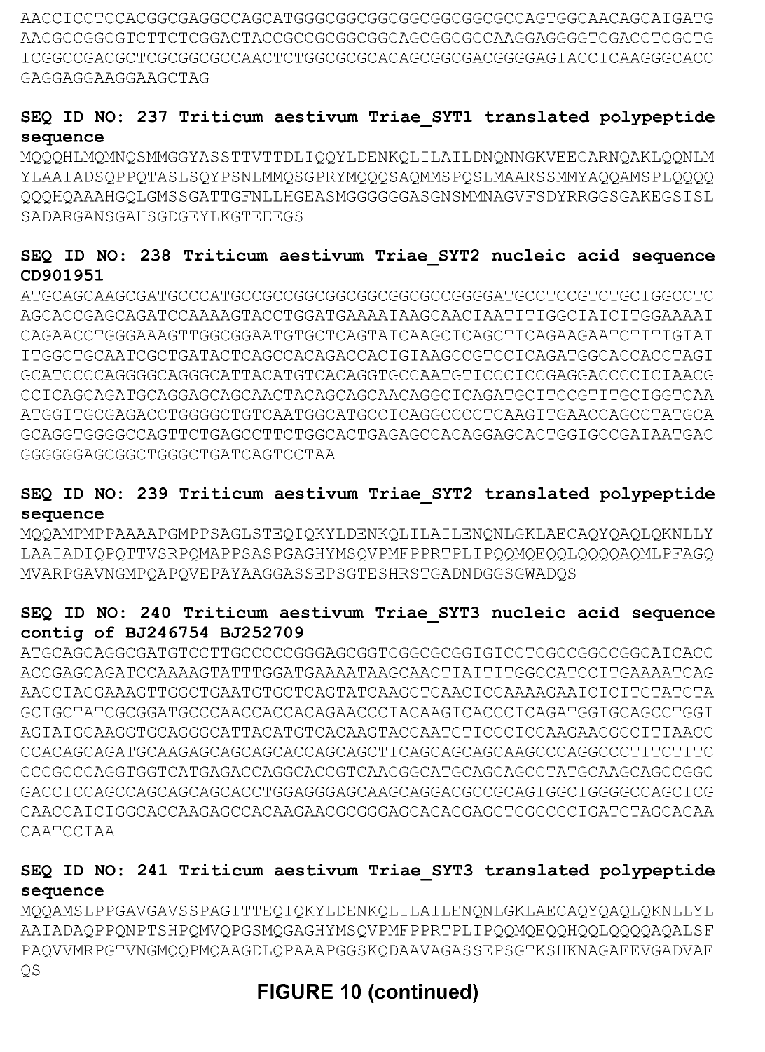

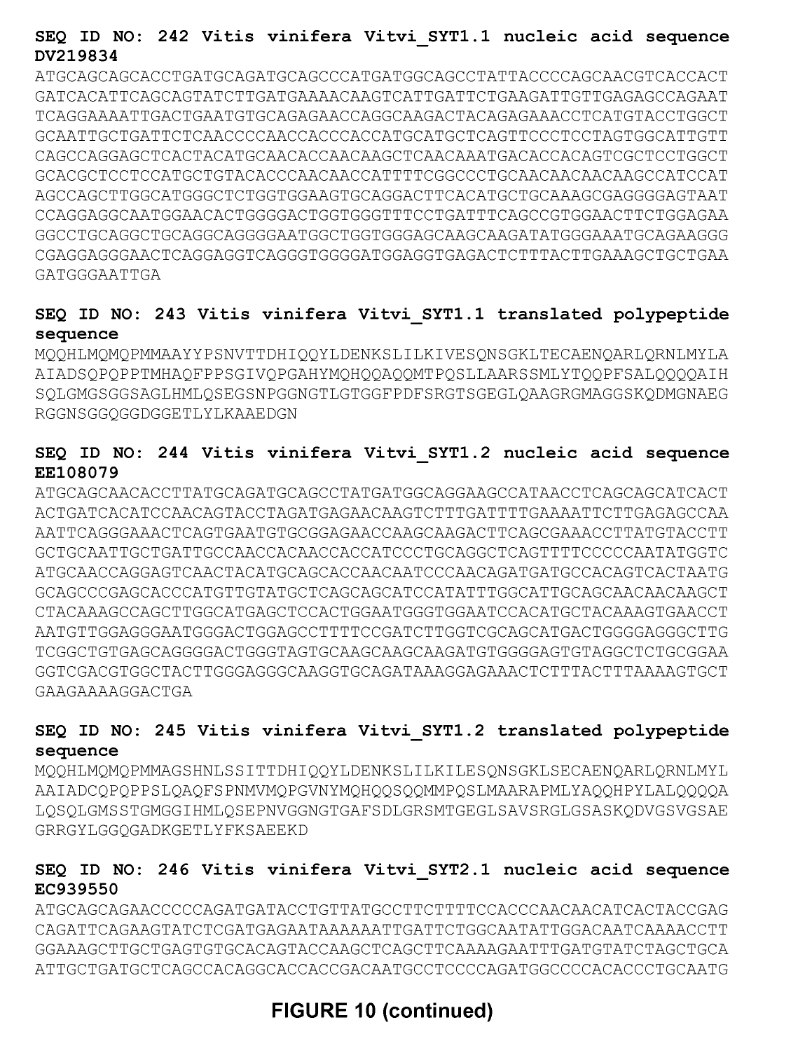

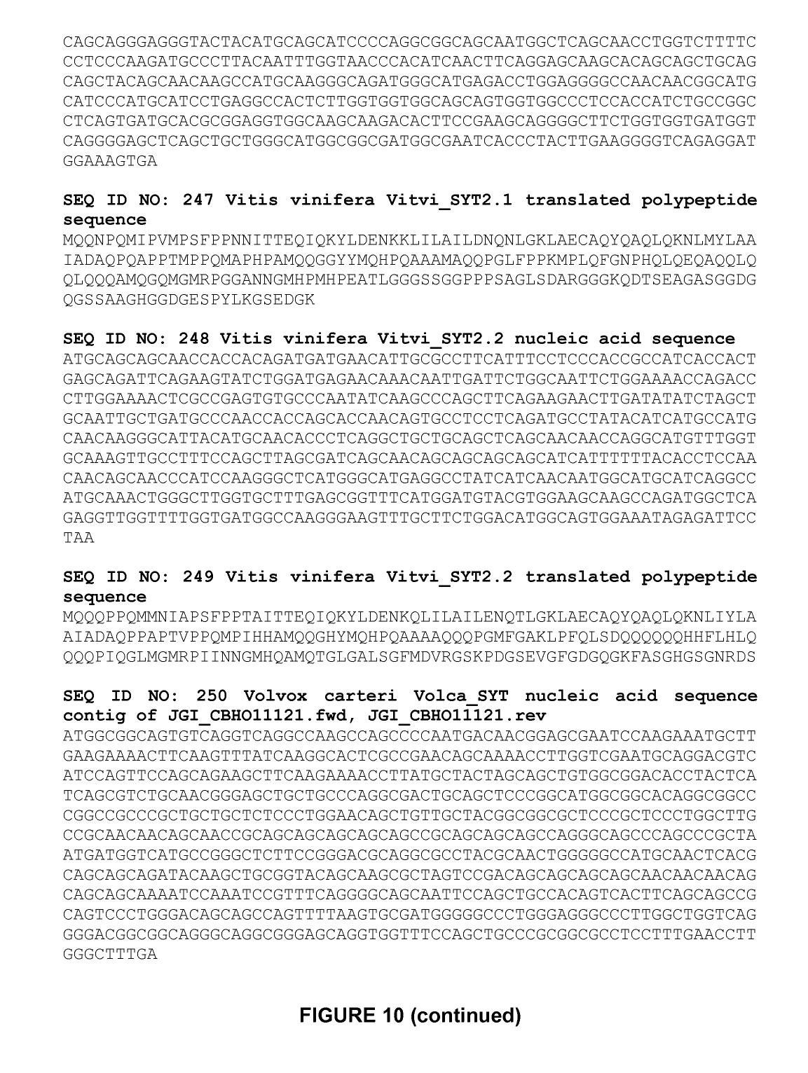

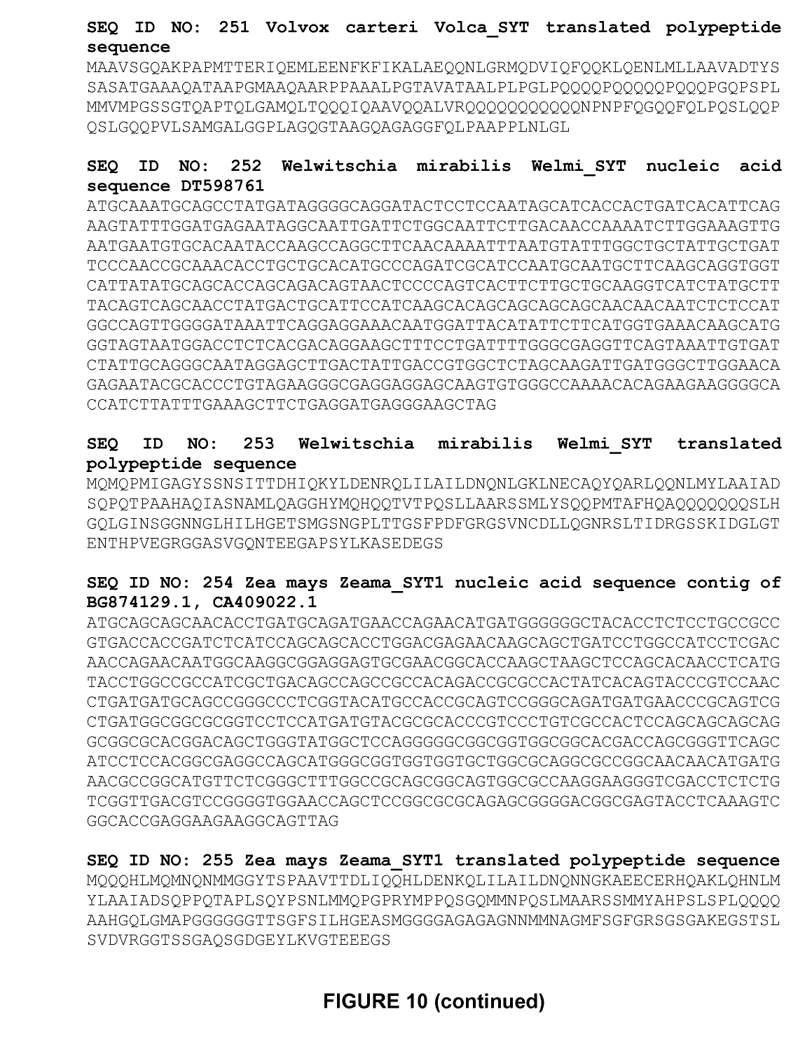

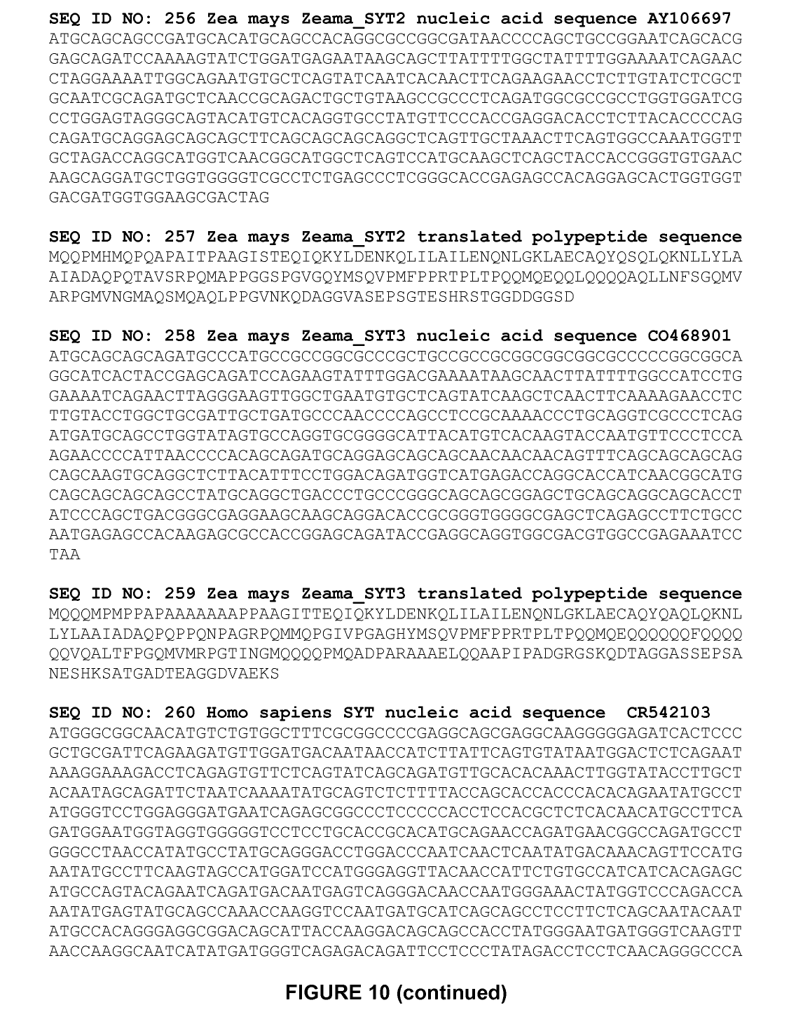

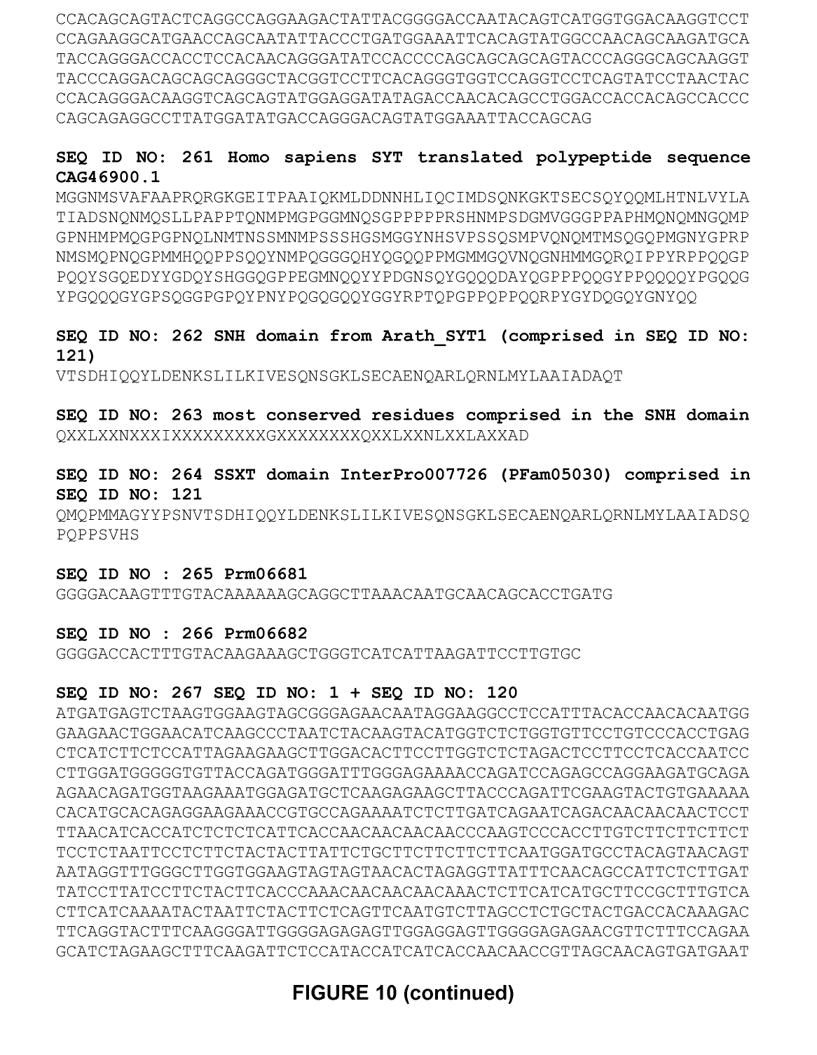

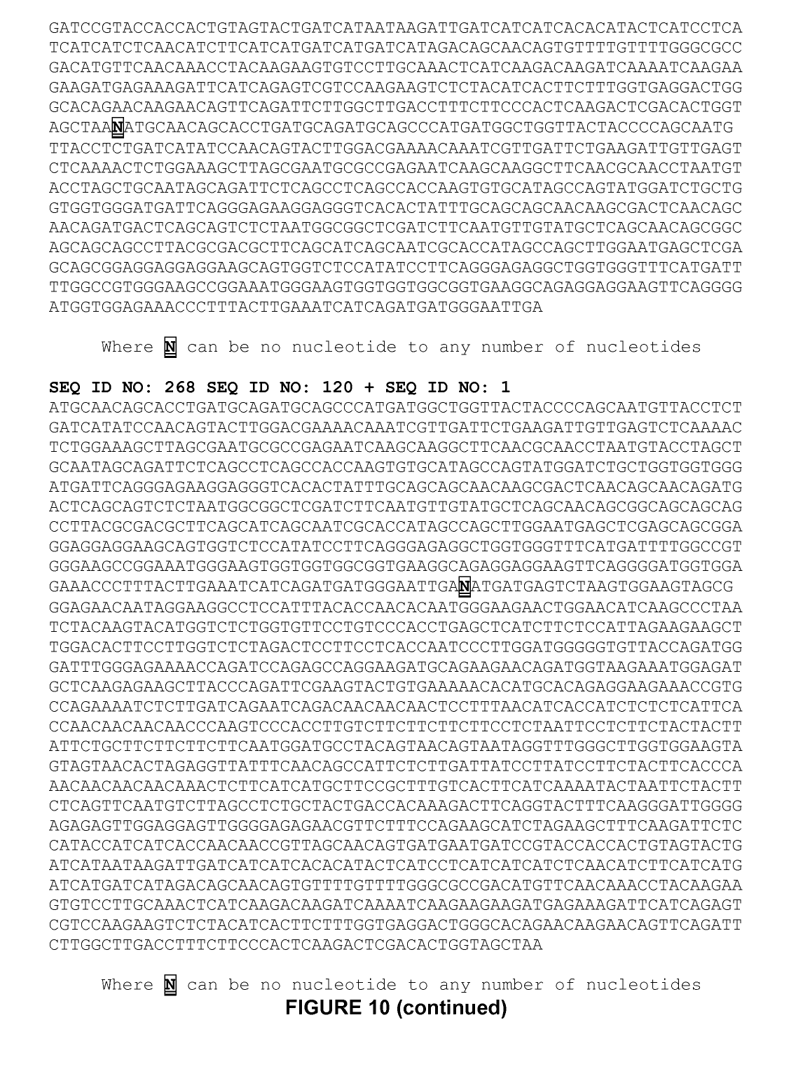

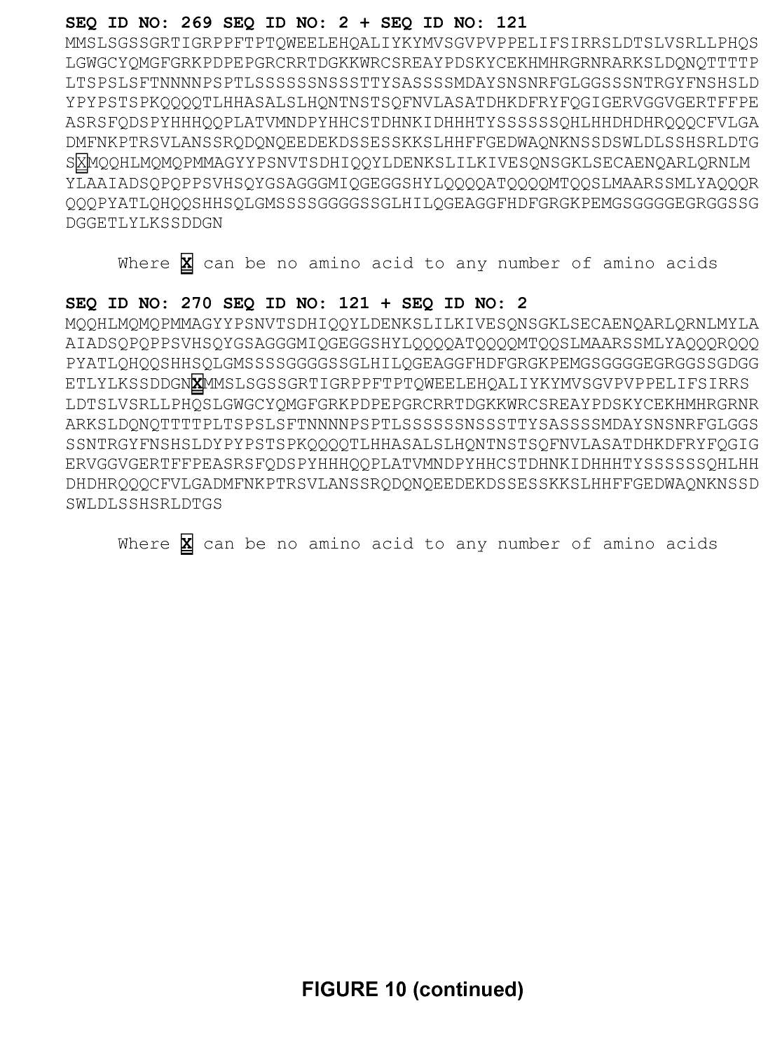

FIG. 7 shows on the left a panicle from a rice plant (Oryza sativa ssp. Japonica cv. Nipponbare) transformed with a control vector, and on the right a panicle from a rice plant (Oryza sativa ssp. Japonica cv. Nipponbare) transformed with two constructs: (1) a nucleic acid sequence encoding a GRF polypeptide under the control of a GOS2 promoter (pGOS2) from rice; and (2) a nucleic acid sequence encoding a SYT polypeptide under the control of a GOS2 promoter (pGOS2) from rice. The rice plants transformed with both constructs are homozygous for both loci. Plant biomass, number of panicles, panicle size, seed number and seed size are clearly increased in the re-transformed rice compared to the same parameters in rice transformed with a control vector.

Seeds harvested from the re-transformed rice plants, and homozygous for both loci, were harvested, and samples of 30 seeds were photographed. FIG. 8 shows on the top row, from left to right, 30 mature rice seeds (Oryza sativa ssp. Japonica cv. Nipponbare) from:
(a) plants transformed with one construct comprising a nucleic acid sequence encoding a SYT polypeptide as represented by SEQ ID NO: 120, under the control of a GOS2 promoter (pGOS2) from rice;
(b) plants transformed with two constructs: (1) a nucleic acid sequence encoding a GRF polypeptide as represented by SEQ ID NO: 2, under the control of a GOS2 promoter (pGOS2) from rice; and (2) a nucleic acid sequence encoding a SYT polypeptide as represented by SEQ ID NO: 120, under the control of a GOS2 promoter (pGOS2) from rice;
(c) plants transformed with one construct comprising a nucleic acid sequence encoding a GRF polypeptide as represented by SEQ ID NO: 2, under the control of a GOS2 promoter (pGOS2) from rice;
(d) nullizygote plants (control plants) from a;
(e) nullizygote plants (control plants) from c;
   An increase in seed size was visible by simple eye inspection.

The homozygous seeds from 6 transgenic events were then imaged to estimate average seed area, average seed length, and average seed width, and then compared to the ame parameters measured in (i) homozygous seeds from plants transformed with one construct comprising a nucleic acid sequence encoding a SYT polypeptide; and in (ii) seeds from control plants (nullizygotes) from (i). Results are shown in the Table E below.

TABLE E
Results of seed area, seed length and seed width measurements
of seeds harvested from homozygous re-transformed rice plants
relative to suitable control seeds.

| | Compared to homozygous | |
|---|---|---|
| | seeds from plants | |
| | transformed with one | Compared to |
| | construct comprising | seeds from |
| | a nucleic acid sequence | control plants |
| | encoding a SYT polypeptide | (nullizygotes) |
| Seed area | At least 11% increase | At least 26% increase |
| Seed length | At least 8% increase | At least 21% increase |
| Seed width | At least 3% increase | At least 6% increase |

### Example 12

### Examples of Transformation of Other Crops

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M patent U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton (Gossypium hirsutum L.) transformation is performed using Agrobacterium tumefaciens, on hypocotyls explants. The commercial cultivars such as Coker 130 or Coker 312 (SeedCo, Lubbock, Tex.) are standard varieties used for transformation, but other varieties can also be used. The seeds are surface sterilized and germinated in the dark. Hypocotyl explants are cut from the germinated seedlings to lengths of about 1-1.5 centimeter. The hypotocyl explant is submersed in the Agrobacterium tumefaciens inoculum containing the expression vector, for 5 minutes then co-cultivated for about 48 hours on MS+1.8 mg/l KNO3+2% glucose at 24° C., in the dark. The explants are transferred the same medium containing appropriate bacterial and plant selectable markers (renewed several times), until embryogenic calli is seen. The calli are separated and subcultured until somatic embryos appear. Plantlets derived from the somatic embryos are matured on rooting medium until roots develop. The rooted shoots are transplanted to potting soil in the greenhouse. T1 seeds are produced from plants that exhibit tolerance to the selection agent and that contain a single copy of the T-DNA insert.

1. A method for increasing plant yield-related traits, comprising increasing expression in a plant of: (i) a nucleic acid sequence encoding a Growth-Regulating Factor (GRF) polypeptide; and of (ii) a nucleic acid sequence encoding a synovial sarcoma translocation (SYT) polypeptide, wherein said yield-related traits are increased relative to plants having increased expression of one of: (i) a nucleic acid sequence encoding a GRF polypeptide, or (ii) a nucleic acid sequence encoding a SYT polypeptide.

2. The method according to claim 1, wherein said GRF polypeptide comprises: (i) a domain having at least 50% amino acid sequence identity to a QLQ domain as represented by SEQ ID NO: 115; and (ii) a domain having at least 50% amino acid sequence identity to a WRC domain as represented by SEQ ID NO: 116.

3. The method according to claim 1, wherein said GRF polypeptide comprises: (i) a QLQ domain with an InterPro accession IPR014978 (PFAM accession PF08880); (ii) a WRC domain with an InterPro accession IPR014977 (PFAM accession PF08879); and (iii) an Effector of Transcription (ET) domain comprising three Cys and one His residues in a conserved spacing (CX₉CX₁₀CX₂H).

4. The method according to claim 1, wherein said GRF polypeptide has at least 50% amino acid sequence identity to the GRF polypeptide as represented by SEQ ID NO: 2 or to any of the polypeptide sequences given in Table A.1 herein.

5. The method according to claim 1, wherein said nucleic acid sequence encoding a GRF polypeptide is represented by any one of the nucleic acid sequence SEQ ID NOs given in Table A.1 or a portion thereof, or a sequence capable of hybridizing with any one of the nucleic acid sequences SEQ ID NOs given in Table A.1.

6. The method according to claim 1, wherein said nucleic acid sequence encodes an orthologue or paralogue of any of the GRF polypeptide sequence SEQ ID NOs given in Table A.1.

7. The method according to claim 1, wherein said nucleic acid sequence encoding a GRF polypeptide is operably linked to a constitutive promoter, a GOS2 promoter, or a GOS2 promoter from rice as represented by SEQ ID NO: 117.

8. The method according to claim 1, wherein said nucleic acid sequence encoding a GRF polypeptide is of plant origin, from a dicotyledonous plant, from the family Brassicaceae, or from Arabidopsis thaliana.

9. The method according to claim 1, wherein said nucleic acid sequence encoding a SYT polypeptide, wherein said SYT polypeptide comprises from N-terminal to C-terminal: (i) an SNH domain having at least 20 sequence identity to the SNH domain of SEQ ID NO: 262; and (ii) a Met-rich domain; and (iii) a QG-rich domain.

10. The method according to claim 1, wherein said SYT polypeptide further comprises the most conserved residues of the SNH domain as represented by SEQ ID NO: 263, and shown in black in FIG. 5.

11. The method according to claim 1, wherein said SYT polypeptide comprises a domain having at least 20% sequence identity to the SSXT domain with an InterPro accession IPR007726 of SEQ ID NO: 264.

12. The method according to claim 1, wherein said SYT polypeptide has at least 20% amino acid sequence identity to the SYT polypeptide as represented by SEQ ID NO: 121 or to any of the full length polypeptide sequences given in Table A.2 herein.

13. The method according to claim 1, wherein said nucleic acid sequence encoding a SYT polypeptide is represented by any one of the nucleic acid sequence SEQ ID NOs given in Table A.2 or a portion thereof, or a sequence capable of hybridizing with any one of the nucleic acid sequences SEQ ID NOs given in Table A.2.

14. The method according to claim 1, wherein said nucleic acid sequence encodes an orthologue or paralogue of any of the SYT polypeptide sequence SEQ ID NOs given in Table A.2.

15. The method according to claim 1, wherein said nucleic acid sequence encoding a SYT polypeptide is operably linked to a constitutive promoter, a GOS2 promoter, or a GOS2 promoter from rice as represented by SEQ ID NO: 117.

16. The method according to claim 1, wherein said nucleic acid sequence encoding a SYT polypeptide is of plant origin, from a dicotyledonous plant, from the family Brassicaceae, or from Arabidopsis thaliana.

17. The method according to claim 1, wherein said increased expression is effected by introducing and expressing in a plant: (i) a nucleic acid sequence encoding a GRF polypeptide; and (ii) a nucleic acid sequence encoding a SYT polypeptide.

18. The method according to claim 17, wherein said nucleic acid sequences of (i) and (ii) are sequentially introduced and expressed in a plant, by crossing, or by re-transformation.

19. The method according to claim 18, wherein said crossing is performed between a female parent plant comprising an introduced and expressed isolated nucleic acid sequence encoding a GRF polypeptide, and a male parent plant comprising an introduced and expressed isolated nucleic acid sequence encoding a SYT polypeptide, or reciprocally, and by selecting in the progeny for the presence and expression of both transgenes, wherein said plant has increased yield-related traits relative to each parent plant.

20. The method according to claim 18, wherein said re-transformation is performed by introducing and expressing a nucleic acid sequence encoding GRF polypeptide into a plant, plant part, or plant cell comprising an introduced and expressing nucleic acid sequence encoding a SYT polypeptide, or reciprocally.

21. The method according to claim 17, wherein said nucleic acid sequences of (i) and (ii) are simultaneously introduced and expressed in a plant.

22. The method according to claim 21, wherein said nucleic acid sequences of (i) and (ii) are comprised in one or more nucleic acid molecules.

23. The method according to claim 1, wherein said increased yield-related trait is one or more of: (i) increased early vigour; (ii) increased aboveground biomass; (iii) increased total seed yield per plant; (iv) increased seed filling rate; (v) increased number of (filled) seeds; (vi) increased harvest index; or (vii) increased thousand kernel weight (TKW).

24. The method according to claim 1, wherein said nucleic acid sequence encoding a GRF polypeptide and said nucleic acid sequence encoding a SYT polypeptide are operably and sequentially linked to a constitutive promoter, a plant constitutive promoter, to a GOS2 promoter, or a GOS2 promoter from rice as represented by SEQ ID NO: 117.

25. Plants, parts thereof (including seeds), or plant cells obtainable by the method according to claim 1, wherein said plants, parts or cells thereof comprise (i) an isolated nucleic acid transgene encoding a GRF polypeptide and (ii) an isolated nucleic acid transgene encoding a SYT polypeptide.

26. A construct comprising:
(a) a nucleic acid sequence encoding a GRF polypeptide, wherein the GRF polypeptide comprises
(i) a domain having at least 50% amino acid sequence identity to a QLQ domain as represented by SEQ ID NO: 115, and a domain having at least 50% amino acid sequence identity to a WRC domain as represented by SEQ ID NO: 116;
(ii) a QLQ domain with an InterPro accession IPR014978 (PFAM accession PF08880), a WRC domain with an InterPro accession IPR014977 (PFAM accession PF08879), and an Effector of Transcription (ET) domain comprising three Cys and one His residues in a conserved spacing (CX₉CX₁₀CX₂H);
(iii) an amino acid sequence having at least 50% identity to the GRF polypeptide as represented by SEQ ID NO: 2 or to any of the polypeptide sequences given in Table A.1 herein;
(iv) a polypeptide encoded by the nucleic acid sequence as defined in claim 5; or
(v) an orthologue or paralogue of any of the GRF polypeptide sequence SEQ ID NOs given in Table A.1;
(b) a nucleic acid sequence encoding a SYT polypeptide, wherein the SYT polypeptide comprises
(i) from N-terminal to C-terminal, an SNH domain having at least 20% sequence identity to the SNH domain of SEQ ID NO: 262, a Met-rich domain, and a QG-rich domain;
(ii) the most conserved residues of the SNH domain as represented by SEQ ID NO: 263, and shown in black in FIG. 5;
(iii) a domain having at least 20% sequence identity to the SSXT domain with an InterPro accession IPR007726 of SEQ ID NO: 264;
(iv) at least 20% amino acid sequence identity to the SYT polypeptide as represented by SEQ ID NO: 121 or to any of the full length poly sequences given in Table A.2 herein; or
(v) a polypeptide encoded by a nucleic acid sequence represented by any one of the nucleic acid sequence SEQ ID NOs given in Table A.2 or a portion thereof, or a sequence capable of hybridizing with any one of the nucleic acid sequences SEQ ID NOs given in Table A.2;
(c) one or more control sequences capable of driving expression of the nucleic acid sequence of (a) and of (b); and optionally
(d) a transcription termination sequence.

27. A construct according to claim 26, wherein said control sequence is at least one constitutive promoter, a GOS2 promoter, or a GOS2 promoter as represented by SEQ ID NO: 117.

28. A mixture of constructs, wherein at least one construct comprises:
(a) a nucleic acid sequence encoding a GRF polypeptide, wherein the GRF polypeptide comprises
(i) a domain having at least 50% amino acid sequence identity to a QLQ domain as represented by SEQ ID NO: 115, and a domain having at least 50% amino acid sequence identity to a WRC domain as represented by SEQ ID NO: 116;
(ii) a QLQ domain with an InterPro accession IPR014978 (PFAM accession PF08880), a WRC domain with an InterPro accession IPR014977 (PFAM accession PF08879), and an Effector of Transcription (ET) domain comprising three Cys and one His residues in a conserved spacing (CX₉CX₁₀CX₂H);
(iii) an amino acid sequence having at least 50% identity to the GRF polypeptide as represented by SEQ ID NO: 2 or to any of the polypeptide sequences given in Table A.1 herein;
(iv) a polypeptide encoded by the nucleic acid sequence as defined in claim 5; or
(v) an orthologue or paralogue of any of the GRF polypeptide sequence SEQ ID NOs given in Table A.1;
(b) one or more control sequences capable of driving expression of the nucleic acid sequence of (a); and optionally
(c) a transcription termination sequence,
and wherein at least one other construct comprises:
(d) a nucleic acid sequence encoding a SYT polypeptide, wherein the SYT polypeptide comprises
(i) from N-terminal to C-terminal, an SNH domain having at least 20% sequence identity to the SNH domain of SEQ ID NO: 262, a Met-rich domain, and a QG-rich domain;
(ii) the most conserved residues of the SNH domain as represented by SEQ ID NO: 263, and shown in black in FIG. 5;
(iii) a domain having at least 20% sequence identity to the SSXT domain with an InterPro accession IPR007726 of SEQ ID NO: 264;
(iv) at least 20% amino acid sequence identity to the SYT polypeptide as represented by SEQ ID NO: 121 or to any of the full length polypeptide sequences given in Table A.2 herein; or
(v) a polypeptide encoded by a nucleic acid sequence represented by any one of the nucleic acid sequence SEQ ID NOs given in Table A.2 or a portion thereof, or a sequence capable of hybridizing with any one of the nucleic acid sequences SEQ ID NOs given in Table A.2;
(e) one or more control sequences capable of driving expression of the nucleic acid sequence of (d); and optionally
(f) a transcription termination sequence.

29. The constructs according to claim 28, wherein said control sequence of (b) and/or (e) is at least one constitutive promoter, GOS2 promoter, or a GOS2 promoter as represented by SEQ ID NO: 117.

30. A method for making plants having increased yield-related traits relative to plants having increased expression of one of: (a) a nucleic acid sequence encoding a GRF polypeptide, or (b) a nucleic acid sequence encoding a SYT polypeptide, which increased yield-related traits are one or more of: (i) increased early vigour; (ii) increased aboveground biomass; (iii) increased total seed yield per plant; (iv) increased seed filling rate; (v) increased number of (filled) seeds; (vi) increased harvest index; or (vii) increased thousand kernel weight (TKW), comprising utilizing at least one construct according to claim 26.

31. A plant, plant part or plant cell transformed with at least one construct according to claim 26.

32. A method for the production of transgenic plants having increased yield-related traits relative to plants having increased expression of one of: (i) a nucleic acid sequence encoding a GRF polypeptide, or (ii) a nucleic acid sequence encoding a SYT polypeptide, comprising:
a. introducing and expressing in a plant, plant part, or plant cell, a nucleic acid sequence encoding a GRF polypeptide under the control of a constitutive promoter, wherein the GRF polypeptide comprises
a domain having at least 50% amino acid sequence identity to a QLQ domain as represented by SEQ ID NO: 115, and a domain having at least 50% amino acid sequence identity to a WRC domain as represented by SEQ ID NO: 116;
(ii) a QLQ domain with an InterPro accession IPR014978 (PFAM accession PF08880), a WRC domain with an InterPro accession IPR014977 (PFAM accession PF08879), and an Effector of Transcription (ET) domain comprising three Cys and one His residues in a conserved spacing (CX₉CX₁₀CX₂H);
(iii) an amino acid sequence having at least 50% identity to the GRF polypeptide as represented by SEQ ID NO: 2 or to any of the polypeptide sequences given in Table A.1 herein;
(iv) a polypeptide encoded by the nucleic acid sequence as defined in claim 5; or
(v) an orthologue or paralogue of any of the GRF polypeptide sequences SEQ ID NOs given in Table A.1; and
b. introducing and expressing in said plant, plant part, or plant cell, a nucleic acid sequence encoding a SYT polypeptide under the control of a constitutive promoter, wherein the SYT polypeptide comprises
from N-terminal to C-terminal, an SNH domain having at least 20% sequence identity to the SNH domain of SEQ ID NO: 262, a Met-rich domain, and a QG-rich domain;
(ii) the most conserved residues of the SNH domain as represented by SEQ ID NO: 263, and shown in black in FIG. 5;
(iii) a domain having at least 20% sequence identity to the SSXT domain with an InterPro accession IPR007726 of SEQ ID NO: 264.
(iv) at least 20% amino acid sequence identity to the SYT polypeptide as represented by SEQ ID NO: 121 or to any of the full length polypeptide sequences given in Table A.2 herein; or
(v) a polypeptide encoded by a nucleic acid sequence represented by any one of the nucleic acid sequence SEQ ID NOs given in Table A.2 or a portion thereof, or a sequence capable of hybridizing with any one of the nucleic acid sequences SEQ ID NOs given in Table A.2; and
c. cultivating the plant cell, plant part, or plant under conditions promoting plant growth and development.

33. A transgenic plant having increased yield-related traits relative to plants having increased expression of one of: (i) a nucleic acid sequence encoding a GRF polypeptide; or (ii) a nucleic acid sequence encoding a SYT polypeptide, resulting from increased expression of:
a nucleic acid sequence encoding a GRF polypeptide, wherein the GRF polypeptide comprises
(a) a domain having at least 50% amino acid sequence identity to a QLQ domain as represented by SEQ ID NO: 115, and a domain having at least 50% amino acid sequence identity to a WRC domain as represented by SEQ ID NO: 116;
(b) a QLQ domain with an InterPro accession IPR014978 (PFAM accession PF08880), a WRC domain with an InterPro accession IPR014977 (PFAM accession PF08879), and an Effector of Transcription (ET) domain comprising three Cys and one His residues in a conserved spacing (CX₉CX₁₀CX₂H);
(c) an amino acid sequence having at least 50% identity to the GRF polypeptide as represented by SEQ ID NO: 2 or to any of the polypeptide sequences given in Table A.1 herein;
(d) a polypeptide encoded by the nucleic acid sequence as defined in claim 5; or
(e) an orthologue or paralogue of any of the GRF polypeptide sequence SEQ ID NOs given in Table A.1; and
(ii) a nucleic acid sequence encoding a SYT polypeptide, wherein the SYT polypeptide comprises
(a) from N-terminal to C-terminal, an SNH domain having at least 20% sequence identity to the SNH domain of SEQ ID NO: 262, a Met-rich domain, and a QG-rich domain;
(b) the most conserved residues of the SNH domain as represented by SEQ ID NO: 263, and shown in black in FIG. 5;
(c) a domain having at least 20% sequence identity to the SSXT domain with an InterPro accession IPR007726 of SEQ ID NO: 264;
(d) at least 20% amino acid sequence identity to the SYT polypeptide as represented by SEQ ID NO: 121 or to any of the full length polypeptide sequences given in Table A.2 herein; or
(e) a polypeptide encoded by a nucleic acid sequence represented by any one of the nucleic acid sequence SEQ ID NOs given in Table A.2 or a portion thereof, or a sequence capable of hybridizing with any one of the nucleic acid sequences SEQ ID NOs given in Table A.2;
or a transgenic plant cell or transgenic plant part derived from said transgenic plant.

34. A transgenic plant according to claim 33, wherein said plant is a crop plant or a monocot or a cereal, such as rice, maize, wheat, barley, millet, rye, triticale, sorghum and oats, or a transgenic plant cell derived from said transgenic plant.

35. Harvestable parts of the transgenic plant according to claim 34, comprising (i) an isolated nucleic acid sequence encoding a GRF polypeptide; and (ii) an isolated nucleic acid sequence encoding a SYT polypeptide, wherein said harvestable parts are preferably seeds.

36. Products derived from the transgenic plant according to claim 34 and/or from harvestable parts of said transgenic plant.

37. The transgenic plant of claim 33, wherein the increased yield-related traits are one or more of: (i) increased early vigour; (ii) increased aboveground biomass; (iii) increased total seed yield per plant; (iv) increased seed filling rate; (v) increased number of (filled) seeds; (vi) increased harvest index; or (vii) increased thousand kernel weight (TKW).


SEQUENCE LISTING (270 sequences)

SEQ ID NO: 1 - DNA, 1194 bp, Arabidopsis thaliana
atgatgagtctaagtggaagtagcgggagaacaataggaaggcctccatttacaccaacacaatgggaagaactggaacatcaagccctaatctacaagtacatggtctctggtgttcctgtcccacctgagctcatcttctccattagaagaagcttggacacttccttggtctctagactccttcctcaccaatcccttggatgggggtgttaccagatgggatttgggagaaaaccagatccagagccaggaagatgcagaagaacagatggtaagaaatggagatgctcaagagaagcttacccagattcgaagtactgtgaaaaacacatgcacagaggaagaaaccgtgccagaaaatctcttgatcagaatcagacaacaacaactcctttaacatcaccatctctctcattcaccaacaacaacaacccaagtcccaccttgtcttcttcttcttcctctaattcctcttctactacttattctgcttcttcttcttcaatggatgcctacagtaacagtaataggtttgggcttggtggaagtagtagtaacactagaggttatttcaacagccattctcttgattatccttatccttctacttcacccaaacaacaacaacaaactcttcatcatgcttccgctttgtcacttcatcaaaatactaattctacttctcagttcaatgtcttagcctctgctactgaccacaaagacttcaggtactttcaagggattggggagagagttggaggagttggggagagaacgttctttccagaagcatctagaagctttcaagattctccataccatcatcaccaacaaccgttagcaacagtgatgaatgatccgtaccaccactgtagtactgatcataataagattgatcatcatcacacatactcatcctcatcatcatctcaacatcttcatcatgatcatgatcatagacagcaacagtgttttgttttgggcgccgacatgttcaacaaacctacaagaagtgtccttgcaaactcatcaagacaagatcaaaatcaagaagaagatgagaaagattcatcagagtcgtccaagaagtctctacatcacttctttggtgaggactgggcacagaacaagaacagttcagattcttggcttgacctttcttcccactcaagactcgacactggtagctaa

SEQ ID NO: 2 - protein, 397 aa, Arabidopsis thaliana
MMSLSGSSGRTIGRPPFTPTQWEELEHQALIYKYMVSGVPVPPELIFSIRRSLDTSLVSRLLPHQSLGWGCYQMGFGRKPDPEPGRCRRTDGKKWRCSREAYPDSKYCEKHMHRGRNRARKSLDQNQTTTTPLTSPSLSFTNNNNPSPTLSSSSSSNSSSTTYSASSSSMDAYSNSNRFGLGGSSSNTRGYFNSHSLDYPYPSTSPKQQQQTLHHASALSLHQNTNSTSQFNVLASATDHKDFRYFQGIGERVGGVGERTFFPEASRSFQDSPYHHHQQPLATVMNDPYHHCSTDHNKIDHHHTYSSSSSSQHLHHDHDHRQQQCFVLGADMFNKPTRSVLANSSRQDQNQEEDEKDSSESSKKSLHHFFGEDWAQNKNSSDSWLDLSSHSRLDTGS

SEQ ID NO: 3 - DNA, 735 bp, Arabidopsis thaliana
atggctacaaggattccattcacagaatcacaatgggaagaacttgaaaaccaagctcttgtgttcaagtacttagctgcaaatatgcctgttccacctcatcttctcttcctcatcaaaagaccctttctcttctcttcttcttcttcttcatcttcttcttcaagcttcttctctcccactctttctccacactttgggtggaatgtgtatgagatgggaatgggaagaaagatagatgcagagccaggaagatgtagaagaactgatggcaagaaatggagatgctctaaagaagcttaccctgactctaagtactgtgagagacatatgcatagaggcaagaaccgttcttcctcaagaaagcctcctcctactcaattcactccaaatctctttctcgactcttcttccagaagaagaagaagtggatacatggatgatttcttctccatagaaccttccgggtcaatcaaaagctgctctggctcagcaatggaagataatgatgatggctcatgtagaggcatcaacaacgaggagaagcagccggatcgacattgcttcatccttggtactgacttgaggacacgtgagaggccattgatgttagaggagaagctgaaacaaagagatcatgataatgaagaagagcaaggaagcaagaggttttataggtttcttgatgaatggccttcttctaaatcttctgtttctacttcactcttcatttga

SEQ ID NO: 4 - protein, 244 aa, Arabidopsis thaliana
MATRIPFTESQWEELENQALVFKYLAANMPVPPHLLFLIKRPFLFSSSSSSSSSSSFFSPTLSPHFGWNVYEMGMGRKIDAEPGRCRRTDGKKWRCSKEAYPDSKYCERHMHRGKNRSSSRKPPPTQFTPNLFLDSSSRRRRSGYMDDFFSIEPSGSIKSCSGSAMEDNDDGSCRGINNEEKQPDRHCFILGTDLRTRERPLMLEEKLKQRDHDNEEEQGSKRFYRFLDEWPSSKSSVSTSLFI

SEQ ID NO: 5 - DNA, 1593 bp, Arabidopsis thaliana
atggatcttggagttcgtgtttctggtcatgaaaccgtttcttctccgggtcaaactgaactcggatctggtttcagtaacaagcaagaaagatccggtttcgatggtgaagattgctggagaagttcaaagctctcacgaacatcaactgatggattctcttcttcccctgcctctgctaaaacgctgtcgtttcatcaaggcatccctttactgagatctaccactattaatgatcctcgtaaaggacaagaacacatgcttagcttctcttctgcttcaggcaaatcagatgtctcaccttatcttcagtactgtagaaactcaggatatggtttaggaggaatgatgaacacaagcaacatgcatggaaacttgttgacaggagtaaaaggacctttttcattgactcagtgggcagagctagagcaacaggcgttgatctataagtatatcacagccaatgtccctgttccatctagtttacttctctctctcaagaaatcttttttcccttatggttccttgcctcctaattcttttggatggggctcttttcatctgggcttttccggtggtaacatggatcccgagccagggagatgtcgccggacagatggaaagaaatggcggtgctcgagggacgctgttcccgatcaaaagtactgtgaacgacatattaacagaggccgccatcgttcaagaaagcctgtggaaggccaaaatggccacaatactaatgctgccgccgctgcttctgctgctgccgcttctaccgctgctgctgtgtccaaagcggcagcggggacttcagctgttgcgatgcgtggatcagataataacaatagccttgccgctgctgttggaacacaacatcataccaataatcaatctacagattctttggctaacagagttcaaaattctcgaggggcttcggtttttcctgccacgatgaacttacagtcgaaggaaactcatccgaaacaaagcaataatccctttgaattcggactcatctcttctgattcgttacttaatccgtcgcataaacaagcctcgtatgcaacctcttccaaaggctttggatcgtatcttgacttcggcaaccaagccaagcacgcggggaatcacaacaatgtcgattcttggcccgaagagctgaaatcggattggactcagctctcaatgtcaatccctatggctccatcttcccctgttcaagataaacttgcactctcacctttaaggttatcgcgtgagtttgaccccgcgatccacatgggattaggcgtcaacaccgagtttcttgaccccgggaaaaagacgaataactggataccaatctcctggggtaataacaactccatgggaggtccactcggcgaggtactaaacagcacgaccaatagtcccaagtttggttcctctccaacaggcgtcttgcaaaagtcgacatttggttctctttctaacagcagctcggcaagcagcaccatcattggcgataacaacaataagaacggtgatggaaaagatccgcttggcccgaccacgctgatgaatacttctgctactgctccttctctgtga

SEQ ID NO: 6 - protein, 530 aa, Arabidopsis thaliana
MDLGVRVSGHETVSSPGQTELGSGFSNKQERSGFDGEDCWRSSKLSRTSTDGFSSSPASAKTLSFHQGIPLLRSTTINDPRKGQEHMLSFSSASGKSDVSPYLQYCRNSGYGLGGMMNTSNMHGNLLTGVKGPFSLTQWAELEQQALIYKYITANVPVPSSLLLSLKKSFFPYGSLPPNSFGWGSFHLGFSGGNMDPEPGRCRRTDGKKWRCSRDAVPDQKYCERHINRGRHRSRKPVEGQNGHNTNAAAAASAAAASTAAAVSKAAAGTSAVAMRGSDNNNSLAAAVGTQHHTNNQSTDSLANRVQNSRGASVFPATMNLQSKETHPKQSNNPFEFGLISSDSLLNPSHKQASYATSSKGFGSYLDFGNQAKHAGNHNNVDSWPEELKSDWTQLSMSIPMAPSSPVQDKLALSPLRLSREFDPAIHMGLGVNTEFLDPGKKTNNWIPISWGNNNSMGGPLGEVLNSTTNSPKFGSSPTGVLQKSTFGSLSNSSSASSTIIGDNNNKNGDGKDPLGPTTLMNTSATAPSL

SEQ ID NO: 7 - DNA, 1197 bp, Arabidopsis thaliana
atggatttgcaactgaaacaatggagaagccagcagcagcaacaacatcagacagagtcagaagaacaaccttctgcagctaagataccaaaacatgtctttgaccagattcattctcacactgcaacttctactgctcttcctctctttacccctgagcctacttcttctaaactctcctctttgtctcctgattcttcctccaggttccccaagatggggagcttctttagctgggcacagtggcaagaacttgaactacaagctctgatctacaggtacatgttggctggtgctgctgttcctcaggagctccttttaccaatcaagaaaagccttctccatctatctccttcctactttcttcaccatcctcttcaacacctacctcattaccaacctgcttggtatttgggaagggcagcgatggatcctgagccaggcagatgcaggagaacggatggtaagaagtggagatgttcaagagacgtcttcgctggccacaagtattgcgagcgccacatgcaccgtggccgcaaccgttcaagaaagcctgtggaaactccaaccaccgtcaatgcaactgccacgtccatggcttcatcagtagcagccgcagccaccactacaacagcaacaacaacatctacgtttgcttttggtggtggtggtggtagtgaggaagtggttggtcaaggaggatctttcttcttctctggctcttctaactcttcatctgaacttctccaccttagtcaaagttgttcggagatgaagcaagaaagcaacaacatgaacaacaagaggccatacgagtcccacatcggattcagtaacaacagatcagatggaggacacatcctgaggcccttctttgacgattggcctcgttcttcgctccaagaagctgacaatagttcaagccccatgagctcagccacttgtctctccatctccatgcccgggaactcttcctcagacgtctctctgaagctgtccacaggcaacgaagagggagcccggagcaacaacaatgggagagatcagcaaaacatgagctggtggagcggtggaggttccaaccaccatcatcacaacatgggcggaccattggccgaagccctgagatcttcttcctcatcttccccaaccagtgttctccatcagcttggtgtctcgacacaagcctttcattga

SEQ ID NO: 8 - protein, 398 aa, Arabidopsis thaliana
MDLQLKQWRSQQQQQHQTESEEQPSAAKIPKHVFDQIHSHTATSTALPLFTPEPTSSKLSSLSPDSSSRFPKMGSFFSWAQWQELELQALIYRYMLAGAAVPQELLLPIKKSLLHLSPSYFLHHPLQHLPHYQPAWYLGRAAMDPEPGRCRRTDGKKWRCSRDVFAGHKYCERHMHRGRNRSRKPVETPTTVNATATSMASSVAAAATTTTATTTSTFAFGGGGGSEEVVGQGGSFFFSGSSNSSSELLHLSQSCSEMKQESNNMNNKRPYESHIGFSNNRSDGGHILRPFFDDWPRSSLQEADNSSSPMSSATCLSISMPGNSSSDVSLKLSTGNEEGARSNNNGRDQQNMSWWSGGGSNHHHHNMGGPLAEALRSSSSSSPTSVLHQLGVSTQAFH

SEQ ID NO: 9 - DNA, 1290 bp, Arabidopsis thaliana
atgcagagccctaaaatggagcaggaggaggttgaggaggagaggatgaggaataagtggccgtggatgaaggcggcgcagttaatggagtttcggatgcaagctttggtgtatagatacatagaggctggtctccgtgtgcctcatcatctcgtggtgcctatttggaacagtcttgctctctcttcttcctccaattacaactatcactcttcttctctgttgagtaacaagggagtaacccatatcgacacgttggaaactgaaccaactaggtgcaggagaacagatgggaagaaatggcgctgtagcaacacggtccttctattcgagaagtactgtgaacggcacatgcatagaggtcgtaaacgttcaagaaagcttgtggaatcttcttctgaggttgcttcatcatcaaccaaatacgacaacacttatggtttggataggtataacgagagtcagagtcatcttcatgggacaatctcgggttctagtaatgcgcaggtagttaccattgcttcactgcctagtgccagatcctgtgaaaatgtcattcgtccgtctttagtgatctctgaattcacaaacaaaagtgtgagtcacggcagaaagaacatggagatgagttatgatgactttattaatgaaaaagaggcgagtatgtgtgttggagttgttcctcttcaaggtgatgagagcaaaccttcggttcaaaagttcttccctgaggtatctgataaatgcttagaagctgcaaaattctcaagcaacaggaagaatgatataattgcaagaagcagagaatggaagaatatgaatgttaatggtggtttgtttcatggtatccacttttctccagacactgttcttcaagaacgtggttgttttcgtttacaaggagttgaaacagacaatgaaccaggaaggtgccgaagaacagatgggaagaagtggagatgcagcaaagatgttttgtctggtcagaagtactgcgataagcacatgcatagaggtatgaagaagaagcatccagttgatactactaactcacatgagaatgccgggtttagcccgttaaccgtggaaacagctgttagatcggttgtgccttgcaaagatggagatgaccagaagcattctgtttcagtcatgggaattacactgccccgagtttctgatgagaagagcactagcagttgcagtaccgacactaccattactgacacagctttaaggggtgaagacgacgatgaggagtacttgtctttgttttcaccaggtgtttag

SEQ ID NO: 10 - protein, 429 aa, Arabidopsis thaliana
MQSPKMEQEEVEEERMRNKWPWMKAAQLMEFRMQALVYRYIEAGLRVPHHLVVPIWNSLALSSSSNYNYHSSSLLSNKGVTHIDTLETEPTRCRRTDGKKWRCSNTVLLFEKYCERHMHRGRKRSRKLVESSSEVASSSTKYDNTYGLDRYNESQSHLHGTISGSSNAQVVTIASLPSARSCENVIRPSLVISEFTNKSVSHGRKNMEMSYDDFINEKEASMCVGVVPLQGDESKPSVQKFFPEVSDKCLEAAKFSSNRKNDIIARSREWKNMNVNGGLFHGIHFSPDTVLQERGCFRLQGVETDNEPGRCRRTDGKKWRCSKDVLSGQKYCDKHMHRGMKKKHPVDTTNSHENAGFSPLTVETAVRSVVPCKDGDDQKHSVSVMGITLPRVSDEKSTSSCSTDTTITDTALRGEDDDEEYLSLFSPGV

SEQ ID NO: 11 - DNA, 1647 bp, Arabidopsis thaliana
atggacttgcaactgaaacaatggagaagtcagcagcagaatgagtcagaagaacaaggctctgctgcaactaagatatcaaactttttctttgatcagattcagtcccaaactgctacttctgctgctgcggctcctcttcctctctttgtccctgaacccacttcttcctcttctttctcttgcttctctcctgactcttctaattcttcttcttcttccaggttcctcaagatgggaaacttcttcagctgggcacagtggcaagaacttgagctacaagcactgatctatagatacatgttggctggtgcttctgttcctcaagagcttctcttacctattaagaaaagtctcctccatcaatctcctatgcatttccttcaccatcctcttcaacatagttttcctcatcaccaaccttcttggtattggggaagaggagcaatggatcctgagccagggaggtgtaagagaactgacggcaagaaatggagatgttcaagggatgttgtagcgggccacaagtattgtgaccgccacattcaccgtggaagaaaccgttcaagaaagcctgtggaaaccgccacaaccaccatcacaacgacagccacaacaaccgcatcttcttttgtcttaggtgaggagcttggtcatggaccaaacaacaaccacttcttctcctctggttcatctcaacctctccaccttagtcatcaacaaagttgttcttcagagatgaaacaagaaagcaacaacaacaagaggccatatgaagctaacagtggattcagcaatggaagatcagacgatggtcacatcttgaggcatttctttgacgattggccacgatcatcagactctacctccagtccaatgagctcatccacttgtcatctttcaatctccatgcccggtaacaacacgtcctcagatgtttctctaaaactttccacaggcaatgaagaagaagaagagaacatgagaaataacaacaatgagagggagcaaatgaattggtggagcaatggagggaatcaccacaacaatatgggaggaccattagctgaggctttgaggtcagcttcttcgacgtcaagtgttcttcatcagatgggaatctctactcaagaaatgaagtatgtgaagccattgagcttattgggtaatgcgctgaagaccaaagtgtcagtccctggtcggtttctgggtttagatgttggtgataagtatgttggattagctatctcagatccttcaaatatggttgcttctccattgagtgttttgctcagaaagaaatcaaacattgacctgatggctacagatttccagaacctggtcaaagcattttctgtgtcgggattagtcgttggttatccatttggcaaactgaacaatgtagaggatgttgtcactgtgaatcttttcattgaggaacttcgtaagaccgaaaaactcaaggatgtgaaatacacatattgggacgagcgattatcatcaaagaccgttgaactgatgttgaagcccttgaatttgcatcctgttcaagagaagacaatgttggacaagttagccgcagtagttatacttcaggagtatttagattacgcgaacaggtatgtaaacactgagccagcagagtaa

SEQ ID NO: 12 - protein, 548 aa, Arabidopsis thaliana
MDLQLKQWRSQQQNESEEQGSAATKISNFFFDQIQSQTATSAAAAPLPLFVPEPTSSSSFSCFSPDSSNSSSSSRFLKMGNFFSWAQWQELELQALIYRYMLAGASVPQELLLPIKKSLLHQSPMHFLHHPLQHSFPHHQPSWYWGRGAMDPEPGRCKRTDGKKWRCSRDVVAGHKYCDRHIHRGRNRSRKPVETATTTITTTATTTASSFVLGEELGHGPNNNHFFSSGSSQPLHLSHQQSCSSEMKQESNNNKRPYEANSGFSNGRSDDGHILRHFFDDWPRSSDSTSSPMSSSTCHLSISMPGNNTSSDVSLKLSTGNEEEEENMRNNNNEREQMNWWSNGGNHHNNMGGPLAEALRSASSTSSVLHQMGISTQEMKYVKPLSLLGNALKTKVSVPGRFLGLDVGDKYVGLAISDPSNMVASPLSVLLRKKSNIDLMATDFQNLVKAFSVSGLVVGYPFGKLNNVEDVVTVNLFIEELRKTEKLKDVKYTYWDERLSSKTVELMLKPLNLHPVQEKTMLDKLAAVVILQEYLDYANRYVNTEPAE

SEQ ID NO: 13 - DNA, 1482 bp, Arabidopsis thaliana
atgaggatgcttcttgggattccttacgtagacaagtcggttctttccaactctgttcttgagagaggcaagcaggataaaagcaaactattgttagtcgacaaatgccattatgagcttgatgttgaagaacgcaaggaagattttgttggtgggtttggatttggtgttgtagaaaattcgcataaagacgttatggtgctacctcatcatcactattatccatcatattcatcaccttcctcttcttctttgtgttactgttctgctggtgttagcgatcccatgttctctgtttctagcaatcaggcttacacttcttctcacagtggtatgttcacacccgccggttctggttctgctgctgtgactgtagcagatccttttttctccttgagctcttcaggggaaatgagaagaagtatgaacgaagatgctggtgcagctttcagcgaagctcaatggcatgagcttgagaggcagaggaatatatacaagtacatgatggcttctgttcctgttcctccagagcttctcacaccctttcccaagaaccaccaatcaaacactaacccggatgtggatacatataggagtggaatgtttagtatttatgctgattacaagaatctgccgttgtctatgtggatgacagtaactgtggcagtggcgacaggaggctcattgcagctggggattgcttcaagcgcaagcaataacacggctgatctggagccatggaggtgcaagagaacagatgggaagaaatggaggtgctctagaaacgtgattcctgatcagaaatactgtgagagacacacacacaagagccgtcctcgttcaagaaagcatgtggaatcatctcaccaatcatctcaccacaatgacattcgtacggctaagaatgatactagccagcttgtgagaacttatcctcagttttacggacaacctataagccagatccctgtgctttctactcttccgtctgcctcctctccatatgatcaccacagaggactgaggtggtttacgaaagaagatgatgccattggaaccttaaacccggagactcaagaagctgtccagctgaaagttggatcaagcagagagctcaaacggggattcgattatgatctgaatttcaggcagaaagagccaatagtagaccagagctttggagcattgcagggtctattaagtctaaaccagacaccacaacataaccaagaaacaagacagtttgttgtagaaggaaagcaagatgaagcgatgggaagctctctgacactctcaatggctggaggaggcatggaggaaacagagggaacaaaccagcatcagtgggttagccatgaaggtccatcatggctctattcaacaacaccaggtggaccattggctgaagcactgtgtctcggtgtctccaacaacccaagttctagtactactactagtagctgcagcagaagctcaagctaa

SEQ ID NO: 14 - protein, 493 aa, Arabidopsis thaliana
MRMLLGIPYVDKSVLSNSVLERGKQDKSKLLLVDKCHYELDVEERKEDFVGGFGFGVVENSHKDVMVLPHHHYYPSYSSPSSSSLCYCSAGVSDPMFSVSSNQAYTSSHSGMFTPAGSGSAAVTVADPFFSLSSSGEMRRSMNEDAGAAFSEAQWHELERQRNIYKYMMASVPVPPELLTPFPKNHQSNTNPDVDTYRSGMFSIYADYKNLPLSMWMTVTVAVATGGSLQLGIASSASNNTADLEPWRCKRTDGKKWRCSRNVIPDQKYCERHTHKSRPRSRKHVESSHQSSHHNDIRTAKNDTSQLVRTYPQFYGQPISQIPVLSTLPSASSPYDHHRGLRWFTKEDDAIGTLNPETQEAVQLKVGSSRELKRGFDYDLNFRQKEPIVDQSFGALQGLLSLNQTPQHNQETRQFVVEGKQDEAMGSSLTLSMAGGGMEETEGTNQHQWVSHEGPSWLYSTTPGGPLAEALCLGVSNNPSSSTTTSSCSRSSS

SEQ ID NO: 15 - DNA, 1608 bp, Arabidopsis thaliana
atggatattggtgttcatgttcttgggtcggttactagtaatgaaaatgagtcacttggtctaaaagagcttataggaactaaacaagatagatccggattcatcggtgaggattgcttgcaacgaagcttgaagctagcaagaacgacaactagagcggaagaagaagaaaacttgtcttcttctgttgcagctgcttattgcaaaacgatgtcgtttcaccaaggcattcctctcatgagatctgcttctcctctttcctctgattctcgccgtcaagaacaaatgcttagcttctcagataaaccagacgctcttgatttcagtaaatatgtcggtttggataatagcagtaataacaagaactctctctcgccgtttcttcaccagattcctccaccttcttactttagaagctcaggaggatatggttctggtggaatgatgatgaacatgagcatgcaagggaacttcacaggtgttaaaggaccttttacattgactcaatgggctgagttagagcaacaggcgttgatctataagtacatcacagccaatgtccctgttccttctagtttgctcatctctatcaagaagtctttttatccttacggatctttgcctcctagttccttcggatggggaactttccatctcggtttcgcaggcggtaacatggaccctgagccagggagatgccgcagaacagatgggaagaaatggcggtgctcaagagacgccgttcctgatcagaaatactgtgaaagacacatcaacagaggccgtcatcgttcaagaaagcctgtggaagtccaatctggccaaaaccaaaccgccgctgctgcatccaaagcggttactacaccacaacagcctgttgtcgctggtaatactaacagaagcaatgcccgtgcatcaagcaaccgcagcctcgccattggaagtcaatatatcaatccttctacagaatctttacctaacaacagaggagtttcgatatatccttccaccgtcaacttacaacccaaggaatctccggttattcatcagaaacacagaaacaacaacaacccttttgagtttggacacatatcctctgattcgttactcaacccgaataccgcaaagacctatggatcatcgttcttggatttcagcagcaaccaagagaagcattcagggaatcacaatcacaattcttggcctgaagagctgacatcagattggacacagctctcaatgtcaattccaatagcatcatcatccccttcctccacacacaacaacaacaatgctcaagaaaaaacaacactctcgcctctcaggctatcccgcgagcttgacctatcgatccaaaccgatgaaacaacaatcgagcctactgtgaaaaaggtgaatacttggataccaatctcatggggaaactccttaggaggtcctctaggtgaagtactaaacagtacaacgaatagtccaacatttggatcttctcctacaggggttttgcaaaagtccacattttgttcactctctaacaacagctccgtgagcagccccattgcagagaacaacagacacaatggcgattactttcattacacaacctga

SEQ ID NO: 16 - protein, 535 aa, Arabidopsis thaliana
MDIGVHVLGSVTSNENESLGLKELIGTKQDRSGFIGEDCLQRSLKLARTTTRAEEEENLSSSVAAAYCKTMSFHQGIPLMRSASPLSSDSRRQEQMLSFSDKPDALDFSKYVGLDNSSNNKNSLSPFLHQIPPPSYFRSSGGYGSGGMMMNMSMQGNFTGVKGPFTLTQWAELEQQALIYKYITANVPVPSSLLISIKKSFYPYGSLPPSSFGWGTFHLGFAGGNMDPEPGRCRRTDGKKWRCSRDAVPDQKYCERHINRGRHRSRKPVEVQSGQNQTAAAASKAVTTPQQPVVAGNTNRSNARASSNRSLAIGSQYINPSTESLPNNRGVSIYPSTVNLQPKESPVIHQKHRNNNNPFEFGHISSDSLLNPNTAKTYGSSFLDFSSNQEKHSGNHNHNSWPEELTSDWTQLSMSIPIASSSPSSTHNNNNAQEKTTLSPLRLSRELDLSIQTDETTIEPTVKKVNTWIPISWGNSLGGPLGEVLNSTTNSPTFGSSPTGVLQKSTFCSLSNNSSVSSPIAENNRHNGDYFHYTT

SEQ ID NO: 17 - DNA, 1098 bp, Arabidopsis thaliana
atggactttctcaaagtttcagacaagacaacaattccatatagaagtgattctttgtttagtttgaatcagcaacaatacaaagagtcttcttttggattcagagacatggagattcatccgcatcctactccatatgcaggaaatggacttttgggttgttattactattaccctttcacaaacgcacaattgaaggagcttgagagacaagcaatgatctacaagtacatgatcgcatctattcctgttcctttcgatctacttgtttcttcaccatcctctgcctctccttgtaacaataaaaacatcgccggagatttagagccgggaagatgccggagaacagacggaaagaaatggagatgcgcgaaagaagtcgtctctaatcacaaatactgtgagaaacacttacacagaggtcgtcctcgttcaagaaagcatgtggaacctccttattctcgccctaacaacaatggtggttctgtgaaaaacagagatctcaaaaagcttcctcaaaagttatctagtagttccatcaaagacaaaacacttgagccaatggaggtttcatcatcaatctcaaactatagagactccagaggaagtgagaaatttactgtattggcaacaacagagcaagagaacaagtatctgaatttcatagatgtatggtccgatggagtaagatcatctgaaaaacagagtacaacttcaacacctgtttcttcttccaatggcaatctctctctttactcgcttgatctctcaatgggaggaaacaacttaatgggccaagacgaaatgggcctgatacaaatgggcttaggtgtaatcgggtcgggtagtgaggatcatcacgggtatggtccttatggtgtgacttcttcactagaggagatgtcaagctggcttgctccgatgtctaccacacctggtggaccattagcggagatactgaggccgagtacgaatttggcgatctctggtgatatcgaatcgtatagcttgatggagactcccactccaagctcgtccccgtctagagtgatgaagaagatgactagttcagtgtccgacgaaagcagccaggtttag

SEQ ID NO: 18 - protein, 365 aa, Arabidopsis thaliana
MDFLKVSDKTTIPYRSDSLFSLNQQQYKESSFGFRDMEIHPHPTPYAGNGLLGCYYYYPFTNAQLKELERQAMIYKYMIASIPVPFDLLVSSPSSASPCNNKNIAGDLEPGRCRRTDGKKWRCAKEVVSNHKYCEKHLHRGRPRSRKHVEPPYSRPNNNGGSVKNRDLKKLPQKLSSSSIKDKTLEPMEVSSSISNYRDSRGSEKFTVLATTEQENKYLNFIDVWSDGVRSSEKQSTTSTPVSSSNGNLSLYSLDLSMGGNNLMGQDEMGLIQMGLGVIGSGSEDHHGYGPYGVTSSLEEMSSWLAPMSTTPGGPLAEILRPSTNLAISGDIESYSLMETPTPSSSPSRVMKKMTSSVSDESSQV

SEQ ID NO: 19 - DNA, 1658 bp, Aquilegia formosa x Aquilegia pubescens
acttaaaagaccagtcttagctttcttcattaattcctactactgttctcagtgttgctctttgagtttatagatatttttcttacaatgatgatgagtgctagaaacagaaatcctttcactgtaactcaatggcaagaacttgaacatcaagctctcatttataagtatatggcttcaggaatgcctataccacctgatctcatcttccctattaagagaagtcttgattcttcaagattctttcctcatcaaccaatggattggggttgttttcagatgggttatggcaggaaagttgatccagaacctggaaggtgcagaagaacagatggaaagaagtggagatgctcaaaggaagcatacccagactcaaagtactgtgagagacacatgcacagaggcagaaaccgttcaagaaagcctgtggaagttaatactacatcaaattcctcattaccactttcatcttttacctctagaactccttctagtaccattacttcaaataccaacccttcttcttattccctttcttcatctctaacatctgacaaatctcagcaagaacatcatcacccttatcataacacccctcttcattcctttctcaatcctagtagaacttcttgttcttctcctagaactcataatattgatttctcacctcatagcaataacaatgccaatttggtattagactctgggtcttactctaactcttatgaagatcacagaaacaggtatgttcatggtctaaaagaagaggtagatgaaagagctttcttttcagaagcatcaggaacattaagaagtgtaccagaatcaactttgaaagatccatggcgtttaacaccattaagaatgagttcttcaactcataaccaaccaaaagatggaaatttttctgatttacaaagagggtattctcagtttcaactccaacataaacaacaacaacagcaacaagaagaagaacagcattgttttattttaggtactgatttcaaatctgacaggtttatgaaaactagtactactactactgagaaagaagaatcacaacaaccacttcgccatttctttgatgaatggccacctaagagtaaagattcttggttgggtttagaagaagatagatcagatcaaggttcacattcaacaactcaactttcaatatctattcctatgtcttctcatgagttctcagtttcaaattccagaacctaacaataagatgatggttgatttacttaaagtgggattattatggaaagattaatgacaacaaggagttgattcaaggttgggttcagtgtctttgtacttgtattgtctttatttaattgatgatgagaagtttaggtagagagtgctatgtgttatttttttttttgttatgtgtgtggagtgattgaaaagtgtgtctttaaacagtaagattcctgtcttgtgttttcttgatagctgttagaactttgtttgaatgactgatgaacaaatatttgggatttggggatttgtttgtatcaatattaggtgttttttctgtctttttggcttcttccatgattgccaaagaccatttgttcaacctaaaaatgataatgaagggggggccaatttgatatcatgagcttggttgtcagttaggaaag

SEQ ID NO: 20 - protein, 377 aa, Aquilegia formosa x Aquilegia pubescens
MMMSARNRNPFTVTQWQELEHQALIYKYMASGMPIPPDLIFPIKRSLDSSRFFPHQPMDWGCFQMGYGRKVDPEPGRCRRTDGKKWRCSKEAYPDSKYCERHMHRGRNRSRKPVEVNTTSNSSLPLSSFTSRTPSSTITSNTNPSSYSLSSSLTSDKSQQEHHHPYHNTPLHSFLNPSRTSCSSPRTHNIDFSPHSNNNANLVLDSGSYSNSYEDHRNRYVHGLKEEVDERAFFSEASGTLRSVPESTLKDPWRLTPLRMSSSTHNQPKDGNFSDLQRGYSQFQLQHKQQQQQQEEEQHCFILGTDFKSDRFMKTSTTTTEKEESQQPLRHFFDEWPPKSKDSWLGLEEDRSDQGSHSTTQLSISIPMSSHEFSVSN

SEQ ID NO: 21 - DNA, 1286 bp, Brassica napus
gaagaaagatgatgggtctaagtggaaatggtgggagaacaatagagaggcctccatttacaccaacacaatggcaagaactggagaatcaagccctaatttacaagtacatggtctcaggagttcctgtcccacctgagctcatcttctccattagaagaagcttggactcttccttggtctctagactcctccctcaccaatccattgggtggggatgctatcagatggggtttggtagaaaaccagatccagaaccaggaaggtgcagaagaacagatggtaagaaatggagatgctcaagagaagcataccctgattcaaagtactgtgaaaaacacatgcacagaggaaggaaccgtgccagaaaatctattgatcagaatcagacaactgctcctttaacatcaccatctctctctttccccaacaacaacaacccaagccctaccttgtcttcttcctcctctacttattcagctgcttcttcatctccttccattgatgcttacagtaatatcaataggcttggtgttggtagtagtaacagtagaggttacttcaacaaccattcccttgactatccttatcctttgtcctcacctaaacagcaacaacaacagcaacaaactcttagtcatgtttctgctttgtcacttcatcaaaacacatctacacctcagctcaatgtctttgcctctgcaactgaccacaaagacttcagatattttcaagggattggggagagagttggagttggggaaagaactttttttccagaagcttctagaagctttcaagattctccataccatcaccaacaaccgttagcaacggtagtggataatccgtacgactgtactactgatcataagtttgatcatcatcatacatactcatcatcatctcaacatcatcatcatgaccaagatcatcgacaacaacaacaatgttttgttttgggcgccgacatgttcaacaaacccacaagaactatcttggaaaacacatcgagacaagattatcttaatcaagaagaggaagagaaagattcatcggacacgaagaagtcccttcatcatttctttggtgaagagtggacacagaacaagaacagttcagattcttggcttgacctttcttcccagtcaagactcgacactggtagctgattgatgaggccagatagcatcagtgatgggtctgcaccaacacacacacaaacacgtttgaagggtcacatttcacatctatttccgtggaacattgagacagacaagacactg

SEQ ID NO: 22 - protein, 387 aa, Brassica napus
MMGLSGNGGRTIERPPFTPTQWQELENQALIYKYMVSGVPVPPELIFSIRRSLDSSLVSRLLPHQSIGWGCYQMGFGRKPDPEPGRCRRTDGKKWRCSREAYPDSKYCEKHMHRGRNRARKSIDQNQTTAPLTSPSLSFPNNNNPSPTLSSSSSTYSAASSSPSIDAYSNINRLGVGSSNSRGYFNNHSLDYPYPLSSPKQQQQQQQTLSHVSALSLHQNTSTPQLNVFASATDHKDFRYFQGIGERVGVGERTFFPEASRSFQDSPYHHQQPLATVVDNPYDCTTDHKFDHHHTYSSSSQHHHHDQDHRQQQQCFVLGADMFNKPTRTILENTSRQDYLNQEEEEKDSSDTKKSLHHFFGEEWTQNKNSSDSWLDLSSQSRLDTGS

SEQ ID NO: 23 - DNA, 1559 bp, Hordeum vulgare
gggcagccgcagccgcagccgcagcagaggagagagagagggagggagaagcatatatggcgatgccctttgcctccctgtcgccggcagccgaccaccaccgctcctcccccatcttccccttctgccgctcctcccctctctactcggtaggggaggaggcggcgcatcagcatcctcatcctcagcagcagcagcagcagcacgcgatgagcggcgcgcggtgggcggcgaggccggcgcccttcacggcggcgcagtacgaggagctggagcagcaggcgctcatctacaagtacctcgtcgccggcgtccccgtcccgcaggacctcctcctccccatccgccgcggcttcgagaccctcgcctcgcgcttctaccaccaccacgcccttgggtacgggtcctacttcgggaagaagctggatccggagccggggcggtgccggcggacggacggcaagaagtggcggtgctccaaggaggccgctcaggactccaagtactgcgagcgccacatgcaccgcggccgcaaccgttcaagaaagcctgtggaaacgcagctcgtcgccagctcccactcccagtcccagcagcacgccaccgccgccttccacaaccactcgccgtatccggcgatcgccactggcggtggctccttcgccctggggtctgctcagctgcacatggacactgctgcgccttacgcgacgaccgccggtgctgccggaaacaaagatttcaggtattctgcctatggagtgaggacgtcggcgatcgaggagcacaaccagttcatcaccgcggccatggacaccgccatggacaactactcgtggcgcctgatgccgtcccaggcctcggcattctcgctctccagctaccccatgctgggcacgctgagcgacctggaccagagcgcgatctgctcgctggccaagactgagagggagccactgtccttcttcggcggcggcggcgacttcgacgacgactcggctgcggtgaagcaggagaaccagacgctgcggcccttcttcgacgagtggcccaaggacagggactcgtggccggagctgcaagaccacgacgccaacaacaacagcaacgccttctcagccaccaagctgtccatctccatgccggtcaccagctccgacttctctggcaccaccgccggctcccgctcgcccaacggtatatactcccggtgaacggcgtcggccggcctgatctctgctgatttgccgtggtcacgacgggcgtcctcaaatcatcacagatgagcgaaccggccgacccgatcgaatgtgtctgtgagccgactgcagcttgcttgctcattttgtatggatcgtcgtgcagcaggaacgaaacactactcctttaatttcctttctttaatttcacaacgttttttctgggttttgccgtgtatcggccggaactgtactaccaagttttctatagcctcgatggtcatgcacgacatcgttgactgtttcccgcgcacttactgttgaaataatcttccatttttggcaaaaaaaaaaaaaaaa

SEQ ID NO: 24 - protein, 385 aa, Hordeum vulgare
MAMPFASLSPAADHHRSSPIFPFCRSSPLYSVGEEAAHQHPHPQQQQQQHAMSGARWAARPAPFTAAQYEELEQQALIYKYLVAGVPVPQDLLLPIRRGFETLASRFYHHHALGYGSYFGKKLDPEPGRCRRTDGKKWRCSKEAAQDSKYCERHMHRGRNRSRKPVETQLVASSHSQSQQHATAAFHNHSPYPAIATGGGSFALGSAQLHMDTAAPYATTAGAAGNKDFRYSAYGVRTSAIEEHNQFITAAMDTAMDNYSWRLMPSQASAFSLSSYPMLGTLSDLDQSAICSLAKTEREPLSFFGGGGDFDDDSAAVKQENQTLRPFFDEWPKDRDSWPELQDHDANNNSNAFSATKLSISMPVTSSDFSGTTAGSRSPNGIYSR

SEQ ID NO: 25 - DNA, 1521 bp, Lycopersicon esculentum
gatgataagaaacacacaaatgacttaactttgcaggtttcaccgcactcgacactgcaaaaaagatacatataaaaaaaaaggtccactcaactctctgcaaaaataaaaaaaattaaaaacttttgtccaagacttaactttctcttcagaaataaatttgccttcacattaatattttgttgttagtaacaaaaatcattctcaatcgaaacatggacttcaatatgaagcaatggagtaatcaacatgagtcagaaaatcaagaatcaccaacaaagttaccaagacttcttcttgacttccactctgtttcttctgattctgcttctgctgctgctctaccattgtttgtatctgaaccaacaacatcaacaacaacttgtaccaaattaatgtcagattcagcaaccactgtcaccaccaaatttccaaggattggaagtggtggtggttacttcagcttggctcaatggcaagaacttgaactacacagtttgatttttaggcattttgtagctggtgcccctgttccttctgaactacttcatcttgttaagaaaagtattattgcttctcctcctcctcctccttcatattactttgctcatccatatcaacagtatcctcattatcaacaagctttgatgcagtcagggtactggggtagagccgccatggatccagaaccaggaaggtgtaggaggactgatggcaagaaatggaggtgctcaagggatgtagtggctggccagaaatactgcgagcgccacgttcatcgtggccgcagccgttcaagaaagcctgtggaaattcccacacctgccaacaatggcagtaaaaacaacaacactgtttctcatcatcaagcctttggaaaaatgactggacatgctcatgctggtggtggtgctcctcagttttctctttcgggacattcaccttccactaatgcgccttttcatctcaatcaaaggccaattaagggtccaccacaagaagtacttcaaaaagatgtatctattggtgatggtaaatcatctagtggccaaatcctacgccatttcttcgacgattggcctagacaacaacttcaagaaggcgacaatgctgcaaccagcctgtccatttcgatgcccggtgtagggggtaacccctcgtcagacttctcgttgaagctttcaactgggaattactatgattcaggtactcaagttagtaatgttgaacggtctacatgggggacgagtcaccaccacgtagcctcaatgggtggtccacttgccgaggccttaaggtcatcaacaactaactcgtcccctactagcgtgttgcatcaattggcacgaggtagcgcgtccgaggccagctatattagcacttgatttctgcaagtgttcttgttaaatgtttttttcttttggactttattgttttttaacttggttgtgttgttgttcattgttctttattggtattgatatacctaactgtcacctgtacaaaaaaaaaaaaaaaaaa

SEQ ID NO: 26 - protein, 389 aa, Lycopersicon esculentum
MDFNMKQWSNQHESENQESPTKLPRLLLDFHSVSSDSASAAALPLFVSEPTTSTTTCTKLMSDSATTVTTKFPRIGSGGGYFSLAQWQELELHSLIFRHFVAGAPVPSELLHLVKKSIIASPPPPPSYYFAHPYQQYPHYQQALMQSGYWGRAAMDPEPGRCRRTDGKKWRCSRDVVAGQKYCERHVHRGRSRSRKPVEIPTPANNGSKNNNTVSHHQAFGKMTGHAHAGGGAPQFSLSGHSPSTNAPFHLNQRPIKGPPQEVLQKDVSIGDGKSSSGQILRHFFDDWPRQQLQEGDNAATSLSISMPGVGGNPSSDFSLKLSTGNYYDSGTQVSNVERSTWGTSHHHVASMGGPLAEALRSSTTNSSPTSVLHQLARGSASEASYIST

SEQ ID NO: 27 - DNA, 1100 bp, Medicago truncatula
atgatgagtgcaagttcaagaaataggtcacttttcacaccaaatcaatggcaagaacttgaacaacaagccctagtttttaaatacatggttactggaacacctattccaccagatctcatatactctattaagagaagtttagacacttcaatatcttcaagaatctttcctcatccaccaattgggtggggatgttttgaaatgggatttggcagaaaagtagacccagagccagggaggtgcagaagaacagatggcaagaaatggagatgctcaaaggaagcatatccagactcaaagtactgtgaaagacacatgcacagaggtagaaaccgttcaagaaagcctgtggaactagtagtttcttcttcaacaacaacaccaacaaataacacaaacacagcatcttcttacagcaacagaaacatctccttgaacaacaacagcagcagcataaactcaccttcttctttccctttctctacttcatccatggcttgtcatgatcagtcacaatctttttcacaatcctaccaaaactcttctttaaacccttactattactctcaatcaattacctctactaacccacttgatcattctcattttcaaactcaagatgctactactcatcacctctttttggactcaacatcttattctcaggatgacaaggactttaggtatgtacaagttcaaggaataagagatggtactgtggatgagagaactttctttccagaagctacaggttcatctaggagctgttatcatgattcatatcaacaacaactatcaatgaatccctttaagtcttactcaagctcacagtttcagaatatcaatgatgataattcaagacaacaacaagaacaacactgttttgttttaggcactgacatcaagtcaacaagaacaacaaacaaggacaaagaaagtgagacaactcagaaaccacttcatcatttctttggtgagtggacaccaaagaacacagattcctggctagatcttgcttctaactccagaattccaacaggttgattatcatttatcatcattcctatgtttttgttttttttttgttattattaata

SEQ ID NO: 28 - protein, 348 aa, Medicago truncatula
MMSASSRNRSLFTPNQWQELEQQALVFKYMVTGTPIPPDLIYSIKRSLDTSISSRIFPHPPIGWGCFEMGFGRKVDPEPGRCRRTDGKKWRCSKEAYPDSKYCERHMHRGRNRSRKPVELVVSSSTTTPTNNTNTASSYSNRNISLNNNSSSINSPSSFPFSTSSMACHDQSQSFSQSYQNSSLNPYYYSQSITSTNPLDHSHFQTQDATTHHLFLDSTSYSQDDKDFRYVQVQGIRDGTVDERTFFPEATGSSRSCYHDSYQQQLSMNPFKSYSSSQFQNINDDNSRQQQEQHCFVLGTDIKSTRTTNKDKESETTQKPLHHFFGEWTPKNTDSWLDLASNSRIPTG

SEQ ID NO: 29 - DNA, 1302 bp, Medicago truncatula
atgcatatgttgacaatggaagctaaacctcttcaacttgttccctcttcacacaacagcacaactggtggtggaccccagatgaagattgagaatggtgaagttgatgaagagaaaagggttgttgttggagtgaaggaagatatagaaaacaagcctttgatcacagaagctcaaaggcgtgaacttgatcatcaagtttttatttttaatcattttgcttataatcttcctcttccttattaccttttgcaatttccaagtaatatgtcagagtacagtcgtcgtgggtctgattatgtgactatggtggatcaagaaccacataggtgtagaagaactgacggaaagaaatggaggtgcggcaaggacacagtacctaatcagaagtattgtgaacgtcacatgcacagaggtcgaaatcgttcaagaaagcttgtggaaacatctcaacttaactctcctttgaaaacaaatcctagtggtggtggcaagtcacatgcaaaactagtcccaaacattaaatcttcagtttcaaatccaaaccctttgattattcatcacaatggcacattctcatacaatccgaggaccttctgcgttgtagatacttcttctgtttgtgatcggtcgagacatgtcatagattatggtgccactgcagtgacaacttcgggaagcacgacatccgtttctttggataacagagtttgtcctaacgtatgcaagcaagatgagcagatcaagaggtgtatcaccgacaacgtgggtattaaaagtggtcggaaaggaagcatatcttgtgaaagtattggcatctctactggaataggcttttccccaaagagtgttcttccagtttctggttgcaatgattcatacctcaacaacagaaacaatatattagaacctgaacccggtagatgccgaagaacagatggtaagaagtggcgatgcaagagtgcggttcttccaggtcagaagtattgtgcaacacatatgcatagaggtgctaaaaggcgttttacaaacctcgaatctcctcctcctgccaccactgttattcctaaaactactgatattagttcagctgttaccattgctcagttgcccgacccttcggctccaatcgacatccagaaagcgaattgttggtctccgagcactaagctttcaatgtcggttcaagaaagtgcgccctttgttgattgtaatgagaaaagtgttagcagcggtgacacggatggtactagtaccaccatcactgacaccatgaatgagtgtagctatctttctttctaa

SEQ ID NO: 30 - protein, 433 aa, Medicago truncatula
MHMLTMEAKPLQLVPSSHNSTTGGGPQMKIENGEVDEEKRVVVGVKEDIENKPLITEAQRRELDHQVFIFNHFAYNLPLPYYLLQFPSNMSEYSRRGSDYVTMVDQEPHRCRRTDGKKWRCGKDTVPNQKYCERHMHRGRNRSRKLVETSQLNSPLKTNPSGGGKSHAKLVPNIKSSVSNPNPLIIHHNGTFSYNPRTFCVVDTSSVCDRSRHVIDYGATAVTTSGSTTSVSLDNRVCPNVCKQDEQIKRCITDNVGIKSGRKGSISCESIGISTGIGFSPKSVLPVSGCNDSYLNNRNNILEPEPGRCRRTDGKKWRCKSAVLPGQKYCATHMHRGAKRRFTNLESPPPATTVIPKTTDISSAVTIAQLPDPSAPIDIQKANCWSPSTKLSMSVQESAPFVDCNEKSVSSGDTDGTSTTITDTMNECSYLSF

SEQ ID NO: 31 - DNA, 1185 bp, Oryza sativa
atggcgatgccgtatgcctccctgtctccggcggtggccgaccaccgctcgtccccggcagccgcgaccgcctccctcctccccttctgccgctccaccccgctctccgcgggcggtggtggcgtcgcgatgggggaggacgcgccgatgaccgcgaggtggccgccggcggcggcggcgaggctgccgccgttcaccgcggcgcagtacgaggagctggagcagcaggcgctcatatacaagtacctggtggcaggcgtgcccgtcccgccggatctcgtgctccccatccgccgcggactcgactccctcgccgcccgcttctacaaccatcccgcccttggatatggtccgtacttcggcaagaagctggacccagagccagggcggtgccggcgtacggacggcaagaaatggcggtgctcgaaggaggccgcgccggattccaagtactgcgagcgccacatgcaccgcggccgcaaccgttcaagaaagcctgtggaaacgcagctggtcgcccagtcccaaccgccctcatctgttgtcggttctgcggcggcgccccttgctgctgcctccaatggcagcagcttccaaaaccactctctttaccctgctattgccggcagcaatggcgggggcggggggaggaacatgcccagctcatttggctcggcgttgggttctcagctgcacatggataatgctgccccttatgcagctgttggtggtggaacaggcaaagatctcaggtatactgcttatggcacaagatctttggcggatgagcagagtcaactcattactgaagctatcaacacatctattgaaaatccatggcggctgctgccatctcagaactcgccatttcccctttcaagctattctcagctgggggcactaagtgaccttggtcagaacacccccagctcactttcaaaggttcagaggcagccactttcgttctttgggaacgactatgcggctgtcgattctgtgaagcaagagaaccagacgctgcgtcccttctttgatgagtggccaaagggaagggattcatggtcagacctcgctgatgagaatgctaatctttcgtcattctcaggcacccaactgtcgatctccataccaatggcatcctctgacttctcggcggccagttctcgatcaactaatggtgactga

SEQ ID NO: 32 - protein, 394 aa, Oryza sativa
MAMPYASLSPAVADHRSSPAAATASLLPFCRSTPLSAGGGGVAMGEDAPMTARWPPAAAARLPPFTAAQYEELEQQALIYKYLVAGVPVPPDLVLPIRRGLDSLAARFYNHPALGYGPYFGKKLDPEPGRCRRTDGKKWRCSKEAAPDSKYCERHMHRGRNRSRKPVETQLVAQSQPPSSVVGSAAAPLAAASNGSSFQNHSLYPAIAGSNGGGGGRNMPSSFGSALGSQLHMDNAAPYAAVGGGTGKDLRYTAYGTRSLADEQSQLITEAINTSIENPWRLLPSQNSPFPLSSYSQLGALSDLGQNTPSSLSKVQRQPLSFFGNDYAAVDSVKQENQTLRPFFDEWPKGRDSWSDLADENANLSSFSGTQLSISIPMASSDFSAASSRSTNGD

SEQ ID NO: 33 - DNA, 1194 bp, Oryza sativa
atgatgatgatgagcggtcgcccgagcggcggcgccggcggaggtcggtacccgttcacggcgtcgcagtggcaggagctggagcaccaggcgctcatctacaagtacatggcgtccgggactcccatcccctccgacctcatcctccccctccgccgcagcttcctcctcgactccgccctcgccacctccccttccctcgccttccctccccaaccttcactggggtggggttgctttggcatggggtttgggcggaaggcggaggacccggagccagggcgatgccggcgtacggacggcaagaagtggcggtgctccaaggaggcgtacccggactccaagtactgcgagaagcacatgcaccgtggcaagaaccgttcaagaaagcctgtggaaatgtccttggccacgccgccgccgccgtcctcctccgccacctccgccgcgtcgaacacctccgccggcgtcgcccccaccaccaccaccacctcctccccggcgccctcctacagccgcccggcgccgcacgacgcggcgccgtaccaggcgctctacggcgggccctacgccgcggccaccgcgcgcacccccgccgccgcggcgtaccacgcgcaggtgagcccgttccacctccagctcgacaccacccacccgcacccgccgccgtcctactactccatggaccacaaggagtacgcgtacgggcacgccaccaaggaggtgcacggcgagcacgccttcttctccgatggcaccgagagggagcaccaccacgccgccgccgggcacggccagtggcagttcaagcagctcggcatggagcccaagcagagcaccacgcctctcttcccgggcgccggctacggccacaccgcggcgtcgccgtacgccattgatctttcaaaagaggacgacgatgagaaagagaggcggcaacagcagcagcagcagcagcagcagcactgcttcctcctgggcgccgacctccgtctggagaagccggcgggccacgaccacgcggcggcggcgcagaaacctctccgccacttcttcgacgagtggccgcatgagaagaacagcaagggctcctggatggggctcgaaggcgagacgcagctgtccatgtccatccccatggccgccaacgacctcccgatcaccaccacctcccgctaccacaatgatgattaa

SEQ ID NO: 34 - protein, 397 aa, Oryza sativa
MMMMSGRPSGGAGGGRYPFTASQWQELEHQALIYKYMASGTPIPSDLILPLRRSFLLDSALATSPSLAFPPQPSLGWGCFGMGFGRKAEDPEPGRCRRTDGKKWRCSKEAYPDSKYCEKHMHRGKNRSRKPVEMSLATPPPPSSSATSAASNTSAGVAPTTTTTSSPAPSYSRPAPHDAAPYQALYGGPYAAATARTPAAAAYHAQVSPFHLQLDTTHPHPPPSYYSMDHKEYAYGHATKEVHGEHAFFSDGTEREHHHAAAGHGQWQFKQLGMEPKQSTTPLFPGAGYGHTAASPYAIDLSKEDDDEKERRQQQQQQQQQHCFLLGADLRLEKPAGHDHAAAAQKPLRHFFDEWPHEKNSKGSWMGLEGETQLSMSIPMAANDLPITTTSRYHNDD

SEQ ID NO: 35 - DNA, 1371 bp, Oryza sativa
atgcagggtgcaatggccagggtgaggggtcccttcacgccgtctcagtggatcgagctggagcaccaggcgctgatatacaagtacttggctgcgaatagccctgtaccacacagcctcctcatccccatcaggaggagcctcacatcgccctactcacctgcctactttggctcaagcacattgggatggggatctttccagctgggctactccggcagcgcggatccggagcccggccggtgccgccggacggacggcaagaaatggcggtgctcgagggatgcggtcgccgaccagaagtactgtgagcgacacatgaaccggggacgccaccgttcaagaaagcatgtggaaggccagcctggccatgccgcgaaagcgatgcccgcggcggtggcagcagccgctgcctctgctacccagcctagtgctccggccgcccacagtggcggagctgttgctggcctcgctatcaaccatcagcaccagcaaatgaagaactacgctgccaacactgccaatccttgctctctgcaatatagcagggatctggcaaacaagcataatgagagtgaacaagtgcaagactcagacagtctctcgatgctgacttccattagcacgagaaatacgggcagcctgtttccgttctcaaaacaacataatccttttgaagtgtccaactcaaggccagattttggcctagtatcacctgattcactgatgagttctcctcatagctccttggagaacgtcaatttgctcacttcgcagagtctgaatgaacaacagagttcagtttcccttcaacactttgtggactggccaaggacacctgcacaaggagctctcgcatggcctgatgctgaagacatgcaagctcagagaagccagctctcaatatctgctccaatggcgtcttctgacctgtcatcagcctcaacatctcccatccatgagaagctgatgttgtcaccacttaaactgagccgtgaatatagtcctattggtctcggttttgcagcaaatagagatgaggttaaccagggagaagcaaactggatgcctatgttccgtgattctttgatgggcggaccattgggagaggttttaaccaagaataacaacatggaagcaaggaattgcctatcggagtctctgaatcttttaaatgatggctgggattcaagctcagggtttgattcatccccagttggtgttctgcagaagaccacctttggatcagtatccagtagcaccggaagcagtcctagactggagaatcatagtgtttatgatggcaacagtaacctgcgggatgatctcggttcagttgttgtaaatcatccgagcatccgcctggtgtga

SEQ ID NO: 36 - protein, 456 aa, Oryza sativa
MQGAMARVRGPFTPSQWIELEHQALIYKYLAANSPVPHSLLIPIRRSLTSPYSPAYFGSSTLGWGSFQLGYSGSADPEPGRCRRTDGKKWRCSRDAVADQKYCERHMNRGRHRSRKHVEGQPGHAAKAMPAAVAAAAASATQPSAPAAHSGGAVAGLAINHQHQQMKNYAANTANPCSLQYSRDLANKHNESEQVQDSDSLSMLTSISTRNTGSLFPFSKQHNPFEVSNSRPDFGLVSPDSLMSSPHSSLENVNLLTSQSLNEQQSSVSLQHFVDWPRTPAQGALAWPDAEDMQAQRSQLSISAPMASSDLSSASTSPIHEKLMLSPLKLSREYSPIGLGFAANRDEVNQGEANWMPMFRDSLMGGPLGEVLTKNNNMEARNCLSESLNLLNDGWDSSSGFDSSPVGVLQKTTFGSVSSSTGSSPRLENHSVYDGNSNLRDDLGSVVVNHPSIRLV

SEQ ID NO: 37 - DNA, 711 bp, Oryza sativa
atgttggccgagggaaggcaagtctacttgccgccgccgccgccgtccaagcttcctcgtctctccggcaccgatccaaccgacggcgtggtgacgatggcagcgccgtcgccgctggttcttgggctgggtctcggtctgggcggcagcggcagcgacagcagtgggagcgacgcggaagcgtctgcggccaccgtgcgggaggcgcggccgccgtcggcgctgacgttcatgcagcggcaggagctggagcagcaggtgctcatctaccgctacttcgccgccggcgcgcctgtgccggttcacctcgtgctgcccatatggaagagcatcgccgccgcctcctcgttcggcccgcaaagctttccctccctgacgggcctggggagcctgtgcttcgactacaggagcagcatggagccggagccggggcggtgccggcgcacggacggcaagaagtggcggtgctcgcgcgacgtggtgccggggcacaagtattgcgagcggcacgtccaccgtggccgcggccgttcaagaaagcctatggaagcctctgcagcagtcgctcccacatatctcccggtccggccggcactccacaccgtcgccaccctcgccaccagcgcgccatcgctgtcgcacctcggtttctcctccgccagcaaagtgctcctcgcccacaccaccaccggcaccacgcgcgctacttga

SEQ ID NO: 38 - protein, 236 aa, Oryza sativa
MLAEGRQVYLPPPPPSKLPRLSGTDPTDGVVTMAAPSPLVLGLGLGLGGSGSDSSGSDAEASAATVREARPPSALTFMQRQELEQQVLIYRYFAAGAPVPVHLVLPIWKSIAAASSFGPQSFPSLTGLGSLCFDYRSSMEPEPGRCRRTDGKKWRCSRDVVPGHKYCERHVHRGRGRSRKPMEASAAVAPTYLPVRPALHTVATLATSAPSLSHLGFSSASKVLLAHTTTGTTRAT

SEQ ID NO: 39 - DNA, 1164 bp, Oryza sativa
atggcgatgccctttgcctccctgtcgccggcagccgaccaccggccctccttcatcttccccttctgccgctcctcccctctctccgcggtcggggaggaggcgcagcagcacatgatgggcgcgaggtgggcggcggcggtggccaggccgccgcccttcacggcggcgcagtacgaggagctggagcagcaggcgctcatatacaagtacctcgtcgccggcgtgcccgtcccggcggatctcctcctccccatccgccgtggcctcgactcactcgcctcgcgcttctaccaccaccctgtccttggatacggttcctacttcggcaagaagctggacccggagcccggacggtgccggcgtacggacggcaagaagtggcggtgctccaaggaggccgcgccggactccaagtactgtgagcgacacatgcaccgcggccgcaaccgttcaagaaagcctgtggaagcgcagctcgtcgccccccactcgcagccccccgccacggcgccggccgccgccgtcacctccaccgccttccagaaccactcgctgtacccggcgattgctaatggcggcggcgccaacggaggcggtggtggtggtggcggtggcggcagcgcgcctggctcgttcgccttggggtctaatactcagctgcacatggacaatgctgcgtcttactcgactgttgctgctggtgccggaaacaaagatttcaggtattctgcttatggagtgagaccattggcagatgagcacagcccactcatcactggagctatggatacctctattgacaattcgtggtgcttgctgccttctcagacctccacattttcagtttcgagctaccctatgcttggaaatctgagtgagctggaccagaacaccatctgctcgctgccgaaggtggagagggagccattgtcattcttcgggagcgactatgtgaccgtcgactccgggaagcaggagaaccagacgctgcgcccctttttcgacgagtggccaaaggcaagggactcctggcctgatctagctgatgacaacagccttgccaccttctctgccactcagctctcgatctccattccaatggcaacctctgacttctcgaccaccagctcacgatcacacaacggtatatactcccgatga

SEQ ID NO: 40 - protein, 387 aa, Oryza sativa
MAMPFASLSPAADHRPSFIFPFCRSSPLSAVGEEAQQHMMGARWAAAVARPPPFTAAQYEELEQQALIYKYLVAGVPVPADLLLPIRRGLDSLASRFYHHPVLGYGSYFGKKLDPEPGRCRRTDGKKWRCSKEAAPDSKYCERHMHRGRNRSRKPVEAQLVAPHSQPPATAPAAAVTSTAFQNHSLYPAIANGGGANGGGGGGGGGGSAPGSFALGSNTQLHMDNAASYSTVAAGAGNKDFRYSAYGVRPLADEHSPLITGAMDTSIDNSWCLLPSQTSTFSVSSYPMLGNLSELDQNTICSLPKVEREPLSFFGSDYVTVDSGKQENQTLRPFFDEWPKARDSWPDLADDNSLATFSATQLSISIPMATSDFSTTSSRSHNGIYSR

SEQ ID NO: 41 - DNA, 1071 bp, Oryza sativa
atgctgagctcgtcgccctcggcggcggcgccggggataggagggtaccagccgcagcgcggggcggcggtcttcacggcggcgcagtgggcggagctggagcagcaggcgctcatttacaagtacctcgtcgccggtgtccccgtcccgggcgatctcctcctcccaatccgcccccactcctccgccgccgccacctactccttcgccaaccccgccgccgcgcccttctaccaccaccaccaccacccctctctgagctattatgcctactatggcaagaagcttgaccctgagccgtggcgttgccgccgcaccgacggcaagaagtggcggtgctccaaggaggcgcaccccgactccaagtactgcgagcgccacatgcaccgtggccgcaaccgttcaagaaagcctgtggaatccaagaccgctgcccctgcgccccagtcgcagccccagctgtccaatgtcacgaccgcgactcacgacaccgatgcgcctctcccgtcactcactgtgggtgctaaaacccacggtctgtcccttggtggtgctggctcgtcgcagttccatgtcgacgcaccatcgtacggcagcaagtactctcttggagctaaagctgatgtgggtgaactgagcttcttctcaggagcatcaggaaacaccaggggcttcaccattgattctccaacagatagctcatggcattcactgccttccagtgtacccccatacccgatgtcaaagccaagggactctggcctcctaccaggtgcctactcctactcccaccttgaaccttcacaggaacttggccaggtcaccatcgcctcgctgtcccaagagcaggagcgccgctcttttggtggtggagcgggggggatgctaggaaatgtgaagcacgagaaccagccgctgaggcctttcttcgatgagtggcctgggaggcgagactcgtggtcggagatggatgaggagaggtccaaccagacctccttctcgacaacccagctctcgatctccatcccgatgcccagatgtgggtcccctatcggtccgcgtctaccttga

SEQ ID NO: 42 - protein, 356 aa, Oryza sativa
MLSSSPSAAAPGIGGYQPQRGAAVFTAAQWAELEQQALIYKYLVAGVPVPGDLLLPIRPHSSAAATYSFANPAAAPFYHHHHHPSLSYYAYYGKKLDPEPWRCRRTDGKKWRCSKEAHPDSKYCERHMHRGRNRSRKPVESKTAAPAPQSQPQLSNVTTATHDTDAPLPSLTVGAKTHGLSLGGAGSSQFHVDAPSYGSKYSLGAKADVGELSFFSGASGNTRGFTIDSPTDSSWHSLPSSVPPYPMSKPRDSGLLPGAYSYSHLEPSQELGQVTIASLSQEQERRSFGGGAGGMLGNVKHENQPLRPFFDEWPGRRDSWSEMDEERSNQTSFSTTQLSISIPMPRCGSPIGPRLP

SEQ ID NO: 43 - DNA, 1230 bp, Oryza sativa
atgctgagctcttgtggtggccatggccatggaaatccaagaagcttgcaagaagaacaccatggcagatgtggtgagcagcaaggtggaggaggaggaggagggcaagagcaagagcaagatgggttcttggtgagagaggcaagggcatccccaccatctccatcttcttcatcatttcttggatccacaagctcttcttgttctggaggaggaggaggagggcagatgttgagcttctcctcccccaatggaacagcagggttgggcttgagctcaggaggaagcatgcagggggtcttggcaagggtcagggggccgttcaccccaacacagtggatggagctggagcaccaggcactgatctacaagcacattgctgcaaatgtttctgtcccttccagcttgctcctccccatcaggagaagcctccatccatggggatggggatcattccctcctggctgtgctgatgtagaacccagaagatgccgccgcacagacggcaagaagtggcggtgctccagagatgctgttggggatcagaagtattgtgagcgacacataaaccgtggtcgccatcgttcaagaaagcatgtggaaggccgaaaggcgacactcaccattgcagaaccatccacggttattgctgctggtgtatcatctcgcggccacactgtggctcggcagaagcaggtgaaaggctcagctgctactgtctctgatcctttctcgagacaatccaacaggaaatttctggagaaacagaacgttgtcgaccaattgtctcccatggattcatttgatttctcatccacacaatcttctccaaactatgacaatgtagcattgtcaccactgaagttgcaccatgatcatgatgaatcttacatcgggcatggagcaggcagttcatcagaaaaaggcagtatgatgtacgaaagtcggttaacagtctctaaggaaacacttgatgatggacctttaggtgaagttttcaaaagaaagaattgccaatcagcttctacagaaatcttaactgaaaaatggactgagaaccccaacttacattgcccatctggaatcctacaaatggctactaagttcaattcaatttccagcggcaacacagtaaatagtggtggcaccgcagtggagaatcttatcactgataatggatatcttactgcaagaatgatgaatcctcatattgtcccaacacttctctaa

SEQ ID NO: 44 - protein, 409 aa, Oryza sativa
MLSSCGGHGHGNPRSLQEEHHGRCGEQQGGGGGGGQEQEQDGFLVREARASPPSPSSSSFLGSTSSSCSGGGGGGQMLSFSSPNGTAGLGLSSGGSMQGVLARVRGPFTPTQWMELEHQALIYKHIAANVSVPSSLLLPIRRSLHPWGWGSFPPGCADVEPRRCRRTDGKKWRCSRDAVGDQKYCERHINRGRHRSRKHVEGRKATLTIAEPSTVIAAGVSSRGHTVARQKQVKGSAATVSDPFSRQSNRKFLEKQNVVDQLSPMDSFDFSSTQSSPNYDNVALSPLKLHHDHDESYIGHGAGSSSEKGSMMYESRLTVSKETLDDGPLGEVFKRKNCQSASTEILTEKWTENPNLHCPSGILQMATKFNSISSGNTVNSGGTAVENLITDNGYLTARMMNPHIVPTLL

SEQ ID NO: 45 - DNA, 1293 bp, Oryza sativa
atggcaatggcgacccctacgaccaacggcagcttccttcttggatcaggtggctatcccggtgcccagattctaagcttctcctcctcaggtcacagcggcaatgggttggattgtggaagctcagatgtggcaagaatgcagggggttttagcaagggttagggggccattcacaccaacacaatggatggagctggagcaccaggctctgatctacaagcacattgtggcgaatgcgccggtaccggccggcttgctcctccccatcaggagaagcctccatccaccagtgttcccacacttctcctctggtggcattcttggctccagctccttgggatgggggtcatttcagctgggctattctgggagtgctgactccgagcccgggagatgccgtcgaaccgatggcaagaaatggcggtgctcgagagacgcagttgtcgaccaaaagtactgcgagcggcacataaaccggggtcgccaccgttcaagaaagcatgtggaaggccaatctagccatgccgcaaaagcaacggttcccgccatagcacaaccacccattggtgcatctaatggcaaattgtcaggcagccatggtgtgtcaaatgagctcacgaaaaccttggctactaacaggatgatgttggataaagcaaatcttattgaacgctcccaggactacactaatcagcaacacaacatcctacagaacaacacaaaaggtgataattggtctgaagagatgtcctcacaagcagactatgcagtaatccctgctggctctctcatgaacacaccgcaatcggcgaatttaaatccaattccccagcaacaacgctgtaagcagtcactctttggcaaagggatacagcatgatgacattcagctgtcgatatccattcccgtggataactccgacttacccactaactacaacaaggctcaaatggaccatgtagtaggcggttcatcgaatggcggaaacaacacgcgagcaagttggataccgggctcctgggaagcgtccataggtggacctctgggtgagttcttcaccaacaccagcagcgcatcagacgacaaaggcaaaagccgccacccgccatctttgaacctcttagctgatggacatactacaagtccacagctgcaatcgcccaccggagtcctgcagatgactagcttcagttcagtgcccagcagcactgttagtagtcctgcaggcagcctctgcaatggcttgctcacttcaggcctggtgaatgcccagactgtccaaacactgtga

SEQ ID NO: 46 - protein, 430 aa, Oryza sativa
MAMATPTTNGSFLLGSGGYPGAQILSFSSSGHSGNGLDCGSSDVARMQGVLARVRGPFTPTQWMELEHQALIYKHIVANAPVPAGLLLPIRRSLHPPVFPHFSSGGILGSSSLGWGSFQLGYSGSADSEPGRCRRTDGKKWRCSRDAVVDQKYCERHINRGRHRSRKHVEGQSSHAAKATVPAIAQPPIGASNGKLSGSHGVSNELTKTLATNRMMLDKANLIERSQDYTNQQHNILQNNTKGDNWSEEMSSQADYAVIPAGSLMNTPQSANLNPIPQQQRCKQSLFGKGIQHDDIQLSISIPVDNSDLPTNYNKAQMDHVVGGSSNGGNNTRASWIPGSWEASIGGPLGEFFTNTSSASDDKGKSRHPPSLNLLADGHTTSPQLQSPTGVLQMTSFSSVPSSTVSSPAGSLCNGLLTSGLVNAQTVQTL

SEQ ID NO: 47 - DNA, 1281 bp, Oryza sativa
atgtttgctgacttctctgctgctgccatggagcttggagaggtgttgggcttgcaaggactcacagtgccatccaccaaggagggtgatctgagcctcatcaagagagctgctgctggtagcttcacccaggctgctgctgcatcatacccttccccctttcttgatgaacagaagatgctcagattcgccaaggctgctcacacattgccatcaggtttggattttgggagggaaaatgagcagaggttcttgttgtctaggaccaagaggcctttcactccctcacagtggatggagctggagcaccaggctctcatttacaagtatctcaatgcaaaggcccctataccttccagcctgctcatttcaatcagcaaaagcttcagatcatcagctaacagaatgagctggaggcctctctatcaaggcttcccaaatgcagactctgacccagaacctggaagatgccgtcgaacagatggcaagaaatggcggtgttcaaaggaggccatggccgaccacaagtattgtgagaggcacatcaacagaaaccgccaccgttcaagaaagcctgtggaaaaccaaagtagaaagactgtgaaagagacaccgtgtgctggctcattgccatcttctgtcgggcagggcagcttcaagaaggcaaaagttaatgaaatgaagccacgcagtatcagctattggacagatagtttgaacaggacaatggcgaacaaagagaaaggaaacaaagctgctgaagaaaacaatggcccactgctaaatttaacgaatcaacagccaacattgtccctgttctctcagttgaagcaacagaacaaaccggagaagttcaatacagcaggagacagtgaatcgatttcttcaaataccatgttgaagccttgggagagcagcaaccagcagaacaacaaaagcattcctttcaccaagatgcatgatcgtggatgccttcagtcagtccttcagaatttcagcttgcctaaggacgagaaaatggagtttcagaaaagcaaagattccaatgtcatgacagttccatcaactttctattcctcgccagaggacccacgcgtcagctgccatgcacctaatatggcacaaatgcaagaggatagcatctcaagttcttgggagatgcctcaaggtggacctctaggtgagatcttgacaaactccaaaaatcctgacgattcaatcatgaaaccagaagcaaggccatatggttggttactgaacctcgaggatcatgcaatgtga

SEQ ID NO: 48 - protein, 426 aa, Oryza sativa
MFADFSAAAMELGEVLGLQGLTVPSTKEGDLSLIKRAAAGSFTQAAAASYPSPFLDEQKMLRFAKAAHTLPSGLDFGRENEQRFLLSRTKRPFTPSQWMELEHQALIYKYLNAKAPIPSSLLISISKSFRSSANRMSWRPLYQGFPNADSDPEPGRCRRTDGKKWRCSKEAMADHKYCERHINRNRHRSRKPVENQSRKTVKETPCAGSLPSSVGQGSFKKAKVNEMKPRSISYWTDSLNRTMANKEKGNKAAEENNGPLLNLTNQQPTLSLFSQLKQQNKPEKFNTAGDSESISSNTMLKPWESSNQQNNKSIPFTKMHDRGCLQSVLQNFSLPKDEKMEFQKSKDSNVMTVPSTFYSSPEDPRVSCHAPNMAQMQEDSISSSWEMPQGGPLGEILTNSKNPDDSIMKPEARPYGWLLNLEDHAM

SEQ ID NO: 49 - DNA, 894 bp, Oryza sativa
gacaactcactgccccccatcttctttcttcattcctcttcccaccaagaaccccaaaccttacctccattgagttcgaaaccgaggagcgaggagttacaagccgagttgtcagaatggatgaggagaaggaagccgactcgccgcagccaccgtccaagctgcctcgcctctccggcgctgacccgaatgccggagtggtgaccatggcagcacccccgccgccggtgggtcttgggctggggcttggactcggcggcgacagccgcggcgagcgtgacgtggaagcgtcggcggcggcggcgcacaaggcgacggcgctgacgttcatgcagcagcaggagctggagcaccaggtgctcatctaccgctacttcgccgcgggcgcgcccgtgccggtgcacctcgtgctccccatctggaagagcgtcgcgtcctcctccttcggcccgcaccgcttcccttccctggcagtgatggggttggggaacctgtgcttcgactaccggagcagcatggagccggacccagggcggtgcaggcgcacggacggcaagaagtggcggtgctcgcgcgacgtggtgccggggcacaagtactgcgagcggcacgtccaccgcggacgcggccgttcaagaaagcctgtggaagcctccgcggccgccaccccggcgaacaacggcggcggcggtggcatcgtcttctcccccaccagcgtcctcctcgcccacggcaccgcgcgcgccacctgaccagtgaccagaccggcgcccgtttgtttgtttgtctcggcgcatgggaaaacccaaatccgcagggattatgtcatgtcctgtaactctttttttcctcgcaacttttgaagccaaaacaattctcaccgtgttcgatggc

SEQ ID NO: 50 - protein, 211 aa, Oryza sativa
MDEEKEADSPQPPSKLPRLSGADPNAGVVTMAAPPPPVGLGLGLGLGGDSRGERDVEASAAAAHKATALTFMQQQELEHQVLIYRYFAAGAPVPVHLVLPIWKSVASSSFGPHRFPSLAVMGLGNLCFDYRSSMEPDPGRCRRTDGKKWRCSRDVVPGHKYCERHVHRGRGRSRKPVEASAAATPANNGGGGGIVFSPTSVLLAHGTARAT

SEQ ID NO: 51 - DNA, 1149 bp, Oryza sativa
atgccctttgcctccctgtcgccggcagccgaccaccggccctccttcatcttccccttctgccgctcctcccctctctccgcggtcggggaggaggcgcagcagcacatgatgggcgcgaggtgggcggcggcggtggccaggccgccgcccttcacggcggcgcagtacgaggagctggagcagcaggcgctcatatacaagtacctcgtcgccggcgtgcccgtcccggcggatctcctcctccccatccgccgtggcctcgactcactcgcctcgcgcttctaccaccaccctgtccttggatacggttcctacttcggcaagaagctggacccggagcccggacggtgccggcgtacggacggcaagaagtggcggtgctccaaggaggccgcgccggactccaagtactgtgagcgacacatgcaccgcggccgcaaccgttcaagaaagcctgtggaagcgcagctcgtcgccccccactcgcagccccccgccacggcgccggccgccgccgtcacctccaccgccttccagaaccactcgctgtacccggcgattgctaatggcggcggcgccaacggaggcggtggtggtggtggcggtggcggcagcgcgcctggctcgttcgccttggggtctaatactcagctgcacatggacaatgctgcgtcttactcgactgttgctgctggtgccggaaacaaagatttcaggtattctgcttatggagtgagaccattggcagatgagcacagcccactcatcactggagctatggatacctctattgacaattcgtggtgcttgctgccttctcagacctccacattttcagtttcgagctaccctatgcttggaaatctgagtgagctggaccagaacaccatctgctcgctgccgaaggtggagagggagccattgtcattcttcgggagcgactatgtgaccgtcgactccgggaagcaggagaaccagacgctgcgcccctttttcgacgagtggccaaaggcaagggactcctggcctgatctagctgatgacaacagccttgccaccttctctgccactcagctctcgatctccattccaatggcaacctctgacttctcgaccaccagctcacgatcacacaacgatgagtga

SEQ ID NO: 52 - protein, 382 aa, Oryza sativa
MPFASLSPAADHRPSFIFPFCRSSPLSAVGEEAQQHMMGARWAAAVARPPPFTAAQYEELEQQALIYKYLVAGVPVPADLLLPIRRGLDSLASRFYHHPVLGYGSYFGKKLDPEPGRCRRTDGKKWRCSKEAAPDSKYCERHMHRGRNRSRKPVEAQLVAPHSQPPATAPAAAVTSTAFQNHSLYPAIANGGGANGGGGGGGGGGSAPGSFALGSNTQLHMDNAASYSTVAAGAGNKDFRYSAYGVRPLADEHSPLITGAMDTSIDNSWCLLPSQTSTFSVSSYPMLGNLSELDQNTICSLPKVEREPLSFFGSDYVTVDSGKQENQTLRPFFDEWPKARDSWPDLADDNSLATFSATQLSISIPMATSDFSTTSSRSHNDE

SEQ ID NO: 53 - DNA, 1416 bp, Oryza sativa
gagagctccgtatcaccggcctctttcccttcccttcccctccgatccaatccccccttctcctcctcgcggcgctcgctgagcatggcggcggagggggaggccaagaaggacagcgccagcaaccctcccgggggaggaggcggcggaggtggaggggaggaggaggaggatagcagcctggctgtcggggaggcggcggtcggggtgggcgaggctggtggaggaggaggaggaggggagaaggcggatcgagaggaggaggaggggaaggaggatgtggaggagggcggcgtgtgtaaggatctggtgctcgtcgaggacgccgtccccgtcgaggatccggaggaagccgcagcaactgcagcacttcaggaagaaatgaaagcgctcgttgaatccgtcccagttggtgctggggcggcattcaccgcgatgcaactacaggagcttgagcagcaatctcgtgtctaccagtatatggctgcccgtgtgcctgtgcctactcatctcgtcttcccaatatggaagagtgttactggtgcatcttctgaaggcgcccagaagtacccgacattgatggggttggcaacactctgcttggactttggaaagaacccagaaccagaacctgggaggtgccggcgaactgatggaaagaagtggcggtgctggagaaatgcaattgcaaatgagaaatattgcgaacgccatatgcaccgtggccgcaagcgtcctgtacagcttgttgtcgaggatgacgagcctgattctacctcagggtcgaaaccagcatctggcaaggccaccgaaggtggcaagaagactgatgacaagagctcaagtagcaagaagcttgcagtggcagcaccagctgctgtggagtctacatgattgatgcagcatttaggagctgcataaagagcataactgtgctggcaattagagttcgcttcttattgtaatcctgaaaagactgtagtctggtctagctataacctcatcaagcaagaaaagtgtctgtggaaagaagccacaaaaactttcatttagctgtcactgaaattttcagtttaggtgtatagtttgatttagctttgccgtgccctctgccttcaggcagatgagcggcattattggataaatcctctctgactgacaatatcgcattgtgactcaagaagccgatggaaggatctgcgagactagatacgaagctatttgttgtgtatcattttatatggcctgcacaattgtgtgattttgtcagttgcataacatgtggaagatccataattttatgcactatggagattcaattaccttcctgaatgtctgagcttcgacatgttattggttattgtaacttaaaagcaacctgagattcaatgtgaaagggttttagattccagcttc

SEQ ID NO: 54 - protein, 269 aa, Oryza sativa
MAAEGEAKKDSASNPPGGGGGGGGGEEEEDSSLAVGEAAVGVGEAGGGGGGGEKADREEEEGKEDVEEGGVCKDLVLVEDAVPVEDPEEAAATAALQEEMKALVESVPVGAGAAFTAMQLQELEQQSRVYQYMAARVPVPTHLVFPIWKSVTGASSEGAQKYPTLMGLATLCLDFGKNPEPEPGRCRRTDGKKWRCWRNAIANEKYCERHMHRGRKRPVQLVVEDDEPDSTSGSKPASGKATEGGKKTDDKSSSSKKLAVAAPAAVEST

SEQ ID NO: 55 - DNA, 1854 bp, Populus tremuloides
tagtgaagctccttctcatgtctcacctcctgagaccaaaccaaagattcttggatctgtgttaagtaagcgagaaagatcagcttcgtctgctcaagatgattactggaggacttcaaagatgccaaaaaatgatgatttttctgtcaccaaaacaatgtcgttgcaccaacccacttctttactgagatctaattacatgctttctgatgattctcgccaacaagagcacatgatgagcttctcttctccaagaccagaaacgactccatttctaagcaaagatggtgagttagtggagagaagcacacaaaaccacactgccttaagctttcgttaccatcagaacacagcttcttcttatattagaagtgcaggttatgacaccggaggcttgaatgcaggcatgcacgggcctcttactggggttagaggaccatttactccatctcagtggatggagcttgaacatcaggccttgatctacaaatacatcactgctcgtgtgcctgtgccttctaatttgatcattcctctcaagaagtctgtctacccttatagcttacctggctcctctactggatccttccctcacaattcattgggatggagcgctttccatcttggttaccctggcaacaacactgatccggagcctggaaggtgtcgtcggactgatgggaagaaatggcggtgctcaagggatgctgtagctgaccaaaagtattgtgaaaggcacataaacagaggccgccatcgttcaagaaagcctgtggaaggccagactggccatgctgccactgggactgccagttcaaaggtggtgccaatgtcgaactcgatgtcaaaattggcaataaccagtggtggtgcctccaacagcattgcgatgaccacgcaacaacagttcaaaattttgcagccggctgctgccaacacttctgcagatgttgatgtcaacagagcacaagatgcacagagcatttctatgatgtcttccaccatcaaccggaaatctgatgagtcctctttctttgttcctaaacaagatatcttaatggagcagtgctctcaaacagagtttggatttgtctcctctgactctctcctcaacccatcgcagaagagctcttacattaactctaagccctacgagtcttttctaaactttaatgacgaagaaagccaagatcagcatccccttcgtcaattcattgatgagtggccgaaggatcaatctaattgttctgtcattagctggccagaagagttgaaatctgactggacccagctctccatgtcaatcccaatggcctcatcagacttctcatcatcatcatcctcacccacacaagagaaacttgccctctcaccaatgagtttatcttgcgagtttgaccctgtacaaatgggtttaagggtgagcgttgaccataatgaatcaagccaaaagcaaaccaactggatacctatctcctgggggacttcaattggtggccctttaggagaggtcttgaccaccagcactagccatgcggattcctgcaagagctcatcagcccttagccttttgagagaaggttgtgatggcagcccacagttgggatcttctccgacgggagtcttgcagaaatcaactttctgttccctttccaatagcagttctgggagcagcccaagagctgagagcaagaaaaacaatgacactgctagtctgtatgaggatgtgggtggttcgataattgcaagttcatcacctattccacccctgtaatcaagcgaactgtaaggatgaaacctgtcaaggaaatgtgaagaagcttggagtttctatttatctgataaattcctgta

SEQ ID NO: 56 - protein, 607 aa, Populus tremuloides
MDFGVLGLEGLVGPETSSEAPSHVSPPETKPKILGSVLSKRERSASSAQDDYWRTSKMPKNDDFSVTKTMSLHQPTSLLRSNYMLSDDSRQQEHMMSFSSPRPETTPFLSKDGELVERSTQNHTALSFRYHQNTASSYIRSAGYDTGGLNAGMHGPLTGVRGPFTPSQWMELEHQALIYKYITARVPVPSNLIIPLKKSVYPYSLPGSSTGSFPHNSLGWSAFHLGYPGNNTDPEPGRCRRTDGKKWRCSRDAVADQKYCERHINRGRHRSRKPVEGQTGHAATGTASSKVVPMSNSMSKLAITSGGASNSIAMTTQQQFKILQPAAANTSADVDVNRAQDAQSISMMSSTINRKSDESSFFVPKQDILMEQCSQTEFGFVSSDSLLNPSQKSSYINSKPYESFLNFNDEESQDQHPLRQFIDEWPKDQSNCSVISWPEELKSDWTQLSMSIPMASSDFSSSSSSPTQEKLALSPMSLSCEFDPVQMGLRVSVDHNESSQKQTNWIPISWGTSIGGPLGEVLTTSTSHADSCKSSSALSLLREGCDGSPQLGSSPTGVLQKSTFCSLSNSSSGSSPRAESKKNNDTASLYEDVGGSIIASSSPIPPL

SEQ ID NO: 57 - DNA, 1848 bp, Populus tremuloides
aagtaatagtggtttcgcttctcttgctagttcagatcctgaagcaaagcagaagtacggatctgggttcctgaagcaagagagatctgccgcagccgatgacgattggaggaactctaaattggccaaaaccgagtcaatgctgcttgaccagagaaacacttttcttctgaaatctagcaacaactctctcttcactgatggacagcagcagcagcagatgctcagcttctcctgtcccaaatcagcttcttcaggggagagaagctccccaaatgccatgttgccatactttcacctcacatcttctgcttgtaatagaaatacaggctacaactctggaatcttcaatgctgccagcatgcatggggttttgactgagactagatggccattcactcaattacaatggatggagcttgaacatcaggccttgatctacaaatacatgactgcaaatgtgcctataccatctaatctgctcatccccattaggaaagctcttgattctgctgggttttctagcttttctggtggacttttcaaacccagtgcattgcaatggggtactttccatatgggtttctccagcaacactgatccggagccaggacggtgtcgaagaacagatgggaagaaatggcggtgctcaagagacgcagttgctgatcagaagtattgtgagcggcacatgaacaggggtcgccatcgttcaagaaagcctgtggaaggacaatcaggccattccgctgcggccaccaccactttaaagccaatggccaatggcacttcctcttttgcatcagcatcagtggtggggcttcgcagcgctgtgtccgacagccacactattgtgcataatcagcagcaacctgccagttcttctaatctttctgccaccaatacgctcagcagggtgttcctcgctacagagaatgtaggtgagagaatgcaagatgcatcgggcttatccatgctaccatccagcattgacctgaaatccaaagaaactccattcttcatatcaaaacaacagaactcttacggtgaatccctgcaaaatgagtttgcacttgtcacctccgactccctcctcaaccattcacagaaaagctcgtccttgatgagttgcagaaattttggttcgtctcaggaccttactgaccaggaatctgtttcacagcactccctccgccaatttatggatgattgtcctaaaagtcattctgatcgctctgctgttgcttggcctggacttgatctgcaatctgagagaacccagctatcaatttcaatccccatggctcctgcagactttgtgtcatccacttcatcttcaaacaatgaaaagatctctctctccccgctgagattatcgcgtgaatttgatccaataaagatggggctgggagtgggagccggtagtgtcgccaatgaaccaaaccaaaggcaagcgaattggattcccatttcttgggaaacttcaatgggtggtccacttggggaggttttgcacaacaccaataataatgcaacagcagaatgcaagaatgaatcatcgctcaacctaatgacagagagatgggacaacagtcctcgggtaggctcatctcctaccggggtcttacaaaagtctgcctttgcttctctttcaaatagcagtgctggaagcagcccaagagcagagaacaagaccattgaaggtggcaatctctgcaatgaccttggatctactatcgtgcattcttcatcattgcctgccttgtaactctctgacctgccatttaagaagtcttcagctgatgccagattatgaataatttgttttttaaagttctcaatcagtct

SEQ ID NO: 58 - protein, 605 aa, Populus tremuloides
MDFGVQVGLDGLVGSDTSNSGFASLASSDPEAKQKYGSGFLKQERSAAADDDWRNSKLAKTESMLLDQRNTFLLKSSNNSLFTDGQQQQQMLSFSCPKSASSGERSSPNAMLPYFHLTSSACNRNTGYNSGIFNAASMHGVLTETRWPFTQLQWMELEHQALIYKYMTANVPIPSNLLIPIRKALDSAGFSSFSGGLFKPSALQWGTFHMGFSSNTDPEPGRCRRTDGKKWRCSRDAVADQKYCERHMNRGRHRSRKPVEGQSGHSAAATTTLKPMANGTSSFASASVVGLRSAVSDSHTIVHNQQQPASSSNLSATNTLSRVFLATENVGERMQDASGLSMLPSSIDLKSKETPFFISKQQNSYGESLQNEFALVTSDSLLNHSQKSSSLMSCRNFGSSQDLTDQESVSQHSLRQFMDDCPKSHSDRSAVAWPGLDLQSERTQLSISIPMAPADFVSSTSSSNNEKISLSPLRLSREFDPIKMGLGVGAGSVANEPNQRQANWIPISWETSMGGPLGEVLHNTNNNATAECKNESSLNLMTERWDNSPRVGSSPTGVLQKSAFASLSNSSAGSSPRAENKTIEGGNLCNDLGSTIVHSSSLPAL

SEQ ID NO: 59 - DNA, 1053 bp, Populus tremuloides
atggcaagagcttgaacaccaagctctcatttacaaatacatggtctctggtgttcctgtcccgccagaactcctctattctgtcaaaagaagcttgggatcttctttggcatcaagactcttccctcaccaacctattgggtggggttgttttcaggcgggttttggcagaaaagcagacccagagccaggaaggtgcagaagaacggatggaaaaaaatggaggtgctcaaaggaagcatacccagactcaaaatattgtgagaggcacatgcacagaggcagaagccgttcaagaaagcctgtggaacttacttcaagtactactacaacagcaacaacaattcctttaacatcaatcaacagaaacctctctaaccccactatttcaccctccagctcctcttattctttctctcacccttcatctgcggaatctgaagtttatgcccatcaaaacccttcgcatggaaccttccttaaccccttcctttatcctcattcttcatcttctggacctcctgattctggtttttcacctctaaatagcacccctcacaacctgtttttggagtctggatcttctcctcaagttgacaaagagcacaggtattatcatggaatgagggaggatgtggatgagagagctttctttccagatggtttagggagtgcaagaggtgttcaagattcatataaccaattgacaatgagttcctacaaaggttactcactgtcacagtttcaaacctttgctgatacttctaaagaagagcagcaacaaccagggcagcactgctttgttttgggcactgatattatcaagtcatcagcaacaaggtcaatcaagttggagaaagaaactgaaaccctgaagccattgcaccatttctttgatgaatgggaaccaaaggacgcagactcttggcttgatcttgcatccagttcaagacctcacacttctgatgattgaagtctcaataatggatctttgtaatatgacgaaaacagtacttgttcgtgggtcatcaatgcctttcttgcctcaaatga

SEQ ID NO: 60 - protein, 340 aa, Populus tremuloides
MLNTTISRNRFPFTATQWQELEHQALIYKYMVSGVPVPPELLYSVKRSLGSSLASRLFPHQPIGWGCFQAGFGRKADPEPGRCRRTDGKKWRCSKEAYPDSKYCERHMHRGRSRSRKPVELTSSTTTTATTIPLTSINRNLSNPTISPSSSSYSFSHPSSAESEVYAHQNPSHGTFLNPFLYPHSSSSGPPDSGFSPLNSTPHNLFLESGSSPQVDKEHRYYHGMREDVDERAFFPDGLGSARGVQDSYNQLTMSSYKGYSLSQFQTFADTSKEEQQQPGQHCFVLGTDIIKSSATRSIKLEKETETLKPLHHFFDEWEPKDADSWLDLASSSRPHTSDD

SEQ ID NO: 61 - DNA, 1265 bp, Populus tremuloides
tcttcttttctcttccagggtaatatgataatgagtggaggaaacaggtttcccttcactgcatcccagtggcaagagcttgagcatcaagccctaatctacaagtacatggtttcaggcatccccatccctcccgatcttcttttcaccatcaaaagaagtggctgcttggactcttcactctcttcaaagctctttccttgccaacctccacatttttcctggggctgttttcagatgggtttgggaaggaaaatagatccagaaccggggaggtgcaggagaactgatggaaagaaatggagatgctcaaaagaagcatacccagattctaagtactgtgagaaacatatgcatagagggaagaaccgttcaagaaagcctgtggaagttgcaacacaatcaataacagcaccaactgtctcatcaatgaccagaaaccactctaataattcactactaacaacatcccccacctctctttcgttattgtcacctaagacccaccaccagaatcaccttcactatcctgctcctgcaggttatcatgcccatccaaatcatcaattcttgtcttcttccagaccccttgggattggtctgtcccctcatgaaaatcctactcacttgcttttggactctggtggttcttctctggccaatacagattacagaagaaacaggaatgtttatgggctgaaagaggaggttgatgagcatgctttcttctcagaaccttcaggttctatgagaagcttgtccggttcatctttggatgatgcttggcaactcaccccactcacaatgaactcttctccttctaccaccaactcttcaaagcaaaggagcttgtctagtttacacaacgaatattcttacttgcagcttcaaagcctgagtgatcccgataccccaaaacaacaaaagcagtgtcaacataactatcttctgggaagtagtgatgtagacagtctagggcccataaaaatggagaaggaaaaatcccaaaagactgttcaccgtttctttgatgaatggccaccaaaggataaagattcatggcttgatttggatgacaaatcatcaaaaagtgcatcagtttcagcaaccggactctcaatatccattccctcctctcatgactttcttccaatcttcagttcaagaactaataatggtggttgattttactctggtgggtttctggcccaagatgtacttggtgggaaggggggggtcaccgccttctgtcaagaggcctcaga

SEQ ID NO: 62 - protein, 386 aa, Populus tremuloides
MIMSGGNRFPFTASQWQELEHQALIYKYMVSGIPIPPDLLFTIKRSGCLDSSLSSKLFPCQPPHFSWGCFQMGLGRKIDPEPGRCRRTDGKKWRCSKEAYPDSKYCEKHMHRGKNRSRKPVEVATQSITAPTVSSMTRNHSNNSLLTTSPTSLSLLSPKTHHQNHLHYPAPAGYHAHPNHQFLSSSRPLGIGLSPHENPTHLLLDSGGSSLANTDYRRNRNVYGLKEEVDEHAFFSEPSGSMRSLSGSSLDDAWQLTPLTMNSSPSTTNSSKQRSLSSLHNEYSYLQLQSLSDPDTPKQQKQCQHNYLLGSSDVDSLGPIKMEKEKSQKTVHRFFDEWPPKDKDSWLDLDDKSSKSASVSATGLSISIPSSHDFLPIFSSRTNNGG

SEQ ID NO: 63 - DNA, 644 bp, Populus tremuloides
ctagggactggactgctaaacggttggaacatggagggaaataggcgcaatggtcggtctcggtcaccttcaattgggctaggagttgagcttggacgtggtggttctagtcaaagaccaataactggctgcaaaaaaccttatgggttcactattcttcaactgcatgagctagaacttcagtctcttatctacaagtatatccaagctggatttcctgtaccttaccatcttgttttacctatatggaaaagtgttactgcttcccttggtggtctcagttcaagcttgtaccagctctaccctagctttatggggtgtaagtgtaacccattatatttggaatataagaaaggaatggaacatgagccagggagatgtaggagaacggatggaaagaagtggaggtgtagcaaagaggttcttccagatcaaaagtactgtgacaggcacatacacagaggacgccagcgttcaagaaagcttgtggaagctgcttctcatagtaatgccagcaccaacctctccatttctctccctggaatcggtagtgctagcgcctagcagtactaatctctctccaatatgttgtctctctccaaagagtgttctccacaagaaatatgtaataggagcagctgcta

SEQ ID NO: 64 - protein, 177 aa, Populus tremuloides
MEGNRRNGRSRSPSIGLGVELGRGGSSQRPITGCKKPYGFTILQLHELELQSLIYKYIQAGFPVPYHLVLPIWKSVTASLGGLSSSLYQLYPSFMGCKCNPLYLEYKKGMEHEPGRCRRTDGKKWRCSKEVLPDQKYCDRHIHRGRQRSRKLVEAASHSNASTNLSISLPGIGSASA

SEQ ID NO: 65 - DNA, 1154 bp, Populus tremuloides
caagtgaagttgaagagagaagtgaaagaaatgagcaactcatcagtcacagtggcgggggtgggatcaagatcaccaccaggtttcacgatgtctcagtggcatgagctggagcatcaagttcttatctttaagtgtttaaatgcagggttacctgtccctccttcccttctccttcctattcgtaagagttttcagcttctttcccctggtttcttgcacccatcaaatttgagctactgttcctattttgggaagaagattgactcagagccagggaggtgtcggaggacagatggcaagaaatggaggtgctccaaagatgctcacccagactccaagtactgtgagcggcatatgaatagaagccgtaaccgttcaagaaagcctgtggaatcacaaactacctctcagtccttgtcaactgtggcatcagaaattgcaactgggagcagcagcattgggagcagagggtatccaactaatcctgggaccttaggtttgggaagtaatatgtcacgttggcagatggagtctatgccttatggtgttaatagtaaagactacaggtctctccatggaccgaagcctgaagcagatgagaaaactttcctaccagaagctttgggaaatacaagaagctttggaatgaactctactgtggacagcacttggcatctcacatcccaagtccctgcaaaccctgtgccagaatcaagaaatggttctcttttgcaaaactacccacaagtacagacactgcaggattttgagcccctaactgttgatgctgcatcgccaaaacaacagcagcagcagcattatttatttggaagggagttcagttcatcaggatctatgaggcgggaaaatcagtctcttcagcctctctttgacgagtggccaaaatgcagggatatggattcccatctcactgatcaaagatctaacaataactcgtctgctgttcagctatcaatggccattccaatggctcctaaccctgctgcgaggagttatcattccccaaatggtgagacaggtttatctggaacatacttttccgcaacctagacgcccccaagacttggtggaaaacaaatagatgagttctaatcctctcatgttataatgcagatgcttgaaaggagcat

SEQ ID NO: 66 - protein, 347 aa, Populus tremuloides
MSNSSVTVAGVGSRSPPGFTMSQWHELEHQVLIFKCLNAGLPVPPSLLLPIRKSFQLLSPGFLHPSNLSYCSYFGKKIDSEPGRCRRTDGKKWRCSKDAHPDSKYCERHMNRSRNRSRKPVESQTTSQSLSTVASEIATGSSSIGSRGYPTNPGTLGLGSNMSRWQMESMPYGVNSKDYRSLHGPKPEADEKTFLPEALGNTRSFGMNSTVDSTWHLTSQVPANPVPESRNGSLLQNYPQVQTLQDFEPLTVDAASPKQQQQQHYLFGREFSSSGSMRRENQSLQPLFDEWPKCRDMDSHLTDQRSNNNSSAVQLSMAIPMAPNPAARSYHSPNGETGLSGTYFSAT

SEQ ID NO: 67 - DNA, 1875 bp, Populus tremuloides
tagtcatgctccttctcatgtctcacttcctgagaccaaaccaaagattcttggatctgtgttaactaagcaagaaagatcatcttcatctgcatcagctcaggatgattactggagggcttcaaagatgccaaaacttgatgatttctcttccaccaaaacaatgccactgcaccaacccgctcctttgctgagacctaattctatgttttctaatgattctcgccaacaagagcacatgctaagcttctcttctccaaaaccagaagctactccatttctcgttaaagatgctggcttggttgagagaaacacacaaaaccacactgccttgagttttccttactaccagcacgcacctctttccgctagcagaagtgcaggttatggcactggaaacttgaatgcaagcatgcaggggccttttactggggttagaggaccatttactccatctcagtggatggagcttgaacaccaggccttgatctacaaatacatcactgcacgtgtgcctgtgccttccaatttaatcattcctctcaagaaatctctcaacccttatggcttacctttttcctctgctggatcattccctcccagttcattgggatggggcactttccaccttggttaccctggcaacaacactgatcaggagcctggaaggtgtcgtcggactgatggcaagaaatggcggtgctcaagggatgctgtagctgaccaaaaatattgtgaaaggcacataaacagaggccgccatcgttcaagaaagcctgtggaaggccagactggccatgctgctactgggactgccagttcaaaggtggtgccaatgtctaactccatgccaacctcgattacaaccagtggcgctacctcgaacagcattgtgatcacacagcaacagttaaaaaattttcagccggctgctgcttccatctcttctgcagatgctcgtgtcaacggagcacaagatgcacggagggtttctatgatgtcttccactatcaaccggaaatctgacgagtctactttctgtattcctagacaagatatcctatttgaacagtgctctcaaacagagtttggacttgtctcctatgattctctcctcaacccatcgcagaagagctcttactttaacgctaaaccctacgagtcttttctaaactttagtgatgaagaaagccatgatcagcatccccttcgtcaattcattgatgactggccgaaggaccaatcaaatcgttctgtcattagctggccagaagagttgaaatctgactgtacccagctctcaatgtcaatctcaatggtctcgtcagacttctcgtcgtcatcatcctcacttctgcgagagaaacttgccttctcaccattgaggttatctcgcgagtttgaccctatacaaatgggtttaagggtgagcggtgaccataatgaatcaagccagaagcaagccaactggatacctatctcttggggaacttcaattggcggccctttaggagaggtcttgaccaccagcgccagccatgcggattcctgcaaaagctcatcagctcttaaccttttaagagagggttgggatggcagcccgcagctgggatcttctccaacaggagtcttgcagaaatcgacttttggttcactttcaaatagcagttcaggtagcagcccaagagcagagagcaagaaaaacaatgaaagtgctagtctgtatgaggatgttgttggttcgataattgcaagtgatcccctattccatccctgtaatcaagaaaatggttaggatgaaacttgtgaagaagaagcttggagttatttatcttattaatttctgcagactgtttctccttgttgcttgttccc

SEQ ID NO: 68 - protein, 555 aa, Populus tremuloides
MPKLDDFSSTKTMPLHQPAPLLRPNSMFSNDSRQQEHMLSFSSPKPEATPFLVKDAGLVERNTQNHTALSFPYYQHAPLSASRSAGYGTGNLNASMQGPFTGVRGPFTPSQWMELEHQALIYKYITARVPVPSNLIIPLKKSLNPYGLPFSSAGSFPPSSLGWGTFHLGYPGNNTDQEPGRCRRTDGKKWRCSRDAVADQKYCERHINRGRHRSRKPVEGQTGHAATGTASSKVVPMSNSMPTSITTSGATSNSIVITQQQLKNFQPAAASISSADARVNGAQDARRVSMMSSTINRKSDESTFCIPRQDILFEQCSQTEFGLVSYDSLLNPSQKSSYFNAKPYESFLNFSDEESHDQHPLRQFIDDWPKDQSNRSVISWPEELKSDCTQLSMSISMVSSDFSSSSSSLLREKLAFSPLRLSREFDPIQMGLRVSGDHNESSQKQANWIPISWGTSIGGPLGEVLTTSASHADSCKSSSALNLLREGWDGSPQLGSSPTGVLQKSTFGSLSNSSSGSSPRAESKKNNESASLYEDVVGSIIASDPLFHPCNQENG

SEQ ID NO: 69 - DNA, 1715 bp, Populus tremuloides
aaaaattattcttctttatttttcatcatgatgacaacagatgatggcttaaacgtttcaaacaaggtagctaaggaaataaacactactagtagtattagtaatgttgattttggtgtgaagctacatcaacctattgatcatcatcaatcatttccttctagtactcctatgatggttcctcatgttaatcaccaccgtccaatgtttgacaatggtcccacatcatcatgtgatagaaacaagtctttgatgaactatataagtgatcgtatataccgtgttgctgctggtggtgctaccagtggcggtgcagttggggttaggaatttgcagccttttgacatttctgaaacaagtatctctacagcagcttctgccttcagatccccaggaggcaacatggcagcgtctttggggtttccttttacaaatacacagtggaaagagcttgaaagacaagccatgatatacaactatataacggcctcagtccctgtgcctcctcaatttctaattccaaccccaatggggaatggattgaatgtaaggttctcaaatggggcagatctagaaccagggaggtgtaggagaacagatgggaagaaatggaggtgctcaagagatgtggcacctgatcagaaatactgtgagcgtcatatgcatagaggcagaccccgttcaagaaagcatgtggaactcaatgctagcaacaataacaacaagaagaaccgccataatcctgctatttgtccagaagctcctgttaccgtggccatttctaaacccacaatcaacaacagcaacagtggctctgcctctcacgatcagttttttgggcctatgcctcagccatatatccagaccccagtttttgtaaacaaaaccagcgagaagacttcaacttatgatgttaatggagcctatggttccacattcaaagaacccaggagcttggactggatgttgaaaggggaagctggtcctatagccaaaaatgatcaacaatggccacatctagtgcacaaagaaattgaactagctactgaaggttcctttaacagtgcttctgttctcaaacagcattaccaaggagagtctttgaatttgaactcatttggaaattttaatgctagagaagaccaacaaagcaatcaatatagtctgtttcttgatgaggctccaaggagttttattgatgcatggtctaatgatgcaatttctagaaacacaagttctgtttcctcagatgggaagctccatctttcccctctcagtctatcaatgggaagcaataggtctactgatgatgaaatgggtcagatccaaatgggtttaggcctaatcaaatcagatcgaaatgaagaatgtggtaacactagcagcgccccaggtggccccttggcagaggtgttacaactgaggacaagcaacaccacaggaaccaatcaatcttcttctatgatggaaaatggtgattctattagtcctccagctactacagtctcttctccatctggggttttgcagaaaacacttgcctcattttctgatagcagtggtaatagcagtccaactcttgccagttcaaggaccaaacctgaaattgccatgctttggttaaatcaaggctaaatgtgccactctctagttagttaagggcacacaaggccataagggcaatataaatttttataagcttgatatatttttat

SEQ ID NO: 70 - protein, 535 aa, Populus tremuloides
MMTTDDGLNVSNKVAKEINTTSSISNVDFGVKLHQPIDHHQSFPSSTPMMVPHVNHHRPMFDNGPTSSCDRNKSLMNYISDRIYRVAAGGATSGGAVGVRNLQPFDISETSISTAASAFRSPGGNMAASLGFPFTNTQWKELERQAMIYNYITASVPVPPQFLIPTPMGNGLNVRFSNGADLEPGRCRRTDGKKWRCSRDVAPDQKYCERHMHRGRPRSRKHVELNASNNNNKKNRHNPAICPEAPVTVAISKPTINNSNSGSASHDQFFGPMPQPYIQTPVFVNKTSEKTSTYDVNGAYGSTFKEPRSLDWMLKGEAGPIAKNDQQWPHLVHKEIELATEGSFNSASVLKQHYQGESLNLNSFGNFNAREDQQSNQYSLFLDEAPRSFIDAWSNDAISRNTSSVSSDGKLHLSPLSLSMGSNRSTDDEMGQIQMGLGLIKSDRNEECGNTSSAPGGPLAEVLQLRTSNTTGTNQSSSMMENGDSISPPATTVSSPSGVLQKTLASFSDSSGNSSPTLASSRTKPEIAMLWLNQG

SEQ ID NO: 71 - DNA, 1053 bp, Populus tremuloides
agcagcggggatggcagctgggggaatggggacagcagcgatgacaatgaggtcaccatttacagtgtcacagtggcaagaactggaacatcaagctttgatctataagtacatggtggcaggtctgcctgttccacctgatcttgtgctccctattcagaggagctttgaatccatttctcatagattcttccaccatcccaccatgagctattgcactttctatggcaagaaggtggatccggaaccaggtcgatgcaggaggaccgacggcaagaagtggaggtgctccaaagatgcctacccagactccaagtactgtgagcgccacatgcaccgtggccgcaaccgttcaagaaagcctgtggaatcacaaaccatgacacagtcatcgtccaccgtgacatcactgactgttacaggaagcagcagtggaactggaagcttccagaaccttccattgcacacatatagcaatccccagggcactgcttctggaactaaccaatcatattatcatatgaactccattccctacggaatcccaaccaaagattacaggtatcttcaagaacttacgcctgaaggtggggagcatagcttcttgtctgaagcctcaggaagcaacaaggggcttcagatagactcacagctggacaatgcatggtctttgatgcaatccagagtctcatcattccccacagagaaatcaactgaaaactcgatgttgcaaagtaatcatccccagcattcatttttcagtagtgatttcaccaccagggaatctgtgaaacaggacgggcagtctcttcgacccttctttgatgagtggcctaaaaaccgagatgcctggtctggcctcgagaatgatagttccaaccagacctcattctctacaacgcagctgtcgatatccattccaatggcctcatctgacttctccacaagttgtcgttctccacgagataactaagaggacactaagaattcacaaaacaaagccaaaggtggtcctggccaaagattaacacatacttgtgttaaatttttgtg

SEQ ID NO: 72 - protein, 320 aa, Populus tremuloides
MAAGGMGTAAMTMRSPFTVSQWQELEHQALIYKYMVAGLPVPPDLVLPIQRSFESISHRFFHHPTMSYCTFYGKKVDPEPGRCRRTDGKKWRCSKDAYPDSKYCERHMHRGRNRSRKPVESQTMTQSSSTVTSLTVTGSSSGTGSFQNLPLHTYSNPQGTASGTNQSYYHMNSIPYGIPTKDYRYLQELTPEGGEHSFLSEASGSNKGLQIDSQLDNAWSLMQSRVSSFPTEKSTENSMLQSNHPQHSFFSSDFTTRESVKQDGQSLRPFFDEWPKNRDAWSGLENDSSNQTSFSTTQLSISIPMASSDFSTSCRSPRDN

SEQ ID NO: 73 - DNA, 1682 bp, Populus tremuloides
gtctgttcttgtttttgtagatacacgacaatggagaaaagagtatctgaagaatcagcgccgtcgatgaaactgtctcttgggattggtgctggtgatcatggtgatgatgatcaagatgatagacacgtgttcccacagttaacagagactcagttgcatgagcttaaacagcaagctttgatattcaagtacatagtagctggtcttcgcgtgcctcctgatcttgtagttcccatttggcatagtgttgctagcagctctcttggttcatttagtggtgctgatatttataggcaattcccaagctttgtgggattaagtcctcagggatttgattatagacaaatgatggatccagaacctgggagatgtagaagaactgatgggaagaaatggaggtgtagcaaggatgtagttgctggtcagaagtattgtgaacgccatatgcatagaggccgtcaacgttcaagaaagcttgtggaagcttctcaaactgctgctgcttctgaaaaaccatcacctcacaattcaagcaagaattcagacaatccaaccactcattcttctaatttagccaaagtaagttcacagataaaagccccacctcttaataatacccccaccattttaaccacttgcaccacaagttgcaattctgacattgaaatcactggcatgagcttggccactactgctaattctgactgcaaaaacccctttacaaccatgactactagcattgttaccggctacaagaacactgcaacaatgatcgctagtgctgttcatgctgacattacagccactggtaacgactacaagagtagcatcaacttaaagagacactacattgatgacagaaacagtaattgcagcaactctgttacttacaagggtataatcgacagaaactgcagcaacaaaaaaataaaaaatgctggtagcaatgtatctcaaggattgaacttctccccgaagagtgttcttcaagttcaaggttgcggcgcctcacacatttacatgaatgatgtggaacttgaactgggaaggtgtagaagaacagatggaaagaagtggcgatgccgcagggatgttgtagctaatcagaagtattgcgagatgcacatgcaccgaggttctaagcagcacttggaagcgtccaaacctgctgcaattcccgctacaatcccatttgtccctgggaatgttcattcatatcctgcaacgaacttgccaagtaaagcagatcgcagaagcttaaacaccgatctctgtatttcgatcccaacaagtcctcaactgatcatgaccaatgatgatacaagaactatcagcaatagcagtgacactaccataagcgacaccatgaggggcaccacaccaggactggtatcaacacatgcaggtagtggggatcccaaaagtcctcctggtggtgacagagccaggttattaaagaaagctgaagttatggaggcggcggtagagatttacgaggaagtacaatccggccgtggggtcagaagttttatccaaggcagaagacttccaacttttccctatgtttatatcacacctctgctgttaatctgcaattaattctagtcaaaccatgtgattaaagttacaagaggtgtaagagaagaataacgtttaattccatcctatgcagacaactg

SEQ ID NO: 74 - protein, 523 aa, Populus tremuloides
MEKRVSEESAPSMKLSLGIGAGDHGDDDQDDRHVFPQLTETQLHELKQQALIFKYIVAGLRVPPDLVVPIWHSVASSSLGSFSGADIYRQFPSFVGLSPQGFDYRQMMDPEPGRCRRTDGKKWRCSKDVVAGQKYCERHMHRGRQRSRKLVEASQTAAASEKPSPHNSSKNSDNPTTHSSNLAKVSSQIKAPPLNNTPTILTTCTTSCNSDIEITGMSLATTANSDCKNPFTTMTTSIVTGYKNTATMIASAVHADITATGNDYKSSINLKRHYIDDRNSNCSNSVTYKGIIDRNCSNKKIKNAGSNVSQGLNFSPKSVLQVQGCGASHIYMNDVELELGRCRRTDGKKWRCRRDVVANQKYCEMHMHRGSKQHLEASKPAAIPATIPFVPGNVHSYPATNLPSKADRRSLNTDLCISIPTSPQLIMTNDDTRTISNSSDTTISDTMRGTTPGLVSTHAGSGDPKSPPGGDRARLLKKAEVMEAAVEIYEEVQSGRGVRSFIQGRRLPTFPYVYITPLLLICN

SEQ ID NO: 75 - DNA, 1812 bp, Populus tremuloides
aagtaatagtggttttgcttctcttgctggctcagatcctgaagcaaagcagaagttgtacggatctgggttcttgaagcaagagagacctggcaacatcgatggtgattggaggagctctaaattgtcgaaaactgagtcaatgctgcttgagcagagtaacacttcacttctgaaatctagctccaactttctcttcgctgatggacagcagcagcagcagcagatgctcagcttctcctatcccagatcagctccttcagcggagagaagctcccaaaatggcacattgccctctggtatctacaatgctgccagcatgcatggggttttgaccgagaccagatggccattcactcaatcacaatggatggagcttgaacatcaggccttgatctacaagtatatagcagcaaatgtgcctataccatctaatctgctccttcccattagaaaagctcttgattctgctgggtttcctagcttttctgctggatttttcaggcccaatacattgccatggggtgctttccatatgggtttctccagcaacactgatccggagccaggacggtgtcgaaggacagatggaaagaaatggcggtgctcaagagatgcagttgccgatcagaagtattgtgagcggcatatgaacaggggccgccatcgttcaagaaagcctgtggaaggccaatcaggccattccgctgcagccgccaccactgtaaagccagccaatggcacttcgtcttctacatcatcatcagtggtggggcttcgcagcactgtgtccgacagcctcactattgctcataatcagcagcaagcagctagtccatctaatctttctgcctctaatacgctcagcaggatgttccttactaaagagaatgtaggtgagagaacgcaggatgcgacagccttgtccatgcttcgatccaacatggatcttaaatctaaagaaactccattcttcatatcaaaacaacaaaactcatatggggaatccttacgaaatgagtttggacttgtcacctccgactccctcctcaatcactcacagaaaagctcatctttaatgagttgcagaaattttggttcgtctcaggacctcactgaccaggaatctgtttcacagcactccctccgccaattcatggatgattggcctaaaagtcagtctgatcgttctgctgtttcttggcctgaacttgatcagcaatctgagagaacccagctatcgatttcaatccccatggctcctgcagacttcatgtcatctacttcctccccaaacaatgaaaaagtcactctctccccattgagattatcacgagaatttgatccaatacagatgggactgggagtgggaggtggaggtggtggtattgccaacgaaccaaaccaaaggcaagccaactggattcccatttcttggggaacttcaatgggtggtccgctcggggaggtcttgcacaacaccaataacaatgcagcagcagagtgcaagaccacgtcatcgctgaacctgatgacctatagatgggacaacagtcctcgtataggttcatctccaactggggtcttacaaaagtcagcgtttgcttccctttcaaatagcagtgcgggaagcagcccaagagcagagaacaagaccaatgaaggtggcagtctctgcaatgacctccttggatccactattgtgcattcttcttcattgcctgccatgtaactctgttgatctgctgccatccaagaagtctcctgtcatcgtagctgacaaaacatggagcactttgtttttgaagttgt

SEQ ID NO: 76 - protein, 529 aa, Populus tremuloides
MLLEQSNTSLLKSSSNFLFADGQQQQQQMLSFSYPRSAPSAERSSQNGTLPSGIYNAASMHGVLTETRWPFTQSQWMELEHQALIYKYIAANVPIPSNLLLPIRKALDSAGFPSFSAGFFRPNTLPWGAFHMGFSSNTDPEPGRCRRTDGKKWRCSRDAVADQKYCERHMNRGRHRSRKPVEGQSGHSAAAATTVKPANGTSSSTSSSVVGLRSTVSDSLTIAHNQQQAASPSNLSASNTLSRMFLTKENVGERTQDATALSMLRSNMDLKSKETPFFISKQQNSYGESLRNEFGLVTSDSLLNHSQKSSSLMSCRNFGSSQDLTDQESVSQHSLRQFMDDWPKSQSDRSAVSWPELDQQSERTQLSISIPMAPADFMSSTSSPNNEKVTLSPLRLSREFDPIQMGLGVGGGGGGIANEPNQRQANWIPISWGTSMGGPLGEVLHNTNNNAAAECKTTSSLNLMTYRWDNSPRIGSSPTGVLQKSAFASLSNSSAGSSPRAENKTNEGGSLCNDLLGSTIVHSSSLPAM

SEQ ID NO: 77 - DNA, 1017 bp, Populus tremuloides
agtgatgaccatgaggtcgccatttacagtatcgcaatggcaagaactggaacatcaagctttgatctataagtacatggtggcaggtctgcctgttcctcctgatcttgtgctccctattcagaggagttttgagtccatttctcatagattcttccaccatcccgccatgggctattgcactttctatgggaagaaggtggatccggagccaggtcaatgcaggaggaccgacggcaagaagtggaggtgctccaaagatgcatacccgggctccaagtactgtgagcgccacatgcaccgtggccgcaaccgttcaagaaaggctgtggaatcacaaaccatgacacagtcatcgtccactgtgacatcactgactgtaacaggaagcagcagtggaacagggagcttccagaaccttccactgcgcacatatggtaatccccagggcactggttctggacctaaccaatcccattatcatatgaactgcattccccgtggaatcccaactaaagattgcaggtatcttcaaggactgaagactgagggtggcgagcatagcttcttgtctgaaccttcaggatgcaaaaggggtctccagaaggactcacagctagacaatgcctggtctttgatgctatccagaggctcatcattccccacagagaaatcgactgacgactcgacgttgaagaatgattatccccagcattcatttttcagtagtgatttcaccaccggagaacccgtgaaacacgaagggcagtctcttcgacccttctttgacgagtggcctgaggaccaggacatttggtctggcctcaaagataatagatccaactccacctcattctctacaacgaagctgtcgatgtccattccaattgcctcatctggcttctccacaagttctcgttctccacaagaaaactgagaggacagaatgagaatttacaaagcacgatcaaaggtgatcctcaaccaaagattggtgtgttaacttgaaaactccta

SEQ ID NO: 78 - protein, 310 aa, Populus tremuloides
MTMRSPFTVSQWQELEHQALIYKYMVAGLPVPPDLVLPIQRSFESISHRFFHHPAMGYCTFYGKKVDPEPGQCRRTDGKKWRCSKDAYPGSKYCERHMHRGRNRSRKAVESQTMTQSSSTVTSLTVTGSSSGTGSFQNLPLRTYGNPQGTGSGPNQSHYHMNCIPRGIPTKDCRYLQGLKTEGGEHSFLSEPSGCKRGLQKDSQLDNAWSLMLSRGSSFPTEKSTDDSTLKNDYPQHSFFSSDFTTGEPVKHEGQSLRPFFDEWPEDQDIWSGLKDNRSNSTSFSTTKLSMSIPIASSGFSTSSRSPQEN

SEQ ID NO: 79 - DNA, 1221 bp, Populus tremuloides
acaaccctctgcaaagatgccaaaactcctcatggatccccatcaaccacaacaacatccacactcatctgggtctgctgccttccctttgtttctacccgagcccagctgcaaaaatagtaacctgtcagcatttcctgattcaaacacagctgcaaacaccagacttcctaagatcatggggaattactttagcctggaacagtggcaagagctagagctgcaggctttgatctacagattcatgttagccggtgcagctattcctccggagctcctccaaccaatcaagaaaacccttcttcattctcacccccctccatatttcctccatcatcctcttcaattacattgctcttattatcagccatcttggtattggggaagagcagccatggatccggagccaggtcggtgccggagaacagatgggaagaaatggcggtgctccagagacgtggtggcagggcacaagtattgcgagcgccacttgcaccgtggccgcaaccgttcaagaaagcctgtggaaaatcccacacctacaatatccactaacatcacttgcattggtattggaggtgcgggtggtaccgcatcagctgctgctttcaattgcagcaccacaccaaccatatcagaggtggtcaatgagactcatttttcgcatacactagaatccccttccattcatctcaatcatagctccaaaactgaaagcaagggcttaattggaccaccacctccaaatgaggttggtaacaggtctgatggccacattctgtggcatttttttgatgactggccacgatccgttgatgaatccgacaatatgaatgctggaagctcaatgaactctttaacctgcctctccgtttcaatgcctggaaactcaccagcatcagatgtgtcattgaaattgtccactgggaataatattgcagaggaggagccggagccagtcccagccccgatccctagaggcaatacaagcaattgggctgctgcaggatggggcacaaaaattacaaaccaggtggtgacttcaatggggggacctcttgctgaggcgctgaggtcctccactaccaaactcatctcccacgaatgttctgcaccagttatgtcgccccactgtttctgaaacttgatctattttaggttagtttgtggtgtagtaacacatgcatgcatacacacacacacacacacacacacacatca

SEQ ID NO: 80 - protein, 377 aa, Populus tremuloides
MDFHLKQWRNQHEESGQQPSAKMPKLLMDPHQPQQHPHSSGSAAFPLFLPEPSCKNSNLSAFPDSNTAANTRLPKIMGNYFSLEQWQELELQALIYRFMLAGAAIPPELLQPIKKTLLHSHPPPYFLHHPLQLHCSYYQPSWYWGRAAMDPEPGRCRRTDGKKWRCSRDVVAGHKYCERHLHRGRNRSRKPVENPTPTISTNITCIGIGELDQTTFSLFCFCFNLLAHPYCSSKTESKGLIGPPPPNEVGNRSDGHILWHFFDDWPRSVDESDNMNAGSSMNSLTCLSVSMPGNSPASDVSLKLSTGNNIAEEEPEPVPAPIPRGNTSNWAAAGWGTKITNQVVTSMGGPLAEALRSSTTKLISHECSAPVMSPHCF

SEQ ID NO: 81 - DNA, 1280 bp, Populus tremuloides
gcgctcgcttgtgttgtggtgacaggagaaatgacaccatctgtgaatgagagagtgctttttacagctgctcagtggcaagaacttgaaagacaaaccacgatttacaagtacatgatggcttctgttcctgtccctcctgaactccttatacccatcaccaaaaatcaatcaaatgtccttcctccacggtctaacagttcactagaactgggaattcctagcctgaactcatcagatgcagaaccatggagatgcaaaagaactgatgggaaaaaatggaggtgttcaagagatgtggcacctgaccagaaatactgtgagaggcactctcataagagccgtccccgttcaagaaagcctgtggaattacacactcatgactccccgaggacattgaccaacaataacactaacaccaacaatagcaattactccactaatccacacctgtttaatcaaaaaccttactttccaagccatttatttatgtttcctagtgccatggccccttctgccagctcatatgatcaacccaggagcttggaatggctcttgaaaggcgagattttacccgttgccagtaattacagccaagaatggcagcatttgaagagagacagcatcaagggtaatggcaaagtgtacaacgtttatggagaagagcagccgctttgctcaaatacatatagaggtggccattcattacaagctcagaggctaaatgatcattgcagcgtgttatcaagtcccaaatcaactactttggaaagggctttaagtcctagcctgacccaagaacaagagacaaggcacttcattgatgcttggtcaactaattcagggagagacgacattggtgggattggtaaaaaaagttacgtttcttcaagtgagaagttagtattgccacattcagctcttacattgtcaatgtcacctggcactggaagtgaaactaataatgaaggaaatgggagtgctcaactgagtagttttgggatcatgggattatcagatagagatcatcagagtgcgagtggcttgagacctcagtggatgatgagtcatggtggttcatggatagtatcaccacctggtggaccattagctgaagccttgtgtcttggcatttccagcaatgcaaaaactgcttccaatttaccatccccttgcagcagtagctgtggccccaattaatgtcaacaaaaaccaaccgctatagtttgttgtaaattctggctgggtagaggccagagggtagaagctaatgtggccca

SEQ ID NO: 82 - protein, 513 aa, Populus tremuloides
MRSSWSRTRSGVFVDDIGLGLRMQDNLESCSGSSKRSVTAMSCDHEPAAHELSSSSCSGGGGGSGPLFYSTSNHVTCLGDIKDVVASVSASGTGTPDAIAESKSLQYPYFISDSSPFTFNSSGEMTPSVNERVLFTAAQWQELERQTTIYKYMMASVPVPPELLIPITKNQSNVLPPRSNSSLELGIPSLNSSDAEPWRCKRTDGKKWRCSRDVAPDQKYCERHSHKSRPRSRKPVELHTHDSPRTLTNNNTNTNNSNYSTNPHLFNQKPYFPSHLFMFPSAMAPSASSYDQPRSLEWLLKGEILPVASNYSQEWQHLKRDSIKGNGKVYNVYGEEQPLCSNTYRGGHSLQAQRLNDHCSVLSSPKSTTLERALSPSLTQEQETRHFIDAWSTNSGRDDIGGIGKKSYVSSSEKLVLPHSALTLSMSPGTGSETNNEGNGSAQLSSFGIMGLSDRDHQSASGLRPQWMMSHGGSWIVSPPGGPLAEALCLGISSNAKTASNLPSPCSSSCGPN

SEQ ID NO: 83 - DNA, 1623 bp, Saccharum officinarum
ccagcatccactctctcatcagcagcctcttcttcttctcccccaaatgagtgctgagttttgtgctgctgcgggcatggagctcggagtcggggatgtgatggggctgcagcaaggcatcgccatcaccgcgccatcgcccaggggcagcggcgacctgggtcttctcaagcgagcagccctcacccaggcagcagctggcccctacccctcccccttcctcgacgaacagaagatgctcaggttctccaaggcggctcacacattgccctcagggctaggcttggattttggaggcccaagcgagcaggctttcctgctgtccaggaccaagaggccatttactccctcgcagtggatggagctggagcaccaggctctgatatacaagtatctcaatgcaaaggcccccataccttccagcctgctcatttcaatcagcaagagcttcagatcatccaatagagtgagctggaggcctctctatcaaggctacacaaatgcagactctgacccagagcctggaagatgccgacgaacagatggaaagaagtggcgatgctccaaggaggcaatggctgatcacaagtactgtgagcggcacatcaacagaaaccgtcaccgttcaagaaagcctgtggaaaaccaacccaaaaagaccaccaaggaggtgcctgctgctgctagctcattgccatgtgctgggccacaaggttgcttgaagaaggcaaaagttaatgactccaagccaggcactgtcagctgttggacagatagtttaaacaggacaatgttgagcagagagaaagcaaacaaaccgacggaggacaactctttgctgcttaattctacgaatagccagcccaccttgtccctgctctctcaactgaagcagcagaacaaaccagataagttaggtcccacactggaaaatgagtcaaactcagacacaatactgaaagcctggggtggcaaccagcctagccacaagagcatttcctccacacagcaccatgatgctgaatccctccaatcagtccttcaaaatttcagcctagcccagaatgagaagatggagtcagaaaagaacaaatattctgattccatgctagtttcatcgactttctattctgcagacggtccacgatctacctgccttacacctaacatgacacaagtgcagcaggattgcatatcaagctcttgggagatgcctcaaggtggacctctaggcgagatcttaaccaactccaagaatagtgaggacttaagcaagtgtgaatcaaggtcatatggttggttattgaatcttgaccatgcaccatgattcctcaatccatggagagcttgacatagatgtctcaccatggaagcaaacaatggtcagaaaaagaaggttcaaatgaccacattgtttgccccatgcatgctcgctatctacatttgtatttctgttttgtagcatttagctagttgaattatcagttcttctgaatctggctgtattttaaacaaattctagtttgtgtcagatgatatcttgcttgctagatgtttcatgtctaactttcaacaggaacttcagagatccattttgatcaacagaaaactgtttgaagaacc

SEQ ID NO: 84 - protein, 426 aa, Saccharum officinarum
MSAEFCAAAGMELGVGDVMGLQQGIAITAPSPRGSGDLGLLKRAALTQAAAGPYPSPFLDEQKMLRFSKAAHTLPSGLGLDFGGPSEQAFLLSRTKRPFTPSQWMELEHQALIYKYLNAKAPIPSSLLISISKSFRSSNRVSWRPLYQGYTNADSDPEPGRCRRTDGKKWRCSKEAMADHKYCERHINRNRHRSRKPVENQPKKTTKEVPAAASSLPCAGPQGCLKKAKVNDSKPGTVSCWTDSLNRTMLSREKANKPTEDNSLLLNSTNSQPTLSLLSQLKQQNKPDKLGPTLENESNSDTILKAWGGNQPSHKSISSTQHHDAESLQSVLQNFSLAQNEKMESEKNKYSDSMLVSSTFYSADGPRSTCLTPNMTQVQQDCISSSWEMPQGGPLGEILTNSKNSEDLSKCESRSYGWLLNLDHAP

SEQ ID NO: 85 - DNA, 1515 bp, Vitis vinifera
atgatgatgagtgcaagaaacaggtctcctttcacagcatcacagtggcaagagcttgaacatcaagctcttatcttcaaatatatagtgtcaggagtaccaatcccagctgatctcatctgcactgtcaaaagaagcttggactcttcattgtcttcaaggctatttcctcaccaacccattgggtggggttgttttcagatggggtttggcaggaaagcagacccagagccagggaggtgcagaagaactgatggcaagaaatggaggtgctccaaagaagcatacccagattcaaaatactgtgagagacacatgcacagaggcaaaaaccgttcaagaaagcctgtggaagttatttcagctacaaacccttcaccaaccatctcatcaatcaactcaaatccttcctccaccaccaccaattcttactctctctctcctctctctcctctctcttcttcaatgacttctgaaacctcccatccccatcaccattcctaccacaacacctctctttatcccttcctctaccctcacccctcctcttctagacctcctggttcttgcctatcacctcaagccaccagcagttacagcacccatcatctgtttttggactctgggtcttattcccaggctgatagggattacaggggtgtggatgagagagctttcttcccagaagcttcagggactgtaaggggcctacatgattcatatactccattaacaatgagttcctccaagggatactctcactttcagtatcaaagccccgctgataatcccaaacagcagcaagaacagcaagagcagcagcactgctttgtcttgggcactgatttcaaatcgtcaaggccaattaaagtagagagagatgatgaagcccagaagcctctccaccatttctttggagagtggcctctaaagaacagagactcctggcttgaccttgaggaggatccaccaacccatgcatcattctccaccacccagctctcaatttcaatcccaatgtcctcacacaagcttcttgcatcggattccagaatccaaactggtacttagacttcacttcagatttggccttcttgccctttattttcccttttctgctaagtctcatcttctacttcatttttcccccttgtgcagatggatgagttctcaatactggttcttctgatcatggccaaaaaagtacttgtactggtgggttcaatgcttttcttgttgattttgtgactgaaggaagtttcttttgccaaatgtgcggatgaataatgtagggcctataggaggattcttgtctttgtgctttctgagttgctaattttcattcttatctttaaagaaacatgtttgaatttgtagacttgtttgtttgacaggaagcatcttgatatggtttttgagtttgagtttgagttgttctctaatctctatgttgttttgtgagttgtggccaccttttcttttccctttggcttctgcttattgtacttcagaaag

SEQ ID NO: 86 - protein, 356 aa, Vitis vinifera
MMMSARNRSPFTASQWQELEHQALIFKYIVSGVPIPADLICTVKRSLDSSLSSRLFPHQPIGWGCFQMGFGRKADPEPGRCRRTDGKKWRCSKEAYPDSKYCERHMHRGKNRSRKPVEVISATNPSPTISSINSNPSSTTTNSYSLSPLSPLSSSMTSETSHPHHHSYHNTSLYPFLYPHPSSSRPPGSCLSPQATSSYSTHHLFLDSGSYSQADRDYRGVDERAFFPEASGTVRGLHDSYTPLTMSSSKGYSHFQYQSPADNPKQQQEQQEQQHCFVLGTDFKSSRPIKVERDDEAQKPLHHFFGEWPLKNRDSWLDLEEDPPTHASFSTTQLSISIPMSSHKLLASDSRIQTGT

SEQ ID NO: 87 - DNA, 945 bp, Zea mays
agagcgccgtatcacctgtctctccgtccaccgccgtctcgatccgcgccaaagatacctttcccccaccccttcctcgcgccgccgtttggtgcgaccatgacggcggagggggaggccaagaacccgtcggccggtggcggaggggataacccccagcaccagcaggctgcgccggcgccggcgccggcacagggggaagtggcgcaggaggctgcagtgcaggggacgggacaagagcaggagcgggacaaggcggatcgagaggtgcagggcggcgcgggggagaaggacgacggcgcgtgcagagatctggtcctggtcgaggatccggaggtcctcgccgtcgaggatccggaggaagctgcagcaaccgcagcactccaggaagaaatgaaagcgcttgtggcatcgatccctgatggtgctggagcagcattcacagccatgcagcttcaggagctagagcagcagtcccgggtgtaccagtacatggctgcccgagtacctgtgcctactcacctcgtcttcccggtatggaagagtgtgaccggtgcatcctctgaaggcgcccagaagtaccctactttgatgggcttagcaacgctctgcttggactttgggaagaacccggaaccagaaccagggaggtgtcggcgaacagatggtaagaaatggcgatgttggagaaacactatcccaaacgagaaatactgcgaacgtcacatgcatcgtggccgcaagcgtcctgtacaggttttcctggaggacgacgagcccgattctgcttcagggtcaaaacccgccgctcctggcaaggctaccgaaggtgccaagaaggccgatgacaagagcccaagcagcaagaagcttgcagtggcagcgcctgccgctgtgcagtctacatagtcaattgcagctttagtagcccgcagaaagagcata

SEQ ID NO: 88 - protein, 269 aa, Zea mays
MTAEGEAKNPSAGGGGDNPQHQQAAPAPAPAQGEVAQEAAVQGTGQEQERDKADREVQGGAGEKDDGACRDLVLVEDPEVLAVEDPEEAAATAALQEEMKALVASIPDGAGAAFTAMQLQELEQQSRVYQYMAARVPVPTHLVFPVWKSVTGASSEGAQKYPTLMGLATLCLDFGKNPEPEPGRCRRTDGKKWRCWRNTIPNEKYCERHMHRGRKRPVQVFLEDDEPDSASGSKPAAPGKATEGAKKADDKSPSSKKLAVAAPAAVQST

SEQ ID NO: 89 - DNA, 1372 bp, Zea mays
gcctctgacaccagcacaaacctggagactactactagtattggagtcccctccacttccacctcccttgccactgaagcgagagctctcggagccgtcgtcctctgtctctcatccttcttcgttgttgagcaaagcgggctcgaggaggagatgatgctgagcgggcacggcggcgggaggcgcctgttcacggcgtcgcagtggcaggagctggagcaccaggcgctcatcttcaaatacatggcctccggcgcgcccgtgccgcacgacctcgtcctgccgctccgcctcgccaccggcgtcgacaccgcgccctccctcgccttcccgccccagccttcgccgtcgctggcgtactggggctgctatggcgcgggggcgccgttcggccgcaaggcggaggacccggagcccgggcggtgccggcggacggacggcaagaagtggcgatgctccagggaggcccacggagactccaagtactgcgagaagcacatccaccgcgggaagagccgttcaagaaagcctgtggaagtgacctcccccgccgcctaccgcccgtccgcgttctccatctcgccgcctcgcgcggccgacgcgccgccgccgccgccgggcctcggccacccgcagcagcagcatctccgccacggcgctctctctccagcaggccgcgcccacgccgctggcgctctccagctccacctcgactcgagcctgcacgcggcgtcgccgccgccgtcctaccacaggtacgcccactcccacgctcactacacgccgccgccgccgccgtcgctctacgactacgggcagtccaaggagcttcgggaggcggcggagctcaggcggcggcacttccacgcgctcggggccgacctgagcctcgacaagccgctggccgacgccggggccgcggagaagcccctgcggcgtttcttcgacgagtggccgcgggagagaggcgacacgaggccgtcgtgggcgggggcggaggacgcgacgcagctctccatctccatccccgcggcttcgccctcctctgaccacgctgcctctgccgccgcgcgatgccacaacgatgggagtgatcggtgcatctcctagctgcaactgcaatgcaagcctgcaaccgcgtggattgttgttgattggtgtagtttcctagctgcaattcaagcctgcaacagcgagcagtgagcagcaaatgcgtggggagggcacgcagctcaggctgatgcgcaaaatccgaagcgagtcaagcagcaataggactctaggtctatgatttgatcttcctttgtagcagtacgttaccaaaatgttagctcgttgttgttcggtgtgacattttcgttcaggttgctcc

SEQ ID NO: 90 - protein, 318 aa, Zea mays
MMLSGHGGGRRLFTASQWQELEHQALIFKYMASGAPVPHDLVLPLRLATGVDTAPSLAFPPQPSPSLAYWGCYGAGAPFGRKAEDPEPGRCRRTDGKKWRCSREAHGDSKYCEKHIHRGKSRSRKPVEVTSPAAYRPSAFSISPPRAADAPPPPPGLGHPQQQHLRHGALSPAGRAHAAGALQLHLDSSLHAASPPPSYHRYAHSHAHYTPPPPPSLYDYGQSKELREAAELRRRHFHALGADLSLDKPLADAGAAEKPLRRFFDEWPRERGDTRPSWAGAEDATQLSISIPAASPSSDHAASAAARCHNDGSDRCIS

SEQ ID NO: 91 - DNA, 1099 bp, Zea mays
cgcatccgttctctatcgaaagggaggaggaggagcgcgcgggagtgggctgggggcccaccgatgctgagctcggcgtcctcggccggggcggccatggggatgggcggcgggtaccaacaccagccgctgccactgccgcagcgcggggcggcggccgcggtcttcaccgccgcgcagtgggcggagctggagcagcaggcgctcatctacaagtacctcatggccggcgtccccgtcccgcccgatctcctccgccccgccccccacgccgccgccttctccttcgccagccccgccgcgtcgcccttctaccatcaccaccaccaccacccgtccctgagttactacgcctactacgggaagaagctggacccggagccgtggcggtgccgccgcaccgacggcaagaagtggcggtgctccaaggaggcgcaccccgactccaagtactgcgagcgccacatgcaccgtggccgcaaccgttcaagaaagcctgtggaatccaagaccgcctcctcgccgccccagctgtccaccgtcgtcaccaccaccaccacccgggaggccgccgccgcgacgcccctcgagtccctcgcgggggcggggggtaaggctcacggcctgtccctcggcggcggggctggctcgtcgcacctcagcgtcgacgcttcgaacactcactttcgctatggcagcaagtaccctcttggagctaaatccgatgctggcgagctgagcttcttctcaggagcaccagggaactccaggggcttcaccattgattctccagcagataactcttggcactccctgccatccaacgtgcccccgtttacactgtccaagggcagagattctggcctcctgcctggagcgccaccagtcgtcgttcagcagcagcggggccggcgctggtgggttgctggggagcgtgaagcaggagaaccagccgctgaggcccttcttcgacgagtggcctgggacgcgggactcgtggtcggagatggacgacgcgaggtccagtaggacctccttctcgacgacccagctctccatctccattccgatgcccagatgtgattgagaacgaagctcg

SEQ ID NO: 92 - protein, 321 aa, Zea mays
MLSSASSAGAAMGMGGGYQHQPLPLPQRGAAAAVFTAAQWAELEQQALIYKYLMAGVPVPPDLLRPAPHAAAFSFASPAASPFYHHHHHHPSLSYYAYYGKKLDPEPWRCRRTDGKKWRCSKEAHPDSKYCERHMHRGRNRSRKPVESKTASSPPQLSTVVTTTTTREAAAATPLESLAGAGGKAHGLSLGGGAGSSHLSVDASNTHFRYGSKYPLGAKSDAGELSFFSGAPGNSRGFTIDSPADNSWHSLPSNVPPFTLSKGRDSGLLPGAPPVVVQQQRGRRWWVAGEREAGEPAAEALLRRVAWDAGLVVGDGRREVQ

SEQ ID NO: 93 - DNA, 1328 bp, Zea mays
cctcccgtcagcctcttcttctccccctgatgagcgctgagttctgtgctgccgccgctggtgctgtggccatggagctcggagtcggggatgtgatggggctgcagcaaggcatcgccgccgccaccgggccatcgtccggagacagcgacctgggtcttctcaagcgagcaggcctcgcccaggcagccacctcctacccctcccctttcctcgaccaacagaagatgctcaggttctccaaggcggcggcggctcacacgtcgccctcaggcctagatttcggaggaggcccaagcgagcaggctttcctgctgtccaggaccaagcggccgttcaccccgtcgcagtggatggagctggagcaccaggctctcatatacaagtatctcaatgccaaggcccccataccttccagcctgctcgtttccatcagcaagagcttcaggtcatccaacagagtgagctggaggcctctttaccaaggctacgcaaacgcagactccgacccagaacctgggaggtgccggcggacagacggaaagaagtggcggtgctctaaggaggcgatgcctgatcacaagtactgcgagcgccacatcaataggaaccgccaccgttcaagaaagcctgtggaaaaccaacctagaaagaccagcaaggaggtgcctaccgctgctgctggctcgttgccgtgtgccgggccacaaggtagcttgaagaaggcaaaagttaatgactccaagccaggcactggcagctattggacagatagcttaaacaggacaatgctgagcagggagaaggcaaacaaaccgacggaagacgagtctttgctgcttagttctacgaagaacagccagcccaccttgtccctgctcactcaactgaagcagcagaacaaaccagataagttaggtcccacaccggaaaatgagccgaactcggacacaatgttgaaagcctggggtggcagccaccacaagaacatttcctccacacagcgccatgacgctgaatccctccaatcagtcctccaaaatttcagcctagcccagaatgacaggttggagtcagaaaagaacagatattctgattccgtgctagtctcatcggctttctattctgcagacggtccacaaactacctgccttacacctaacatgacacaagtgcagcaggactgcatatcaagctcctgggagatgcctcaaggtggacctctaggcgagatcttaacgaactccaagattagtgaggactcaagcaagtgtggatctaggtcatatggttggctattgaatcttgaccatgcaccatgattcctc

SEQ ID NO: 94 - protein, 430 aa, Zea mays
MSAEFCAAAAGAVAMELGVGDVMGLQQGIAAATGPSSGDSDLGLLKRAGLAQAATSYPSPFLDQQKMLRFSKAAAAHTSPSGLDFGGGPSEQAFLLSRTKRPFTPSQWMELEHQALIYKYLNAKAPIPSSLLVSISKSFRSSNRVSWRPLYQGYANADSDPEPGRCRRTDGKKWRCSKEAMPDHKYCERHINRNRHRSRKPVENQPRKTSKEVPTAAAGSLPCAGPQGSLKKAKVNDSKPGTGSYWTDSLNRTMLSREKANKPTEDESLLLSSTKNSQPTLSLLTQLKQQNKPDKLGPTPENEPNSDTMLKAWGGSHHKNISSTQRHDAESLQSVLQNFSLAQNDRLESEKNRYSDSVLVSSAFYSADGPQTTCLTPNMTQVQQDCISSSWEMPQGGPLGEILTNSKISEDSSKCGSRSYGWLLNLDHAP

SEQ ID NO: 95 - DNA, 1440 bp, Zea mays
gccaccaagagccctccaacacacacctgacctccccttcccccctctctccgccgcccgttccccgcgcctccgcccgtacgtcccgttcccggtcggccggccggtccaaagggaggggaggaggaggggcgcgggagtcggggcccgcaccgatgctgagctcggcatcctcggccgcgggggcggccatggggatgggcggcggcgggtacgcgcaccagcccccgccacagcgcgcggtcttcaccgccgcgcagtgggcggagctggagcagcaggcgctcatctacaagtacctcatggccggcgtccccgtcccgcccgacctcctcctccccgtccgccccggccccgccgccgccttctccttcgccggccccgccgccgcgtcgcccttctaccaccaacaccacccgtccctgagctactacgcctactacggcaagaagctggacccggagccgtggcggtgccgccgcaccgacggcaagaagtggcggtgctccaaggaggcgcaccccgactccaagtactgcgagcgccacatgcaccgtggccgcaaccgttcaagaaagcctgtggaatccaagaccgcctcgtcgtcgtcgcccgcgcacccgtcgccgccccagctgtccaccgtcaccaccaccgcgcctctcgagccccttgcagcggcggggggcaaggtccacggcctgtccctcggcggcggcgctgctggctcgtcgcacctcggcgtcgatgcttcgaatgctcactatcgttatggtagcaacaggtaccctctcggagctaaaccggacggcggcgagttgagcttcttctcaggagcgtcatcggggaacaactcgaggggtggcttcaccatcgactctccatcagataacaactcgtggcactccgccctggcgtccagcgtgcccccgttcacgctgtcgacgaagagcggggactccggcctcctgcccggcgcctacgcctcctactcccagtcccactcccacatggagccgccgcgggagctcgggcaggtcaccatcgcctcgctggcgcaggagcaggagcgccagcagccgttcagtggtgggatgctcgggaacgtgaagcaggagaaccagaaccagccgctgcggcccttcttcgacgagtggcccgggacgcgggcggactcgtggccgccggagatggacggcgcgccgcgggccggcaggacctccttctcctcctccaccacccagctctccatctccatcccgatgcccagatgtgagctgcatctcagaaaccagaactcttaattctgttcgctgcccgaatcatgcttgaccgaaacttgttttctgcaggcgactgacgaggaaccgtcgatcgggcggccactagacggtggacgctcacgctcactagtgcgctgtcgcctggagtggagatcga

SEQ ID NO: 96 - protein, 382 aa, Zea mays
MLSSASSAAGAAMGMGGGGYAHQPPPQRAVFTAAQWAELEQQALIYKYLMAGVPVPPDLLLPVRPGPAAAFSFAGPAAASPFYHQHHPSLSYYAYYGKKLDPEPWRCRRTDGKKWRCSKEAHPDSKYCERHMHRGRNRSRKPVESKTASSSSPAHPSPPQLSTVTTTAPLEPLAAAGGKVHGLSLGGGAAGSSHLGVDASNAHYRYGSNRYPLGAKPDGGELSFFSGASSGNNSRGGFTIDSPSDNNSWHSALASSVPPFTLSTKSGDSGLLPGAYASYSQSHSHMEPPRELGQVTIASLAQEQERQQPFSGGMLGNVKQENQNQPLRPFFDEWPGTRADSWPPEMDGAPRAGRTSFSSSTTQLSISIPMPRCELHLRNQNS

SEQ ID NO: 97 - DNA, 1388 bp, Zea mays
gacaggttgagatggcgatgccgtatgcctctctttccccggcaggcgccgccgaccaccgctcctccacagccacggcgtccctcgtccccttctgccgctccaccccgctctccgcgggcggcgggctgggggaggaggacgcccaggcgagcgcgaggtggccggccgcgaggccggtggtgccgttcacgccggcgcagtaccaggagctggagcagcaggcgctcatatacaagtacctggtggccggcgtgcccgttccgccggatctcgtggttccaatccgccgcggcctcgactccctcgctacccgcttctacggccaacccacactcgggtacggaccgtacctggggaggaaactggatccggagcccggccggtgccggcgaacggacggcaagaagtggcggtgctccaaggaagccgccccggactccaagtactgcgagcgccacatgcaccgcggccgcaaccgttcaagaaagcctgtggaaacgcagctcgcgccccagtcccaaccgcccgccgccgcggccgtctccgccgctccgcccctggcagccgccgccgccgccgccaccaacggcagcggcttccagaaccactctctctacccggccatcgccggcagcactggtggtggaggaggagttggcgggtccggcaatatctcctccccgttctcctcgtcgatggggggatcgtctcagctgcacatggacagtgttgccagctactcctacgcagctcttggtggtggaactgcaaaggatctcaggtacaacgcttacggaataagatctctggcggacgagcacaaccagctgatcgcagaagccatcgactcgtcgatagagagccagaggcgcctccccagctcgtcgttcccgctctcgagctacccacatctcggggcgctgggcgacctgggcggccagaacagcacggtgagctcgctgccgaagatggagaagcagcagccgccctcgtccttcctagggaacgacaccggggccggcatggccatgggctccgcctccgcgaagcaggagggccagacgctgcggcacttcttcgacgagtggcccaaggcgcgggactcctggccgggcctctccgacgagaccgccagcctcgcctcgtcccccccggcgacccagctgtcgatgtccatacccatggcgtcctccgacttctccgtggccagctcccagtcgcccaacgatgactaatggtgcgtggatcgtcgcgttctggccctttgtctatctcccctccagtcctccacccaccgcgcagtagtagctgcggaaacagcccatgctcctgtatatttgtcggtcattttccgtgtcagatctgtgtaccaaaccaagcggcgg

SEQ ID NO: 98 - protein, 408 aa, Zea mays
MAMPYASLSPAGAADHRSSTATASLVPFCRSTPLSAGGGLGEEDAQASARWPAARPVVPFTPAQYQELEQQALIYKYLVAGVPVPPDLVVPIRRGLDSLATRFYGQPTLGYGPYLGRKLDPEPGRCRRTDGKKWRCSKEAAPDSKYCERHMHRGRNRSRKPVETQLAPQSQPPAAAAVSAAPPLAAAAAAATNGSGFQNHSLYPAIAGSTGGGGGVGGSGNISSPFSSSMGGSSQLHMDSVASYSYAALGGGTAKDLRYNAYGIRSLADEHNQLIAEAIDSSIESQRRLPSSSFPLSSYPHLGALGDLGGQNSTVSSLPKMEKQQPPSSFLGNDTGAGMAMGSASAKQEGQTLRHFFDEWPKARDSWPGLSDETASLASSPPATQLSMSIPMASSDFSVASSQSPNDD

SEQ ID NO: 99 - DNA, 1506 bp, Zea mays
ccatctggccatctccccttcccctgctcccccgaagcagcaagccagcctgcccacccgcagccatcacctccgccgctctccaccatgaatcccatccaccagcacgacatcgtacccaatccttcgtgactgttgcctccgcgcatctccgggagcaatggaaggaggccgagatgtgttcttaggtgcggcggcaagggcgccgccgccgccgccgtcttgcccgtttcacggatccgctaccgccacccgctccggtggagcgcagatgctcagcttctcctccaatggcgtagcagggttgggtctgtgctcaggtgccagcaagatgcagggtgtgttgtcgagggtgaggaggcccttcactccgacgcagtggatggagctggagcaccaggccctgatctacaagcacttcgctgtgaatgcccctgtgccgtccagcttgctcctccctatcaaaagaagcctcaatccatggagcagccttggctccagctcattgggatgggcaccatttcgttccggctctgctgatgcagaaccaggaagatgccgccgcacagatggcaagaagtggcggtgctctagagatgctgtcggggaccaaaaatactgtgagcgatacataaaacgtggttgccaccgttcaagaaagcatgtggaaggccgaaaggcaacaccgaccactgcagatccaaccatggctgtttctggtggttcattgttgcacagccatgctgttgcttggcagcagcagggcaaaagctcagctgctaatgtgactgatccattctcactagggtccaacaggaatttgctggataagcagaatctaggtgaccagttctctgtatccacttccatggactcctttgacttctcatcatcacattcttccccaaaccaagccaaagttgcattttcaccggtggccatgcagcacgaacatgatcagctgtatcttgtgcatggagccggcagctcagcagaaaacgttaacaagtctcaggatggtcagctgctagtctcgagggaaacaattgacgacggacctctgggcgaggtgttcaagggcaagagttgccagtcagcatccgcagacatcttaactgaccattggacttcgactcgtgacttgcgtcctccaaccggagtcctacaaatgtctagcagcaacacagtgccagcagagaatcacacgagtaacagtagctatctcatggcgaggatggcgaattctcagaccgtcccaacactccactgagtgttcatcaggctggtctttgttgggaccacaaaataactgaagccatgttgatgtcctgagtttgctgatacagtgatactaggttttcagtcgagtcttgtaactcctgttttagagttgttatatgttcacgtcatgttgcctttcattttcggtttcattcagatgggtgtactaataatttctttccttcttacctgtgaaggatttgagttccaatctgagacgtgggt

SEQ ID NO: 100 - protein, 369 aa, Zea mays
MEGGRDVFLGAAARAPPPPPSCPFHGSATATRSGGAQMLSFSSNGVAGLGLCSGASKMQGVLSRVRRPFTPTQWMELEHQALIYKHFAVNAPVPSSLLLPIKRSLNPWSSLGSSSLGWAPFRSGSADAEPGRCRRTDGKKWRCSRDAVGDQKYCERYIKRGCHRSRKHVEGRKATPTTADPTMAVSGGSLLHSHAVAWQQQGKSSAANVTDPFSLGSNRNLLDKQNLGDQFSVSTSMDSFDFSSSHSSPNQAKVAFSPVAMQHEHDQLYLVHGAGSSAENVNKSQDGQLLVSRETIDDGPLGEVFKGKSCQSASADILTDHWTSTRDLRPPTGVLQMSSSNTVPAENHTSNSSYLMARMANSQTVPTLH

SEQ ID NO: 101 - DNA, 1350 bp, Zea mays
tagccgtgctccgctcaccttctctcgcgctacagtctcaaggggtagctagccaagctaccaagctcgtcaggaacgagagaaagaggccggcggtgcgcggggatgatgatgatgagcagcggccgggcgggcggcggggccaccgcggggcggtacccgttcacggcgtcgcagtggcaggagctggagcaccaggcgctcatctacaagtgcctggcgtccggcaagcccatcccttcctacctcatgccgccgctccgccgcatcctcgactccgccctcgccacgtcgccgtccctcgcctacccgccgcaaccctcgctgggctggggctgcttcgggatgggcttcacccggaaggccgacgaggacccggagcccgggcggtgccggcgcacggacggcaagaagtggcgctgctccaaggaggcgtacccggactccaagtactgcgagaagcacatgcaccggggcaagaaccgttcaagaaagcctgtggaaatgtccttggccacgccggccccggcgccggcccccgccgccgccacaaccgccaccgccacctcatccccggcgccgtcctaccaccgcccggcccacgacgccacgccgtctccgtaccacgcgctgtatggaggcggcggcggcggcggcggtagcccttactcggcgtcggcacgcccaggagcaaccggaggcggcggcgcgtaccaccacgcgcagcatgtgagccccttccacctccacctcgagaccacccacccgcacccgccgccgccctacaactactccgccgaccagagggactacgcgtacgggcacgcggccgccaaggaggtcggcgagcacgccttcttctcggacggcgcgggcgagcgggtcgaccgccaggccgcggcggggcagtggcagttcaggcagctcggggtggagacgaagccgggccccacgccgctgttccccgtcgccgggtacgggcacggcgcggcgtcgccgtacggcgtcgagctgggcaaggacgacgacgagcaggaggagaggcgccgccagcactgcttcgttcttggagccgacctgcggctggagcggccgtcgtcgggccatggccatggccatggccatgaccatgacgacgccgccgccgcgcagaagccgctccggcccttcttcgacgagtggccgcaccagaagggggacaaggccgggtcgtggatggggctcgacggcgagacgcagctctccatgtccatccccatggccgctaccgacctccccgtcacctcccgcttccgtaacgacgagtgatgccacatcaaacctggcgctggaaactcggaacgtatggtg

SEQ ID NO: 102 - protein, 400 aa, Zea mays
MMMMSSGRAGGGATAGRYPFTASQWQELEHQALIYKCLASGKPIPSYLMPPLRRILDSALATSPSLAYPPQPSLGWGCFGMGFTRKADEDPEPGRCRRTDGKKWRCSKEAYPDSKYCEKHMHRGKNRSRKPVEMSLATPAPAPAPAAATTATATSSPAPSYHRPAHDATPSPYHALYGGGGGGGGSPYSASARPGATGGGGAYHHAQHVSPFHLHLETTHPHPPPPYNYSADQRDYAYGHAAAKEVGEHAFFSDGAGERVDRQAAAGQWQFRQLGVETKPGPTPLFPVAGYGHGAASPYGVELGKDDDEQEERRRQHCFVLGADLRLERPSSGHGHGHGHDHDDAAAAQKPLRPFFDEWPHQKGDKAGSWMGLDGETQLSMSIPMAATDLPVTSRFRNDE

SEQ ID NO: 103 - DNA, 1028 bp, Zea mays
tcccttcaccgctgcctcgacccgcgccgaaagatacctttcccccccttcctctcgcgccgccgttttggtgcgaccatggcggcggagggggaggccaagaacccgtccggcggtggcgaagggggtaacccccagcaccagcaggcagtgcaggctgcgccggcggagccgccaatggcacagggggaagcggtgcaggaggctggagcgcaggcgacgggacaagagccggagggggagaaggcgaatcgagatggggagggaagcgcgggggagaaggacgacggcgcgtgcagagatctggttctggttgaggatccggaggtgctcgccgtcgaggacccggaggaagctgcagcaaccgcagcactccaggaagaaatgaaagcgctcgtggcatccgtccctgacggtgctggggcagcattcacagccatgcagcttcaggagctagagcagcagtcccgggtttatcagtacatggctgcccgagtacctgtgcctactcacctcgtcttccccgtatggaagagtgtaaccggtgcatcctctgaaggcgcccagaagtaccctactttgttgggcttagcaacactctgcttggacttcgggaagaaccctgaaccagaaccagggaggtgccggcgaacggatggcaaaaaatggcgatgttggagaaacactattccaaacgagaagtactgcgaacgccgcatgcatcgcggtcgcaagcgtcctgtacaggtcgtcgaggaagccgagcctgactctgcttcaggctcaaaatctgctcccggcaaggccaccgaaggcgccaagaaggttggcgacaagagcccaggtagcaagaagcttgccgtggcggcggcagctgcagctgctgcgcagtctacgtaattgatgcagcattttagtagtcgcaggaagagcatggcggcgctggcaactagcgccttcttttcattgcatgtgatctttagctataacctcatttagcacactcccagtggtgtccgtgggaggag

SEQ ID NO: 104 - protein, 273 aa, Zea mays
MAAEGEAKNPSGGGEGGNPQHQQAVQAAPAEPPMAQGEAVQEAGAQATGQEPEGEKANRDGEGSAGEKDDGACRDLVLVEDPEVLAVEDPEEAAATAALQEEMKALVASVPDGAGAAFTAMQLQELEQQSRVYQYMAARVPVPTHLVFPVWKSVTGASSEGAQKYPTLLGLATLCLDFGKNPEPEPGRCRRTDGKKWRCWRNTIPNEKYCERRMHRGRKRPVQVVEEAEPDSASGSKSAPGKATEGAKKVGDKSPGSKKLAVAAAAAAAAQST

SEQ ID NO: 105 - DNA, 1374 bp, Zea mays
cagccaggtaaggcaaaagagagagggcggaagcagcggcagagcggagagggagagagaagagcatatatgggcatggcgatgccctttgcctccccgtctccggcagccgaccaccgcccctcctccctcctccccttctgccgcgccgcccctctctccgcggcgggagaggacgccgcgcagcagcacgcgatgagcggcaggtgggccgcgaggccggcgctcttcacggcggcgcagtacgaggagctggagcaccaggcgctcatatacaagtacctcgtcgccggcgtgcccgtcccgccggacctcctcctccccctgcgccgaggcttcgtcttccaccagccacccgcccttgggtacggcccctacttcggcaagaaggtggacccggagcccgggcggtgccggcgtacggacggcaagaagtggcggtgctccaaggaggccgccccggactccaagtactgcgagcgccacatgcaccgcggccgcaaccgttcaagaaagcctgtggaagcgcagctcgcgcccccgccgcacgcccagccgccgcagcagcagcaggcccccgcgcccgctgctggcttccagaaccactcgctgtacccgtcgatcctcaacggcaacggcggcggcgggttaggtgctggtgctggtggtggcacgttcggcctggggcccacctctcagctgcacatggacagtgccgctgcctacgcgactgctgccggtggagggagcaaatatctcaggtactctgcatacggggtgaaatctctgtcggacgagcacagcacgctcttgtcgggcggcatggatccgtcgatgatggacaactcgtggcgccttctgccatcccaaaacaacacattccaagccacaagctaccctgtgttcggcacgctgagtgggctagacgagagcaccatcgcgtcgctgccgaagacccagagggagcccctctctttcttcgggagcgacttcgtgaccgccgccaagcaggagaaccagacgctgcgccctttcttcgacgagtggcccaagtcgagggactcgtggccggagctgggcgaggacggcagcctcggcttctcggccacccagctctccatctccattcccatggcgacctccgacttctccaacaccagctccagatcgccgggtggaataccgtcgagatgaacgagtaccgtgcatgtggatcccagcgtcttagggttgacgactcttcggtgctggcctcatcgtatcatgctcctaaattttcgaacgatatatgccttatgtaacgctatttctctcattgttacaacaccctttacccgtttggaattgtgttgaagtggatggtctgcgttgctc

SEQ ID NO: 106 - protein, 374 aa, Zea mays
MGMAMPFASPSPAADHRPSSLLPFCRAAPLSAAGEDAAQQHAMSGRWAARPALFTAAQYEELEHQALIYKYLVAGVPVPPDLLLPLRRGFVFHQPPALGYGPYFGKKVDPEPGRCRRTDGKKWRCSKEAAPDSKYCERHMHRGRNRSRKPVEAQLAPPPHAQPPQQQQAPAPAAGFQNHSLYPSILNGNGGGGLGAGAGGGTFGLGPTSQLHMDSAAAYATAAGGGSKYLRYSAYGVKSLSDEHSTLLSGGMDPSMMDNSWRLLPSQNNTFQATSYPVFGTLSGLDESTIASLPKTQREPLSFFGSDFVTAAKQENQTLRPFFDEWPKSRDSWPELGEDGSLGFSATQLSISIPMATSDFSNTSSRSPGGIPSR

SEQ ID NO: 107 - DNA, 1172 bp, Zea mays
gatatatggcgatgccctttgcctccctgtctccggcagccgaccaccgcccctcctccctcctcccctactgccgcgccgcccctctctccgcggtgggagaggacgccgccgcgcaggcgcagcagcagcagcagcagcacgctatgagcggcaggtgggcagcgaggccgccggcgctcttcacagcggcgcagtacgaggagctggagcaccaggcgctcatatacaagtacctcgtcgccggcgtgcccgtcccgccggacctcctcctccccctacgccgaggcttcgtctaccaccaacccgcccttgggtacgggccctacttcggcaagaaggtggacccggagcccgggcggtgccggcgtacggacggcaagaagtggcggtgctccaaggaggccgccccggactccaagtactgcgagcgccacatgcaccgcggccgcaaccgttcaagaaagcctgtggaagcgcagctcgtgcccccgccgcacgcccagccgcagcagcaggcccccgcgcccaccgctggcttccagagccaccccatgtacccatccatcctcgccggcaacggcggcggcggcggcggggtaggtggcggtgctggcggtggcacgttcggcctgggccccacctctcagctgcgcatggacagtgccgctgcttacgcgactgctgctgatggagggagcaaagatctcaggtactctgcctacggggtgaagtcactgtcggacgagcacagccagctcttgcccggcggcggcggcggcatggacgcgtcaatggacaactcgtggcgcctgttgccgtcccaaaccgccgccacgttccaagccacaagctaccctctgttcggcgcgctgagcggtctggacgagagcaccatcgcctcgctgcccaagacgcagagggagcccctctccttcttcgggagcgacttcgtgaccccgaagcaggagaaccagacgctgcgccccttcttcgacgagtggcccaagtcgagggactcgtggccggagctgaacgaggacaacagcctcggctcctcggccacccagctctccacctccatccccatggcgccctccgacttcaacaccagctccagatcgccgaatggaataccgtcaagatgaacctgagtaaccatgcggacccca

SEQ ID NO: 108 - protein, 380 aa, Zea mays
MAMPFASLSPAADHRPSSLLPYCRAAPLSAVGEDAAAQAQQQQQQHAMSGRWAARPPALFTAAQYEELEHQALIYKYLVAGVPVPPDLLLPLRRGFVYHQPALGYGPYFGKKVDPEPGRCRRTDGKKWRCSKEAAPDSKYCERHMHRGRNRSRKPVEAQLVPPPHAQPQQQAPAPTAGFQSHPMYPSILAGNGGGGGGVGGGAGGGTFGLGPTSQLRMDSAAAYATAADGGSKDLRYSAYGVKSLSDEHSQLLPGGGGGMDASMDNSWRLLPSQTAATFQATSYPLFGALSGLDESTIASLPKTQREPLSFFGSDFVTPKQENQTLRPFFDEWPKSRDSWPELNEDNSLGSSATQLSTSIPMAPSDFNTSSRSPNGIPSR

SEQ ID NO: 109 - DNA, 1154 bp, Zea mays
agcgtgcattgttgagcgagtgcggccaagcaacgcgggctcgaggagatgatgctgagcgggcacggcggcgggaggcgcctgttcacggcgtcgcagtggcaggagctcgagcaccaggcgctcatcttcaagtacatggcctcgggcgcgcccgtgccgcacgacctcgtcctaccgctccgcctcgccaccggcgtcgacaccgcgccctccctcgccttcccgccccagccttcgccgtcgctggcgtactggggctgctacggcgcgggggcgccgttcgtcggccgcaaggcggcggaggacacggagccggggcggtgccggcggacggacggcaagaagtggcggtgctccagggaggcccacggcgactccaagtactgcgagaagcacattcaccgcgggaagagccgttcaagaaagcctgtggaagtgacctcctcccccgccgccggcgccgctgcggcgtaccgaccgtccgcgatctccaccatctcgccgccccgcgcggccgacgcgccgccgccgagcctcgcctacccgcagcagcatctcctccacggcgcctcctcctccgcagcagcccgcgcccccgctggcgctctccagctccacctcgacgcgagcctgcacgcggcggcggcgtcgccatcgccgccgccgtcctaccacaggtacgcccactacacaccgccagcgtcgtcgctcttcccgggcggcggctacggctacgactacgactacgggcagtccaaggagctcaggcgacggcacttccacgcgctcggggccgacctgagcctcgacaagccgctgcccgagcccgacaccggctccgacgagaagcagcccctgcggcgtttcttcgacgagtggccgcgggagagcggcgacatggcggcggacgacgcgacgcagctttccatctccatccccgcggcttcgccctccgacctcgctgctacctccgcctccgccgccgccgcgcgattccacaacggggagtgatcggtccatctcctagctgcagccctgcaacagcgtggattgaccgctgcatttcctggctgcaatgcaagcctgcaacagcgagcagtaagccagtgacgtggatgcatctcgtagcggcaaaccctgcttctgcctct

SEQ ID NO: 110 - protein, 321 aa, Zea mays
MMLSGHGGGRRLFTASQWQELEHQALIFKYMASGAPVPHDLVLPLRLATGVDTAPSLAFPPQPSPSLAYWGCYGAGAPFVGRKAAEDTEPGRCRRTDGKKWRCSREAHGDSKYCEKHIHRGKSRSRKPVEVTSSPAAGAAAAYRPSAISTISPPRAADAPPPSLAYPQQHLLHGASSSAAARAPAGALQLHLDASLHAAAASPSPPPSYHRYAHYTPPASSLFPGGGYGYDYDYGQSKELRRRHFHALGADLSLDKPLPEPDTGSDEKQPLRRFFDEWPRESGDMAADDATQLSISIPAASPSDLAATSASAAAARFHNGE

SEQ ID NO: 111 - DNA, 1516 bp, Zea mays
ttcggcacgacccaacaatgcacaccaacatccactccctcgtcaggctcctctcccccaaatgagcgctgagttctgcgctgctgcgggtgtcgtggccatggagctcggggtcggagatgcgctggggctgcagcaaggcatcgcaatcaccgcgccatcgcccagggacagcgacctgggtcttctcaagcgagcaggcctcacccaggctgcggctgctgccccctacccctcccccttccttgacggggagaagatgctcaggttctccaaggcggctcacacatcgcactcaggcttggattttggaggcccaggtgagcaggctttcctgctgtccaggaccaagatgccatttactccctcgcagtggatggagctggggcaccaggctctgatatacaagtacctcaatgcaaaggcccccataccttccagcctgctcatttcaatcagcaagagcttcagatcatccaatagagtgagctggaggcctctgtatcaaggctacacaaatgcagactctgacccagaacctgggagatgccgacgaacggatggaaagaagtggcggtgctccaaggaagcaatggctgatcacaagtactgtgagcggcacatcaacagaaaccgtcaccgttcaagaaagcctgtggaaaatcaacctaagaagaccaccaaggaggtgcctgctgctgctggctcattaccatgtgctgggccacaaggtagcttgaagaaggcaaaagttaatgactccaagccaggcactgtcagctattgggcagatagtttaaacaggacaatgttgagcagagagaaagcaaacaaaccgacggaagatagctctttgctgcttacttctacgaacagccaacccacctggtccctgctctctcagctgaagcagcaaaacaaaccagataagttaggccccacactggaaaatgagtcaaacccagacacaatattgaaagcctggggtggcaaccagcctagccacaagagcatttcctctacagagcgccatgatgctgaatccctccaatcagtccttcaaaatctcagcctagcccagaatgagaagatggagtcagaaaaggacaaatattctgattccgtgctagtttcgtcgactttctattctgcaggcggtccaagagctacctgccttacacctaacatgacacaggtgaagcaggattgcatatcaagctcttgggagatgcctcaaggtggacctctaggcgaaatcttaacgaactccaagaatagcaaggacttaagcaagtgcaaaccaaggtcatatggttggttgttgaatcttgaccatgcaccatgattcctcaatccatgaagagcttgacatagatgtcccatcatgtaggcaaacaatggtcagaaaaaggttatgaccacattgcttgccccatgcatgcttgctatctacatttgtatttctgttgcgtagcatttagctagttgaattatcagttcttctggatacggctgt

SEQ ID NO: 112 - protein, 427 aa, Zea mays
MSAEFCAAAGVVAMELGVGDALGLQQGIAITAPSPRDSDLGLLKRAGLTQAAAAAPYPSPFLDGEKMLRFSKAAHTSHSGLDFGGPGEQAFLLSRTKMPFTPSQWMELGHQALIYKYLNAKAPIPSSLLISISKSFRSSNRVSWRPLYQGYTNADSDPEPGRCRRTDGKKWRCSKEAMADHKYCERHINRNRHRSRKPVENQPKKTTKEVPAAAGSLPCAGPQGSLKKAKVNDSKPGTVSYWADSLNRTMLSREKANKPTEDSSLLLTSTNSQPTWSLLSQLKQQNKPDKLGPTLENESNPDTILKAWGGNQPSHKSISSTERHDAESLQSVLQNLSLAQNEKMESEKDKYSDSVLVSSTFYSAGGPRATCLTPNMTQVKQDCISSSWEMPQGGPLGEILTNSKNSKDLSKCKPRSYGWLLNLDHAP

SEQ ID NO: 113 - DNA, 1139 bp, Zea mays
gtaggtcgttcgcaggtaggtaaccgtaacctagctagctcgtcgggatgatgatgatgagcggtcgagcggccaccgcggggcggtacccgttcacggcgtcgcagtggcaggagctggagcaccaggcgctcatctacaagtgcctggcgtccggcaagcccatcccgtcctacctcatgccaccgctccgccgcatcctcgactccgccctcgccacgtcgccgtcgctcgccgccttccagccgcaaccctcgctggggtgggggggctgcttcgggatgggcttcagcaggaagcccgccgacgaggacccggagcccgggcggtgccggcgcacggacggcaagaagtggcgctgctccaaggaggcgtacccggactccaagtactgcgagaagcacatgcaccggggcaagaaccgttcaagaaagcctgtggaaatgtccttggccacgccggcgccgccggcctcctccgctgccaccacctcgacgtccccggcgccgtcctaccaccgcccggcccccgccgcgcacgacgccgtgccgtaccacgcgccctacggcgccgcgtaccatcacacgcagacgcaggtgatgagccccttccacctccacctcgagaccacccacccgcacccgccgccgccgccgccctactactacgcggaccagagggactacgcctacggcaaggaggtcggcgagcgcgccttcttctccgacggcgcgggggagagggaccgccagcagcaggccgcggggcagtggcagttcaagcagctcgggacgatggaggcgacgaagccgtgccccacccccacgccgctgctccccgccgccgggtacggcgtcggtcaggccaaggaagacgaggaggaggaaacgcggcggcagcagcagcagcactgcttcgttcttggcgccgacctgcggctggcggagcggccgtcgggggcacatgacgacgccgcgcagaagccgctccggcatttcttcgacgagtggccgcacgagaaagggagcaaggcggggtggtggattgggggactcgacggcgagacgacgcagctctccatgtccatcccgatggcggccgctgccgacctccccgtcacctcccgctaccgtacgtga

SEQ ID NO: 114 - protein, 363 aa, Zea mays
MMMMSGRAATAGRYPFTASQWQELEHQALIYKCLASGKPIPSYLMPPLRRILDSALATSPSLAAFQPQPSLGWGGCFGMGFSRKPADEDPEPGRCRRTDGKKWRCSKEAYPDSKYCEKHMHRGKNRSRKPVEMSLATPAPPASSAATTSTSPAPSYHRPAPAAHDAVPYHAPYGAAYHHTQTQVMSPFHLHLETTHPHPPPPPPYYYADQRDYAYGKEVGERAFFSDGAGERDRQQQAAGQWQFKQLGTMEATKPCPTPTPLLPAAGYGVGQAKEDEEEETRRQQQQHCFVLGADLRLAERPSGAHDDAAQKPLRHFFDEWPHEKGSKAGWWIGGLDGETTQLSMSIPMAAAADLPVTSRYRT

SEQ ID NO: 115 - protein, 39 aa, Artificial sequence
  QLQ domain
RPPFTPTQWEELEHQALIYKYMVSGVPVPPELIFSIRRS

SEQ ID NO: 116 - protein, 44 aa, Artificial sequence
  WRC domain
DPEPGRCRRTDGKKWRCSREAYPDSKYCEKHMHRGRNRARKSLD

SEQ ID NO: 117 - DNA, 2194 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctccttctcccatctataaattcctccccccttttcccctctctatataggaggcatccaagccaagaagagggagagcaccaaggacacgcgactagcagaagccgagcgaccgccttctcgatccatatcttccggtcgagttcttggtcgatctcttccctcctccacctcctcctcacagggtatgtgcctcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggatcggaatcttgcgattttgtgagtaccttttgtttgaggtaaaatcagagcaccggtgattttgcttggtgtaataaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc

SEQ ID NO: 118 - DNA, 58 bp, Artificial sequence
  primer prm10010
ggggacaagtttgtacaaaaaagcaggcttaaacaatgatgagtctaagtggaagtag

SEQ ID NO: 119 - DNA, 52 bp, Artificial sequence
  primer prm10011
ggggaccactttgtacaagaaagctgggtagctctacttaattagctaccag

SEQ ID NO: 120 - DNA, 633 bp, Arabidopsis thaliana
atgcaacagcacctgatgcagatgcagcccatgatggctggttactaccccagcaatgttacctctgatcatatccaacagtacttggacgaaaacaaatcgttgattctgaagattgttgagtctcaaaactctggaaagcttagcgaatgcgccgagaatcaagcaaggcttcaacgcaacctaatgtacctagctgcaatagcagattctcagcctcagccaccaagtgtgcatagccagtatggatctgctggtggtgggatgattcagggagaaggagggtcacactatttgcagcagcaacaagcgactcaacagcaacagatgactcagcagtctctaatggcggctcgatcttcaatgttgtatgctcagcaacagcggcagcagcagccttacgcgacgcttcagcatcagcaatcgcaccatagccagcttggaatgagctcgagcagcggaggaggaggaagcagtggtctccatatccttcagggagaggctggtgggtttcatgattttggccgtgggaagccggaaatgggaagtggtggtggcggtgaaggcagaggaggaagttcaggggatggtggagaaaccctttacttgaaatcatcagatgatgggaattga

SEQ ID NO: 121 - protein, 210 aa, Arabidopsis thaliana
MQQHLMQMQPMMAGYYPSNVTSDHIQQYLDENKSLILKIVESQNSGKLSECAENQARLQRNLMYLAAIADSQPQPPSVHSQYGSAGGGMIQGEGGSHYLQQQQATQQQQMTQQSLMAARSSMLYAQQQRQQQPYATLQHQQSHHSQLGMSSSSGGGGSSGLHILQGEAGGFHDFGRGKPEMGSGGGGEGRGGSSGDGGETLYLKSSDDGN

SEQ ID NO: 122 - DNA, 588 bp, Arabidopsis thaliana
atgcagcagcagcagtctccgcaaatgtttccgatggttccgtcgattccccctgctaacaacatcactaccgaacagatccaaaagtaccttgatgagaacaagaagctgattatggccatcatggaaaaccagaatctcggtaaacttgctgagtgcgcccagtaccaagctcttctccagaagaacttgatgtatcttgctgcaattgctgatgctcaacccccaccacctacgccaggaccttcaccatctacagctgtcgctgcccagatggcaacaccgcattctgggatgcaaccacctagctacttcatgcaacacccacaagcatcccctgcagggattttcgctccaaggggtcctttacagtttggtagcccactccagtttcaggatccgcaacagcagcagcagatacatcagcaagctatgcaaggacacatggggattagaccaatgggtatgaccaacaacgggatgcagcatgcgatgcaacaaccagaaaccggtcttggaggaaacgtggggcttagaggaggaaagcaagatggagcagatggacaaggaaaagatgatggcaagtga

SEQ ID NO: 123 - protein, 195 aa, Arabidopsis thaliana
MQQQQSPQMFPMVPSIPPANNITTEQIQKYLDENKKLIMAIMENQNLGKLAECAQYQALLQKNLMYLAAIADAQPPPPTPGPSPSTAVAAQMATPHSGMQPPSYFMQHPQASPAGIFAPRGPLQFGSPLQFQDPQQQQQIHQQAMQGHMGIRPMGMTNNGMQHAMQQPETGLGGNVGLRGGKQDGADGQGKDDGK

SEQ ID NO: 124 - DNA, 672 bp, Arabidopsis thaliana
atgcagcaatctccacagatgattccgatggttcttccttcatttccgcccaccaataatatcaccaccgaacagatccaaaagtatcttgatgagaacaagaagctgataatggcgatcttggaaaatcagaacctcggtaaacttgcagaatgtgctcagtatcaagctcttctccagaagaatttgatgtatctcgctgcaattgcggatgctcaacctcagccaccagcagctacactaacatcaggagccatgactccccaagcaatggctcctaatccgtcatcaatgcagccaccaccaagctacttcatgcagcaacatcaagctgtgggaatggctcaacaaatacctcctgggattttccctcctagaggtccattgcaatttggtagcccgcatcagtttctggatccgcagcaacagttacatcaacaagctatgcaagggcacatggggattagaccaatgggtttgaataataacaacggactgcaacatcaaatgcaccaccatgaaactgctcttgccgcaaacaatgcgggtcctaacgatgctagtggaggaggtaaaccggatgggaccaatatgagccagagtggagctgatgggcaaggtggctcagccgctagacatggcggtggtgatgcaaaaactgaaggaaaatga

SEQ ID NO: 125 - protein, 223 aa, Arabidopsis thaliana
MQQSPQMIPMVLPSFPPTNNITTEQIQKYLDENKKLIMAILENQNLGKLAECAQYQALLQKNLMYLAAIADAQPQPPAATLTSGAMTPQAMAPNPSSMQPPPSYFMQQHQAVGMAQQIPPGIFPPRGPLQFGSPHQFLDPQQQLHQQAMQGHMGIRPMGLNNNNGLQHQMHHHETALAANNAGPNDASGGGKPDGTNMSQSGADGQGGSAARHGGGDAKTEGK

SEQ ID NO: 126 - DNA, 627 bp, Allium cepa
atgcagcagccgcagccagcgatgggaaccatgggctcggtgccacctactagcatcaccaccgaacagattcaaaggtacttggatgagaacaaacagttaatattggcaattttggataatcaaaatttaggaagactgaatgagtgtgctcaatatcaagctcagcttcaaaagaatctgctttacctggcagcaatagctgatgctcagcctcagtctcctgcggtgcgtctgcagatgatgcctcaaggtgcagctgccacgcctcaagctggaaaccaatttatgcagcagcagagccctaatttccctcccaaaacaggaatgcaatttactcctcaacaagtacaagaattgcagcagcaacagctacaacatcagccacatatgatgcctccatttcaaggtcaaatgggtatgagacctatgaatggaatgcaggcagcaatgcatgcagattcatctcttgcttataacactaacaataagcaagatgcaggaaacgcagcttatgaaaatactgctgccaacacagatggttccattcaaaagaaaacagcaaatgatgatttagacccttctgcagcaaaccctagaaggtctgaagatgccaaatcatcatga

SEQ ID NO: 127 - protein, 208 aa, Allium cepa
MQQPQPAMGTMGSVPPTSITTEQIQRYLDENKQLILAILDNQNLGRLNECAQYQAQLQKNLLYLAAIADAQPQSPAVRLQMMPQGAAATPQAGNQFMQQQSPNFPPKTGMQFTPQQVQELQQQQLQHQPHMMPPFQGQMGMRPMNGMQAAMHADSSLAYNTNNKQDAGNAAYENTAANTDGSIQKKTANDDLDPSAANPRRSEDAKSS

SEQ ID NO: 128 - DNA, 633 bp, Aquilegia formosa x Aquilegia pubescens
atgcaacacatgcagatgcagcccatgatgccaccttatagtgccaacagcgtcactactgatcatatccaacagtacttggatgaaaataaggcgttgattctgaagatacttgagaaccaaaattcgggaaaagttagtgaatgtgcagagaaccaagcaagacttcaacgaaatcttatgtatctggctgcaattgctgattctcaaccacagcctcccaatatgcatgctcagtactctaatgcgggtataccacctggtgcacattacctacaacaccaacaggcccaacagatgacacaacagtcgctcatggctgctcgatcaaatatgctgtatgctcagccaatcacaggaatgcagcaacagcaagcaatgcatagccagcttggcatgagctctggtggtaacagtggactccacatgatgcacaatgagggcagcatgggaggtagtggggcacttggaagctattctgattatggccgtggcagtggtggtggagtaactatcgctagcaaacaagatggtggaagtggttctggtgaaggacgaggtggaaactctggaggccaaagtgcagatggaggtgaatctctttacctgaaaaacagtgacgaagggaactaa

SEQ ID NO: 129 - protein, 210 aa, Aquilegia formosa x Aquilegia pubescens
MQHMQMQPMMPPYSANSVTTDHIQQYLDENKALILKILENQNSGKVSECAENQARLQRNLMYLAAIADSQPQPPNMHAQYSNAGIPPGAHYLQHQQAQQMTQQSLMAARSNMLYAQPITGMQQQQAMHSQLGMSSGGNSGLHMMHNEGSMGGSGALGSYSDYGRGSGGGVTIASKQDGGSGSGEGRGGNSGGQSADGGESLYLKNSDEGN

SEQ ID NO: 130 - DNA, 668 bp, Aquilegia formosa x Aquilegia pubescens
atgcagcaacctccgccaatgatgaacatggtcccaccatttcctcctactaacattacaactgaacagattcaaaagtatctggatgagaacaaaacactgattttggcaatattagacaatcagaatcttggaaaattagccgaatgtgctcagtaccaagctcagcttcagaagaatctgatgtatcttgctgcaattgctgatgcccaaccgcaagctcctgcagttccctctcagatgccaacacatccggcaatgcaacagggaggacattatatgcaacatccgcaagcagctatgcctcaacagcccagtggttttccacccaagtcccccatgcaatttaaccctcagcaaatgcaagagcagcaacggctgcagctacaacagcaacaccaacaggcacttcaaggtcatatgggcattcgacctggagtcaacaatggtttgcaaatgcatggggatggtaatgttggaggcagcagcagcggtggcccatcatcaaccggtaacttacctgatttctcacgcagtggtgccggtcctggtgctggttctagttcattggatgcacgggaaggtaagcaagatggagtggaagcgggtgctggtgatggtcaaggcaattcagcagccagaaataatggttcaaatggggatacatg

SEQ ID NO: 131 - protein, 222 aa, Aquilegia formosa x Aquilegia pubescens
MQQPPPMMNMVPPFPPTNITTEQIQKYLDENKTLILAILDNQNLGKLAECAQYQAQLQKNLMYLAAIADAQPQAPAVPSQMPTHPAMQQGGHYMQHPQAAMPQQPSGFPPKSPMQFNPQQMQEQQRLQLQQQHQQALQGHMGIRPGVNNGLQMHGDGNVGGSSSGGPSSTGNLPDFSRSGAGPGAGSSSLDAREGKQDGVEAGAGDGQGNSAARNNGSNGDT

SEQ ID NO: 132 - DNA, 633 bp, Aspergillus officinalis
atgcagcagcacctgatgcagatgcagcccatgatggcaacctacggttcaccgaatcaggtcaccaccgatatcattcagcagtatctggacgagaacaagcagttgattctggctattcttgaaaaccaaaattcaggaaaagctgatgaatgtgctgagaatcaggctaagcttcagaggaatctgatgtatcttgcagccattgcggatagccagccccaagttcctaccattgctcagtatcctcccaacgctgttgctgctatgcaatcgagtgctcgctacatgcaacaacaccaagcagctcaacagatgacccctcaatctctcatggctgctcgctcctcaatgctctactcacagtccccaatgtctgcactccagcagcaacagcagcaagcagcaatgcatagccagctcgccatgagctccggaggcaacaacagcagcaccggaggattcaccattcttcatggtgaagctagcataggaggcaatggctcaatgaattctggtggagtctttggagattttggacggagcagcggtgggaagcaagagactgggagcgaagggcacgggacagagactcctatgtacctgaaaggctctgaagaagaaggaaactga

SEQ ID NO: 133 - protein, 210 aa, Aspergillus officinalis
MQQHLMQMQPMMATYGSPNQVTTDIIQQYLDENKQLILAILENQNSGKADECAENQAKLQRNLMYLAAIADSQPQVPTIAQYPPNAVAAMQSSARYMQQHQAAQQMTPQSLMAARSSMLYSQSPMSALQQQQQQAAMHSQLAMSSGGNNSSTGGFTILHGEASIGGNGSMNSGGVFGDFGRSSGGKQETGSEGHGTETPMYLKGSEEEGN

SEQ ID NO: 134 - DNA, 558 bp, Beta vulgaris
atgcagcaacaatcacctcaaatgttcaaccacccaccttcacaacccacaattactaccgaacaaattcaaaagtatcttgatgagaacaagcagttgattttggcaattatggaaagtcaaaactctggaaaaatgaatgaatgtgcccagtatcaagctcagctgcagaaaaacttgatgtacttggctgcaattgctgatgctcagccaccagcacctacagggccctctcagtctcagccgcagaattctcagatgcctatgcaatcgaccattccacaaggcccttttatgccgcctcctaaacctgcagttaccccacagcaaacaggtcccagattgccctttgctctacagtcgtttgatcagcagtcaccccatatgcaaatgcaataccaacagtccatggcaggctccatgggtatgagaatgggtgggaataatgttttacgcccttccatccagaccggatatggagctccaacacattttatggatgcacggaacagacaagatggttctgacgcaagtctcggtgatgatcatggaaagtaa

SEQ ID NO: 135 - protein, 185 aa, Beta vulgaris
MQQQSPQMFNHPPSQPTITTEQIQKYLDENKQLILAIMESQNSGKMNECAQYQAQLQKNLMYLAAIADAQPPAPTGPSQSQPQNSQMPMQSTIPQGPFMPPPKPAVTPQQTGPRLPFALQSFDQQSPHMQMQYQQSMAGSMGMRMGGNNVLRPSIQTGYGAPTHFMDARNRQDGSDASLGDDHGK

SEQ ID NO: 136 - DNA, 615 bp, Brachypodium distachyon
atgcagcaggcgatgtccatgtccccggggtcggccggcgcggtgccgcctccggccggcatcaccacagagcagatccaaaagtatttggatgaaaataagcaacttattttggccatcctggaaaatcagaacctaggaaagttgactgaatgtgctcagtatcaagctcaacttcagaagaatctcttgtatctggctgccattgcggatgcccaaccaccacagaaccctggaagtcgcccccagatggtgcagcctggtggtatgccaggtgcagggcattacatgtcgcaagtaccaatgttccctccaagaacccctttaaccccacaacagatgcaagagcaacagcaccagcagcttcagcagcagcaagcacaggctcttgctttccccagccagatggtcatgagaccaggtactgtgaacggcatgcagcctatgcaagctgatctccaagcagcagcagcagcacctggcctggcagacagccgaggaagtaagcaggacgcagcggtagctggggccatctcggaaccttctggcaccgagagtcacaagagtacaggagcggatcatgaggcaggtggcgatgtagctgagcaatcctaa

SEQ ID NO: 137 - protein, 204 aa, Brachypodium distachyon
MQQAMSMSPGSAGAVPPPAGITTEQIQKYLDENKQLILAILENQNLGKLTECAQYQAQLQKNLLYLAAIADAQPPQNPGSRPQMVQPGGMPGAGHYMSQVPMFPPRTPLTPQQMQEQQHQQLQQQQAQALAFPSQMVMRPGTVNGMQPMQADLQAAAAAPGLADSRGSKQDAAVAGAISEPSGTESHKSTGADHEAGGDVAEQS

SEQ ID NO: 138 - DNA, 591 bp, Brassica napus
atgcagcccatgatggctggttactaccccagcaatgtcacctctgatcatatccagcagtacttggatgagaacaagtctttgattctgaagatagttgagtctcaaaactcaggaaagctcagcgagtgtgccgagaatcaggcaaggcttcaacgcaacctcatgtacttggctgcaatagcagattctcagcctcaacctccaagcgtgcatagccagtatggatctgctggtggtgggttgattcagggagaaggagcgtcacactatttgcagcagcaacaggcgactcaacagcagcagatgactcagcagtctcttatggcagctcgttcttcaatgatgtatcagcagcagcaacagccttatgcaacgcttcagcatcagcagttgcaccatagccagcttgggatgagctctagcagcggaggaggaagcagtggtctccatatccttcagggagaggctggtgggtttcatgaatttggccgtgggaagccggagatgggaagtggtgaaggcaggggtggaagctcaggggatggtggagaaacactctacttgaagtcatcagatgatgggaactga

SEQ ID NO: 139 - protein, 203 aa, Brassica napus
MQQHLMQMQPMMAGYYPSNVTSDHIQQYLDENKSLILKIVESQNSGKLSECAENQARLQRNLMYLAAIADSQPQPPSVHSQYGSAGGGLIQGEGASHYLQQQQATQQQQMTQQSLMAARSSMMYQQQQQPYATLQHQQLHHSQLGMSSSSGGGSSGLHILQGEAGGFHEFGRGKPEMGSGEGRGGSSGDGGETLYLKSSDDGN

SEQ ID NO: 140 - DNA, 636 bp, Brassica napus
atgcagcagcagcagcagcagcagcagcagcctccgcaaatgtttccgatggctccttcgatgccgccaactaacatcaccaccgaacagatccaaaagtaccttgaggagaacaagaagctgataatggcaatcatggaaaatcagaatcttggcaagcttgcagagtgtgcacagtaccaagctcttctccagaagaacttaatgtacctcgctgctattgctgatgctcaacctcctccatctaccgctggagctacaccaccaccagctatggcttcccagatgggggcaccgcatcctgggatgcaaccgccgagctactttatgcaacacccacaagcttcagggatggctcaacaagcaccacccgctggtatcttccctccgagaggtcctttgcagtttggtagcccacaccagcttcaggatccgcaacagcagcatatgcatcaacaggctatgcaaggacacatggggatgcgaccaatgggtatcaacaacaacaatgggatgcagcatcagatgcagcaacaacaaccagaaacctctcttggaggaagcgctgcaaacgtggggcttagaggtggaaagcaagatggagcagatggacaaggaaaagatgatggcaaatga

SEQ ID NO: 141 - protein, 203 aa, Brassica napus; identical to SEQ ID NO: 139

SEQ ID NO: 142 - DNA, 552 bp, Chlamydomonas reinhardtii
atggcggcagcctcaaaacctccaccgatgacgaccgacaaaatacaagacatgctggaggagaatttcaagttcattaaagccattgccgagcagcaaaacttgggccgggttcaagaagtgcaccagtaccagcagaagctgcaggagaacctgatgctgctggccgcagtggcagacacctactccaactcagcagcagcagcacagccggggggagaagctggcgcagccgcacccgcggcggccacggcacgaccacccaccgcgcctggagcgccgctgggggcaccgggagcaccgccgcccgcggctccggctctcacaccacagcagatccacgcggccgtgcagcaggcactggccatgaagcagcagcagcagcagcagcagcagcaacagccgcagcagccgcagcagtcggcggtggcgcagtaccagcaaccgcctcaagcggggctgcccataccgggcgggatggcgcccgggcaaggcgcgccgccaggcttcacgctaccggcgccaccgcccctgaacctagccgggcagtag

SEQ ID NO: 143 - protein, 183 aa, Chlamydomonas reinhardtii
MAAASKPPPMTTDKIQDMLEENFKFIKAIAEQQNLGRVQEVHQYQQKLQENLMLLAAVADTYSNSAAAAQPGGEAGAAAPAAATARPPTAPGAPLGAPGAPPPAAPALTPQQIHAAVQQALAMKQQQQQQQQQQPQQPQQSAVAQYQQPPQAGLPIPGGMAPGQGAPPGFTLPAPPPLNLAGQ

SEQ ID NO: 144 - DNA, 663 bp, Citrus sinensis
atgcaacagcacctgatgcagatgcagcccatgatggcagcttattatcccaacaacgtcactactgaccacattcaacagtatctagatgagaacaaatcattgattttgaagattgttgagagccagaattcagggaaactgagcgagtgtgcagagaaccaggcaagattgcagcggaatctcatgtacctggctgctattgctgatgctcaaccccaaccacctagcgttcatgcccagttctcttctggtggcattatgcagccaggagctcactatatgcaacaccagcaatctcagccaatgacaccacagtcacttatggctgcacgctcatccatggtgtactctcaacagcaattttcagtgcttcagcaacagcaagccttgcatggtcagcttggcatgagctctggtggtagctcaggacttcacatgctgcaaagtgagggtagtactgcaggaggtagtggttcacttgggggtgggggattccctgattttggccgtggctcatctggtgaaggcttgcactcaaggggaatggggagcaagcatgatataggcagttctggatctgctgaaggacgaggagggagctcaggaagccaagatggaggcgaaactctctacttgaaaggggctgatgatggaaattaa

SEQ ID NO: 145 - protein, 219 aa, Citrus sinensis
MQQHLMQMQPMMAAYYPNNVTTDHIQQYLDENKSLILKIVESQNSGKLSECAENQARLQRNLMYLAAIADAQPQPPSVHAQFSSGGIMQPGAHYMQHQQSQPMTPQSLMAARSSMVYSQQQFSVLQQQQALHGQLGMSSGGSSGLHMLQSEGSTAGGSGSLGGGGFPDFGRGSSGEGLHSRGMGSKHDIGSSGSAEGRGGSSGSQDGGETLYLKGADDG

SEQ ID NO: 146 - DNA, 636 bp, Citrus sinensis
atgcagcagccaccgcaaatgatccctgttatgccttcatttccacccaccaacatcaccacagagcagattcaaaagtaccttgatgagaacaaaaagttgattttggcaattttggacaatcaaaatcttggaaagcttacagaatgtgcccactatcaagctcagcttcaaaagaatttaatgtatttagctgcaattgctgatgcacaaccacaagcaccaacaatgcctcctcagatggctccacatcctgcaatgcaagctagtgggtattacatgcaacatcctcaggcggcagcaatggctcagcaacaaggaatctttccccaaaagatgccattacaattcaataaccctcatcaactacaggatcctcaacagcagctacaccaacatcaagccatgcaagcacaaatgggaatgagaccgggtgccactaacaatggtatgcatcccatgcatgctgaaagctctcttggaggtggcagcagtggaggacccccttcagcatcaggcccaggtgacatacgtggtggaaataagcaagatgcctcggaggctgggactactggtgctgatggccagggcagttcggctggtgggcatggtggggatggagaggaggcaaagtga

SEQ ID NO: 147 - protein, 211 aa, Citrus sinensis
MQQPPQMIPVMPSFPPTNITTEQIQKYLDENKKLILAILDNQNLGKLTECAHYQAQLQKNLMYLAAIADAQPQAPTMPPQMAPHPAMQASGYYMQHPQAAAMAQQQGIFPQKMPLQFNNPHQLQDPQQQLHQHQAMQAQMGMRPGATNNGMHPMHAESSLGGGSSGGPPSASGPGDIRGGNKQDASEAGTTGADGQGSSAGGHGGDGEEAK

SEQ ID NO: 148 - DNA, 729 bp, Cryptomeria japonica
atgcagcagcatctcatgcaaatgcagccgatgatggcagcagcttacgcttctaacaacattaccactgatcacattcaaaagtacttggatgagaacaagcagttgatattagcaattatggacaatcaaaatctgggaaagcttaatgaatgtgcacagtaccaagcaaaacttcaacagaacttgatgtatctagctgctattgctgattctcagcctcaagttccggctgcacatgctcagattcctcctaatgcggttgtgcagtctggtgggcttttcatgcagcaccagcaagcacagcagcaagttactcctcagtctcttatggcagctagatcttccatgttgtatacccagcagccgatggctgctttgcatcaagcccagcagcagcagcaacaacaatctcttcacagccatcttggtataagttctggaggaagcaatggcttgcacatgttgcatggtgaagcaaacatgggaggtaacgggcctctctcatctggaggcttccctgacttttcacgtggaactggggcctctggtgaaggcattcaggccaataggggcatgtgtatagatcgtggtgcaaataagcatgatggcgctggaacggagaatgctcatccaggcccaggggatgggcgagggagttcgactggaggccagaatacagatgggtcagaacaatcatacctgaaagcctcagaagaggggaactag

SEQ ID NO: 149 - protein, 242 aa, Cryptomeria japonica
MQQHLMQMQPMMAAAYASNNITTDHIQKYLDENKQLILAIMDNQNLGKLNECAQYQAKLQQNLMYLAAIADSQPQVPAAHAQIPPNAVVQSGGLFMQHQQAQQQVTPQSLMAARSSMLYTQQPMAALHQAQQQQQQQSLHSHLGISSGGSNGLHMLHGEANMGGNGPLSSGGFPDFSRGTGASGEGIQANRGMCIDRGANKHDGAGTENAHPGPGDGRGSSTGGQNTDGSEQSYLKASEEGN

SEQ ID NO: 150 - DNA, 651 bp, Curcuma longa
atgcagcaatctccacattcgctagcccccatgtcagcagcccctgttgcgaatattacaacagaacaaattcaaaagtacttggatgagaataagcagctcattttggcaatattggaaaatcagaaccttgggaaattggctgaatgtgctcagtaccaagcgcagcttcagaaaaatctactttatcttgctgcaattgctgatgctcaacctaatgcacctgcagttcgtccccagcagatcatgccacacggtacgataccacagggaagccctttcatgcaacaatcacccatcttccctcgaggtcctcttccatataatcctcaacaaatgcaagggcagctacatccccaacccccaggaatggtgttcccaggccatatgggcattaggcccggcgctgtcaacggcttacatggctcgcatactgaaccatctcatggtggcactgctaatcccctcacaactccaagcttgtctggattcccaccaaccaactcagatggacgtgggagtaagcaagaagccggcatcgccatggtacctgccgtagctgagagccacaggaactcaggaagtgagcctgtcagtggggatgctgatcaatcacatgctaaaagaccagaggatacaaagacaccatga

SEQ ID NO: 151 - protein, 216 aa, Curcuma longa
MQQSPHSLAPMSAAPVANITTEQIQKYLDENKQLILAILENQNLGKLAECAQYQAQLQKNLLYLAAIADAQPNAPAVRPQQIMPHGTIPQGSPFMQQSPIFPRGPLPYNPQQMQGQLHPQPPGMVFPGHMGIRPGAVNGLHGSHTEPSHGGTANPLTTPSLSGFPPTNSDGRGSKQEAGIAMVPAVAESHRNSGSEPVSGDADQSHAKRPEDTKTP

SEQ ID NO: 152 - DNA, 597 bp, Euphorbia esula
atgcagcagcaaccgcagatgatgcctatgatgccttcatatccaccagcaaacattaccacggagcaaatccaaaagtatcttgatgaaaataaaaaattgattttggcgatcttggataatcaaaatcttggaaaactcgctgagtgtgcacagtatcaagccctgctgcaaaaaaatctgatgtatttagccgcaattgctgatgcacaaccccagaccccacccatgccacctcagatgtccccacatccggctatgcaacaaggagcatattacatgcaacatcctcaggctgcagcagcagcaatggctcatcagtcgggtattttcccaccaaagatgtctccgttacaattcaataatcctcatcaaatacaggacccccagcagttacatcaagcagccctccaagggcaaatgggaatgaggcccatggggcccaataacgggatgcatccgatgcaccccgaggcaaatcttggaggatctaatgatggtcgtggaggaaacaaacaggatgctccggagacgggagcatcgggaggtgatgggcaaggcaattctggtggtgatggggctgaagatgggaaatga

SEQ ID NO: 153 - protein, 198 aa, Euphorbia esula
MQQQPQMMPMMPSYPPANITTEQIQKYLDENKKLILAILDNQNLGKLAECAQYQALLQKNLMYLAAIADAQPQTPPMPPQMSPHPAMQQGAYYMQHPQAAAAAMAHQSGIFPPKMSPLQFNNPHQIQDPQQLHQAALQGQMGMRPMGPNNGMHPMHPEANLGGSNDGRGGNKQDAPETGASGGDGQGNSGGDGAEDGK

SEQ ID NO: 154 - DNA, 597 bp, Fragaria vesca
atgcagcagcagccacagcagatgatgcccaacatgacttcgcttcctcccaataccatcaccaccgagcaaattcagaagtgccttgatgagaacaaaaagttgattctagcaatattggacaatcaaaaccttggaaaacttgctgagtgtgcccagtaccaaactcagcttcaaaagaatctcatgtatttagcagcaattgctgatgcacaaccacaaccacaaccacaagcaccagcaatgccggcccagcagctggccccgcatcctgcgatgcaacaagctggatattacatgcagcatcctcaggctgcagcagcaatggctcagcaacagggtcttttccctcaaaagatgcaaatgcagtttaatagcccacaacaaatgcacgagatgcagcagcagttacaccaacaggccatgcatggccagatggggatgagacctggaggggccaatgggatgccttcaatgcatcatactgagaacacccatggaggaagcaagcaagacaactcagaggctggggcaggtggtgatggccaggggaactcagccggtggccacagaagtggcgacggagaggacaagtga

SEQ ID NO: 155 - protein, 198 aa, Fragaria vesca
MQQQPQQMMPNMTSLPPNTITTEQIQKCLDENKKLILAILDNQNLGKLAECAQYQTQLQKNLMYLAAIADAQPQPQPQAPAMPAQQLAPHPAMQQAGYYMQHPQAAAAMAQQQGLFPQKMQMQFNSPQQMHEMQQQLHQQAMHGQMGMRPGGANGMPSMHHTENTHGGSKQDNSEAGAGGDGQGNSAGGHRSGDGEDK

SEQ ID NO: 156 - DNA, 639 bp, Glycine max
atgcagcagcacctgatgcagatgcagcccatgatggctgcctactaccccaacaacgtcaccactgatcacattcaacagtacctggatgagaacaagtccttgattctgaagattgttgaaagccagaattctggcaagctgagcgagtgtgccgagaaccaatcaaggctgcagagaaatctcatgtacctagctgcaatagctgattctcaaccacaaccatctccattggctggtcagtatccttctagtggacttgtgcagcagggagcacactacatgcaggctcaacaggctcagcagatgtcacaacaacagctaatggcttcgcgctcctcgctcctgtactcccaacagcctttctcagtgcttcaacagcagcaaggcatgcacagccaacttggcatgagctccagtggaagtcaaggcctccacatgctgcaaagtgaagccactaatgttggaggcaatgcaaccataggaaccggaggagggtttccggactttgtacgcattggtagtggcaagcaagatattggaatctctggtgaaggcagaggaggaaactctagtggccactctggtgatggtggtgagacacttaattacctgaaagctgctggtgatggaaactga

SEQ ID NO: 157 - protein, 212 aa, Glycine max
MQQHLMQMQPMMAAYYPNNVTTDHIQQYLDENKSLILKIVESQNSGKLSECAENQSRLQRNLMYLAAIADSQPQPSPLAGQYPSSGLVQQGAHYMQAQQAQQMSQQQLMASRSSLLYSQQPFSVLQQQQGMHSQLGMSSSGSQGLHMLQSEATNVGGNATIGTGGGFPDFVRIGSGKQDIGISGEGRGGNSSGHSGDGGETLNYLKAAGDGN

SEQ ID NO: 158 - DNA, 633 bp, Glycine max
atgcagcagcacctgatgcagatgcagcccatgatggcaggctactaccccaacaacgtcaccactgatcacattcagcagtatctggatgagaacaagtccttaattctgaagattgttgaaagccagaattctggcaagctgagcgagtgtgccgagaaccaagcaaggcttcagagaaatctcatgtacttagctgcaatagctgattctcaaccccaaccacccaccatgtctggtcagtaccctccgagtgggatgatgcagcagggagcacagtacatgcaggctcaacaacaggcacagcagatgacaccacaacaactaatggcagcacgctcatctcttttgtacgcacagcagccgtactcagcacttcaacagcagcaagccatgcacagtgcactggggtcgagttcggggctccacatgctgcaaagtgaaggcagcaatgtgaatgtgggaggagggtttcctgactttgtgcgtggcggcagctccacaggggagggtttgcacagtggtggaaggggtatcattggaagtagcaagcaggaaatggggggttcaagtgaaggccgcggtgaagggggtgaaaacctctacctcaaagttgctgatgatggaaactag

SEQ ID NO: 159 - protein, 210 aa, Glycine max
MQQHLMQMQPMMAGYYPNNVTTDHIQQYLDENKSLILKIVESQNSGKLSECAENQARLQRNLMYLAAIADSQPQPPTMSGQYPPSGMMQQGAQYMQAQQQAQQMTPQQLMAARSSLLYAQQPYSALQQQQAMHSALGSSSGLHMLQSEGSNVNVGGGFPDFVRGGSSTGEGLHSGGRGIIGSSKQEMGGSSEGRGEGGENLYLKVADDGN

SEQ ID NO: 160 - DNA, 642 bp, Glycine max
atgcagcagacaccgccaatgattcctatgatgccttctttcccacctacgaacataaccaccgagcagattcaaaaataccttgatgagaacaagaagctgattctggcaatattggacaatcaaaatcttggaaaacttgcagaatgtgcccagtaccaagctcagcttcaaaagaatttgatgtatttagctgcaattgctgatgcccagcctcaaaccccggccatgcctccgcagatggcaccgcaccctgccatgcaaccaggattctatatgcaacatcctcaggctgctgcagcagcaatggctcagcagcagcaaggaatgttcccccagaaaatgccattgcaatttggcaatccacatcaaatgcaggaacaacaacagcagctacaccagcaggccatccaaggtcaaatgggacttagacctggagatataaataatggcatgcatccaatgcacagtgaggctgctcttggaggtggaaacagcggtggtccaccttcggctactggtccaaacgatgcacgtggtggaagcaagcaagatgcctctgaggctggaacagctggtggagacggccaaggcagctccgcggctgctcataacagtggagatggtgaagaggcaaagtga

SEQ ID NO: 161 - protein, 213 aa, Glycine max
MQQTPPMIPMMPSFPPTNITTEQIQKYLDENKKLILAILDNQNLGKLAECAQYQAQLQKNLMYLAAIADAQPQTPAMPPQMAPHPAMQPGFYMQHPQAAAAAMAQQQQGMFPQKMPLQFGNPHQMQEQQQQLHQQAIQGQMGLRPGDINNGMHPMHSEAALGGGNSGGPPSATGPNDARGGSKQDASEAGTAGGDGQGSSAAAHNSGDGEEAK

SEQ ID NO: 162 - DNA, 633 bp, Glycine max
atgcagcagacaccgcctatgattcctatgatgccttcgttcccacctacgaacataaccaccgagcagattcaaaaataccttgatgagaacaagaagctgattctggcaatattggacaatcaaaatcttggaaaacttgcagaatgtgcccagtaccaagctcagcttcaaaagaatttgatgtatttagctgcaattgctgatgcccagcctcaaacaccagccatgcctccacagatggcaccacaccctgccatgcaaccaggattctatatgcaacatcctcaggctgcagcagcagcaatggctcagcagcagcagcaaggaatgttcccccagaaaatgccattgcaatttggcaatccacatcaaatgcaggaacaacagcagcagctacaccagcaagccatccaaggtcaaatgggactgagacctggaggaataaataatggcatgcatccaatgcacaatgagggcggcaacagcggtggtccaccctcggctaccggtccgaacgacgcacgtggtggaagcaagcaagatgcttctgaggctggaacagctggtggagatggccaaggcagctctgcagctgctcataacagtggagatggtgaagaggcaaagtga

SEQ ID NO: 163 - protein, 210 aa, Glycine max
MQQTPPMIPMMPSFPPTNITTEQIQKYLDENKKLILAILDNQNLGKLAECAQYQAQLQKNLMYLAAIADAQPQTPAMPPQMAPHPAMQPGFYMQHPQAAAAAMAQQQQQGMFPQKMPLQFGNPHQMQEQQQQLHQQAIQGQMGLRPGGINNGMHPMHNEGGNSGGPPSATGPNDARGGSKQDASEAGTAGGDGQGSSAAAHNSGDGEEAK

SEQ ID NO: 164 - DNA, 633 bp, Glycine soya; identical to SEQ ID NO: 162

SEQ ID NO: 165 - protein, 210 aa, Glycine soya; identical to SEQ ID NO: 163

SEQ ID NO: 166 - DNA, 660 bp, Gossypium arboreum
  misc_feature (309)..(309): n is a, c, g, or t
atgcagcagcacctgatgcagatgcagcccatgatggcagcttattatcccaacaacgtcactactgatcatattcaacagtatctcgatgagaacaagtcattgatcttaaagattgttgagagccagaattctgggaaattgagtgaatgtgctgagaaccaagcaaggctgcagcgaaacctcatgtacctggctgccattgcggattctcaaccccaaccacccaccgtgcatgcacagtttccatctggtggtatcatgcagcaaggagctgggcactacatgcagcaccaacaagctcaacanatgacacaacagtcgcttatggctgctcggtcctcaatgttgtattctcagcaaccattttctgcactgcaacaacaacaacaacaaggctttgcacagtcagcttggcatgagctctggcgggagcacaggcctttcatatgctgcaaactgaatctagtactgcagggggcagtgagacaccttgggcccgagggttgtcctgatttggacgggggtcttttggagaggcatccctggtggcaggccaatggccgggggaacaaccaaaaatccggggaggccggctcacctaagggccgggaggagcccttggggcaggggggggtgatggggggaacctcttcttaa

SEQ ID NO: 167 - protein, 219 aa, Gossypium arboreum
  UNSURE (103)..(103): Xaa can be any naturally ocurring amino acid
MQQHLMQMQPMMAAYYPNNVTTDHIQQYLDENKSLILKIVESQNSGKLSECAENQARLQRNLMYLAAIADSQPQPPTVHAQFPSGGIMQQGAGHYMQHQQAQXMTQQSLMAARSSMLYSQQPFSALQQQQQQGFAQSAWHELWREHRPFICCKLNLVLQGAVRHLGPEGCPDLDGGLLERHPWWQANGRGNNQKSGEAGSPKGREEPLGQGGVMGGTSS

SEQ ID NO: 168 - DNA, 690 bp, Gossypium hirsutum
atgcagcagcacctgatgcagatgcagcccatgatggcagcttattatcccaacaacgtcactactgatcatattcaacagtatctcgatgagaacaagtcattgatcttaaagattgttgagagccagaattctgggaaattgagtgaatgtgctgagaaccaagcaaggctgcagcgaaacctcatgtacctggctgccattgcggattctcaaccccaaccacccaccgtgcatgcacagtttccatctggtggtatcatgcagccaggagctgggcactacatgcagcaccaacaagctcaacaaatgacacaacagtcgcttatggctgctcggtcctcaatgttgtattctcagcaaccattttctgcactgcaacaacaacagcagcaagctttgcacagtcagcttggcatgagctctggcggaagcacaggccttcatatgctgcaaactgaatctagtactgcaggtggcagtggagcacttggggccggagggtttcctgattttggacgtggttcttctggagaaggcatccatggtggcaggccaatggcaggtggaagcaagcaagatatcgggagtgccggctcagctgaaggtcgtggaggaagctctggtggtcagggtggtggtgatgggggtgaaaccctttacttaaaagcagccgatgatgggaactga

SEQ ID NO: 169 - protein, 229 aa, Gossypium hirsutum
MQQHLMQMQPMMAAYYPNNVTTDHIQQYLDENKSLILKIVESQNSGKLSECAENQARLQRNLMYLAAIADSQPQPPTVHAQFPSGGIMQPGAGHYMQHQQAQQMTQQSLMAARSSMLYSQQPFSALQQQQQQALHSQLGMSSGGSTGLHMLQTESSTAGGSGALGAGGFPDFGRGSSGEGIHGGRPMAGGSKQDIGSAGSAEGRGGSSGGQGGGDGGETLYLKAADDGN

SEQ ID NO: 170 - DNA, 642 bp, Gossypium hirsutum
atgccgcagccaccgcaaatgattcctgtgatgccttcatatccacctactaatatcactactgaacagattcagaagtaccttgatgagaataagaagttgattttggcaattttggacaatcagaatcttggaaaactcgctgaatgcgcccagtatcaagctcagctgcaaaagaatttgatgtatttagctgcaattgcggatgctcaacctcaatcaacgccagcaatgtcgcctcagatggcaccgcatccagcaatgcaacccggaggatattttatgcaacatcctcaagctgctgcaatgtcacagcaacctggcatgtaccctcaaaaggtgccattgcaattcaatagtccgcatcaaatgcaggaccctcagcacctcctatatcagcagcatcaacaagcaatgcaaggtcaaatgggaatcaggcctgggggacccaataatagcatgcatcccatgcattcagaggctagccttggaggcggcagcagtggtggtccccctcaaccttcaggcccaagtgatggacgtgctggaaacaagcaagagggctccgaagctggtggtaatgggcagggcagcacaactggtgggcatggtggcggtgatggagcggatgaggcaaagtga

SEQ ID NO: 171 - protein, 213 aa, Gossypium hirsutum
MPQPPQMIPVMPSYPPTNITTEQIQKYLDENKKLILAILDNQNLGKLAECAQYQAQLQKNLMYLAAIADAQPQSTPAMSPQMAPHPAMQPGGYFMQHPQAAAMSQQPGMYPQKVPLQFNSPHQMQDPQHLLYQQHQQAMQGQMGIRPGGPNNSMHPMHSEASLGGGSSGGPPQPSGPSDGRAGNKQEGSEAGGNGQGSTTGGHGGGDGADEAK

SEQ ID NO: 172 - DNA, 630 bp, Helianthus annuus
atgcagcagcacctgatgcagatgcagcccatgatggcagcctattatcccaccaacaacgtcactactgatcatattcaacagtacttggatgaaaacaagtctctgatcttgaagattgttgagagccaaaactctgggaaaatggctgaatgtgcagaacatcaggccaagcttcagagaaaccttatgtaccttgctgcaattgctgattctcaacctcaagcacctagtcttcactctcagtatcctcaaggtgggatgatgcagcagcaggctggaagtcactacatgcagcagcaccaacaggcacaacagatgtcaccacaagcactcatggctgcacgctcatccatgatgtacagtcagcagcagtactcttcactacagcagcaagcaatgcatagccatctgggcatgagttctggaactggaaccagtggacttcacatgctgcagaccgacaataatagcgcgggtgtcagtgggacccacctaagtggtgggttccccgactttggtcgtaagcaagatattggtcccaccggtgaggggcggggtggtggtagctctggcggtggagacggtggcgagacgctctacttgaagtcgcctgataaaggtaactga

SEQ ID NO: 173 - protein, 209 aa, Helianthus annuus
MQQHLMQMQPMMAAYYPTNNVTTDHIQQYLDENKSLILKIVESQNSGKMAECAEHQAKLQRNLMYLAAIADSQPQAPSLHSQYPQGGMMQQQAGSHYMQQHQQAQQMSPQALMAARSSMMYSQQQYSSLQQQAMHSHLGMSSGTGTSGLHMLQTDNNSAGVSGTHLSGGFPDFGRKQDIGPTGEGRGGGSSGGGDGGETLYLKSPDKGN

SEQ ID NO: 174 - DNA, 561 bp, Hordeum vulgare
atgcagcaagcgatgcccatgccgccggcggcggcggcgcctgggatgcctccttctgccggcctcagcaccgagcagatccaaaagtacctggatgaaaataaacaactaattttggctatcttggaaaatcagaacctgggaaagttggcggaatgtgctcagtatcaagctcagcttcagaagaatcttttgtatttggctgcgattgctgatactcagccacagacctctgtaagccgtcctcagatggcaccacctgctgcatccccaggggcagggcattacatgtcacaggtgccaatgttccctccgaggacccctctaacgcctcagcagatgcaggagcagcaactacagcaacaacaggctcagatgcttccgtttgctggtcaaatggttgcgagacccggggctgtcaatggcattccccaggcccctcaagttgaacaaccagcctatgcagcaggtggggccagttccgagccttctggcaccgagagccacaggagcactggcgccgataacgatggtgggagcggcttggctgaccagtcctaa

SEQ ID NO: 175 - protein, 186 aa, Hordeum vulgare
MQQAMPMPPAAAAPGMPPSAGLSTEQIQKYLDENKQLILAILENQNLGKLAECAQYQAQLQKNLLYLAAIADTQPQTSVSRPQMAPPAASPGAGHYMSQVPMFPPRTPLTPQQMQEQQLQQQQAQMLPFAGQMVARPGAVNGIPQAPQVEQPAYAAGGASSEPSGTESHRSTGADNDGGSGLADQS

SEQ ID NO: 176 - DNA, 555 bp, Lactuca serriola
  misc_feature (253)..(253): n is a, c, g, or t
atgaagcagccgatgatgccgaatccaatgatgtcttcttcgtttcctcctacaaacatcaccaccgatcagatccaaaagttccttgatgaaaacaagcaactaattatagcaataatgagcaacctaaatcttggaaagcttgctgaatgtgcccagtaccaagctctactccaaaaaaatttgatgtatctagcagccattgcagatgctcaaccacctacacctacaccaacactaaatatctcttatnagatgggcccggttccacatccagggatgccacagcaaggtggattttacatggcgcagcagcaccctcaggcggctgtaatgacggctcagccaccttctggttttccacaaccgatgcctggtatgcaatttaacagcccacaggctattcaagggcagatgggcgggaggtccggtgggccgccaagctcagccgctagtgatgtctggagaggaagcatgcaagatggtggtggtggtgctgctgctgatggtggtaaggatggtcatgctggcggtggacctgaggaagcaaagtaa

SEQ ID NO: 177 - protein, 184 aa, Lactuca serriola
  UNSURE (85)..(85): Xaa can be any naturally ocurring amino acid
MKQPMMPNPMMSSSFPPTNITTDQIQKFLDENKQLIIAIMSNLNLGKLAECAQYQALLQKNLMYLAAIADAQPPTPTPTLNISYXMGPVPHPGMPQQGGFYMAQQHPQAAVMTAQPPSGFPQPMPGMQFNSPQAIQGQMGGRSGGPPSSAASDVWRGSMQDGGGGAAADGGKDGHAGGGPEEAK

SEQ ID NO: 178 - DNA, 627 bp, Lycopersicon esculentum
atgcagcagcacctgatgcagatgcagcccatgatggcagcttactatccaacgaacgtcactactgaccatattcaacagtatttggatgaaaacaaatcactcattctgaagattgttgagagccagaactctgggaaactcagtgaatgtgcggagaaccaagctaggcttcagaggaatctgatgtaccttgctgcgattgctgattcacaacctcaaccttctagcatgcattctcagttctcttctggtgggatgatgcagccagggacacacagttacttgcagcagcagcagcagcaacaacaagcgcaacaaatggcaacacaacaactcatggctgcaagatcctcgtcgatgctctatggacaacagcagcagcaatctcagttatcgcaatatcaacaaggcttgcatagtagccaactcggcatgagttctggcagtggcggaagcactggacttcatcacatgcttcaaagtgaatcatcacctcatggtggtggtttctctcatgacttcggccgcgcaaataagcaagacattgggagtagtatgtctgctgaagggcgcggcggaagttcaggtggtgagaatctttatctgaaagcttctgaggattga

SEQ ID NO: 179 - protein, 208 aa, Lycopersicon esculentum
MQQHLMQMQPMMAAYYPTNVTTDHIQQYLDENKSLILKIVESQNSGKLSECAENQARLQRNLMYLAAIADSQPQPSSMHSQFSSGGMMQPGTHSYLQQQQQQQQAQQMATQQLMAARSSSMLYGQQQQQSQLSQYQQGLHSSQLGMSSGSGGSTGLHHMLQSESSPHGGGFSHDFGRANKQDIGSSMSAEGRGGSSGGENLYLKASED

SEQ ID NO: 180 - DNA, 624 bp, Malus domestica
atgcagcagccaccacaaatgatccccgtcatgccttcatttcctcccaccaacatcaccaccgaacaaattcagaagtaccttgatgacaacaaaaagttgattctggcaatattggataatcaaaatcttggaaaacttgctgagtgtgctcagtaccaggctctgcttcaaaagaatctgatgtatttagcagcaattgccgatgcgcaaccacaggcaccagctgcccctccccagatggccccacatcctgctatgcaacaggcaggatattacatgcaacatcctcaggcagcagcaatggctcagcaacagggtattttctccccaaagatgccgatgcaattcaataacatgcatcaaatgcacgatccacagcagcaccaacaagccatgcaagggcaaatgggaatgagacctggagggcctaacggcatgccttccatgcttcatactgaggccacacatggtggtggtagtggcggcccaaattcagctggagacccaaatgatgggcgtggaggaagcaagcaagacgcctctgagtctggggcaggtggtgatggccaggggacctcagccggcgggcgtggaactggtgatggagaggacggcaagtga

SEQ ID NO: 181 - protein, 207 aa, Malus domestica
MQQPPQMIPVMPSFPPTNITTEQIQKYLDDNKKLILAILDNQNLGKLAECAQYQALLQKNLMYLAAIADAQPQAPAAPPQMAPHPAMQQAGYYMQHPQAAAMAQQQGIFSPKMPMQFNNMHQMHDPQQHQQAMQGQMGMRPGGPNGMPSMLHTEATHGGGSGGPNSAGDPNDGRGGSKQDASESGAGGDGQGTSAGGRGTGDGEDGK

SEQ ID NO: 182 - DNA, 636 bp, Medicago trunculata
atgcagcagcacctgatgcagatgcagcccatgatggcagcttactatcctaacaacgtcactactgatcatattcaacagtatcttgatgagaacaagtccttgattctcaagattgttgaaagccagaacactggcaagctcaccgagtgtgctgagaaccaatcaaggcttcagagaaatctcatgtacctagctgcaatagctgattctcaaccccaaccacctactatgcctggccagtacccttcaagtggaatgatgcagcagggaggacactacatgcaggctcaacaagctcagcagatgacacaacaacaattaatggctgcacgttcctctcttatgtatgctcaacagcttcaacagcagcaagccttgcaaagccaacttggtatgaattccagtggaagtcaaggccttcacatgttgcatagtgaaggggctaatgttggaggcaattcatctctaggggctggttttcctgattttggccgtagctcagccggtgatggtttgcacggcagtggtaagcaagacattggaagcactgatggccgcggtggaagctctagtggtcactctggtgatggcggcgaaacactttacctgaaatcttctggtgatgggaattag

SEQ ID NO: 183 - protein, 211 aa, Medicago trunculata
MQQHLMQMQPMMAAYYPNNVTTDHIQQYLDENKSLILKIVESQNTGKLTECAENQSRLQRNLMYLAAIADSQPQPPTMPGQYPSSGMMQQGGHYMQAQQAQQMTQQQLMAARSSLMYAQQLQQQQALQSQLGMNSSGSQGLHMLHSEGANVGGNSSLGAGFPDFGRSSAGDGLHGSGKQDIGSTDGRGGSSSGHSGDGGETLYLKSSGDGN

SEQ ID NO: 184 - DNA, 639 bp, Medicago trunculata
atgcagcagacacctcaaatgattcctatgatgccttcattcccacaacaaacaaacataaccactgagcagattcaaaaatatcttgatgagaacaagaagctgatcctggcaatattggacaatcaaaatcttggaaaacttgcagaatgtgcccagtaccaagctcagcttcagaagaatttgatgtatttagctgcaattgctgacgcgcagccacaaacaccggccttgcctccacagatggccccgcaccctgcgatgcaacaaggattctatatgcaacatcctcaggctgcagcaatggctcagcaacaaggaatgttcccccaaaaaatgccaatgcagttcggtaatccgcatcaaatgcaggatcagcagcatcagcagcaacaacagcagctacatcagcaagctatgcaaggtcaaatgggacttagacctggagggataaataacggcatgcatccaatgcacaacgaggctgctctcggaggtagcggcagtggtggtcaaatgacgggcgtggtggtggagcaagcaagatgcttcggagctgggacagccggcggtgatggtcaaggaacctctgccgcagctgcgcacaacagtggagatgcttcagaagaaggaaagtaa

SEQ ID NO: 185 - protein, 213 aa, Medicago trunculata
MQQTPQMIPMMPSFPQQTNITTEQIQKYLDENKKLILAILDNQNLGKLAECAQYQAQLQKNLMYLAAIADAQPQTPALPPQMAPHPAMQQGFYMQHPQAAAMAQQQGMFPQKMPMQFGNPHQMQDQQHQQQQQQLHQQAMQGQMGLRPGGINNGMHPMHNEAALGGSGSGGPNDGRGGGSKQDASEAGTAGGDGQGTSAAAAHNSGDASEEGK

SEQ ID NO: 186 - DNA, 684 bp, Oryza sativa
atgcagcagcaacacctgatgcagatgaaccagggcatgatggggggatatgcttcccctaccaccgtcaccactgatctcattcagcagtatctggatgagaacaagcagctgatcctggccatccttgacaaccagaacaatgggaaggtggaagagtgcgctcggaaccaagctaagctccagcacaatctcatgtacctcgccgccatcgccgacagccagccgccgcagacggccgccatgtcccagtatccgtcgaacctgatgatgcagtccggggcgaggtacatgccgcagcagtcggcgcagatgatggcgccgcagtcgctgatggcggcgaggtcttcgatgatgtacgcgcagccggcgctgtcgccgctccagcagcagcagcagcagcaggcggcggcggcgcacgggcagctgggcatgggctcggggggcaccaccagcgggttcagcatcctccacggcgaggccagcatgggcggcggcggcggcggcggtggcgccggtaacagcatgatgaacgccggcgtgttctccgacttcggacgcggcggcggcggcggcggcaaggaggggtccacctcgctgtccgtcgacgtccggggcgccaactccggcgcccagagcggcgacggggagtacctcaagggcaccgaggaggaaggcagctag

SEQ ID NO: 187 - protein, 227 aa, Oryza sativa
MQQQHLMQMNQGMMGGYASPTTVTTDLIQQYLDENKQLILAILDNQNNGKVEECARNQAKLQHNLMYLAAIADSQPPQTAAMSQYPSNLMMQSGARYMPQQSAQMMAPQSLMAARSSMMYAQPALSPLQQQQQQQAAAAHGQLGMGSGGTTSGFSILHGEASMGGGGGGGGAGNSMMNAGVFSDFGRGGGGGGKEGSTSLSVDVRGANSGAQSGDGEYLKGTEEEGS

SEQ ID NO: 188 - DNA, 558 bp, Oryza sativa
atgcagcagcagccgatgccgatgcccgcgcaggcgccgccgacggccggaatcaccaccgagcagatccaaaagtatctggatgaaaacaagcagcttattttggctattttggaaaatcagaatctgggaaagttggcagaatgtgctcagtatcaagcgcagcttcagaagaatctcttgtacttggctgcaattgctgatactcaaccgcagaccactataagccgtccccagatggtgccgcatggtgcatcgccggggttaggggggcaatacatgtcgcaggtgccaatgttcccccccaggacccctctaacgccccagcagatgcaggagcagcagctgcagcaacagcaagcccagctgctctcgttcggcggtcagatggttatgaggcctggcgttgtgaatggcattcctcagcttctgcaaggcgaaatgcaccgcggagcagatcaccagaacgctggcggggccacctcggagccttccgagagccacaggagcaccggcaccgaaaatgacggtggaagcgacttcggcgatcaatcctaa

SEQ ID NO: 189 - protein, 185 aa, Oryza sativa
MQQQPMPMPAQAPPTAGITTEQIQKYLDENKQLILAILENQNLGKLAECAQYQAQLQKNLLYLAAIADTQPQTTISRPQMVPHGASPGLGGQYMSQVPMFPPRTPLTPQQMQEQQLQQQQAQLLSFGGQMVMRPGVVNGIPQLLQGEMHRGADHQNAGGATSEPSESHRSTGTENDGGSDFGDQS

SEQ ID NO: 190 - DNA, 618 bp, Oryza sativa
atgcagcagcagatggccatgccggcgggggccgccgccgccgcggtgccgccggcggccggcatcaccaccgagcagatccaaaagtatttggatgaaaataaacagctaattttggccatcctggaaaatcaaaacctagggaagttggctgaatgtgctcagtaccaagctcagcttcaaaagaatctcttgtatctggctgccattgcagatgcccaaccacctcagaatccaggaagtcgccctcagatgatgcagcctggtgctaccccaggtgctgggcattacatgtcccaagtaccgatgttccctccaagaactcccttaaccccacaacagatgcaagagcagcagcagcagcaactccagcaacagcaagctcaggctctagccttccccggccagatgctaatgagaccaggtactgtcaatggcatgcaatctatcccagttgctgaccctgctcgcgcagccgatcttcagacggcagcaccgggctcggtagatggccgaggaaacaagcaggatgcaacctcggagccttccgggaccgagagccacaagagtgcgggagcagataacgacgcaggcggtgacatagcggagaagtcctga

SEQ ID NO: 191 - protein, 205 aa, Oryza sativa
MQQQMAMPAGAAAAAVPPAAGITTEQIQKYLDENKQLILAILENQNLGKLAECAQYQAQLQKNLLYLAAIADAQPPQNPGSRPQMMQPGATPGAGHYMSQVPMFPPRTPLTPQQMQEQQQQQLQQQQAQALAFPGQMLMRPGTVNGMQSIPVADPARAADLQTAAPGSVDGRGNKQDATSEPSGTESHKSAGADNDAGGDIAEKS

SEQ ID NO: 192 - DNA, 624 bp, Panicum virgatum
atgcagcagcagatgcccatgcagtcggcgcccccggcgaccggcatcaccaccgagcagatccaaaagtatttggatgaaaataagcagcttattttggccatcctggaaaatcagaacttaggaaagttggctgaatgtgctcagtatcaagctcagcttcaaaagaatctcttgtacctggctgcgattgcagatgcccaaccccaaccaccacagaaccctgcaagtcgcccacagatgatgcaacctggcatggtaccaggtgcagggcattacatgtcccaagtaccaatgttcccgccaagaacaccattaaccccgcaacagatgcaagaacagcagcagcagcagcagcagcttcaacagcagcaagcacaggctcttgctttcccgggacagatggtcatgagacctaccattaatggcatgcagcctatgcaagccgaccctgctgccgccgccgccagcctacagcagtcagcacctggccctactgatgggcgaggaggcaagcaagatgcaactgctggggtgagcacagagccttctggcaccgagagccacaagagcacaaccgcagcagatcacgatgtgggcactgatgtcgcggagaaatcctaa

SEQ ID NO: 193 - protein, 207 aa, Panicum virgatum
MQQQMPMQSAPPATGITTEQIQKYLDENKQLILAILENQNLGKLAECAQYQAQLQKNLLYLAAIADAQPQPPQNPASRPQMMQPGMVPGAGHYMSQVPMFPPRTPLTPQQMQEQQQQQQQLQQQQAQALAFPGQMVMRPTINGMQPMQADPAAAAASLQQSAPGPTDGRGGKQDATAGVSTEPSGTESHKSTTAADHDVGTDVAEKS

SEQ ID NO: 194 - DNA, 645 bp, Physcomitrella patens
atgcagcaaatggcggcgtatacggggacgtctattaccacggagctaatccagaagtatctggacgagaacaagcagcttatcctcgcgattcttgacaatcaaaaccttggaaagctgaacgaatgcgctatgtatcaagcaaagttgcagcagaatcttatgtacctcgcagccattgcggatgcacaacctcaagtgagccaaaactcaactcaggtctcatcaggacaatctatgcagccctctcagcaatatattcaacagcagcagcagcagcagatgatgatgatgaatcagcgaaactcaatcccacaatacatgcaacaaagccaacaggggtcaccaaacgcaccatcaccgcagcagcagtcctaccacagccagcagccgcaaggtatggttccccagagaaactcagacatgcacttggtgaaaaactctacaggaggcaacggtaatcagacaggaggtagtgtatccgagtatggaaagcctgaggaatcccgggaggggaccccaacaagcttaagcacaagaaacgatggtccacaggcgggggcttctccgttgggacaagcgagagaaggcaatggagctgctggagaggactctgaggcttcttacttgaaaagctccgactaa

SEQ ID NO: 195 - protein, 214 aa, Physcomitrella patens
MQQMAAYTGTSITTELIQKYLDENKQLILAILDNQNLGKLNECAMYQAKLQQNLMYLAAIADAQPQVSQNSTQVSSGQSMQPSQQYIQQQQQQQMMMMNQRNSIPQYMQQSQQGSPNAPSPQQQSYHSQQPQGMVPQRNSDMHLVKNSTGGNGNQTGGSVSEYGKPEESREGTPTSLSTRNDGPQAGASPLGQAREGNGAAGEDSEASYLKSSD

SEQ ID NO: 196 - DNA, 609 bp, Physcomitrella patens
atgcagcaaatggcgccgtatgcggggacatctatcactactgagctcatccagaagtacctggacgagaacaagcagctgattctcgcaattctcgataatcaaaaccttggaaagctgaacgaatgtgctacgtatcaagcaaagttgcagcagaatcttatgtacctcgcagctattgcagacgcacaaccccaagttccagcagcacaaccaatgcaaccatcacagcaatatattcagcagcagcagcagcagatgatgatgaatcaacgaaatcagtacttgcagcaaaatcaacaaggagtacaaaacgcaccatcaccgcagtcgcagcagtcgtatcacaaccagcagccaggcatggtctcccagggaaactcggggatgcacatggtgaatagttccatgggtggtaatggcaaccagacaggaggaaatgtttccgagtatgggaaaccagaagattcccgggaggggactccaacaagcttgaatacaaggaatgaaggtccacaagcgggggcttccccactgggacaagcaagagaagggaatggtgcgccaggagaggattcagaggcttcatacttaaaaagctccgagtga

SEQ ID NO: 197 - protein, 202 aa, Physcomitrella patens
MQQMAPYAGTSITTELIQKYLDENKQLILAILDNQNLGKLNECATYQAKLQQNLMYLAAIADAQPQVPAAQPMQPSQQYIQQQQQQMMMNQRNQYLQQNQQGVQNAPSPQSQQSYHNQQPGMVSQGNSGMHMVNSSMGGNGNQTGGNVSEYGKPEDSREGTPTSLNTRNEGPQAGASPLGQAREGNGAPGEDSEASYLKSSE

SEQ ID NO: 198 - DNA, 654 bp, Physcomitrella patens
atgatgcagcacatggcgacgtatgccagctccaacatcacaacggagctcattcagaagtacttggacgagaataagcagttgattctcgctatcctcgacaaccaaaacctcggcaagctcaatgagtgtgcaacgtatcaagcgaagttgcagcagaatctcatgtatttggctgccatagctgatgctcagccacaaggcccatcttcgcagatgccagcacctgcgccggcgccaaccatgcaaccagctcagcagtacatgcaacagcagcaacaactccggatgatgagccaacagaacagtatgatcccttcctaccttcagcacagccaacaagcatcgcagcagaacttctacagtcaacaaagcctgctctccgggggagggggctcaatacacatgatgtccacggaccgcggcatcggaggcaatggatcccaggcttcgccaggatattctgatggagggcgggatcagagccaaatgggtttggcaatgcagggcgacatgcatggtggaaacgggacggacctggggtgctcctccccgcttggccaccgaggagacggtggtaacggccacggccaggggaccgatgactctgaagcttcctacttgaagggatctgatgggagcagtctgaattaa

SEQ ID NO: 199 - protein, 217 aa, Physcomitrella patens
MMQHMATYASSNITTELIQKYLDENKQLILAILDNQNLGKLNECATYQAKLQQNLMYLAAIADAQPQGPSSQMPAPAPAPTMQPAQQYMQQQQQLRMMSQQNSMIPSYLQHSQQASQQNFYSQQSLLSGGGGSIHMMSTDRGIGGNGSQASPGYSDGGRDQSQMGLAMQGDMHGGNGTDLGCSSPLGHRGDGGNGHGQGTDDSEASYLKGSDGSSLN

SEQ ID NO: 200 - DNA, 648 bp, Physcomitrella patens
atgatgcagcacatgacgacctatgccagctccaacatcaccacggagctcattcagaagtacttggacgagaataaacagctgattctcgccattctcgacaaccaaaacctcggcaagctcaatgagtgtgcgacgtatcaagcgaagctgcagcagaacctcatgtatctggccgctatagctgatgcccagccacaaggcccatctacgcagatgccagcgcccgcgccaactatgcaaccagctcaacaatacatgcaacagcagcaacagctccgcatgatgagccaacaaaatgccatgatcccctcctacctgcagcaaagccaacaagtttcccagcagaacttctacagccaacagagcctgcttaccggcggtggcagctccatccacatgatgtccactgatcgcggcatgggaggcaatgggtcacaagcctcacctggatattctgacggagggcgagatcagaaccaattgggtatgacgatgcagggcgacatgcatggtggaaacggcactgacttgggctgctcctcacctctcggccaccggggagatggcggtggcggccacggccagggcaacgacgactctgaagcttcttacttgaagggttccgatggcagcagtctgaactag

SEQ ID NO: 201 - protein, 215 aa, Physcomitrella patens
MMQHMTTYASSNITTELIQKYLDENKQLILAILDNQNLGKLNECATYQAKLQQNLMYLAAIADAQPQGPSTQMPAPAPTMQPAQQYMQQQQQLRMMSQQNAMIPSYLQQSQQVSQQNFYSQQSLLTGGGSSIHMMSTDRGMGGNGSQASPGYSDGGRDQNQLGMTMQGDMHGGNGTDLGCSSPLGHRGDGGGGHGQGNDDSEASYLKGSDGSSLN

SEQ ID NO: 202 - DNA, 747 bp, Picea sitchensis
atgcagcagcatctcatgcaaatgcagcccatgatggcggcatacgcctccaacaacatcaccactgatcacatccagaagtacctggatgagaacaagcagttgattctggcaattctggacaaccaaaatcttggaaagctcaatgagtgtgctcagtaccaagcaaaacttcagcagaatttgatgtatctggctgcgattgctgattctcaaccacaagcacaaactgcacatgctcagattcctcctaatgcagtgatgcagtctggtgggcattacatgcagcaccagcaggcacagcaacaagtgactcctcagtctctgatggcagctagatcttccatgctgtattctcagcagccgatggctgctttgcatcaagctcagcaacaacagcagcagcagcatcagcagcaacaacaatctcttcacagccagcttggcataaattctggaggaagcagtggattgcatatgttgcatggtgagacaaacatgggatgtaatgggcctctctcatctgggggcttccctgaatttgggcgtgggtctgctacctctgctgaaggtatgcaggccaacaggggcttcactatagatcgtggttcaaataagcaggatggagtaggatcagagaatgcccatccaggtgctggtgatggaagagggagttcaactggagggcagaatgcagatgagtcagaaccatcatacctgaaagcctccgaagaagaaggaaactag

SEQ ID NO: 203 - protein, 248 aa, Picea sitchensis
MQQHLMQMQPMMAAYASNNITTDHIQKYLDENKQLILAILDNQNLGKLNECAQYQAKLQQNLMYLAAIADSQPQAQTAHAQIPPNAVMQSGGHYMQHQQAQQQVTPQSLMAARSSMLYSQQPMAALHQAQQQQQQQHQQQQQSLHSQLGINSGGSSGLHMLHGETNMGCNGPLSSGGFPEFGRGSATSAEGMQANRGFTIDRGSNKQDGVGSENAHPGAGDGRGSSTGGQNADESEPSYLKASEEEGN

SEQ ID NO: 204 - DNA, 735 bp, Pinus taeda
atgcagcagcacctcatgcaaatgcagcccatgatggcggcctacgcctccaacaatatcaccactgatcacatccagaagtacctggatgagaacaagcagttgattctggcaattttggacaaccaaaatctcggaaagctcaatgagtgtgctcaataccaagcaaaacttcagcagaatttgatgtatctggctgctattgctgattctcaacctcaagcacaaactgcacatgctcagattcctccaaatgcggtgatgcagtctggtgggcattacatgcagcatcaacaggcacagcaacaagttactcctcagtctctgatggcagctagatcttccatactgtatgctcagcaacaacagcagcagcagcatcagcagcatcagcagcaacagcagcaacaacagtctcttcacagccagcttggcataaattctggaggaagcagcggtttgcatatgttgcatggtgagacaaacatgggatgtaatgggcctctgtcatctgggggattccctgaatttgggcgtgggtctgctacctctgctgatggtatgcaggtgaacaggggctttgctatagatcgtggttcaaacaagcaggatggagttggatcagagaatgcccatgctggtgctggtgatggaagagggagttcaactggagggcagaatgcagatgagtcagaaccatcatacctgaaggcctccgaggaagaaggaaactag

SEQ ID NO: 205 - protein, 244 aa, Pinus taeda
MQQHLMQMQPMMAAYASNNITTDHIQKYLDENKQLILAILDNQNLGKLNECAQYQAKLQQNLMYLAAIADSQPQAQTAHAQIPPNAVMQSGGHYMQHQQAQQQVTPQSLMAARSSILYAQQQQQQQHQQHQQQQQQQQSLHSQLGINSGGSSGLHMLHGETNMGCNGPLSSGGFPEFGRGSATSADGMQVNRGFAIDRGSNKQDGVGSENAHAGAGDGRGSSTGGQNADESEPSYLKASEEEGN

SEQ ID NO: 206 - DNA, 663 bp, Populus trichocarpa
atgcaacagcacctgatgcagatgcagcccatgatggcagcctattaccccagcaacgtcactactgatcatattcaacagtatctggacgaaaacaagtcattgattttgaagattgttgagagccagaattcagggaaactcagtgagtgtgcagagaaccaagcaagactgcaacaaaatctcatgtacttggctgcaattgctgattgtcagccccaaccacctaccatgcatgcccagttcccttccagcggcattatgcagccaggagcacattacatgcagcatcaacaagctcaacagatgacaccacaagcccttatggctgcacgctcttctatgctgcagtatgctcaacagccattctcagcgcttcaacaacagcaagccttacacagccagctcggcatgagctctggtggaagcgcaggacttcatatgatgcaaagcgaggctaacactgcaggaggcagtggagctcttggtgctggacgatttcctgattttggcatggatgcctccagtagaggaatcgcaagtgggagcaagcaagatattcggagtgcagggtctagtgaagggcgaggaggaagctctggaggccagggtggtgatggaggtgaaaccctttacttgaaatctgctgatgatgggaactga

SEQ ID NO: 207 - protein, 220 aa, Populus trichocarpa
MQQHLMQMQPMMAAYYPSNVTTDHIQQYLDENKSLILKIVESQNSGKLSECAENQARLQQNLMYLAAIADCQPQPPTMHAQFPSSGIMQPGAHYMQHQQAQQMTPQALMAARSSMLQYAQQPFSALQQQQALHSQLGMSSGGSAGLHMMQSEANTAGGSGALGAGRFPDFGMDASSRGIASGSKQDIRSAGSSEGRGGSSGGQGGDGGETLYLKSADDGN

SEQ ID NO: 208 - DNA, 627 bp, Populus trichocarpa
atgcagcagccaccgcaaatgattcctgtcatttctccatttccaccaacaaacatcaccactgagcagatccaaaagtaccttgacgaaaacaaaaagttgattttggctatattggacaaccaaaaccttggaaaacttgctgaatgtgcccagtatcaagcccagctgcagaagaatttgatgtatttggctgcaattgctgatgcccaaccacaggcaccagcaatgcctccccagatggccccgcatcctgcaatgcaacaaggggcatattacatgcaacatcctcaggcagcagcaatggctcagcagccaggtgttttcccccaaaagatgctattacaattcaatgctggacatcaaatgcaggatcctcagcagttacaccaacaagccatgcaagggcaaataggaattagacctataggggctaacaatggcatgcatcccatgcacgctgagattgctcttggaagcagtggcccttcagcaagtgctggcacaaatgatgtacgtgggggaagcaaacaggatgcctctgaggctggcacaaccggtgctgatggcctagggggctctgctgctgggcataatggtgctgacggttccgaggatgcaaaatga

SEQ ID NO: 209 - protein, 208 aa, Populus trichocarpa
MQQPPQMIPVISPFPPTNITTEQIQKYLDENKKLILAILDNQNLGKLAECAQYQAQLQKNLMYLAAIADAQPQAPAMPPQMAPHPAMQQGAYYMQHPQAAAMAQQPGVFPQKMLLQFNAGHQMQDPQQLHQQAMQGQIGIRPIGANNGMHPMHAEIALGSSGPSASAGTNDVRGGSKQDASEAGTTGADGLGGSAAGHNGADGSEDAK

SEQ ID NO: 210 - DNA, 438 bp, Populus trichocarpa
atgcagcagtcaccgcaacaaatgttgagcatcaccactgagcagattcaaaagtacttagaagagaacaagcagctgattatggctatactggagaatcagaacaagggaaacgtttctgaatgtgcttcgtatcaagcccagttacagcagaacctgatgtacctagcaagaattgctgatgcccaaccacaaggaaccacaatgccttctcagatgccccctcagcagcccgcagtgaagcaagagcagtacatgcagccatctcaagttgctatgactcagcaaccaattttcttcaatcagaagctccctttccaaacgaactttcagcatgagcagcagcaacagctgccaccacacctccaacagcaacacttcacccaaggacagatgagaatgagacccggtgtcactgatcaagattctgatgcctaa

SEQ ID NO: 211 - protein, 145 aa, Populus trichocarpa
MQQSPQQMLSITTEQIQKYLEENKQLIMAILENQNKGNVSECASYQAQLQQNLMYLARIADAQPQGTTMPSQMPPQQPAVKQEQYMQPSQVAMTQQPIFFNQKLPFQTNFQHEQQQQLPPHLQQQHFTQGQMRMRPGVTDQDSDA

SEQ ID NO: 212 - DNA, 645 bp, Prunus persica
atgcagcagccacagcaaatgatccctgtgatgcctacttcatttccacccactaacatcaccaccgagcaaattcagaagtaccttgacgagaacaaaaaattgattctggcaatattggataatcaaaaccttggaaaacttgctgagtgtgcccagtaccaagctcagcttcaaaagaatctgatgtatttagcagctattgctgatgcacaaccacaggcaccaacagtgcctgctcagatggccccacatcctgctatgcaacaagcaggatattacatgcaacatcctcaggcagcagcaatggctcagcaacagggtattttccccccaaagatgccattgcagttcaataacccgcaccaaatgcatgatgcagcacagcagctacaccagcagcaccaacaagccatgcaagggcaaatgggaatgagagctggaggggccaatggcatgccttccatgcatcatactgaagccacacttggtggtggtagtggtggccccacttcaggtggagggggtccaaacgatgggcgtggaggaaagcagcaagactactcagaggctgggacaggtggtgatggccaggggagctcagccggcgggcatggcaatggtgatggagaagatggaaagtga

SEQ ID NO: 213 - protein, 214 aa, Prunus persica
MQQPQQMIPVMPTSFPPTNITTEQIQKYLDENKKLILAILDNQNLGKLAECAQYQAQLQKNLMYLAAIADAQPQAPTVPAQMAPHPAMQQAGYYMQHPQAAAMAQQQGIFPPKMPLQFNNPHQMHDAAQQLHQQHQQAMQGQMGMRAGGANGMPSMHHTEATLGGGSGGPTSGGGGPNDGRGGKQQDYSEAGTGGDGQGSSAGGHGNGDGEDGK

SEQ ID NO: 214 - DNA, 678 bp, Saccharum officinarum
atgcagcagcaacacctgatgcagatgaaccagaacatgattgggggctacacctctcctgccgctgtgacaaccgatctcatccagcagtacctggatgagaacaagcagctgatcctggccatcctcgacaaccagaacaatggcaaggtggaggagtgcgaacggcaccaagctaagctccagcacaacctcatgtacctggccgccatcgccgacagccagccaccacagactgcaccactatcacaatacccgtccaacctgatgatgcagccgggccctcggtacatgccaccgcagtccgggcagatgatgagcccgcagtcgctaatggcggcgcggtcctccatgatgtacgcgcacccgtccatgtcaccactccagcagcagcaggcagcgcacgggcagctgggcatggcttcagggggcggcggtggcacgaccagtgggttcaacatcctccatggcgaggccagtatgggcggtgctggtggcgcttgtgccggcaacaacatgatgaacgccggcatgttctcaggctttggccgcagcggcagtggcgccaaggagggatcgacctcgctgtcggttgacgtccgtggtggcaccagctccggcgcgcaaagcggggacggcgagtacctgaaagcaggcaccgaggaagaaggcagttaa

SEQ ID NO: 215 - protein, 225 aa, Saccharum officinarum
MQQQHLMQMNQNMIGGYTSPAAVTTDLIQQYLDENKQLILAILDNQNNGKVEECERHQAKLQHNLMYLAAIADSQPPQTAPLSQYPSNLMMQPGPRYMPPQSGQMMSPQSLMAARSSMMYAHPSMSPLQQQQAAHGQLGMASGGGGGTTSGFNILHGEASMGGAGGACAGNNMMNAGMFSGFGRSGSGAKEGSTSLSVDVRGGTSSGAQSGDGEYLKAGTEEEGS

SEQ ID NO: 216 - DNA, 561 bp, Saccharum officinarum
atgcagcagccgatgcccatgcagccgcaggcgccggagatgaccccggccgccggaatcaccacggagcagatccaaaagtatctggatgagaataagcagcttattttggctattttggaaaatcagaacctaggaaaattggcagaatgtgctcagtatcaatcacaacttcagaagaacctcttgtatctcgctgcaatcgcagatgcccaaccacagactgctgtaagccgccctcagatggcgccgcctggtgcattgcctggagtagggcagtacatgtcacaggtgcctatgttcccaccgaggacacctctaacaccccagcagatgcaggagcagcaacttcagcagcagcaggctcagctgctaaatttcagtggcctaatggttgctagacctggcatggtcaacggcatgcctcagtccattcaagttcagcaagctcagccaccaccagcagggaacaaacaggatgctggtggggtcgcctcggagccctcgggcattgagaaccacaggagcactggtggtgataatgatggtggaagcgactag

SEQ ID NO: 217 - protein, 186 aa, Saccharum officinarum
MQQPMPMQPQAPEMTPAAGITTEQIQKYLDENKQLILAILENQNLGKLAECAQYQSQLQKNLLYLAAIADAQPQTAVSRPQMAPPGALPGVGQYMSQVPMFPPRTPLTPQQMQEQQLQQQQAQLLNFSGLMVARPGMVNGMPQSIQVQQAQPPPAGNKQDAGGVASEPSGIENHRSTGGDNDGGSD

SEQ ID NO: 218 - DNA, 642 bp, Saccharum officinarum
atgcagcagcagatgcccatgccgccggcgcccgctgcggcggcggcgcccccggcggccggcatcaccaccgagcagatccaaaagtatttggacgaaaataagcaacttattttggccatcctggaaaatcagaacttaggaaagttggctgaatgtgctcagtatcaagctcaacttcaaaagaacctcttgtacctggctgcgattgctgatgcccaaccccagccaccacaaaaccctgcaggtcgccctcagatgatgcaacctggtatagtgccaggtgcggggcattacatgtcacaagtaccaatgttccctccaagaactccattaaccccacagcagatgcaagagcagcagcagcaacagcttcagcagcagcaagcgcaggctcttacattccctggacagatggtcatgagaccagctaccatcaacggcatacagcagcctatgcaagctgaccctgcccgggcagcggagctgcaacaaccaccacctatcccagctgacgggcgagtaagcaagcagcaggacacaacggctggcgtgagctcagagccttctgccaatgagagccacaagaccacaactggagcagatagtgaggcaggtggtgacgtggcggagaaatcctaa

SEQ ID NO: 219 - protein, 213 aa, Saccharum officinarum
MQQQMPMPPAPAAAAAPPAAGITTEQIQKYLDENKQLILAILENQNLGKLAECAQYQAQLQKNLLYLAAIADAQPQPPQNPAGRPQMMQPGIVPGAGHYMSQVPMFPPRTPLTPQQMQEQQQQQLQQQQAQALTFPGQMVMRPATINGIQQPMQADPARAAELQQPPPIPADGRVSKQQDTTAGVSSEPSANESHKTTTGADSEAGGDVAEKS

SEQ ID NO: 220 - DNA, 645 bp, Solanum tuberosum
atgcagcagcacctgatgcagatgcagcccatgatggcagcttactatccaacgaacgtcactactgaccatattcaacagtatttggatgagaacaaatcactcattctgaaaattgttgagagccaaaactcgggaaaactcagtgaatgtgcagagaaccaagctaggcttcagaggaatctgatgtaccttgctgctattgctgattcacaacctcagccttctagcatgcattctcagttctcttctggtgggatgatgcagccagggacacacagttacctgcagcagcagcagcagcaacaacaagcgcaacaaatggcaacacaacaactcatggctgcaagatcctcatcaatgctctatggacaacaacagcagcagcagcagcagtctcagttatcacaatttcaacaaggcttgcatagtagccaacttggcatgagttctggcagtggtggaagcactggacttcatcacatgcttcaaagtgaatcatcacctcatggtggtggtttctctcatgacttcggccgtgcaaataagcaagacattgggagtagtatgtctgctgaagggcgcggcggaagctcaggtggtgatggtggtgagaatctttatctgaaagcttctgaggattga

SEQ ID NO: 221 - protein, 214 aa, Solanum tuberosum
MQQHLMQMQPMMAAYYPTNVTTDHIQQYLDENKSLILKIVESQNSGKLSECAENQARLQRNLMYLAAIADSQPQPSSMHSQFSSGGMMQPGTHSYLQQQQQQQQAQQMATQQLMAARSSSMLYGQQQQQQQQSQLSQFQQGLHSSQLGMSSGSGGSTGLHHMLQSESSPHGGGFSHDFGRANKQDIGSSMSAEGRGGSSGGDGGENLYLKASED

SEQ ID NO: 222 - DNA, 681 bp, Solanum tuberosum
atgcagcagcagcacctgatgcagatgcagcccatgatggcagcctattatcccaacaatgtcactactgatcatattcaacagttcctggatgagaacaaatcacttattctgaagattgttgagagccagaactctgggaaaataagtgaatgtgcagagtcccaagctaaacttcagagaaatcttatgtaccttgcagctattgctgattcacagccccagcctcctagtatgcattcacagttagcttctggtgggatgatgcagggaggggcacattatatgcagcaacaacaagctcaacaactcacaacgcaatcgcttatggctgcagcaagatcctcctcctcaatgctctatggacaacaacaacaacaacaacaacaacaactatcatcattgcaacaacagcaagcagcctttcatagccagcaactcggaatgagcagctctggtggaggaagcagtagtggacttcacatgctacaaagcgaaaacactcatagtgctagcactggtggtggtggtttccctgactttggcagaggattaggcagtggaaacaagcatgaaatgggaagttctatgtctgatcaaggacggggcggaagctcgagtggtcatggtggtgatggaggtgagaatctttacttgaaatcttctgaagatgggaattag

SEQ ID NO: 223 - protein, 226 aa, Solanum tuberosum
MQQQHLMQMQPMMAAYYPNNVTTDHIQQFLDENKSLILKIVESQNSGKISECAESQAKLQRNLMYLAAIADSQPQPPSMHSQLASGGMMQGGAHYMQQQQAQQLTTQSLMAAARSSSSMLYGQQQQQQQQQLSSLQQQQAAFHSQQLGMSSSGGGSSSGLHMLQSENTHSASTGGGGFPDFGRGLGSGNKHEMGSSMSDQGRGGSSSGHGGDGGENLYLKSSEDGN

SEQ ID NO: 224 - DNA, 615 bp, Solanum tuberosum
atgcagcaaccaccacccatgattccaatgatgccctcttttccttctcctaatatcactactgagcagattcaaaagtacctggacgagaacaagacattgattttggccatattggaccatcaaaatcttgggaaactagctgaatgtgcacagtaccaggctaaacttcagaagaacttgatgtacttggccgctattgctgatgctcaaccacaatcaccagctattccaacgcaaatggctcctcatcctgcaatgcaacaaggaggattttacatgcagcaccctcaggctgcagccatgactcaacaacaaggtatgtttacttcaaagatgccactgcagttcaacaacccacagcaactacacgatcagcagcagcttcaacatcaacatcaacatcaacaactacagcgacagcagcaaggtatgcaacttggaggtgccaacagtggaatgcactccactcttggtagtacaagtaatgttagccagcttacaacttcaggtgctggtgatgcacgcggaggaaacaaacaagacaactctgaagcgggtgctgatggtcaggctagctcagtgactgcccaagtctcggaagaacgcaagtga

SEQ ID NO: 225 - protein, 204 aa, Solanum tuberosum
MQQPPPMIPMMPSFPSPNITTEQIQKYLDENKTLILAILDHQNLGKLAECAQYQAKLQKNLMYLAAIADAQPQSPAIPTQMAPHPAMQQGGFYMQHPQAAAMTQQQGMFTSKMPLQFNNPQQLHDQQQLQHQHQHQQLQRQQQGMQLGGANSGMHSTLGSTSNVSQLTTSGAGDARGGNKQDNSEAGADGQASSVTAQVSEERK

SEQ ID NO: 226 - DNA, 678 bp, Sorghum bicolor
atgcagcagcaacacctgatgcagatgaaccagaacatgattgggggctacacctctcctgccgctgtgaccaccgatctcatccagcagtacctggatgagaacaagcagctgatcctggccatcctcgacaaccagaacaatggaaaggtggaggagtgcgaacggcaccaagctaagctccagcacaacctcatgtacctggcggccatcgctgacagccagccaccacagactgcaccactatcacagtacccgtccaacctgatgatgcagccaggccctcggtacatgccaccgcagtccgggcagatgatgagcccgcagtcgctaatggcggcgcggtcctccatgatgtacgcgcacccgtccatgtcgccactccagcagcagcaggcagcgcacggccagctgggcatggcttcagggggcggcggtggcacgaccagtgggttcagcatcctccacggcgaggccagcatgggcggtgctgctggcgcaggcaccggcaacagcatgatgaacgccggcatgttctcaggctttggccgcagcggcagtggcgccaaggagggatcgacctcgctgtctgttgacgtccgtggtggcaccagctccggcgcgcagagcggggacggcgagtacctgaaagcaggcaccgaggaagaaggcagttaa

SEQ ID NO: 227 - protein, 225 aa, Sorghum bicolor
MQQQHLMQMNQNMIGGYTSPAAVTTDLIQQYLDENKQLILAILDNQNNGKVEECERHQAKLQHNLMYLAAIADSQPPQTAPLSQYPSNLMMQPGPRYMPPQSGQMMSPQSLMAARSSMMYAHPSMSPLQQQQAAHGQLGMASGGGGGTTSGFSILHGEASMGGAAGAGTGNSMMNAGMFSGFGRSGSGAKEGSTSLSVDVRGGTSSGAQSGDGEYLKAGTEEEGS

SEQ ID NO: 228 - DNA, 561 bp, Sorghum bicolor
atgcagcagccgatgcccatgcagccgcaggcgccggcgatgaccccggccgccggaatcaccacggagcagatccaaaagtatctggatgagaataagcagcttattttggctattttggaaaatcagaacctaggaaaattggcagaatgtgctcagtatcaatcacaacttcagaagaacctcttgtatctcgctgcaatcgcagatgcccaaccacagactgctgtaagccgccctcagatggcgccgcctggtgcattgcctggagtagggcagtacatgtcacaggtgcctatgttcccaccaaggacacctctcacaccccaacagatgcaggagcagcaacttcagcagcagcaggctcagttgctaaatttcagtggccagatggttgctagacctggcatggtcaacggcatgcctcagtccattcaagctcaacaagctcagccatcaccagcattgaacaaacaggatgctggtggagtcgcctcagagccctcgggcactgagagccacaggagcactggtggtgataatgatggtggaagcgactag

SEQ ID NO: 229 - protein, 186 aa, Sorghum bicolor
MQQPMPMQPQAPAMTPAAGITTEQIQKYLDENKQLILAILENQNLGKLAECAQYQSQLQKNLLYLAAIADAQPQTAVSRPQMAPPGALPGVGQYMSQVPMFPPRTPLTPQQMQEQQLQQQQAQLLNFSGQMVARPGMVNGMPQSIQAQQAQPSPALNKQDAGGVASEPSGTESHRSTGGDNDGGSD

SEQ ID NO: 230 - DNA, 645 bp, Sorghum bicolor
atgcagcagcagatgcccatgccgccggcgcccgctgcggcggcggcgacggcgcccccggcggccggcatcaccaccgagcagatccagaagtatttggacgaaaataagcaacttattttggccatcctagaaaatcagaacttaggaaagttggctgaatgtgctcagtatcaagctcaacttcaaaagaacctcttgtacctggctgcgattgctgatgcccaaccccgaccaccgcaaaaccctgcaggtcgccctcagatgatgcaacctggtatagtgccaggtgcagggcattacatgtcacaagtaccaatgttccctccaagaactccattaaccccacagcaaatgcaagagcagcagcagcaacagcttcagcagcagcaagcgcaggctcttgcattccctgggcagatggtcatgagaccagctaccatcaacggcatgcagcagcctatgcaggctgaccctgcccgggcagcggagctgcaacagccagcatctgtcccagccgacgggcgagtaagcaagcaggacacagcggctggggtgagctcagagccttctgccaatgagagccacaagaccacaaccggagcagatagtgaggcaggtggagacgtggcggagaaatcctaa

SEQ ID NO: 231 - protein, 214 aa, Sorghum bicolor
MQQQMPMPPAPAAAAATAPPAAGITTEQIQKYLDENKQLILAILENQNLGKLAECAQYQAQLQKNLLYLAAIADAQPRPPQNPAGRPQMMQPGIVPGAGHYMSQVPMFPPRTPLTPQQMQEQQQQQLQQQQAQALAFPGQMVMRPATINGMQQPMQADPARAAELQQPASVPADGRVSKQDTAAGVSSEPSANESHKTTTGADSEAGGDVAEKS

SEQ ID NO: 232 - DNA, 552 bp, Taraxacum officinale
atgaagcagccgatgatgcctaatccaatgatgtcttctccgtttcctccttccaacatcaccaccgatcagatccaaaagttcctagacgaaaacaagcaactgatattagcaataatgaacaaccaaaacctaggaaagcttgctgaatgtgcccagtatcaagctctactccaaaagaatttaatgtatctagcagccattgcagatgctcaaccaccaacaccaacaccaacaccaaatatctcatctcagatgggcccggttccacatccagggatgccacaacaaggtggattctacatggggcagcaccctcaagcggcagtaatggcggctcagccaccttctggtttcccacaaccaatgccaggcatgcagtttaataccccacagggtatccaaggtcagatgggcgggaggtccggtgggccaccaaactcagctggcggcgatgtttggagaggaagcatgcaagatggtggtggtggcggtgttgatggtggtaaggatggtcatgctggcggtggtccaccggaggaaggaaagtga

SEQ ID NO: 233 - protein, 183 aa, Taraxacum officinale
MKQPMMPNPMMSSPFPPSNITTDQIQKFLDENKQLILAIMNNQNLGKLAECAQYQALLQKNLMYLAAIADAQPPTPTPTPNISSQMGPVPHPGMPQQGGFYMGQHPQAAVMAAQPPSGFPQPMPGMQFNTPQGIQGQMGGRSGGPPNSAGGDVWRGSMQDGGGGGVDGGKDGHAGGGPPEEGK

SEQ ID NO: 234 - DNA, 636 bp, Taraxacum officinale
atgcagcagcaacagcatcaacaaccaccgcaatcgcaactgcaaccgcccaccttaaactccggtgctccattttctcccaatgcgatcacctccgaccagattcaaaagtgtctggatgacaacgagaacctgattatagcaatattggaaaatcaaaatcttgggaaatttcaggagtgtgctcagtatcaagccattctccaaaagaacttaatgtatttagctgcaattgctgatgctcaaccaccaacacaacaaccatcaactcctcaaatgccaccaaattccattcctcaacaaccaaacaattacatgcaacaacaacaccaaatcaccgcccctcaacaaggcggcataggtggtggtggcggtgttccaaaactaccctttcaacttaatgcccttcgtacacaagatcaacaacaacaattactccaatttcaacaacaacaacaacttcaagcacaaatggggatgagacctagttcccaagatgggatgcttggaatgcatcaagctatgcagtctgcactcgcgggcaatccgggcagtttgatggatggtagagggaacaagcaagatggatcagaggcggcggcttctggtggtggtggtggtaatagagaatcatga

SEQ ID NO: 235 - protein, 211 aa, Taraxacum officinale
MQQQQHQQPPQSQLQPPTLNSGAPFSPNAITSDQIQKCLDDNENLIIAILENQNLGKFQECAQYQAILQKNLMYLAAIADAQPPTQQPSTPQMPPNSIPQQPNNYMQQQHQITAPQQGGIGGGGGVPKLPFQLNALRTQDQQQQLLQFQQQQQLQAQMGMRPSSQDGMLGMHQAMQSALAGNPGSLMDGRGNKQDGSEAAASGGGGGNRES

SEQ ID NO: 236 - DNA, 678 bp, Triticum aestivum
atgcagcagcaacacctgatgcagatgaaccagagcatgatggggggctacgcttcctctaccactgtcaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcaccaggcggccgcgcacggccagctggggatgtcctccggcgcgaccaccgggttcaacctcctccacggcgaggccagcatgggcggcggcggcggcggcgccagtggcaacagcatgatgaacgccggcgtcttctcggactaccgccgcggcggcagcggcgccaaggaggggtcgacctcgctgtcggccgacgctcgcggcgccaactctggcgcgcacagcggcgacggggagtacctcaagggcaccgaggaggaaggaagctag

SEQ ID NO: 237 - protein, 225 aa, Triticum aestivum
MQQQHLMQMNQSMMGGYASSTTVTTDLIQQYLDENKQLILAILDNQNNGKVEECARNQAKLQQNLMYLAAIADSQPPQTASLSQYPSNLMMQSGPRYMQQQSAQMMSPQSLMAARSSMMYAQQAMSPLQQQQQQQHQAAAHGQLGMSSGATTGFNLLHGEASMGGGGGGASGNSMMNAGVFSDYRRGGSGAKEGSTSLSADARGANSGAHSGDGEYLKGTEEEGS

SEQ ID NO: 238 - DNA, 558 bp, Triticum aestivum
atgcagcaagcgatgcccatgccgccggcggcggcggcgccggggatgcctccgtctgctggcctcagcaccgagcagatccaaaagtacctggatgaaaataagcaactaattttggctatcttggaaaatcagaacctgggaaagttggcggaatgtgctcagtatcaagctcagcttcagaagaatcttttgtatttggctgcaatcgctgatactcagccacagaccactgtaagccgtcctcagatggcaccacctagtgcatccccaggggcagggcattacatgtcacaggtgccaatgttccctccgaggacccctctaacgcctcagcagatgcaggagcagcaactacagcagcaacaggctcagatgcttccgtttgctggtcaaatggttgcgagacctggggctgtcaatggcatgcctcaggcccctcaagttgaaccagcctatgcagcaggtggggccagttctgagccttctggcactgagagccacaggagcactggtgccgataatgacggggggagcggctgggctgatcagtcctaa

SEQ ID NO: 239 - protein, 185 aa, Triticum aestivum
MQQAMPMPPAAAAPGMPPSAGLSTEQIQKYLDENKQLILAILENQNLGKLAECAQYQAQLQKNLLYLAAIADTQPQTTVSRPQMAPPSASPGAGHYMSQVPMFPPRTPLTPQQMQEQQLQQQQAQMLPFAGQMVARPGAVNGMPQAPQVEPAYAAGGASSEPSGTESHRSTGADNDGGSGWADQS

SEQ ID NO: 240 - DNA, 603 bp, Triticum aestivum
atgcagcaggcgatgtccttgcccccgggagcggtcggcgcggtgtcctcgccggccggcatcaccaccgagcagatccaaaagtatttggatgaaaataagcaacttattttggccatccttgaaaatcagaacctaggaaagttggctgaatgtgctcagtatcaagctcaactccaaaagaatctcttgtatctagctgctatcgcggatgcccaaccaccacagaaccctacaagtcaccctcagatggtgcagcctggtagtatgcaaggtgcagggcattacatgtcacaagtaccaatgttccctccaagaacgcctttaaccccacagcagatgcaagagcagcagcaccagcagcttcagcagcagcaagcccaggccctttctttccccgcccaggtggtcatgagaccaggcaccgtcaacggcatgcagcagcctatgcaagcagccggcgacctccagccagcagcagcacctggagggagcaagcaggacgccgcagtggctggggccagctcggaaccatctggcaccaagagccacaagaacgcgggagcagaggaggtgggcgctgatgtagcagaacaatcctaa

SEQ ID NO: 241 - protein, 200 aa, Triticum aestivum
MQQAMSLPPGAVGAVSSPAGITTEQIQKYLDENKQLILAILENQNLGKLAECAQYQAQLQKNLLYLAAIADAQPPQNPTSHPQMVQPGSMQGAGHYMSQVPMFPPRTPLTPQQMQEQQHQQLQQQQAQALSFPAQVVMRPGTVNGMQQPMQAAGDLQPAAAPGGSKQDAAVAGASSEPSGTKSHKNAGAEEVGADVAEQS

SEQ ID NO: 242 - DNA, 672 bp, Vitis vinifera
atgcagcagcacctgatgcagatgcagcccatgatggcagcctattaccccagcaacgtcaccactgatcacattcagcagtatcttgatgaaaacaagtcattgattctgaagattgttgagagccagaattcaggaaaattgactgaatgtgcagagaaccaggcaagactacagagaaacctcatgtacctggctgcaattgctgattctcaaccccaaccacccaccatgcatgctcagttccctcctagtggcattgttcagccaggagctcactacatgcaacaccaacaagctcaacaaatgacaccacagtcgctcctggctgcacgctcctccatgctgtacacccaacaaccattttcggccctgcaacaacaacaagccatccatagccagcttggcatgggctctggtggaagtgcaggacttcacatgctgcaaagcgaggggagtaatccaggaggcaatggaacactggggactggtgggtttcctgatttcagccgtggaacttctggagaaggcctgcaggctgcaggcaggggaatggctggtgggagcaagcaagatatgggaaatgcagaagggcgaggagggaactcaggaggtcagggtggggatggaggtgagactctttacttgaaagctgctgaagatgggaattga

SEQ ID NO: 243 - protein, 223 aa, Vitis vinifera
MQQHLMQMQPMMAAYYPSNVTTDHIQQYLDENKSLILKIVESQNSGKLTECAENQARLQRNLMYLAAIADSQPQPPTMHAQFPPSGIVQPGAHYMQHQQAQQMTPQSLLAARSSMLYTQQPFSALQQQQAIHSQLGMGSGGSAGLHMLQSEGSNPGGNGTLGTGGFPDFSRGTSGEGLQAAGRGMAGGSKQDMGNAEGRGGNSGGQGGDGGETLYLKAAEDGN

SEQ ID NO: 244 - DNA, 675 bp, Vitis vinifera
atgcagcaacaccttatgcagatgcagcctatgatggcaggaagccataacctcagcagcatcactactgatcacatccaacagtacctagatgagaacaagtctttgattttgaaaattcttgagagccaaaattcagggaaactcagtgaatgtgcggagaaccaagcaagacttcagcgaaaccttatgtaccttgctgcaattgctgattgccaaccacaaccaccatccctgcaggctcagttttcccccaatatggtcatgcaaccaggagtcaactacatgcagcaccaacaatcccaacagatgatgccacagtcactaatggcagcccgagcacccatgttgtatgctcagcagcatccatatttggcattgcagcaacaacaagctctacaaagccagcttggcatgagctccactggaatgggtggaatccacatgctacaaagtgaacctaatgttggagggaatgggactggagccttttccgatcttggtcgcagcatgactggggagggcttgtcggctgtgagcaggggactgggtagtgcaagcaagcaagatgtggggagtgtaggctctgcggaaggtcgacgtggctacttgggagggcaaggtgcagataaaggagaaactctttactttaaaagtgctgaagaaaaggactga

SEQ ID NO: 245 - protein, 224 aa, Vitis vinifera
MQQHLMQMQPMMAGSHNLSSITTDHIQQYLDENKSLILKILESQNSGKLSECAENQARLQRNLMYLAAIADCQPQPPSLQAQFSPNMVMQPGVNYMQHQQSQQMMPQSLMAARAPMLYAQQHPYLALQQQQALQSQLGMSSTGMGGIHMLQSEPNVGGNGTGAFSDLGRSMTGEGLSAVSRGLGSASKQDVGSVGSAEGRRGYLGGQGADKGETLYFKSAEEKD

SEQ ID NO: 246 - DNA, 669 bp, Vitis vinifera
atgcagcagaacccccagatgatacctgttatgccttcttttccacccaacaacatcactaccgagcagattcagaagtatctcgatgagaataaaaaattgattctggcaatattggacaatcaaaaccttggaaagcttgctgagtgtgcacagtaccaagctcagcttcaaaagaatttgatgtatctagctgcaattgctgatgctcagccacaggcaccaccgacaatgcctccccagatggccccacaccctgcaatgcagcagggagggtactacatgcagcatccccaggcggcagcaatggctcagcaacctggtcttttccctcccaagatgcccttacaatttggtaacccacatcaacttcaggagcaagcacagcagctgcagcagctacagcaacaagccatgcaagggcagatgggcatgagacctggaggggccaacaacggcatgcatcccatgcatcctgaggccactcttggtggtggcagcagtggtggccctccaccatctgccggcctcagtgatgcacgcggaggtggcaagcaagacacttccgaagcaggggcttctggtggtgatggtcaggggagctcagctgctgggcatggcggcgatggcgaatcaccctacttgaaggggtcagaggatggaaagtga

SEQ ID NO: 247 - protein, 222 aa, Vitis vinifera
MQQNPQMIPVMPSFPPNNITTEQIQKYLDENKKLILAILDNQNLGKLAECAQYQAQLQKNLMYLAAIADAQPQAPPTMPPQMAPHPAMQQGGYYMQHPQAAAMAQQPGLFPPKMPLQFGNPHQLQEQAQQLQQLQQQAMQGQMGMRPGGANNGMHPMHPEATLGGGSSGGPPPSAGLSDARGGGKQDTSEAGASGGDGQGSSAAGHGGDGESPYLKGSEDGK

SEQ ID NO: 248 - DNA, 597 bp, Vitis vinifera
atgcagcagcaaccaccacagatgatgaacattgcgccttcatttcctcccaccgccatcaccactgagcagattcagaagtatctggatgagaacaaacaattgattctggcaattctggaaaaccagacccttggaaaactcgccgagtgtgcccaatatcaagcccagcttcagaagaacttgatatatctagctgcaattgctgatgcccaaccaccagcaccaacagtgcctcctcagatgcctatacatcatgccatgcaacaagggcattacatgcaacaccctcaggctgctgcagctcagcaacaaccaggcatgtttggtgcaaagttgcctttccagcttagcgatcagcaacagcagcagcagcatcattttttacacctccaacaacagcaacccatccaagggctcatgggcatgaggcctatcatcaacaatggcatgcatcaggccatgcaaactgggcttggtgctttgagcggtttcatggatgtacgtggaagcaagccagatggctcagaggttggttttggtgatggccaagggaagtttgcttctggacatggcagtggaaatagagattcctaa

SEQ ID NO: 249 - protein, 198 aa, Vitis vinifera
MQQQPPQMMNIAPSFPPTAITTEQIQKYLDENKQLILAILENQTLGKLAECAQYQAQLQKNLIYLAAIADAQPPAPTVPPQMPIHHAMQQGHYMQHPQAAAAQQQPGMFGAKLPFQLSDQQQQQQHHFLHLQQQQPIQGLMGMRPIINNGMHQAMQTGLGALSGFMDVRGSKPDGSEVGFGDGQGKFASGHGSGNRDS

SEQ ID NO: 250 - DNA, 735 bp, Volvox carteri
atggcggcagtgtcaggtcaggccaagccagccccaatgacaacggagcgaatccaagaaatgcttgaagaaaacttcaagtttatcaaggcactcgccgaacagcaaaaccttggtcgaatgcaggacgtcatccagttccagcagaagcttcaagaaaaccttatgctactagcagctgtggcggacacctactcatcagcgtctgcaacgggagctgctgcccaggcgactgcagctcccggcatggcggcacaggcggcccggccgcccgctgctgctctccctggaacagctgttgctacggcggcgctcccgctccctggcttgccgcaacaacagcaaccgcagcagcagcagcagccgcagcagcagccagggcagcccagcccgctaatgatggtcatgccgggctcttccgggacgcaggcgcctacgcaactgggggccatgcaactcacgcagcagcagatacaagctgcggtacagcaagcgctagtccgacagcagcagcagcaacaacaacagcagcagcaaaatccaaatccgtttcaggggcagcaattccagctgccacagtcacttcagcagccgcagtccctgggacagcagccagttttaagtgcgatgggggccctgggagggcccttggctggtcaggggacggcggcagggcaggcgggagcaggtggtttccagctgcccgcggcgcctcctttgaaccttgggctttga

SEQ ID NO: 251 - protein, 244 aa, Volvox carteri
MAAVSGQAKPAPMTTERIQEMLEENFKFIKALAEQQNLGRMQDVIQFQQKLQENLMLLAAVADTYSSASATGAAAQATAAPGMAAQAARPPAAALPGTAVATAALPLPGLPQQQQPQQQQQPQQQPGQPSPLMMVMPGSSGTQAPTQLGAMQLTQQQIQAAVQQALVRQQQQQQQQQQQNPNPFQGQQFQLPQSLQQPQSLGQQPVLSAMGALGGPLAGQGTAAGQAGAGGFQLPAAPPLNLGL

SEQ ID NO: 252 - DNA, 699 bp, Welwitschia mirabilis
atgcaaatgcagcctatgataggggcaggatactcctccaatagcatcaccactgatcacattcagaagtatttggatgagaataggcaattgattctggcaattcttgacaaccaaaatcttggaaagttgaatgaatgtgcacaataccaagccaggcttcaacaaaatttaatgtatttggctgctattgctgattcccaaccgcaaacacctgctgcacatgcccagatcgcatccaatgcaatgcttcaagcaggtggtcattatatgcagcaccagcagacagtaactccccagtcacttcttgctgcaaggtcatctatgctttacagtcagcaacctatgactgcattccatcaagcacagcagcagcagcaacaacaatctctccatggccagttggggataaattcaggaggaaacaatggattacatattcttcatggtgaaacaagcatgggtagtaatggacctctcacgacaggaagctttcctgattttgggcgaggttcagtaaattgtgatctattgcagggcaataggagcttgactattgaccgtggctctagcaagattgatgggcttggaacagagaatacgcaccctgtagaagggcgaggaggagcaagtgtgggccaaaacacagaagaaggggcaccatcttatttgaaagcttctgaggatgagggaagctag

SEQ ID NO: 253 - protein, 232 aa, Welwitschia mirabilis
MQMQPMIGAGYSSNSITTDHIQKYLDENRQLILAILDNQNLGKLNECAQYQARLQQNLMYLAAIADSQPQTPAAHAQIASNAMLQAGGHYMQHQQTVTPQSLLAARSSMLYSQQPMTAFHQAQQQQQQQSLHGQLGINSGGNNGLHILHGETSMGSNGPLTTGSFPDFGRGSVNCDLLQGNRSLTIDRGSSKIDGLGTENTHPVEGRGGASVGQNTEEGAPSYLKASEDEGS

SEQ ID NO: 254 - DNA, 684 bp, Zea mays
atgcagcagcaacacctgatgcagatgaaccagaacatgatggggggctacacctctcctgccgccgtgaccaccgatctcatccagcagcacctggacgagaacaagcagctgatcctggccatcctcgacaaccagaacaatggcaaggcggaggagtgcgaacggcaccaagctaagctccagcacaacctcatgtacctggccgccatcgctgacagccagccgccacagaccgcgccactatcacagtacccgtccaacctgatgatgcagccgggccctcggtacatgccaccgcagtccgggcagatgatgaacccgcagtcgctgatggcggcgcggtcctccatgatgtacgcgcacccgtccctgtcgccactccagcagcagcaggcggcgcacggacagctgggtatggctccagggggcggcggtggcggcacgaccagcgggttcagcatcctccacggcgaggccagcatgggcggtggtggtgctggcgcaggcgccggcaacaacatgatgaacgccggcatgttctcgggctttggccgcagcggcagtggcgccaaggaagggtcgacctctctgtcggttgacgtccggggtggaaccagctccggcgcgcagagcggggacggcgagtacctcaaagtcggcaccgaggaagaaggcagttag

SEQ ID NO: 255 - protein, 227 aa, Zea mays
MQQQHLMQMNQNMMGGYTSPAAVTTDLIQQHLDENKQLILAILDNQNNGKAEECERHQAKLQHNLMYLAAIADSQPPQTAPLSQYPSNLMMQPGPRYMPPQSGQMMNPQSLMAARSSMMYAHPSLSPLQQQQAAHGQLGMAPGGGGGGTTSGFSILHGEASMGGGGAGAGAGNNMMNAGMFSGFGRSGSGAKEGSTSLSVDVRGGTSSGAQSGDGEYLKVGTEEEGS

SEQ ID NO: 256 - DNA, 549 bp, Zea mays
atgcagcagccgatgcacatgcagccacaggcgccggcgataaccccagctgccggaatcagcacggagcagatccaaaagtatctggatgagaataagcagcttattttggctattttggaaaatcagaacctaggaaaattggcagaatgtgctcagtatcaatcacaacttcagaagaacctcttgtatctcgctgcaatcgcagatgctcaaccgcagactgctgtaagccgccctcagatggcgccgcctggtggatcgcctggagtagggcagtacatgtcacaggtgcctatgttcccaccgaggacacctcttacaccccagcagatgcaggagcagcagcttcagcagcagcaggctcagttgctaaacttcagtggccaaatggttgctagaccaggcatggtcaacggcatggctcagtccatgcaagctcagctaccaccgggtgtgaacaagcaggatgctggtggggtcgcctctgagccctcgggcaccgagagccacaggagcactggtggtgacgatggtggaagcgactag

SEQ ID NO: 257 - protein, 182 aa, Zea mays
MQQPMHMQPQAPAITPAAGISTEQIQKYLDENKQLILAILENQNLGKLAECAQYQSQLQKNLLYLAAIADAQPQTAVSRPQMAPPGGSPGVGQYMSQVPMFPPRTPLTPQQMQEQQLQQQQAQLLNFSGQMVARPGMVNGMAQSMQAQLPPGVNKQDAGGVASEPSGTESHRSTGGDDGGSD

SEQ ID NO: 258 - DNA, 663 bp, Zea mays
atgcagcagcagatgcccatgccgccggcgcccgctgccgccgcggcggcggcgcccccggcggcaggcatcactaccgagcagatccagaagtatttggacgaaaataagcaacttattttggccatcctggaaaatcagaacttagggaagttggctgaatgtgctcagtatcaagctcaacttcaaaagaacctcttgtacctggctgcgattgctgatgcccaaccccagcctccgcaaaaccctgcaggtcgccctcagatgatgcagcctggtatagtgccaggtgcggggcattacatgtcacaagtaccaatgttccctccaagaaccccattaaccccacagcagatgcaggagcagcagcaacaacaacagtttcagcagcagcagcagcaagtgcaggctcttacatttcctggacagatggtcatgagaccaggcaccatcaacggcatgcagcagcagcagcctatgcaggctgaccctgcccgggcagcagcggagctgcagcaggcagcacctatcccagctgacgggcgaggaagcaagcaggacaccgcgggtggggcgagctcagagccttctgccaatgagagccacaagagcgccaccggagcagataccgaggcaggtggcgacgtggccgagaaatcctaa

SEQ ID NO: 259 - protein, 220 aa, Zea mays
MQQQMPMPPAPAAAAAAAPPAAGITTEQIQKYLDENKQLILAILENQNLGKLAECAQYQAQLQKNLLYLAAIADAQPQPPQNPAGRPQMMQPGIVPGAGHYMSQVPMFPPRTPLTPQQMQEQQQQQQFQQQQQQVQALTFPGQMVMRPGTINGMQQQQPMQADPARAAAELQQAAPIPADGRGSKQDTAGGASSEPSANESHKSATGADTEAGGDVAEKS

SEQ ID NO: 260 - DNA, 1173 bp, Homo sapiens
atgggcggcaacatgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcag

SEQ ID NO: 261 - protein, 391 aa, Homo sapiens
MGGNMSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 262 - protein, 54 aa, Artificial sequence
  SNH domain from Arath_SYT1 (comprised in SEQ ID NO 121)
VTSDHIQQYLDENKSLILKIVESQNSGKLSECAENQARLQRNLMYLAAIADAQT

SEQ ID NO: 263 - protein, 45 aa, Artificial sequence
  most conserved residues comprised in the SNH domain
QXXLXXNXXXIXXXXXXXXXGXXXXXXXXQXXLXXNLXXLAXXAD

SEQ ID NO: 264 - protein, 74 aa, Artificial sequence
  SSXT domain InterPro007726 (PFam05030) comprised in SEQ ID NO 121
QMQPMMAGYYPSNVTSDHIQQYLDENKSLILKIVESQNSGKLSECAENQARLQRNLMYLAAIADSQPQPPSVHS

SEQ ID NO: 265 - DNA, 53 bp, Artificial sequence
  primer prm06681
ggggacaagtttgtacaaaaaagcaggcttaaacaatgcaacagcacctgatg

SEQ ID NO: 266 - DNA, 50 bp, Artificial sequence
  primer prm06682
ggggaccactttgtacaagaaagctgggtcatcattaagattccttgtgc

SEQ ID NO: 267 - DNA, 1828 bp, Artificial sequence
  SEQ ID NO 1 + SEQ ID NO 120
atgatgagtctaagtggaagtagcgggagaacaataggaaggcctccatttacaccaacacaatgggaagaactggaacatcaagccctaatctacaagtacatggtctctggtgttcctgtcccacctgagctcatcttctccattagaagaagcttggacacttccttggtctctagactccttcctcaccaatcccttggatgggggtgttaccagatgggatttgggagaaaaccagatccagagccaggaagatgcagaagaacagatggtaagaaatggagatgctcaagagaagcttacccagattcgaagtactgtgaaaaacacatgcacagaggaagaaaccgtgccagaaaatctcttgatcagaatcagacaacaacaactcctttaacatcaccatctctctcattcaccaacaacaacaacccaagtcccaccttgtcttcttcttcttcctctaattcctcttctactacttattctgcttcttcttcttcaatggatgcctacagtaacagtaataggtttgggcttggtggaagtagtagtaacactagaggttatttcaacagccattctcttgattatccttatccttctacttcacccaaacaacaacaacaaactcttcatcatgcttccgctttgtcacttcatcaaaatactaattctacttctcagttcaatgtcttagcctctgctactgaccacaaagacttcaggtactttcaagggattggggagagagttggaggagttggggagagaacgttctttccagaagcatctagaagctttcaagattctccataccatcatcaccaacaaccgttagcaacagtgatgaatgatccgtaccaccactgtagtactgatcataataagattgatcatcatcacacatactcatcctcatcatcatctcaacatcttcatcatgatcatgatcatagacagcaacagtgttttgttttgggcgccgacatgttcaacaaacctacaagaagtgtccttgcaaactcatcaagacaagatcaaaatcaagaagaagatgagaaagattcatcagagtcgtccaagaagtctctacatcacttctttggtgaggactgggcacagaacaagaacagttcagattcttggcttgacctttcttcccactcaagactcgacactggtagctaanatgcaacagcacctgatgcagatgcagcccatgatggctggttactaccccagcaatgttacctctgatcatatccaacagtacttggacgaaaacaaatcgttgattctgaagattgttgagtctcaaaactctggaaagcttagcgaatgcgccgagaatcaagcaaggcttcaacgcaacctaatgtacctagctgcaatagcagattctcagcctcagccaccaagtgtgcatagccagtatggatctgctggtggtgggatgattcagggagaaggagggtcacactatttgcagcagcaacaagcgactcaacagcaacagatgactcagcagtctctaatggcggctcgatcttcaatgttgtatgctcagcaacagcggcagcagcagccttacgcgacgcttcagcatcagcaatcgcaccatagccagcttggaatgagctcgagcagcggaggaggaggaagcagtggtctccatatccttcagggagaggctggtgggtttcatgattttggccgtgggaagccggaaatgggaagtggtggtggcggtgaaggcagaggaggaagttcaggggatggtggagaaaccctttacttgaaatcatcagatgatgggaattga

SEQ ID NO: 268 - DNA, 1828 bp, Artificial sequence
  SEQ ID NO 120 + SEQ ID NO 1
atgcaacagcacctgatgcagatgcagcccatgatggctggttactaccccagcaatgttacctctgatcatatccaacagtacttggacgaaaacaaatcgttgattctgaagattgttgagtctcaaaactctggaaagcttagcgaatgcgccgagaatcaagcaaggcttcaacgcaacctaatgtacctagctgcaatagcagattctcagcctcagccaccaagtgtgcatagccagtatggatctgctggtggtgggatgattcagggagaaggagggtcacactatttgcagcagcaacaagcgactcaacagcaacagatgactcagcagtctctaatggcggctcgatcttcaatgttgtatgctcagcaacagcggcagcagcagccttacgcgacgcttcagcatcagcaatcgcaccatagccagcttggaatgagctcgagcagcggaggaggaggaagcagtggtctccatatccttcagggagaggctggtgggtttcatgattttggccgtgggaagccggaaatgggaagtggtggtggcggtgaaggcagaggaggaagttcaggggatggtggagaaaccctttacttgaaatcatcagatgatgggaattganatgatgagtctaagtggaagtagcgggagaacaataggaaggcctccatttacaccaacacaatgggaagaactggaacatcaagccctaatctacaagtacatggtctctggtgttcctgtcccacctgagctcatcttctccattagaagaagcttggacacttccttggtctctagactccttcctcaccaatcccttggatgggggtgttaccagatgggatttgggagaaaaccagatccagagccaggaagatgcagaagaacagatggtaagaaatggagatgctcaagagaagcttacccagattcgaagtactgtgaaaaacacatgcacagaggaagaaaccgtgccagaaaatctcttgatcagaatcagacaacaacaactcctttaacatcaccatctctctcattcaccaacaacaacaacccaagtcccaccttgtcttcttcttcttcctctaattcctcttctactacttattctgcttcttcttcttcaatggatgcctacagtaacagtaataggtttgggcttggtggaagtagtagtaacactagaggttatttcaacagccattctcttgattatccttatccttctacttcacccaaacaacaacaacaaactcttcatcatgcttccgctttgtcacttcatcaaaatactaattctacttctcagttcaatgtcttagcctctgctactgaccacaaagacttcaggtactttcaagggattggggagagagttggaggagttggggagagaacgttctttccagaagcatctagaagctttcaagattctccataccatcatcaccaacaaccgttagcaacagtgatgaatgatccgtaccaccactgtagtactgatcataataagattgatcatcatcacacatactcatcctcatcatcatctcaacatcttcatcatgatcatgatcatagacagcaacagtgttttgttttgggcgccgacatgttcaacaaacctacaagaagtgtccttgcaaactcatcaagacaagatcaaaatcaagaagaagatgagaaagattcatcagagtcgtccaagaagtctctacatcacttctttggtgaggactgggcacagaacaagaacagttcagattcttggcttgacctttcttcccactcaagactcgacactggtagctaa

SEQ ID NO: 269 - protein, 608 aa, Artificial sequence
  SEQ ID NO 2 + SEQ ID NO 121
MMSLSGSSGRTIGRPPFTPTQWEELEHQALIYKYMVSGVPVPPELIFSIRRSLDTSLVSRLLPHQSLGWGCYQMGFGRKPDPEPGRCRRTDGKKWRCSREAYPDSKYCEKHMHRGRNRARKSLDQNQTTTTPLTSPSLSFTNNNNPSPTLSSSSSSNSSSTTYSASSSSMDAYSNSNRFGLGGSSSNTRGYFNSHSLDYPYPSTSPKQQQQTLHHASALSLHQNTNSTSQFNVLASATDHKDFRYFQGIGERVGGVGERTFFPEASRSFQDSPYHHHQQPLATVMNDPYHHCSTDHNKIDHHHTYSSSSSSQHLHHDHDHRQQQCFVLGADMFNKPTRSVLANSSRQDQNQEEDEKDSSESSKKSLHHFFGEDWAQNKNSSDSWLDLSSHSRLDTGSXMQQHLMQMQPMMAGYYPSNVTSDHIQQYLDENKSLILKIVESQNSGKLSECAENQARLQRNLMYLAAIADSQPQPPSVHSQYGSAGGGMIQGEGGSHYLQQQQATQQQQMTQQSLMAARSSMLYAQQQRQQQPYATLQHQQSHHSQLGMSSSSGGGGSSGLHILQGEAGGFHDFGRGKPEMGSGGGGEGRGGSSGDGGETLYLKSSDDGN

SEQ ID NO: 270 - protein, 608 aa, Artificial sequence
  SEQ ID NO 121 + SEQ ID NO 2
MQQHLMQMQPMMAGYYPSNVTSDHIQQYLDENKSLILKIVESQNSGKLSECAENQARLQRNLMYLAAIADSQPQPPSVHSQYGSAGGGMIQGEGGSHYLQQQQATQQQQMTQQSLMAARSSMLYAQQQRQQQPYATLQHQQSHHSQLGMSSSSGGGGSSGLHILQGEAGGFHDFGRGKPEMGSGGGGEGRGGSSGDGGETLYLKSSDDGNXMMSLSGSSGRTIGRPPFTPTQWEELEHQALIYKYMVSGVPVPPELIFSIRRSLDTSLVSRLLPHQSLGWGCYQMGFGRKPDPEPGRCRRTDGKKWRCSREAYPDSKYCEKHMHRGRNRARKSLDQNQTTTTPLTSPSLSFTNNNNPSPTLSSSSSSNSSSTTYSASSSSMDAYSNSNRFGLGGSSSNTRGYFNSHSLDYPYPSTSPKQQQQTLHHASALSLHQNTNSTSQFNVLASATDHKDFRYFQGIGERVGGVGERTFFPEASRSFQDSPYHHHQQPLATVMNDPYHHCSTDHNKIDHHHTYSSSSSSQHLHHDHDHRQQQCFVLGADMFNKPTRSVLANSSRQDQNQEEDEKDSSESSKKSLHHFFGEDWAQNKNSSDSWLDLSSHSRLDTGS